SEQUENCE LISTING (29 sequences)

SEQ ID NO: 1 - protein, 9 aa, Unknown
  laminin peptide 11
  MOD_RES (9)..(9): AMIDATION
CDPGYIGSR

SEQ ID NO: 2 - protein, 12 aa, Unknown
  cage interior surface peptide, specific for L10 phases of CoPt
KTHEIHSPLLHK

SEQ ID NO: 3 - protein, 12 aa, Unknown
  cage interior surface peptide, specific for FePt
HNKHLPSTQPLA

SEQ ID NO: 4 - protein, 4 aa, Unknown
  cathepsin K cleavage site
  MOD_RES (1)..(1): May be benzyloxycarbonylated
  MOD_RES (4)..(4): May have an attached aminomethylcoumarin moiety
AGPR

SEQ ID NO: 5 - protein, 9 aa, Unknown
  RGD-4C peptide which binds selectively to integrins
CDCRGDCFC

SEQ ID NO: 6 - protein, 10 aa, Unknown
  laminin peptide 11
CDPGYIGSRC

SEQ ID NO: 7 - protein, 11 aa, Unknown
  peptide targeting cell surface nucleolin
KDEPQRRSARL

SEQ ID NO: 8 - protein, 9 aa, Unknown
  peptide targeting cell surface nucleolin
KPKKAPAKK

SEQ ID NO: 9 - protein, 9 aa, Unknown
  peptide targeting cell surface nucleolin
CGNKRTRGC

SEQ ID NO: 10 - protein, 9 aa, Unknown
  peptide targeting unknown target
CGNKRTRGC

SEQ ID NO: 11 - protein, 7 aa, Unknown
  peptide targetting aminopeptidase N
CNGRCVS

SEQ ID NO: 12 - protein, 6 aa, Unknown
  peptide targetting aminopeptidase N
GCAGRC

SEQ ID NO: 13 - protein, 7 aa, Simian virus 40
PKKKRKV

SEQ ID NO: 14 - protein, 6 aa, Homo sapiens
ARRRRP

SEQ ID NO: 15 - protein, 10 aa, Homo sapiens
EEVQRKRQKL

SEQ ID NO: 16 - protein, 9 aa, Homo sapiens
EEKRKRTYE

SEQ ID NO: 17 - protein, 20 aa, Xenopus sp.
AVKRPAATKKAGQAKKKKLD

SEQ ID NO: 18 - protein, 13 aa, Unknown
  modified RGD-4C peptide
SGGCDCRGDCFCG

SEQ ID NO: 19 - DNA, 47 bp, Artificial Sequence
  primer encoding modified RGD-4C
gatctggaggatgcgactgccgcggagactgcttctgcggataagga

SEQ ID NO: 20 - DNA, 42 bp, Artificial Sequence
  RGD-4C mutagenesis primer
gaagaagatatacatatgacgtccgcgtccacctcgcaggtg

SEQ ID NO: 21 - DNA, 42 bp, Artificial Sequence
  RGD-4C mutagenesis primer
cacctgcgaggtggacgcggacgtcatatgtatatctccttc

SEQ ID NO: 22 - DNA, 39 bp, Artificial Sequence
  primer for plasmid AatII site insertion
gcgactgccgcggagactgcttctgcggaggcggaacgt

SEQ ID NO: 23 - DNA, 39 bp, Artificial Sequence
  primer for plasmid AatII site insertion
tccgcctccgcagaagcagtctccgcggcagtcgcacgt

SEQ ID NO: 24 - protein, 5 aa, Unknown
  original HFn sequence
MTTAS

SEQ ID NO: 25 - protein, 18 aa, Unknown
  mutated HFn sequence
MTCDCRGDCFCGGGTSAS

SEQ ID NO: 26 - DNA, 85 bp, Artificial Sequence
  mutagenesis primer used for Calmodulin insertion into CCMV capsid protein
cgaggaattcatgtctacagacaaaagatggtgatggatggttagaattcgaagagggtgggggcgaagagaacgaggagaacac

SEQ ID NO: 27 - protein, 15 aa, Unknown
  Calmodulin partial sequence
DKDGDGWLEFEEGGG

SEQ ID NO: 28 - protein, 20 aa, Cowpea chlorotic mottle virus
MSTVGTGELTEAQEEAAAEE

SEQ ID NO: 29 - protein, 20 aa, Artificial Sequence
  CCMV capsid-Calmodulin fusion protein partial sequence
MSTDKDGDGWLEFEEGGGEE

(19) United States
(12) Patent Application Publication
(10) Pub. No.: US 2007/0258889 A1
(43) Pub. Date: Nov. 8, 2007

(54) NOVEL NANOPARTICLES AND USE THEREOF

(75) Inventors: Trevor Douglas, Bozeman, MT (US); Peter Suci, Bozeman, MT (US); Mark J. Young, Bozeman, MT (US)

Correspondence Address: DORSEY & WHITNEY LLP, 555 CALIFORNIA STREET, SUITE 1000, SUITE 1000, SAN FRANCISCO, CA, 94104, US

(73) Assignee: Montana State University, Bozman, MT (US)

(21) Appl. No.: 11/558,393

(22) Filed: Nov. 9, 2006

Related U.S. Application Data
(60) Provisional application No. 60/736,041, filed on Nov. 9, 2005.
(60) Provisional application No. 60/831,109, filed on Jul. 14, 2006.

(51) Int. Cl. A61K 9/14; A61K 49/14; A61K 51/08

(52) U.S. Cl. 424/1.37; 424/499; 424/9.34; 977/773

(57) ABSTRACT

The present invention is directed to novel compositions and methods utilizing delivery agents or nanoparticles that include protein cages with modified and/or unmodified subunits and various agents, such as therapeutic and/or imaging agents located on the interior and/or exterior surface of the protein cages.

### CROSS-REFERENCE TO RELATED APPLICATIONS

This application claims the benefit under 35 U.S.C. § 119(e) of U.S. Ser. No. 60/736,041 filed Nov. 9, 2005 and U.S. Ser. No. 60/831,109 filed Jul. 14, 2006, each of which is hereby incorporated by reference in its entirety.

### FIELD OF THE INVENTION

The present invention is directed to novel compositions and methods utilizing nanoparticles comprising protein cages having various features including externally and/or internally located targeting moieties, disassembly mechanisms, therapeutic agents, medical imaging agents, and combinations thereof.

### BACKGROUND OF THE INVENTION

There is considerable interest in the controlled formation of size constrained and nanophase inorganic and organic materials for a variety of technological applications such as magnetism, semiconductors, ceramics, as well as medical therapeutics and diagnostics. However, conventional solution methods often produce materials having a range of particle sizes. Since the properties of nanophase materials are intimately related to their dimensions, this implies a heterogeneity of physical properties; this heterogenity limits their usefulness. Alternative syntheses using a biomimetic approach (Mann, S. ed.; Biomimetic Materials Chemistry (VCH Publishers: New York, 1996)) have utilized organized molecular assemblies for materials synthesis, such as micelles, microemulsions, surfactant vesicles, Langmuir monolayers (and multilayers) and the protein cage of the iron storage protein ferritin. All these have proven to be versatile reaction environments and a wide range of inorganic materials have been synthesized using these systems (Douglas, T. et al. Molecular Biology and Biotechnology, R. A. Meyers, Ed.; (VCH publishers: New York, 1995) p. 466-469). However, there are severe limitations to these systems. Micelles for example are dynamic structures with fluctuations in size, whereas vesicles often have limited stability with regard to aggregation and hydrolysis. A major limitation to the existing synthetic methods, utilizing this biomimetic approach, has been the inability to vary particle size over a wide range while maintaining a narrow particle size distribution.

Ferritin is a protein involved in the regulation of iron in biological systems. In nature, ferritin consists of a protein shell, having 24 structurally equivalent protein subunits surrounding a near spherical core of hydrous ferric oxide (“ferrihydrite”). The core is disclosed as being any organic or inorganic material with the exception of ferrihydrite. Once a core has formed in the process of these patents, the protein coat can be removed and the freed core particles isolated. The process is disclosed as providing for particles approximately 5 to 8 nanometers in diameter. However, this system is size constrained, such that homogeneous particles of smaller or larger sizes are not possible.

In a recent development, U.S. Pat. Nos. 5,304,382, 5,358,722, and 5,491,219, disclose the use of apoferritin devoid of ferrihydride as another solution to the problem of producing small particles. These ferritin analogs consist of an apoferritin shell and an inorganic core, and are thought to be useful in the production of ultrafine particles for high performance ceramics, drug delivery, and other uses.

A general review of systems employing the apoferritin/core nanoscale particle production system is provided by Douglas, “Biomimetic Synthesis of Nanoscale Particles in Organized Protein Cages,” Biomimetic Materials Chemistry, S. Mann (ed.) VCH Publishers, New York (1996). In addition, different types of protein cage-core composites have been described, including an composite containing apoferritin and iron oxide cores that include magnetite and/or maghemite (Douglas et al., American Chemical Society, ACS Symposium Series: Hybrid Organic-inorganic Composites, J. Mark, C. Y-C Lee, P. A. Bianconi (eds.) (1995); Meldrum et al., Science 257: 522-523 (1992)); ferritin shells containing iron sulfide cores (Douglas et al., Science 269: 54-57 (1995)); magnetoferritin which includes an apoferritin shell and an iron oxide core (Bulte et al., JMRI, May/June 1994, pp. 497-505; Bulte et al., Acad. Radiol., 2: 871-878 (1995); Bulte et al., Investigative Radiobiology 20 (Supplement 2): S214-S216 (1994)); apoferritin shells containing manganese oxide cores (Meldrum et al., J. Inorg. Biochem., 58: 59-68 (1995))

The protein ferritin has provided a remarkably robust alternative for inorganic material synthesis (Douglas, T. et al., Biomimetic Approaches in Materials Science, S. Mann, Ed.; (VCH Publishers: New York, 1996) p. 91-115) Ferritins play a central role in the sequestration and storage of iron in biological systems. There is a high degree of structural conservation among ferritin proteins from different sources and all ferritins assemble, from multiple subunits, into a symmetrical cage-like structure. This protein cage acts to sequester Fe as a constrained nanoparticle of ferric oxyhydroxide (usually ferrihydrite). The reactions to form the mineral particle include the oxidation of Fe(II) and its subsequent hydrolytic polymerization to form the mineral. These reactions are catalyzed by the protein in two distinct ways. An enzymatic active site (ferroxidase) in the protein catalyzes the oxidation of Fe(II). The Fe(III) rapidly forms a small mineral core within the protein shell. This mineral surface will itself catalyze the oxidation of Fe(II) via the 4 electron reduction of O₂ to H₂O, Nucleation of the mineral particle inside the protein cage of ferritin occurs at symmetry related clusters of glutamic acid residues, which create a protein surface of high charge density. In the absence of the feroxidase site this highly charged interface is sufficient to induce oxidative hydrolysis and mineral formation within the confines of the protein cage.

There is also interest in the chemical design and construction of self-assembling systems that can be used as delivery vehicles for various therapeutic and/or imaging agents or molecules. For example, viral capsid proteins are able to self assemble into highly symmetrical structures in a wide range of sizes from a very small number of basic building blocks (W. Chiu, R. Burnett, and R. Garcea (eds) (1997) “Structural Biology of Viruses Oxford”, University, New York).

There is high degree of structural similarity between the basic building blocks of many icosahedral viruses regardless of whether they infect animals, plants, insects, fungi or bacteria (Rossman, M. G., and J. E. Johnson, 1989, Annu. Rev. Biochem. 58:533-573). Most of the viral coat protein subunits have the β-barrel motif and assemble into hexameric and pentameric capsomer units. The ratio of hexamers to pentamers determines the curvature of the overall structure and ultimately the size of the final virion (Johnson, J. E., 1996, Proc. Natl. Acad. Sci. USA 93:27-33; Johnson, J. E., and J. A. Speir, 1998, J. Mol. Bio. 269:665-675; and Rossman and Johnson, 1989, supra). This is geometrically very similar to the assembly of fullerenes such as the so-called “buckyballs” (Smalley, R. E., 1992, Acc. Chem. Res. 25:98-105). The higher the hexamer to pentamer ratio, the larger the diameter of the structure. Spherical viruses typically range in size from 18-500 nm, while rod shaped viruses of >900 nm are known. At least conceptually, the natural variation in virus particle size and shape provides a wealth of potential protein cages.

The small spherical virus cowpea chlorotic mottle virus (CCMV) is an ideal model system for developing viral protein cages for cell-targeted bioimaging and therapeutic delivery. Other protein cages that may be useful for cell-targeted bioimaging and therapeutic delivery include apoferritin and the heat shock protein from Methanococcus jannaschii.

Accordingly, there is a need for delivery vehicles in the form of protein cages containing one or more agents, such as therapeutic and/or imaging agents, and methods of using the same.

### SUMMARY OF THE INVENTION

The present invention is directed to protein cages that are self assembling and that include a plurality of subunits with at least one of such subunits being a modified subunit, a first agent, and a targeting moiety. The first agent may be a therapeutic agent, a photosensitizing agent, or a thermal ablation agent. The thermal ablation agent may include a magnetic material, which itself may include a ferromagnetic material.

The protein cage may have a modified subunit that is chemically modified. The modified subunit may be a genetically modified subunit. In some cases, the protein cage will include at least one chemically modified subunit and at least one genetically modified subunit. A chemically modified subunit may also include a linker. The modified subunit may include one or more proteins, which may be antibodies and/or peptides. In some cases, the protein may further include a radioisotope. In other cases, the protein is either a targeting moiety or a therapeutic agent. Alternatively, the protein may also act as both a targeting moiety and a therapeutic agent.

The first agent of a protein cage of the present invention may be an imaging agent. Imaging agents may be MRI agents. In some cases, the protein cages may have a modified subunit that includes a binding site for a chelator, which may be a chelator for a paramagnetic metal ion. In addition, the chelator binding site may include a peptide. The paramagnetic metal ion used with the protein cages may be gadolinium. The imaging agents used with the protein cages may also be optical agents.

The protein cages may also further include a disassembly mechanism. The mechanism may be a reversible switch and/or one or more enzymatic cleavage sites. A disassembly mechanism involving enzymatic cleavage site(s) may also be referred to as a “gating mechanism.” The enzymatic cleavage site may be a hydrolase cleavage site. The hydrolase may be a protease, a carbohydrase, or a lipase. The hydrolase cleavage site may be a protease cleavage site, which may be a cathepsin cleavage site.

The protein cages may contain viral and/or non-viral protein subunits. Viral subunits include the cowpea cholorotic mottle virus (CCMV) and the MS2 capsid. Non-viral subunits include heat shock proteins, such as those from Methannococcus jannaschii; Dps-like proteins, such those from Sulfolobus solfataricus, Pyrococcus furiosus, and Listeria innocua; and a mammalian ferritin protein, such as those from a mouse or a human.

### BRIEF DESCRIPTION OF THE DRAWINGS

FIG. 1 is a table representing the possible matrix of protein cages and their corresponding features as contemplated by the present invention.

FIG. 2 illustrates the structures of the sHsp cage (top) and CCMV cage (bottom), as well as a schematic of the available sites for genetic and chemical modifications.

FIG. 3 shows the modification of doxorubicin attachment to the interior (engineered —SH) of sHsp. Cleavable linkers (—S—S— or ester) are incorporated into the linker.

FIG. 4 shows the high relaxivities on a per protein cage and per Gd³⁺ basis for a CCMV protein cage.

FIG. 5 shows that Gd³⁺ binding sites may be incorporated into CCMV and/or Hsp protein cages.

FIG. 6 shows fluorescence microscopy of RGD-4C targeted cages to C32 melanoma.

FIG. 7 shows shows peptide targeting in cell culture (A) and in tumor xenographs (B). (A) Fluorescent F3-peptide conjugate internalized into nuclei of tumor cells. (B) Fluorescent Lyp-1 peptide conjugate localized to tumor in whole mouse imaging and tissue section (inset)

FIG. 8 (A-C) illustrates the basic principle of introducing soluble material such as a medical imaging agent into a protein cage.

FIG. 9 illustrates an engineered redox-dependent gating. On the top is a cryo TEM image reconstruction of closed (left) and open (right) forms of a CCMV protein cage. The site of protein engineering between coat protein subunits is shown (lower left) with an enlarged view of introduced cys residues (yellow, lower right). The TEM of protein cages with redox dependent gating in closed conformation is also shown. (inset)

FIG. 10 illustrates the use of an in vitro assembly system in which differentially modified cage subunits can be reassembled to control multifunctional ligand presentation.

FIG. 11 shows a schematic of a programmed cage aggregation at a targeted cell surface.

FIG. 12 shows a programmed sequential cage aggregation at target cell surface. a) protein cages bi-functionalized cell targeting peptide (green) and agrregation ligand (biotin, red circles) b) addition of second cage with complementary aggregation ligand (avidin) c) programmed aggregation at cell surface d) additional aggregation layers could be sequentially programmed.

FIG. 13 shows the TEM of CoPt synthesized within the sHsp cage using specific peptides engineered onto the interior surface of the cage (upper right). Electron diffraction (top left) confirms the L₁0 phase and the magnetometry (lower right) indicates ferromagnetic behavior at 300K.

FIG. 14 illustrates examples of different materials entrapped/crystallized within the CCMV protein cage. FIG. 14A is an unstained sample of H₂WO₄₂¹⁰⁻ cores. FIG. 14B is a negative stain sample of H₂WO₄₂¹⁰⁻ cores showing protein cages. FIG. 14C is a negative sample of encapsulated polyanetholesulphonic acid. FIG. 14D is an unstained sample of ferric oxide cores in P. pastoris expressed protein cages.

FIG. 15 (A-C) illustrates the expression of a targeting moiety on the exterior surface of a CCMV protein cage. FIG. 15A is a TEM of peptide 11 protein cages from the P. pastoris system. FIG. 15B is a PCR digest of coat protein. Lane 1, protein cage with peptide 11 protein; lane 2, wild type coat protein, and lane 3, no peptide 11 control. FIG. 15C is a westron blot of P. pastoris expressing peptide 11 coat protein (lane1); wild type coat protein (lane 2) and control (lane 3).

FIG. 16 shows the deconvoluted Mass spectrum of HspG41CRGD-4C.

FIG. 17 illustrates the characterization of the ‘tumor targeting’ HspG41CRGD-4C protein cage architecture. FIG. 17A displays the size exclusion chromatography elution profile of HspG41CRGD-4C cages (Abs 280 nm), FIG. 17B displays the dynamic light scattering analysis of HspG41CRGD-4C (average diameter 15.4+0.3 nm), and FIG. 17C displays the transmission electron micrograph of the HspG41CRGD-4C cages negatively stained with 2% uranyl acetate.

FIG. 18 demonstrates the covalent linkage of cargo molecules by SDS-PAGE of HspG41CRGD-4C subunits.

FIG. 19 shows the deconvoluted mass spectrum for HspG41CRGD-4C-Fluorescein.

FIG. 20 shows the epifluorescence microscopy of C32 melanoma cells with Hsp cage-fluorescein conjugates.

FIG. 21 shows the specific binding of ‘tumor targeted’ HspG41CRGD4C-fluorescein labeled cages to C32 Melanoma Cells.

FIG. 22 shows the specific binding of anti-CD4 mAb conjugated HspG41C-fluorescein cages (Ab-HspG41C-Fl) to CD4+ lymphocytes.

FIG. 23 (A-D) shows the binding of ‘tumor targeted’ HspG41CRGD4C-fluorescein labeled cages to C32 melanoma cells relative to an anti-αvβ3 antibody-fluorescein positive control.

FIG. 24 shows the characterization of fluorescein labeled HspG41CRGD-4C cages.

FIG. 25 shows the characterization of HspG41CRGD-4C cages bound to doxorubicin.

FIG. 26 shows the deconvoluted mass spectrum of HspG41CRGD-4C-Doxorubicin.

FIG. 27 shows the dynamic light scattering analysis of anti-CD4 mAb HspG41C.

FIG. 28 shows TEM images and DLS analysis (insets are the corresponding correlation functions) of empty HFn and RGD4C-Fn. Both HFn and RGD4C-Fn show 12-14 nm in diameter.

FIG. 29 shows TEM images and size distribution histograms as measured by TEM of mineralized RGD4C-Fn, HFn and HoSpFn under various loading factors of Fe. The values shown on the histograms are mean ±standard deviation. (A) RGD4C-Fn 1000 Fe/cage, (B) RGD4C-Fn 3000 Fe/cage, (C) RGD4C-Fn 5000 Fe/cage, (D) HFn 3000 Fe/cage. (E) HoSpFn 1000 Fe/cage. The size of the particles formed inside of RGD4C-Fn increase with increasing loading factor of Fe per cage. There is no significant difference in size between particles formed inside of HFn and RGD4C-Fn under same loading factor. Particles formed within HoSpFn are larger in size and wider distribution than those formed within RGD4C-Fn.

FIG. 30 shows (A) EELS of mineralized RGD4C-Fn with loading of factor 3000 Fe/cage. (B) Selected area electron diffraction pattern of mineralized RGD4C-Fn with loading factor of 3000 Fe/cage.

FIG. 31 shows the SEC of RGD4C-Fn (A) before mineralization reaction (B) after mineralization with 3000 Fe/cage. Elution was monitored at both 280 nm (protein) and 410 nm (iron oxide mineral). Co-elution of protein and mineral in profile (B) indicate the composite nature of the mineralized protein cage.

FIG. 32 shows (A) ACMS measurement of mineralized RGD4C-Fn with various loading factors under 10 Oe field at 1000 Hz. (B) Plot of blocking temperature (Tb) at 1000 Hz against particle volume for mineralized core.

FIG. 33 shows Neel-Arrhenius fits showing the frequency dependence of the blocking temperature of each sample, according to equation (1). Here, the inverse blocking temperatures of the 3000 Fe and 5000 Fe/cage samples have been scaled by 2 to more clearly display the linearity of the data. The linear behavior of each data set indicates that the particles are non-interacting.

FIG. 34 shows hysteresis loops (magnetization versus applied field) at 5 K for mineralized RGD4C-Fn.

FIG. 35 shows a TEM micrograph of C32 cell incubated with mineralized RGD4C-Fn with loading factor of 3000 Fe/cage for 30 min. Arrows indicate mineralized ferritin.

FIG. 36 shows the FACS analysis of C32 melanoma cells incubated with fluorescence labeled protein cages. The data are plotted as histograms with their corresponding geometric (geo.) mean fluorescence values. Blue solid line indicates cells not incubated with RGD4C-Fn or HFn (geo. mean=36). Red dashed line indicates cells incubated with fluorescein labeled HFn (geo. mean=568). Green filled plot indicates cells incubated with fluorescein labeled RGD4C-Fn (geo. mean=1972). The increased level of fluorescence intensity of the cells incubated with fluorescein labeled RGD4C-Fn indicates specific binding of the cages to C32 melanoma cells.

FIG. 37 shows spacefilling representation of the exterior surface of CCMV (left) with reactive surface exposed lysines indicated in red illustrating their highly symmetric presentation on the icosahedral protein cage.

FIG. 38 shows a schematic for the assembly of asymmetrically functionalized particles.

FIG. 39 shows a mixed reassembly assay. I) Biotin functionalized CCMV was removed from a complex mixture by binding to streptavidin agarose (SA). II) Subsequent Westernanalysis (FIGS. 40 and 41 48) detected biotin (B) and/or digoxigenin (D) functionalized protein bound to streptavidin agarose.

FIG. 40 shows mixed reassembly assays probed by Western blot analysis. (A): SDS-PAGE of total protein in mixtures (input) before binding to streptavidin agarose. (B): Western blot analysis of reassembled protein cages bound to streptavidin agarose. Lane 1) molecular weight markers; Lane 2) wild type protein subunits, no label; Lane 3) biotin labeled subunits; Lane 4) digoxigenin labeled subunits; Lane 5) mixture of biotin and digoxigenin labeled subunits combined prior to reassembly.

FIG. 41 shows the analysis of reassembly of varying amounts of labeled subunit. A: Western blots of streptavidin bound CCMV reassembly reactions probed with antibodies specific to digoxigenin (ab-dig) or biotin (ab-bio). The ratio of digoxigenin to biotin functionalized subunits present during assembly is indicated. B: The ratio of band intensities (digoxigenin:biotin) from Western blots versus the ratio of biotin to digoxigenin subunits in the assembly mixture (input); intercept=0.232, slope=2.002, r 2=0.993. C: Western blot of streptavidin bound CCMV reassembled from modified subunits in ratios from 1:1 to 800:1 (dig:bio), probed with antibodies specific to digoxigennin (ab-dig) or biotin (ab-bio).

FIG. 42 shows that the 28 nm capsid of CCMV is made of 180 individual subunit proteins (20 hexaminers and 12 pentamers).

FIG. 43 shows (A) an inside view of CCMV's viral capsid. Blue highlights are residue 27 in the 6 fold environment and red highlights are residue 42 in the 5-fold environments and (B) the first twenty amino acids for both the unmodified and genetically modified subunit. The underlined residues are responsible for metal binding.

FIG. 44 shows the reaction scheme to attach DOTA-Gd3+ to the CCMV viral capsid. Endogenous lysines on the viral capsid are reacted to a DOTA/NHS conjugation. Next GdCl3 is added to produce a viral capsid conjugated with Gd3+ ions.

FIG. 45 shows data from routine virus capsid characterization of CCMV-CAL or CCMV-DOTA. (A) shows size exclusion chromatogram showing three main components. The small left peak is aggregated virus capsids. The large middle peak is intact capsids eluting at the correct retention volume and the peaks to the right are buffer molecules. (B) Dynamic light scattering indicates a viral capsid mean diameter of 30 nm. (C) Transmission electron micrograph of negatively stained viral particles. (D) The deconvoluted spectrum shows the correct subunit mass. Inset is the raw electrospray mass spectrum of viral capsid subunits.

FIG. 46 shows the binding isotherms for CCMV CAL binding Tb3+ ions. Duplicate binding experiments are shown for both the decrease in 340 nm peak and increase of the 550 nm peak. All four data sets were used for the fit.

FIG. 47 shows a stoichiometric titration of CCMV CAL with Tb3+ ions. Two replicate sets are shown.

FIG. 48 shows a typical deconvoluted electrospray mass spectra of CCMV capsid subunits. CCMV capsids were reacted with NHS-DOTA and then GdCl3 was added. Unlabeled subunits and subunits with one to three DOTA-Gd were detected.

FIG. 49 shows a cutaway view of the interior of CCMV. GdDOTA was modeled to be attached through Lys 45. The red highlighted view is a close up of Lys 45 in the 6-fold environment while blue highlighted view is of Lys 45 in a 5-fold environment.

FIG. 50 shows (A) r1 values for two CCMV capsids conjugated with Gd3+ ions and (B) r2 values for two CCMV capsids conjugated with Gd3+ ions. The squares represent CCMV-Gd with Gd3+ ions bound at an endogenous Ca2+ binding site. The triangles represent CCMV-CAL-Gd, Gd3+ ions bound in the inserted and endogenous metal binding sequence.

FIG. 51 shows (A) r1 values for both CCMV-DOTA-Gd (red squares) and GdDOTA (blue circles) and (B) r2 values for both CCMV-DOTA-Gd (red squares) and GdDOTA (blue circles)

### DETAILED DESCRIPTION OF THE INVENTION

The present invention is directed to the discovery that a variety of nanoparticles comprising protein cages can be made and mixed to produce materials with both a variety of new applications as well as “tunable” applications, e.g. the ability to alter material properties, e.g. different magnetic properties, by the incorporation of different elements in the nanoparticles and nanoparticle cores. This allows the directed synthesis of nanophase magnetic particulate materials whose magnetic properties are tailored by the size and composition of the particles, and by their assembly into mono- and multi-component two-dimensional ordered arrays. Thus, new magnetic materials are made whose component constituents are magnetic clusters that can be tightly tailored in size and magnetic composition, and whose mesoscopic magnetic properties (individual cluster moment, anisotropy, etc.) can be independently varied over a broad range. Furthermore, through the use of an appropriate interstitial material or derivatization of the shell materials, the assembly of these magnetic building blocks into ordered two-dimensional arrays allows for tunable and externally controllable inter-particle interactions that modify the macroscopic material properties for future application as superior performance magnetic memory, sensors, and ultra-high speed device architectures.

The protein cages of the present invention can be thought of as molecular “lego” sets. That is, they can be assembled with precision from a predetermined number of protein subunits. In one embodiment, these protein cage assemblies are spherically symmetrical structures that provide precisely defined exterior and interior surfaces. In nature these architectures serve a diversity of roles from nucleic acid storage and transport in viruses to iron mineralization and sequestration in ferritins. In some embodiments, the present invention provides compositions and methods of making the same that impart functional capabilities to these architectures for application in targeted therapeutic agent delivery and medical imaging.

In one aspect, the present invention utilizes self-assembling protein cage architectures. It is generally known in the art that self-assembling protein cages may have particular features involved in the self-assembling process (see Padilla, J. E. et al. (2001) Proc. Nat'l Acad. Sci. 98(5):2217-2221, which is incorporated herein by reference in its entirety). In one embodiment, the protein cages may include protein subunits that are capable of self-assembling protein cages.

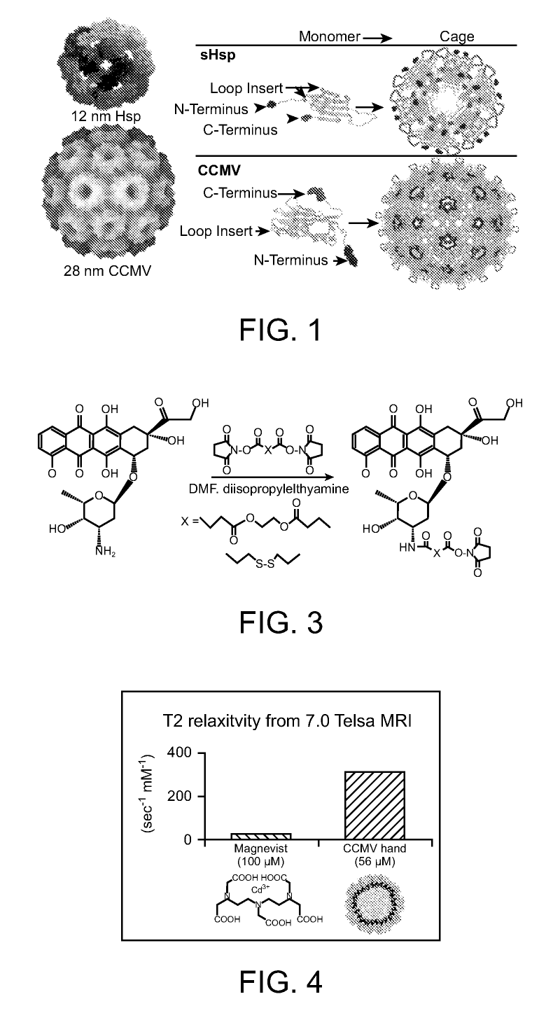

A wide variety of protein cages are contemplated by the present invention. The cages may be derived from different sources (as described below) and engineered to have various features, including without limitation therapeutic and/or imaging agents, targeting moieties, disassembly mechanisms, and other features described herein. FIG. 1 is a protein cage matrix illustrating the variety of different protein cages contemplated by the present invention. However, the number of protein cage possibility should not be taken to be limited to those disclosed in FIG. 1.

I. Protein Cages Nanoparticles

Previous work has utilized several different types of protein “shells” that can be loaded with different types of materials. For example, as outlined above and herein, virion nanoparticles comprising a shell of coat protein(s) that encapsulate a non-viral material have been described; see U.S. Pat. No. 6,180,389, hereby incorporated by reference in its entirety. Similarly, as described above and in references outlined in the bibliography, mammalian ferritin protein cages have been used that can be loaded with certain uniform materials. Mammalian ferritin protein cages have a 413/2 octahedral symmetry, include two types of subunits: an H and an L subunit, and can accommodate about 4500 iron atoms within the cage (See Klem, M. T. et al., Materials Today, September 2005; 28-37).

Accordingly, the present invention provides compositions comprising a plurality of nanoparticles. By “nanoparticle” or “protein cage” herein is meant a composition of a proteinaceous shell that self-assembles to form a protein cage (e.g. a structure with an interior cavity which is either naturally accessible to the solvent or can be made to be so by altering solvent concentration, pH, equilibria ratios, etc.), which cage has been loaded with a material as discussed below. That is, a “nanoparticle” includes both the shell (e.g. protein cage) and the nanoparticle core. As outlined herein, different protein cages lead to different sized cores. In some embodiments, the present invention utilizes cores ranging from about 1 nm to about 30 nm (e.g. the internal diameter of the shells), from about 5 nm to about 24 nm (representing 8.5 to 28 nm outer shell diameters, in general, particularly when non-viral protein cages are used), from about 7 nm to about 20 nm, from about 10 nm to about 15 nm. In other embodiments, a protein cage of about 12 nm (particularly where non-viral cages are utilized) or about 24 nm (particularly wherein viral cages are utilized) are utilized.

In one embodiment, a set of conditions are used to control the assembly size of a specific protein subunit type, for example with CCMV as described herein. Different chemical conditions can yield different number of subunits in cage with different sizes.

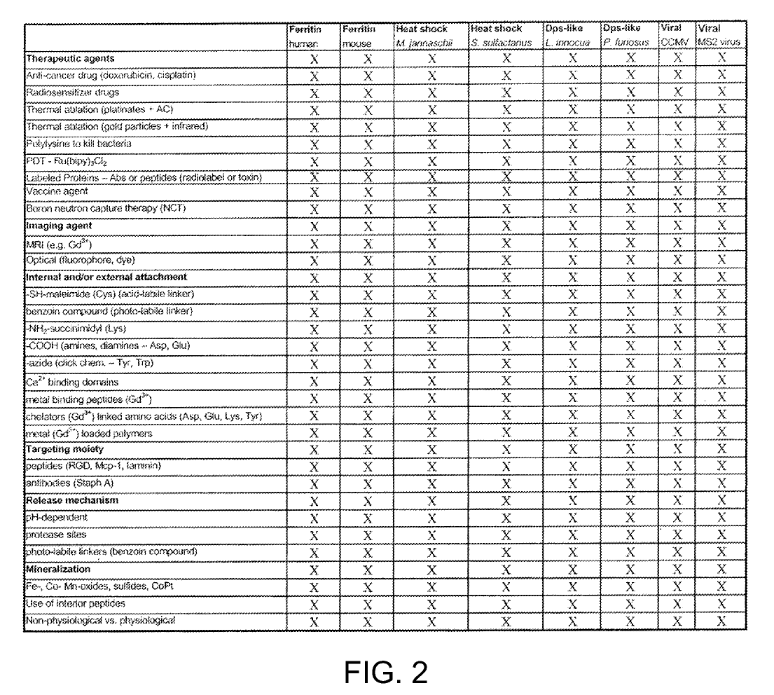

As illustrated in FIG. 2, viral and non-viral protein cages may be used.

A. Non-Viral Protein Cages.

Non-viral protein cages of the invention include without limitation ferritins and apoferritins, both eukaryotic and prokaryotic species, in particular mammalian and bacteria, particularly 12 and 24 subunit ferritins. Also included are the 24 subunit heat shock proteins that form an internal core space. In particular, Methanococcus jannaschii assembles into a 24 subunit cage with 432 symmetry (see Kim, K. K. et al., 1998, Nature 394:595-599; Kim, K. K. et al., 1998, J. Struct. Biol. 121:76-80; and Kim, K. K. et al., 1998, PNAS 95:9129-9133). Protein cages formed from the small heat shock protein (Hsp) of M. jannaschii have a macromolecular structure of about a 12 nm exterior diameter and about a 6.5 nm interior diameter. These Hsp cages can be heated up to about 65° C. and are stable at a pH from about 6 to about 9. The superstructure of the Hsp cage includes 8.3 nm pores that render the interior cavity very accessible for interaction with various types of agents, as described herein.

The non-viral protein cage architectures contemplated also include without limitation ferritin (Chasteen, N. D., et al., 1999 J. Struct. Biol. 126:182-194), heat shock proteins (Kim, K. et al, supra 1998), lumazine synthase (Shenton, W., et al., 2001. Angewandte Chemie-International Edition 40:442-445), and Dps (Reindel, S., et al., 2002. Biochimica Et Biophysica Acta-Proteins and Proteomics 1598:140-146). Mammalian ferritin is a metalloprotein complex formed from a roughly spherical core containing about 3,000 inorganic atoms such as iron, and a shell of 24 identical subunits each having a molecular weight of about 20 kD. The outer diameter of mammalian ferritin is roughly 12 nm and the core is roughly 8 nm. Ferritin without the iron core molecules is called apoferritin. Listeria innocua has a ferritin-like structure that catalyzes the oxidation of Fe(II) and is a dodecameric (12 subunits, rather than 24) protein. There are a variety of other self-assembling “shells” known, including the dodecameric Dsp heat shock protein of E. coli and the MrgA protein as well as others known in the art.

In one embodiment of the present invention, protein cages may comprise proteins from the Dps (DNA-binding protein from starved cells) family. Dps proteins can be found in the Gram-positive bacterium Listeria innocua. (See Ilari, A. et al., (1999) Acta Crystallogr. D55, 552-553; Stefanini, S. et al. (1999) Biochem. J. 338, 71-75; Bozzi, M. et al. (1997) J. Biol. Chem. 272, 3259-3265; Su, M. et al., Biochemistry 2005, 44, 5572-5578).

In addition, Dps or Dps-like proteins can be found in a variety of bacteria including, but not limited to, E. coli (Almiron, M. (1992) Genes Dev. 6, 2646-54; Ilari, A. et al., (2002) J. Biol. Chem., Vol. 277, Issue 40, 37619-37623); Helicobacter pylori (Tonello, F. et al., (1999) Mol. Microbiol. 34, 238-246); Halobacterium salinarum (Zeth, K. et al., Proc Natl Acad Sci USA. 2004 Sep. 21; 101(38): 13780-13785); and Bacillus anthracis (Papinutto, E. et al., Proc Natl Acad Sci USA. 2002 Apr. 26; 277(17): 15093-15098). Other Dps or Dps-like proteins will be appreciated by those of ordinary skill in the art and as described in Wiedenheft, B. et al., Proc Natl Acad Sci USA. 2005 Jul. 26; 102(30):10551-6. Epub 2005 Jul. 15, and Ramsay, B. et al., J. Inorganic Biochemistry 100 (2006) 1061-68, each of which is incorporated herein by reference in its entirety.

In another embodiment, the protein cage includes one or more Sulfolobus solfataricus protein subunits encoded by the ssdps gene. S. solfataricus proteins are known to be a Dps-like proteins. Such cages self-assemble into a hollow dodecameric protein cage having tetrahedral symmetry. The outer shell diameter is ˜10 nm, and the interior diameter is ˜5 nm. Dps proteins have been shown to protect nucleic acids by physically shielding DNA against oxidative damage and by consuming constituents involved in Fenton chemistry. In vitro, the assembled archaeal protein efficiently uses H₂O₂ to oxidize Fe(II) to Fe(III) and stores the oxide as a mineral core on the interior surface of the protein cage. The ssdps gene is up-regulated in S. solfataricus cultures grown in iron-depleted media and upon H₂O₂ stress, but is not induced by other stresses. SsDps-mediated reduction of hydrogen peroxide and possible DNA-binding capabilities of this archaeal Dps protein are mechanisms by which S. solfataricus mitigates oxidative damage. (see Wiedenheft, B. et al., Proc Natl Acad Sci USA. 2005 Jul. 26; 102(30):10551-6. Epub 2005 Jul. 15, incorporated herein by reference in its entirety)

In another embodiment, the protein cage includes one or more Pyrococcus furiosus protein subunits encoded by the PfDps gene. P. furiosus proteins are known to be Dps-like proteins (Ramsay, B. et., J. Inorganic Biochem. 100 (2006) 1061-1068, incorporated herein by reference in its entirety). Such cages self-assemble into a 12 subunit quaternary structure with an outer shell diameter of ˜10 nm and an interior diameter of −5 nm. Dps proteins functionally manage the toxicity of oxidative stress by sequestering intracellular ferrous iron and using it to reduce H2O2 in a two electron process to form water. The iron is converted to a benign form as Fe(III) within the protein cage. This Dps-mediated reduction of hydrogen peroxide, coupled with the protein's capacity to sequester iron, contributes to its service as a multifunctional antioxidant.

In another embodiment, the Dps protein contained in a protein cage of the present invention may be from the Listeria innocua bacteria. L. innocua has a ferritin-like structure that catalyzes the oxidation of Fe(II) and is a dodecameric (12 subunits, rather than 24) protein. The L. innocua protein cage structure has 3/2 tetrahedral symmetry. The internal diameter of such cages is about 40 angstroms.

Such Dps-like proteins are known to protect DNA against damage from toxic oxygen species such as O₂, H₂O₂ and —OH. In addition, (See Su (2005) Biochemistry supra) In general, Dps protein cage structures include 12 subunits. (See supra Klem, M. T. et al.). In one embodiment, the present invention provides compositions that contain a protein cage having one or more Dps proteins as described herein. Such cages may include an agent as described herein. Additionally, the Dps protein-containing cage may include one or more modified subunits.

There are a variety of other self-assembling “shells” known, including the dodecameric Dsp heat shock protein of E. coli and the MrgA protein as well as others known in the art.

In some embodiments, the present invention provides protein cages with a non-viral origin as discussed above having having one or more modified subunits as described below.

B. Viral Protein Cages

Viral capsids have evolved to encapsulate nucleic acids for protection, transport, and delivery to appropriate cells. Thus, in chemical terms, viral capsids can be viewed as delivery vehicles for a variety of agents. The vehicle-agent property is amenable to synthetic approaches to making molecular cage-like structures. The delivery vehicles are characterized by clearly defined interiors and exteriors, i.e. interfaces that interacts with the agents on their interior and/or exterior surfaces. The interior and exterior interfaces are chemically and geometrically different and it is these differences which provide specificity and function to a protein cage (Kang, J. et al. 1997, Nature 385:50-52; Sherman, J. C. et al. 1989, J. Am. Chem. Soc. 111:4527-4528). The agent attached to the delivery vehicle on the other hand has properties which allow it to interact specifically with the interior interface of the vehicle. This molecular recognition is usually dependent on weak H-bonding, van der Waal's, and/or electrostatic interactions (Rebek, J., 1996, Chem. Soc. Rev. 25:255-264).

The capsid structures of viruses are a near perfect example of a highly evolved vehicle-agent system functioning to store, transport, and release viral genomes and associated proteins. Capsids come in two basic geometric shapes: roughly spherical (usually based on icosahedral symmetry) and rod shaped (usually based on helical symmetry). All capsids have curvature which defines the overall size and shape of the host. Many viruses are pleomorphic and are able to assemble in a range of geometric configurations (icosahedrons, flat sheets, tubes etc.). In addition, many capsid structures of viruses undergo reversible structural transitions that play a role in the packaging or release of nucleic acids.

Suitable viral protein cages can be obtained from any animal or plant virus from which empty viral particles can be produced. For example, empty viral particle can be obtained from viruses belonging to the bromovirus group of the Bromoviridae (Ahlquist, P., 1992, Curr. Opin. Gen. and Dev. 2:71-76; Dasgupta, R., and P. Kaesberg, 1982, Nucleic Acid Res. 5:987-998; and Lane, L. C., 1981, The Bromoviruses. In E. Kurstak (ed.), “Handbook of plant virus infection and comparative diagnosis”, Elsevier/North-Holland, Amsterdam) and from the family Caliciviridae. Viruses suitable for use in the invention include cowpea chlorotic mottle virus (CCMV) and the Norwalk virus.

Viral capsids are large protein assemblies. The most common spherical capsids range between 18-100 nm in diameter. Protein cages can also serve as robust synthetic platforms that are chemically and genetically malleable and can be readily modified. Previous studies have explored the use of protein cages as therapeutic or imaging delivery agents (Yamada T et al. Nat Biotechnol 2003; 21(8):885-890; Anderson E A, et al Nano Letters 2006; 6(6):1160-1164; Lewis J D, et al. Nat Med 2006; 12(3):354-360). Cell targeting has been achieved by utilizing capsids with natural affinities for cellular receptors or by chemically linking peptides or antibodies to protein cage architectures (Flenniken M L, et al. Chem Biol 2006; 13(2):161-170; Singh P et al. J Nanobiotechnology 2006; 4:2). In addition, targeted protein cages incorporating a therapeutic payload (doxorubicin) have been constructed, demonstrating the multifunctional capacity for biomedical applications (Flenniken M L, et al. Chem Commun (Camb) 2005(4):447-449).

Protein cages have the potential to serve as extremely efficient contrast agents for the following reasons: 1) protein cages are large and relatively rigid molecular structures with large rotational correlation times, resulting in increased relaxivity rates; 2) protein cages can serve as robust platforms where multiple functional motifs can be added through genetic or chemical modifications (Allen M, et al. Adv Mater 2002; 14(21):1562; Allen M et al. Inorg Chem 2003; 42(20):6300-6305; Douglas T et al. Adv Mater 2002; 14(6):415; Douglas T et al. Nature 1998; 393(6681):152-155; Douglas T et al. Adv Mater 1999; 11(8):679; Douglas T et al. Science 2006; 312(5775):873-875; Flenniken M L et al. Nano Letters 2003; 3(11):1573-1576; Gillitzer E et al. Chem Commun 2002(20):2390-2391; Klem M T et al. Adv Funct Mater 2005; 15(9):1489-1494; Klem M T et al. J Am Chem Soc 2003; 125(36):10806-10807; Meunier S et al. Chem Biol 2004; 11(3):319-326; Rae C S et al. Virology 2005; 343(2):224-235; Raja K S et al. Biomacromolecules 2003; 4(3):472-476; Sen Gupta S et al. Bioconjug Chem 2005; 16(6):1572-1579; Sen Gupta S et al. Chem Commun (Camb) 2005(34):4315-4317; Varpness Z et al. Nano Letters 2005; 5(11):2306-2309; Khor I W et al. J Virol 2002; 76(9):4412-4419; Portney N G et al. Langmuir 2005; 21(6):2098-2103).

These modifications could potentially result in the attachment of both Gd³⁺ binding and site specific targeting functionalities and 3) protein cages can potentially carry hundreds (if not thousands) of Gd³⁺ ions and the contrast from an individual cage will increase significantly with the number of Gd³⁺ ions it carries. As a result, viral protein cages have been investigated as MRI contrast agents including the Cowpea chlorotic mottle virus (CCMV) capsid with bound Gd³⁺ at endogenous metal bind sites and the MS2 virus capsid with GdDTPA chemically attached (Anderson supra; Allen M et al. Magnet Reson Med 2005; 54(4):807-812; Hooker J M et al. (2004). J Am Chem Soc 126, 3718-9) Dendrimers, liposomes as well as other supermolecular structures maintain properties 2 and 3 mentioned above and therefore have also been developed as potential contrast agents (Kobayashi H et al. Mol Imaging 2003; 2(1):1-10; Mulder W J et al. NMR Biomed 2006; 19(1):142-164).

CCMV is a member of the bromovirus group of the Bromoviridae (a member of the alpha family supergroup) (Ahlquist, P., 1992, Curr. Opin. Gen. and Dev. 2:71-76; Dasgupta, R., and P. Kaesberg, 1982, Nucleic Acid Res. 5:987-998; and Lane, L. C., 1981, The Bromoviruses. In E. Kurstak (ed.), “Handbook of plant virus infection and comparative diagnosis”, Elsevier/North-Holland, Amsterdam). Bromovirues are 25-28 nm icosahedral viruses with a four component (+) sense single stranded RNA genome. CCMV has been used as a model system for viral assembly since 1967 when Bancroft and Hiebert demonstrated that purified RNA and coat protein self-assemble in vitro to produce infectious virions (Bancroft, J. B., et al., 1969, Virology 38:324-335; Bancroft, J. B., and E. Hiebert, 1967, Virology 32:354-356; Bancroft, J. B., et al., 1968, Virology 36:146-149; Hiebert, E., and J. B. Bancroft, 1969, Virology 39:296-311; and Hiebert, E., et al., 1968, Virology 34:492-508).

The present invention provides protein cages derived from the 28 nm capsid of Cowpea chlorotic mottle virus (CCMV) (Speir, J. A., et al., 1995, Structure 3:63-78). Its structure is shown in FIG. 42. CCMV undergoes a reversible pH-dependent structural transition between a closed and open form resulting in the opening of 60, 2 nm pores allowing access between the interior and exterior environments. In one embodiment, empty viral particles are obtained from CCMV. A 3.2 Å resolution structure of CCMV is available that can be used to predict the role of individual amino acids in controlling virion assembly, stability, and disassembly (Speir, J. A., et al., 1995, Structure 3:63-78). The virion is made up of 180 copies of the coat protein subunit arranged with a T=3 quasi-symmetry and organized in 20 hexamer and 12 pentameric capsomers. A striking feature of the coat protein subunit is the presence of N- and C-terminal ‘arms’ that extend away from the central, eight-stranded, antiparallel b-barrel core. Each coat protein consists of a canonical β-barrel fold (formed by amino acids 52-176) from which long N-terminal (residues 1-51; 1-27 are not ordered in the crystal structure) and C-terminal arms (residues 176-190) extend in opposite directions. These N- and C-terminal arms provide an intricate network of ‘ropes’ which ‘tie’ subunits together. The first 25 amino acids are found lining the interior surface of the virion (Rao, A. L. and G. L. Grantham, 1996, Virology 226:294-305; and, Zhao, X., et al., 1995, Virology, 207:486-494). These 25 amino acids are thought to be highly mobile and to be required for viral RNA packaging. Nine of the first 25 amino acids are basic (Arg, Lys) and are thought to neutralize the negatively charged RNA. The first 25 amino acids are not required for empty virion assembly (devoid of viral RNA) and thus can be modified to change the electrostatic nature of the virion's interior surface, etc. The orientation of the coat protein β-barrel fold is nearly parallel to the five-fold and quasi six-fold axes. This orientation results in five exterior surface-exposed loops, βB-βC, βD-βE, βF-βG, βC-αCD1, βH-βI. Surrounding each of the 60 quasi three-fold axes located on the interface between hexamer and pentamer capsomers are Ca²⁺ binding sites. There are 180 Ca²⁺ binding sites per virion. Each Ca²⁺ binding site consists of five residues (Glu81, Gln85, Glu148 from one subunit; Gln 149 and Asp 153 from an adjacent subunit) in an ideal position to coordinate Ca²⁺ binding.

In one embodiment, a yeast-based heterologous protein expression system (Pichia pastoris) for the large scale production of modified CCMV protein cages (see Example 5) Alternatively, an E. coli-based CCMV coat protein expression system can be used (Zhao, X., et al., 1995. Virology 207:486-494). Using the E. coli system, denatured coat protein can be purified to 90% homogeneity, renatured, and assembled into empty particles which are indistinguishable from native particles (Fox, J. M., et al., 1998, Virology 244:212-218; and Zhao, X., et al., 1995, Virology 207:486-494).

The present invention provides protein cages containing proteins from the MS2 virus capsid. (Anderson (2006) Nano Lett. supra; Allen M et al. Magnet Reson Med 2005; 54(4):807-812)

II. Delivery Agents

Accordingly, the present invention provides delivery agents. By “delivery agent”, “delivery vehicle” or “protein carrier” herein is meant a proteinaceous shell that self-assembles to form a protein cage (e.g. a structure with an interior cavity which is either naturally accessible to a solvent or can be made to be so by altering solvent concentration, pH, equilibria ratios, etc.), and contains imaging and therapeutic agents as discussed below. The protein cage may be obtained from a non-viral or viral source.

Any number of different materials, including organic, inorganic, and metallorganic materials, and mixtures thereof may combined with the protein cages. The combination may be the loading of an agent or material into the interior space of the protein cage. It may also be the attachment of materials and/or agents to one of the surfaces of the protein cages. The combination may include loading and/or attachment of materials and/or agents to the cage. In one embodiment, combinations of medical imaging agents and therapeutic agents are provided for use as imaging and therapeutic agents.

In one embodiment, protein cages are used as reaction vessels for the constrained crystallization of materials, such as the agents described herein. Based on purely electrostatic interactions, (NH₄)₁₀H₂W₁₂O₄₂, Fe₃O₄, and CO₂O₃ have been crystallized at the protein interface from supersaturated solutions (Allen, M., et al. 2002. Advanced Materials 14:1562−+), (Douglas, T., et al. 2002. Advanced Materials 14:415−+; Douglas, T., et al. 1998. Nature 393:152-155; Douglas, T., et al. 1999. Advanced Materials 11:679−+), (Flenniken, M. L., et al. 2003. Nano Letters 3:1573-1576). In one embodiment, the present invention provides a method including providing an interface for molecular aggregation based on complementary electrostatic interactions, which creates high local concentration at the protein interface. In another embodiment, the method is directed to the constrained synthesis of drug nanocrystals. In one embodiment, the drug is doxorubicin.

In another embodiment, the interaction between the protein cage and drug molecules is engineered through genetic and chemical manipulation of the interior protein interface. In some embodiments, phage display peptides (Mao, C. B., et al. 2003. PNAS USA 100:6946-6951) (Mao, C. B., et al. 2004. Science 303:213-217), (Seeman, N. C., et al. 2002. PNAS USA 99:6451-6455), (Whaley, S. R., et al. 2000. Nature 405:665-668) that are active towards crystallites of a drug, such as for example the anti-cancer drug doxorubicin, may be genetically incorporated into the protein subunit to selectively initiate the crystallization of the anti-cancer drug. In other embodiments, the drug has an inherently low aqueous solubility to control of the level of supersaturation and the drug-specific peptide may provide a nucleation site for crystal growth. In one embodiment, the drug is doxorubicin.

A. Therapeutic Agents

In one embodiment, a therapeutic agent is introduced into the protein cage. By “therapeutic agent” or “drug moiety” or therapeutically active agent” herein is meant an agent capable of effecting a therapeutic effect, i.e. it alters a biological function of a physiological target substance. By “causing a therapeutic effect” or “therapeutically effective” or grammatical equivalents herein is meant that the agent alters the biological function of its intended physiological target in a manner sufficient to cause a therapeutic and phenotypic effect. By “alters” or “modulates the biological function” herein is meant that the physiological target undergoes a change in either the quality or quantity of its biological activity; this includes increases or decreases in activity. Thus, therapeutically active agents include a wide variety of drugs, including antagonists, for example enzyme inhibitors, and agonists, for example a transcription factor which results in an increase in the expression of a desirable gene product (although as will be appreciated by those in the art, antagonistic transcription factors may also be used), are all included.

In addition, a “therapeutic agent” includes those agents capable of direct toxicity and/or capable of inducing toxicity towards healthy and/or unhealthy cells in the body. Also, the therapeutic agent may be capable of inducing and/or priming the immune system against potential pathogens. A number of mechanisms are possible including without limitation, (i) a radioisotope linked to a protein as is the case with a radiolabled protein, (ii) an antibody linked to an enzyme that metabolizes a substance, such as a produg, thus rendering it active in vivo, (iii) an antibody linked to a small molecule therapeutic agent, (iv) a radioisotope, (v) a carbohydrate, (vi) a lipid, (vii) a thermal ablation agent, (viii) a photosensitizing agent, and (ix) a vaccine agent.

1. Small Molecules and Drugs

In one aspect, the protein cages of the present invention include therapeutic agents including without limitation small molecules or drugs. In one embodiment, a drug is anchored to the interior protein interface of the cages to provide an alternative nucleation site for spatially selective crystallization. In crystallization, once an initial aggregate has formed the crystal growth process is self-perpetuating because of the high affinity the molecules have for the crystal surface and the ever-increasing surface area of the growing crystal. Thus, the protein interface acts only as a nucleation catalyst by providing an interface favorable for aggregation (either from drug specific-specific peptide or anchored drug) and a size constrained crystallization environment. In one embodiment, the drug is doxorubicin.

In one embodiment, doxorubicin may be substituted with a doxorubicin analog such as fluorescein. The spectroscopic signature of fluorescein (UV absorbance (229 nm), visible absorbance (495 nm) and strong fluorescence (520 nm)) makes it an inexpensive and easy molecule to monitor. The solubility of fluorescein (and doxorubicin) is moderate at room temperature in solutions in which the protein cages are stable (saturation ˜2 μM, 1% DMF) and shows a dramatic temperature dependence. Lowering the temperature even a few degrees will induce a condition of supersaturation, from which it is thermodynamically possible for crystallites to form. Experiments in the absence of protein cages may be performed to determine the conditions for “bulk” crystallization on a reasonable timescale (i.e. a few hours). Then, the empty protein cages (0.5 mg/ml) may be incubated with a saturated solution of fluorescein (or doxorubicin) at room temperature. The temperature of this solution may be lowered and before bulk crystallization occurs (monitored by light scattering) the protein cages may be isolated. Once the nano-crystal of the organic material form and the DMF removed (to ensure the non-dissolution of the crystallite) the crystal-encapsulated cages may be isolated by gradient centrifugation or column chromatography.

In one embodiment, targeting peptides may be covalently attached to the drug-nanoparticle cages as described herein. In another embodiment, the drug encapsulation/crystallization may be performed on cages to which the targeting peptides have been genetically incorporated. In vitro evaluation of the drug release may be through gel filtration chromatography, equilibrium dialysis, and LC/MS as a function of solution pH and subsequently studied in cell culture assays as described herein.

In some embodiments, the number of drug molecules per cage can fall within the range of about 1 to about 180, about 10 to about 170, about 20 to about 160, about 30 to about 150, about 40 to about 140, about 50 to about 130, about 60 to about 120, about 70 to about 110, about 80 to about 100, and about 90 to about 95. In other embodiments, other small molecules are covalently attached to a protein cage, including without limitation fluorescein, bipyridine, photofrin, and Gd³⁺-cheating agents.

In one embodiment, the present invention provides therapeutic agents incorporated into tumor cell targeted protein cages, and methods of making thereof. An anti-cancer drug may be anchored to the interior of the protein cage architecture. In other embodiments, the anti-cancer drug is attached via an ester linkage. Upon internalization through a non-endosomal pathway the ester will be susceptible to cellular esterases that are abundant in mammalian cytosol, as recently demonstrated for uptake and activation of fluorescent dyes (see Chandran, S. S., et al., 2005. J Am Chem Soc 127:1652-3) The ester of an anti-cancer drug, for example doxorubicin, (Chen, Q., et al. 2003. Synthetic Communications 33:2401-2421) is synthesized by reaction of ethylene glycol bis(succinimidylsuccinate) with doxorubicin, as shown in FIG. 3 and Table 1 below, and attached to the protein interior through coupling reactions to engineered amine groups, as previously demonstrated. Disulfide groups may also be incorporated into the linker to allow cleavage of the linker and release of doxorubicin upon exposure to the reducing intracellular environment.

TABLE 1

| Reactive species | Chemical linkers | Labels |
|---|---|---|
| Lysine (—NH₂) | succinic anhydride | RGD, NGR, F3, Lyp-1 |
| | NHS-esters, succinimidyl | fluorophores, doxorubicin |
| | esters | |
| Glutamate, | diamines | RGD, NGR, F3, Lyp-1 |
| Aspartate | amines | peptides, proteins |
| (COOH—) | | |
| Cysteine (—SH) | maleimide, | RGD, NGR, F3, Lyp-1 |
| | iodoacetimide BMPH | fluorophores, doxorubicin |
| Tyrosine | Azides, —SH, —NH₂ | RGD, NGR, F3, Lyp-1 |
| | | fluorophores, drugs |

In another embodiment, the anti-cancer drug may be attached to engineered thiol groups placed at a modified N-terminus of the protein cage subunits. The first 20-30 residues of the N-termini of both cages, have been shown to be very sensitive to trypsin cleavage in vitro. Cleavage sites (-Phe-Leu-Gly-) for cathepsin B, an intracellular cysteine protease found extracellularly near metatastic tumors, may be engineered into these regions to facilitate protease selective release of the anti-cancer drug. In one embodiment, the anti-cancer drug is doxorubicin (See Dubowchik, G. M. et al., (2002a) Bioconjug Chem 13:855-69; Dubowchik, G. M., et al., (2002b). Bioorg Med Chem Lett 12:1529-32).

The nature of the therapeutic effect between the therapeutically active moiety and the physiological target substance will depend on the both the physiological target substance and the nature of the effect. In general, suitable physiological target substances include, but are not limited to, proteins (including peptides and oligopeptides) including ion channels and enzymes; nucleic acids; ions such as Ca+2, Mg+2, Zn+2, K+, Cl−, Na+, and toxic ions including those of Fe, Pb, Hg and Se; cAMP; receptors including G-protein coupled receptors and cell-surface receptors and ligands; hormones; antigens; antibodies; ATP; NADH; NADPH; FADH2; FNNH2; coenzyme A (acyl CoA and acetyl CoA); and biotin, among others. Physiological target substances include enzymes and proteins associated with a wide variety of viruses including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like. Similarly, bacterial targets can come from a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. Y. pestis, Pseudomonas, e.g. P. aeruginosa, P. putida; Chlamydia, e.g. C. trachomatis; Bordetella, e.g. B. pertussis. Finally other targets can include Treponema, e.g. T. palladium; G. lamblia and the like.

In another embodiment, the present invention provides protein cages that contain vaccine agents, as described in section II.A.8. Vaccine Agents) for the above-mentioned viruses and bacterial targets.

In one embodiment, the target is a physiological target protein. The physiological target protein may be an enzyme. As will be appreciated by those skilled in the art, the possible enzyme target substances are quite broad. Suitable classes of enzymes include, but are not limited to, hydrolases such as proteases, carbohydrases, lipases and nucleases; isomerases such as racemases, epimerases, tautomerases, or mutases; transferases, kinases and phophatases. Enzymes associated with the generation or maintenance of arterioschlerotic plaques and lesions within the circulatory system, inflammation, wounds, immune response, tumors, apoptosis, exocytosis, etc. may all be treated using the present invention. Enzymes such as lactase, maltase, sucrase or invertase, cellulase, α-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; and caspases, such as caspase-3, -5, -8 and other caspases of the apoptotic pathway, such as interleukin-converting enzyme (ICE). Similarly, bacterial and viral infections may be detected via characteristic bacterial and viral enzymes. As will be appreciated in the art, this list is not meant to be limiting.

Once the target enzyme is identified or chosen, enzyme inhibitor therapeutically active agents can be designed using well known parameters of enzyme substrate specificities. As outlined above, the inhibitor may be another metal ion complex such as the cobalt complexes described above. Other suitable enzyme inhibitors include, but are not limited to, the cysteine protease inhibitors described in PCT US95102252, PCT/US96/03844 and PCT/US96/08559, and known protease inhibitors that are used as drugs such as inhibitors of HIV proteases.

In another embodiment, the physiological target is a protein that contains a histidine residue that is important for the protein's bioactivity. In this case, the therapeutically active agent can be a metal ion complex (not to be confused with the metal ion complexes of the imaging agents), such as is generally described in PCT US95116377, PCT US95/16377, PCT US96/19900, PCT US96/15527, and references cited within, all of which are expressly incorporated by reference. These cobalt complexes have been shown to be efficacious in decreasing the bioactivity of proteins, particularly enzymes, with a biologically important histidine residue. These cobalt complexes appear to derive their biological activity by the substitution or addition of ligands in the axial positions. The biological activity of these compounds results from the binding of a new axial ligand, for example the nitrogen atom of imidazole of the side chain of histidine which is required by the target protein for its biological activity. Thus, proteins such as enzymes that utilize a histidine in the active site, or proteins that use histidine, for example, to bind essential metal ions, can be inactivated by the binding of the histidine in an axial ligand position of the cobalt compound, thus preventing the histidine from participating in its normal biological function.

In one other embodiment, the physiological target substance is a physiologically active ion, and the therapeutically active agent is an ion binding ligand or chelate. For example, toxic metal ions could be chelated to decrease toxicity, using a wide variety of known chelators including, for example, crown ethers.

Once the physiological target substance has been identified, a corresponding therapeutically active agent is chosen. These agents will be any of a wide variety of drugs, including, but not limited to, enzyme inhibitors, hormones, cytokines, growth factors, receptor ligands, antibodies, antigens, ion binding compounds including crown ethers and other chelators, substantially complementary nucleic acids, nucleic acid binding proteins including transcription factors, toxins, etc. Suitable drugs include cytokines such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone, testosterone, toxins including ricin, and any drugs as outlined in the Physician's Desk Reference, Medical Economics Data Production Company, Montvale, N.J., 1998 and the Merck Index, 11th Edition (especially pages Ther-1 to Ther-29), both of which are expressly incorporated by reference.

In another embodiment, the therapeutically active compound is a drug used to treat cancer. Suitable cancer drugs include, but are not limited to, antineoplastic drugs, including alkylating agents such as alkyl sulfonates (busulfan, improsulfan, piposulfan); aziridines (benzodepa, carboquone, meturedepa, uredepa); ethylenimines and methylmelamines (altretamine, triethylenemelamine, triethylenephosphoramide, triethylenethiophosphoramide, trimethylolmelamine); nitrogen mustards (chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard); nitrosoureas (carmustine, chlorozotocin, fotenmustine, lomustine, nimustine, ranimustine); dacarbazine, mannomustine, mitobranitol, mitolactol; pipobroman; doxorubicin, carboplatin, oxaliplatin, and cisplatin, (including derivatives).

In some embodiments, the therapeutically active compound is an antiviral or antibacterial drug, including aclacinomycins, actinomycin, anthramycin, azaserine, bleomycins, cuctinomycin, carubicin, carzinophilin, chromomycins, ductinomycin, daunorubicin, 6-diazo-5-oxn-I-norieucine, duxorubicin, epirubicin, mitomycins, mycophenolic acid, nogalumycin, olivomycins, peplomycin, plicamycin, porfiromycin, puromycin, streptonigrin, streptozocin, tubercidin, ubenimex, zinostatin, zorubicin; aminoglycosides and polyene and macrolide antibiotics.

In other embodiments, the therapeutically active compound is a radio-sensitizer drug, which sensitizes cells to radiation. In one embodiment, the cells sensitized are tumor cells. These drugs may be used in conjunction with radiation therapy for cancer treatment. Radiosensitizer drugs include without limitation halogenated pyrimidines such as bromodeoxyuridine and 5-Iododeoxyuridine (IUdR), caffeine, and hypoxic cell sensitizers such as isometronidazole.

In another embodiment, the therapeutic agent in a radioprotectant or radioprotector, which protects normal cells, such as non-tumor cells from any damage caused by radiation therapy of tumor cells. Examples of radioprotectants include without limitation amifostine (Ethyol®).

In some embodiments, the therapeutically active compound is an anti-inflammatory drug (either steroidal or non-steroidal).

In one embodiment, the therapeutically active compound is involved in angiogenesis. Suitable moieties include, but are not limited to, endostatin, angiostatin, interferons, platelet factor 4 (PF4), thrombospondin, transforming growth factor beta, tissue inhibitors of metalloproteinase-1, -2 and -3 (TIMP-1, -2 and -3), TNP-470, Marimastat, Neovastat, BMS-275291, COL-3, AG3340, Thalidomide, Squalamine, Combrestastatin, SU5416, SU6668, IFN-α, EMD121974, CAI, IL-12 abnd IM862.

In addition, the material may be any number of organic species, including but not limited to organic molecules and salts thereof, as well as biomolecules, including, but not limited to, proteins, nucleic acids, lipids, carbohydrates, and small molecule materials, such as drugs, specifically including hormones, cytokines, antibodies, cellular membrane antigens and receptors (neural, hormonal, nutrient, and cell surface receptors) or their ligands, etc). The present invention finds particular use in the delivery of therapeutic moieties to organisms, including tissues and cells; for example, the shell component of the nanoparticle can serve as a type of “controlled release” delivery system. As will be appreciated by those in the art, any number of suitable drugs such as those found in the Physician's Desk Reference can be used. In addition, as further described below, the moieties defined below as suitable agents may also serve as “targeting moieties” when attached to the surface of the shell and/or nanoparticle.

2. Nucleic Acids

In one embodiment, the therapeutically active agent is a nucleic acid, for example to do gene therapy or antisense therapy. By “nucleic acid” or “oligonucleotide” or grammatical equivalents herein means at least two nucleotides covalently linked together. A nucleic acid of the present invention will generally contain phosphodiester bonds, although in some cases, for example when therapeutic antisense molecules are to be included in the nanoparticle core, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of labels, or to increase the stability and half-life of such molecules in physiological environments.

In one embodiment, the nucleic acids suitable as agents are short interfering nucleic acid (siNA) molecules that act by invoking RNA interference. RNA interference mechanisms recognize RNA as “foreign” due to its existence in a double-stranded form. This results in the degradation of the double-stranded RNA, along with single-stranded RNA having the same sequence. Short interfering RNAs, or “siRNAs”, are an intermediate in the RNAi process in which the long double-stranded RNA has been cut up into short (−21 nucleotides) double-stranded RNA. The siRNA stimulates the cellular machinery to cut up other single-stranded RNA having the same sequence as the siRNA.

In some embodiments, the siNAs are siRNAs; in others, nucleotide analogs can be used. See the extensive discussion in US publication 2006/0160757, hereby incorporated by reference in its entirety, with particular reference to suitable chemically modified nucleosides, and the use of “blunt” and/or “overhang” sequences. In some embodiments, the siNAs are directed to a portion of the transmembrane domain (e.g. the 1^{st}, 2^{nd}, 3^{rd}, 4^{th}, 5^{th}, 6^{th} or 7^{th} transmembrane spanning region), a portion of the extracellular domain, a portion of the cytoplasmic domain, or any junction thereof. A siNA of the invention can be unmodified or chemically-modified. A siNA of the instant invention can be chemically synthesized, expressed from a vector or enzymatically synthesized.

In one embodiment of the invention a siNA molecule comprises an antisense strand having about 19 to about 29 (e.g., about 18, 19, 20, 21, 22, 23, 24, 25, 26, 27, 28, 29 or 30) nucleotides, wherein the antisense strand is complementary to a RNA sequence encoding a DC-STAMP protein, and wherein said siNA further comprises a sense strand having about 19 to about 29 (e.g., about 18, 19, 20, 21, 22, 23, 24, 25, 26, 27, 28, 29 or 30) nucleotides, and wherein said sense strand and said antisense strand are distinct nucleotide sequences with at least about 19 complementary nucleotides (these are referred to as double stranded siNAs, or dssiNAs).

In one embodiment, the proteins cages of the present invention include one or more siNAs that are antisense nucleic acids (generally described in US publication 2006/0172957, hereby incorporated by reference in its entirety, and with particular reference to suitable chemically modified nucleosides and nucleic acids for use in antisense technologies and mechanisms) that serve to inhibit the activity of a physiological target as described herein. Antisense mechanisms are processes in which the antisense compound specifically hybridizes to it's target RNA to form a duplex. The formation of this duplex prevents the RNA from functioning normally and from producing a protein product. In general, antisense molecules can be from 5 to 100 basepairs in length, with from about 8 to about 50 bases being preferred.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, depending on its ultimate use, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

In another embodiment, the nucleic acid may be single-stranded or double stranded. The physiological target molecule can be a substantially complementary nucleic acid or a nucleic acid binding moiety, such as a protein.

In another embodiment, the protein cages having a nucleic acid as the therapeutic agent are suitable for use as vaccines, as further discussed below.

3. Proteins

In one aspect, the therapeutically active agent is a protein. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In one embodiment, the amino acids are in the (S) or L-configuration.

In another embodiment, the protein is an antibody. The term “antibody” includes monoclonal antibodies, polyclonal antibodies, and antibody fragments thereof. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et al., 2000, Methods Enzymol. 326:461-479; WO94/13804; Holliger et al., 1993, Proc. Natl. Acad. Sci. U.S.A. 90:6444-6448). The antibody fragments may be modified. For example, the molecules may be stabilized by the incorporation of disulphide bridges linking the VH and VL domains (Reiter et al., 1996, Nature Biotech. 14:1239-1245). Antibodies may also include chimeric antibodies, either produced by the modification of whole antibodies or those synthesized de novo using recombinant DNA technologies. Those of ordinary skill in the art will appreciate the antibodies suitable for use with the present invention. In one embodiment, the protein cages of the present invention include an antibody fragment, that is, that is a fragment of any of the antibodies outlined herein that retain binding specificity to an antigen.

In one embodiment, the protein included with a protein cage is a monoclonal antibody. Suitable monoclonal antibodies and/or antibody fragments include without limitation, rituximab against the CD20 antigen on B cells (Rituxin®), trastuzumab against the HER2 protein (Herceptin®), alemtuzumab against the CD52 antigen present on both B cells and T cells (Campath®), cetuximab against the EGFR protein (Erbitux®), bevacizumab against the VEGF protein (Avastin®), panitumumab against EGFR (Vectibix®), abciximab against the GP IIb/IIIa receptor (ReoPro®), infliximab against TNF-alpha (Remicade®), adalimumab against TNF-alpha (Humira®)), eculizumab against the complement protein C5 (Alexion®), omalizumab against human immunoglobulin E (Xolair®), efalizumab against CD11a (Raptiva®), ranibizumab against VEGF (Lucentis®), palivizumab against the F protein of RSV (Synagis®), muromonab against CD3 (Orthocline Okt3®), edrecolomab against the 17-IA antigen (Panorex®), basiliximab against CD-25 (Simulect®), daclizumab against the IL-2 receptor (Zenapax®), natalizumab against alpha-4-integrin (Antegren®), CDP-571 against TNF-alpha (Humicade®), epratuzumab against CD-22 (Lymphocide®), oregovomab against CA125 (OvaRex®), visilizumab against CD3 (Nuvion®), volociximab against alpha-5-beta-1 integrin (M200), sevirumab against cytomegalovirus (CMV) (Protovir®), and an efalizumab against the LFA1 receptor (Raptiva®).

In another aspect, the therapeutic agent is a protein that includes a label. In one embodiment, the labeled protein is a labeled antibody. The label may be a radioisotope and/or another protein such as an enzyme.

The labeled antibody may be an antibody labeled with an isotope. By “isotope” is meant atoms with the same number of protons and hence of the same element but with different numbers of neutrons (e.g., ¹H vs. ²H or D). The term “isotope” includes “stable isotopes”, e.g. non-radioactive isotopes, as well as “radioactive isotopes”, e.g. those that decay over time. In one embodiment, the protein cages of the present invention include a monoclonal antibodies labeled with a radioisotope, which are useful in radioimmunotherapy. Suitable radioisotopes include without limitation an alpha-emitter, a beta-emitter, and an Auger electron-emitter (Behrt, T. et al., (2000). Eur. J. Nuclear Med. vol. 27 (7):753-765; Vallabhajosula, S. et al., J. Nucl. Med. (2005) April; 46(4):634-41). Such radioisotopes include without limitation [65]Zinc, [140]neodymium, [177]lutetium, [179]lutetium, [176m]lutetium, [67]gallium, [159]gallium, [161]terbium, [153]samarium, [169]erbium, [175]ytterbium, [161]holmium, [166]holmium, [167]thulium, [142]praseodymium, [143]praseodymium, [145]praseodymium, [149]promethium, [150]europium, [165]dysprosium, [111]indium, [131]iodine, [125]iodine, [123]iodine, [88]yttrium and [90]yttrium.

As appreciated by those of skill in the art, a number of radiolabeled antibodies may be suitable for use in the present invention. In one embodiment, the radiolabeled antibody may be a monoclonal antibody. Suitable radiolabeled monoclonal antibodies and/or antibody fragments include, without limitation, 90-yttrium-ibritumomab tiuxetan (Zevalin®), iodine-131 tosiumomab (Bexxar®) ((Lewington V. (2005). Semin. Oncol. February; 32(1 Suppl 1):S36-43), 131-iodine Lym-1 (Oncolym®), and yttrium-90-pemtumomab.

In other embodiments, the labeled antibody may be an antibody labeled with a small molecule. The small molecule may be a cytotoxic cancer drug. Suitable drug-labeled antibodies include without limitation gemtuzumab against CD33 (Mylotarg®) and CYT-500 which uses the same antibody from Prostascint® (described below) but includes a therapeutic instead of a radioisotope.

In some other embodiments, the protein cage includes a targeting moiety on the exterior of the cage and a therapeutic agent on the interior of the cage. For example, the protein cage may include an antibody as the targeting moiety and a drug, isotope, or isotope labeled protein on the interior as the therapeutica agent.

In some embodiments, therapeutic agent and targeting moiety can be the same. In another embodiment, laminin peptide 11 may be attached to a protein cage and utilized as both a therapeutic agent and a targeting moiety. Laminin peptide 11 (CDPGYIGSR-NH2), is a segment of laminin which blocks tumor cell invasion. A high affinity laminin receptor in tumor cells is thought to be blocked by the carboxyl-terminal YIGSR (See Ostheimer, G. J. et al., (1992) J. Biol. Chem. December 15; 267(35):25120-8).

In another aspect of the present invention, the protein is a peptide. In one embodiment the peptides have a label. In other embodiments, the peptides have an isotope label, such as a radioisotope as described herein. The peptide may be a radiolabeled Arg-Gly-Asp (RGD) peptide, which binds alpha(v)beta(3) integrin and is known to be useful in targeting tumor cells. The RGD peptide may be labeled with [111]In and/or [99m]technetium (Janssen, M. L. et al., (2002) Cancer Res. November 1; 62(21):6146-51). A protein cage having a radiolabeled peptide may also include an imaging agent, such as a chelate-paramagnetic metal ion, as described herein. In another embodiment, the radiolabeled peptide is a peptide with affinity for the gastrin releasing peptide receptor (GRPR, also known as BB2), which is overexpressed in certain tumors. The peptide may be bombesin and/or derivatives thereof labeled with a rhenium radioisotope (Moustapha, M. E. et al., (2006). Nucl. Med. Biol. 2006 January; 33(1):81-9). Those skilled in the art will appreciate the number of radiolabeled peptides suitable for use with the present invention, including without limitation, indium-111-pentetreotide, indium-111-DTPA-octreotide (Octreoscan), which binds to somatostatin receptors (SSTRs), echnetium-99m-depreotide (NeoTect), a 99mTc-labeled SSTR-analog, Yttrium-90-DOTA-Phe1-Tyr3-octreotide, yttrium-90-DOTA-lanreotide, Lutetium-177-DOTA-octreotate, rhenium-188-P2045, yttrium-90-alpha(v)beta3 antagonist, and peptides with affinity for the bombesin receptor, alpha-melanocyte-stimulating hormone receptor, neurotensin receptor, and the integrin alpha(v)beta3. (see Weiner R. E. et al., (2005) BioDrugs. 19(3):145-63).

In another embodiment, the therapeutically active compound is a peptide used to treat cancer. The peptide may be the laminin peptide 11 (see above).

In one other embodiment, the protein is a subunit vaccine as described in Section II.A.8. Vaccine agents.

In one embodiment, the protein cage includes an agent derived from the ADEPT concept (See Duncan et al., U.S. 6372205, incorporated herein by reference in its entirety). In ADEPT, an antibody is linked to an enzyme that can metabolize a substrate in vivo, which is not normally metabolized by the subject. The substrate is typically an inactive prodrug. In some embodiments, the antibody is chemically linked to enzyme, such as. It will be appreciated by those of ordinary skill that several enzymes are suitable for use in the present invention, including without limitation, carboxypeptidase G2, penicillin amidase, beta-lactamase, beta-glucuronidase, cytosine deaminase, nitroreductase and alkaline phosphatase. In one embodiment, a protein cage includes an antibody that can bind a tumor-specific antigen and an enzyme suitable for metabolizing a substrate.

In some embodiments, the invention provides delivery agents comprising catalytic centers. That is, either in addition to or instead of the imaging agents of the invention, the cages of the invention include a catalytic center that delivers an activity to the cell or tissue that is then used to generate a desirable result. For example, enzymes, including enzyme mimics, can be delivered in this way. One example of an enzyme mimic is a complex of copper bound to phenanthroline, which acts as a non-specific hydrolase of nucleic acids; thus it may be used to hydrolyze exogeneous nucleic acid in a cell, for example in the case of viral infection (see Sigman, D. S. 1986, Acc. Chem. Res. 19:180-186; and Davies, R. R. and Distefano, M. D., 1997, J. Am. Chem. Soc. 119:11643-11652). Similarly, any of the enzymatic activities outlined above can be delivered as well, for any number of purposes. Furthermore, metal-based catalysts are used in a wide variety of contexts that can be included in the delivery agents of the invention, for example to turn prodrugs into drugs.

4. Radioisotopes as Agents

In one aspect, the present invention provides protein cages where the therapeutic agent is an isotope as described herein. In one embodiment, the isotope is a radioisotope located within the protein cage using one of the modifications described in section III.

5. Carbohydrate and Lipid Therapeutic Agents

In one embodiment, the therapeutic agent is a carbohydrate. By “carbohydrate” herein is meant a compound with the general formula Cx(H₂O)y. Monosaccharides, disaccharides, and oligo- or polysaccharides are all included within the definition and comprise polymers of various sugar molecules linked via glycosidic linkages. Suitable carbohydrates (particularly in the case of targeting moieties, described below) are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates.

In one embodiment, the therapeutic agent is poly-L-lysine. For example, the epsilon-poly-lysine has been shown to have antimicrobial activity (see Shima, S. et al. (1984) J. Antibiot (Tokyo) November; 37(11):1449-55 incorporated herein by reference in its entirety). In one other embodiment, the protein cages of the present invention include poly-L-lysine as a therapeutic agent. Poly-L-lysine may be attached to the exterior and/or interior surface of the protein cage as described herein.

In one other embodiment, the therapeutic agent is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

6. Inorganic Material Agents

In a one embodiment, the present invention provides a range of inorganic materials that can be synthesized within the protein cage architectures, many of which have biomedical applications. The syntheses are based on exploiting electrostatic interactions at the protein interface. The inorganic materials synthesized include without limitation: Fe₂O₃, Fe₃O₄, Mn₂O₃, Co₃O₄, CO₂O₃, TiO₂₋ₓ(OH)_{x, Eu}₂O₃, ZnSe, ZnS, and metallic particles such as Pt, Au, FePt and CoPt (Allen, M., et al. 2002. Advanced Materials 14:1562−+), (Allen, M., et al. 2003. Inorg. Chem. 42:6300-6305; Douglas, T. 1996. In S. Mann (ed.), Biomimetic Materials Chemistry. VCH Publishers, New York; Douglas, T., et al. 1995. In J. E. Mark and P. Bianconi (ed.), Hybrid Organic-inorganic Composites. American Chemical Society, Washington, D.C.; Douglas, T., et al. 1995. Science 269:54-57; Douglas, T., et al. 1996. VCH publishers, New York; Douglas, T., et al. 2000. Inorg. Chem. 39:1828-1830; Douglas, T., et al. 2002. Advanced Materials 14:415−+; Douglas, T., et al. 1998. Nature 393:152-155; Douglas, T., et al. 1999. Advanced Materials 11:679−+), (Ensign, D., et al. 2004. Inorganic Chemistry 43:3441-3446), (Gider, S., et al. 1995. Science 268:77-80), (Gilmore, K., et al. Journal of Applied Physics), (Klem, M. T., et al. 2005. Adv. Funct. Mater. submitted), (Klem, M. T., et al. 2003. J. Am. Chem. Soc. 125:1056-1057), (Liepold, L., et al. 2005. in preparation), (Meldrum, F. C., et al. 1995. Journal of Inorganic Biochemistry 58:59-68), (Mosolf, J., et al. 2004. in preparation), (Shenton, W., et al. 1999. Advanced Materials 11:253−+), (Varpness, Z., et al. 2004. in preparation). These inorganic material agents may be used in various applications.

In one aspect, the inorganic material agents can serve as agents for hyperthermia applications. In one embodiment, the present invention provides protein cages with inorganic materials as magnetic nanoparticles for use as a hyperthermia treatment agent. These hyperthermia treatment agents may also be referred to as “thermal ablation agents.” In one embodiment, the magnetic nanoparticles are Fe₃O₄ particles within the CCMV and sHsp cages made using controlled oxidation of Fe²⁺, to obtain the protein encapsulated materials. It has been shown that the increase in local temperature caused by magnetic hyperthermia is the result of specific loss power (SLP) due to hysteresis, relaxational losses, and resonance losses (Hergt, R., R. et al., 2004. Journal of Magnetism and Magnetic Materials 270:345-357). SLP is a function of particle size (Hergt, R., R. et al., 2004. Journal of Magnetism and Magnetic Materials 280:358-368) and while 7 nm particles of maghemite can adequately heat a tissue sample, 20 nm maghemite particles, such as those synthesized inside CCMV protein cage, have been shown to be optimal. In addition, through introduction of dopants such as Zn²⁺ the heating capacity of the particles can be tuned to minimize damage to healthy tissue (Giri, J., A. et al., 2003. Bio-Medical Materials and Engineering 13:387-399). These materials may be synthesized by varying the ratio of Fe²⁺ to Zn²⁺ in the synthesis reaction to form the optimal composition. The synthesized materials may be characterized by TEM, electron diffraction, dynamic light scattering, and magnetometry.

In another aspect, the present invention provides protein encapsulated inorganic material agents as magnetic nanoparticles for use in targeted hyperthermia. This application may also be referred to as “thermal ablation.” Magnetic nano-particles may be used for the controlled heating of tissues and/or cells to induce cell death. In combination with selective tissue targeting this approach may be utilized as a therapy for cancer treatment. Using a biomimetic encapsulation approach, control of magnetic nano-particle synthesis within both the CCMV and sHsp protein cages is achieved. The protein cages offer the advantage of constrained particle synthesis (size and shape) and a platform for presentation of cell specific ligands for tissue targeting. In addition, the magnetic nano-particles encapsulated within the protein container can be combined with drug encapsulation-delivery and diagnostic imaging (MRI) to provide a synergistic multifaceted treatment strategy.

In one aspect, the present invention provides protein cages as a platform for synthesis of high magnetic moment materials that are biocompatible. In particular, well-defined highly magnetic materials may be formed.

In one embodiment, the present invention provides a protein cage where the inorganic material agent is a platinate suitable for use as a thermal ablation agent. Suitable platinates include without limitation metal platinates. In one embodiment, a protein cage includes a metal platinate such as an iron platinate or a cobalt platinate. The metal platinate may be provided with a protein cage through the use of a peptide on the interior surface of the cage. The peptides may be specific for a particular inorganic material onto the interior of the protein cage. In one embodiment, the protein cages of the present invention include a peptide specific for the L1₀ phases of CoPt, such as a peptide having the amino acid sequence KTHEIHSPLLHK, on the interior surface of the cage. The protein cage may be a sHsp cage or a CCMV cage. It is known that specific nucleation of CoPt and size contrained particle growth may be achieved. The resulting monodisperse CoPt particles (6±0.8 nm) in a M. Jannaschii protein cage show ferromagnetic behavior, and high saturation moments even prior to thermal annealing (Klem et al. (2005). Adv. Funct. Mater. 15, 1489-94). In another embodiment, the protein cages as described herein may include a peptide specific for FePt, such as a peptide having the amino acid sequence HNKHLPSTQPLA (Mao, C. B. et al. (2004) Science. 303:213-217), where both components of the alloy (Fe and Pt) exhibit very limited cyto-toxicity. It will be appreciated by those skilled in the art that a number of peptides are suitable for use with the protein cages (Seeman, N. C. et al. (2002) Proc. Nat'l Acad. Sci. 99:6451-6455; Whaley, S. R. et al. (2000) Nature, 405:665-668).

In some embodiments, protein cages containing thermal ablation agents may also include targeting moieties as described herein. A protein cage of the present invention may include an both an antibody as a targeting moiety and an inorganic material, such as an iron-oxide. In one embodiment, the antibody may be against a cancer cell membrane antigen (see DeNardo, S. J. et al. (2005) Clinical Cancer Res. 11; 7087s-7092s). In another embodiment, once the protein cage having an inorganic material (with or without a targeting moieity) has been administered, an externally applied alternating magnetic field (AMF) may be applied to inductively heat the protein cage and thereby provide thermal ablation therapy (see id.). The thermal ablation may be directed towards cancer cells that have been targeted by the protein cage by a targeting moiety as discussed herein.

Tissue may be targeted for thermal ablation in this manner. In one embodiment, the protein cage is targeted to a tumor cell using a protein and tumor cells are thermally ablated by hyperthermia. Irradiation of protein cages containing thermal ablation agents with an AC field may be performed to induce hyperthermia.

In early developments of hyperthermia, magnetic particles were injected into a patient and guided to a target site with an external magnetic field. Alternatively, targeted cellular delivery can be achieved through the incorporation of cell-specific recognition molecules, such as peptides, expressed on the exterior surface of the protein cage nano-particles. Characterization of the magnetic properties of previously synthesized materials make the protein cages of the present invention suitable for applications in targeted hyperthermia for tumor necrosis (Jordan, A. R. et al. (1997) International Journal of Hyperthermia 13:587-605).

In one embodiment, magnetic nano-particles are used in the controlled heating of tissue to induce cell death. In another embodiment, magnetic nano-particles are selective for a target tissue. In one embodiment, the target tissue is a cancerous tissue.

In one embodiment, Fe₃O₄ particles may be synthesized within a protein cage, such as the CCMV and/or sHsp cages using controlled oxidation of Fe²⁺ to obtain the protein encapsulated materials by methods described herein. The increase in local temperature caused by magnetic hyperthermia is the result of specific loss power (SLP) due to hysteresis, relaxational losses, and resonance losses (Hergt, T. et al. (2004) Journal of Magnetism and Magnetic Materials 270:345-357) and SLP is a function of particle size (Hergt, R. et al. (2004) Journal of Magnetism and Magnetic Materials 280:358-368).

In some embodiments, the particle size suitable for adequate heating of a tissue sample is in the range of about 7 nm to about 24 nm, about 10 nm to about 20 nm, and about 12 nm to about 17 nm. In other embodiments, the particle size suitable for adequate heating of a tissue sample is about 7 nm, about 12 nm, about 20 nm or about 24 nm.

In addition, through introduction of dopants such as Zn²⁺ the heating capacity of the particles can be tuned to minimize damage to healthy tissue (Giri, J. et al. (2003) Bio-Medical Materials and Engineering 13:387-399). These materials will be synthesized by varying the ratio of Fe²⁺ to Zn²⁺ in the synthesis reaction to form the optimal composition. The synthesized materials will all be characterized by TEM, electron diffraction, dynamic light scattering, and magnetometry.

In another embodiment, peptides for a particular inorganic material may be genetically engineered onto the interior surface of a protein cage. These peptides include those known in the art as previously described above (see Seeman (2002) PNAS supra; Whaley (2000) Nature supra)

In one aspect, the protein cages of the present invention include gold particles. Those of skill in the art will appreciate suitable methods for attaching gold to the protein cages, including the use of reagents such as Nanoprobes' gold labeling reagent Nanogold®. It has previously been shown that gold coated nanoparticles can absorb near infrared (NIR) light (about 700 nm to about 1100 nm) and therefore may be suitable for thermal ablative applications. For example, the selective induction of photothermal destruction of tumor cells through the application of NIR light in the presence of nanoshells (such as silica shells) coated with gold particles has been demonstrated (see Loo, C. et al., (2004) Technology in Cancer Research & Treatment. Feb. 3(1); 33-40; Hirsch, L. R. (2006) Annals Biomed. Engineer. 34(1); 15-22; O'Neal, D. P. et al. (2004) Cancer Lett. 209:171-176; Paciotti, G. F. et al. (2006) Drug Development Res. 67:47-54). In addition, gold-labeled protein cages may be suitable for imaging as discussed below. In another embodiment, protein cages with gold particles may allow for optical imaging as well as thermal ablation.

In one embodiment, the NIR light suitable for use with a protein cage having a thermal ablation agent described herein is from about 700 nm to about 1100 nm, from about 750 nm to about 1050 nm, from about 800 nm to about 1000 nm, from about 850 nm to about 950 nm, and about 900 nm. In addition, the NIR light may be about 700 nm, about 750 nm, about 800 nm, about 850 nm, about 900 nm, about 950 nm, about 1000 nm, about 1050 nm, and about 100 nm.

In one embodiment, the approach may be used for the synthesis of biocompatible, high magnetic moment materials. For example, well-defined highly magnetic materials such as of FePt (HNKHLPSTQPLA) (see Mao (2004) Science supra) where both components of the alloy (Fe and Pt) exhibit very limited cyto-toxicity may be appropriate biocompatible materials. These magnetic particles have application in the assessment of in vivo distribution and deposition of targeted protein cages, using MRI, as a critical step in their development as clinically relevant agents.

The magnetic characterization of these materials can be achieved through the study of the magnetic susceptibility, specific heat and power dissipation to optimize hyperthermia performance. There are three complimentary approaches to measuring the power loss of protein encapsulated ferro(i)magnetic nanoparticles: alternating-current magnetic susceptibility (ACMS), electron paramagnetic resonance (EPR), and calorimetry in an RF field. Lyophilized samples may be studied by ACMS at frequencies varying from 10-10000 Hz under applied fields and solution based samples will be measured by EPR at 4.0, 9.8, 35.5, and 94.4 GHz at variable temperatures. These methods are limited by the size of the nano-particle and the type of magnetism displayed by the protein encapsulated nano-particles. Time dependent calorimetry studies in an applied RF field will also be used to provide a consistent comparison of the specific power loss across a range of particle sizes and types (Hiergeist, R. et al. (1999) Journal of Magnetism and Magnetic Materials 201:420-422).

In another embodiment, targeting peptides may be covalently attached to the magnetic nano-particle cages as described herein The in vitro evaluation of the hyperthermia induced by the cage encapsulated magnetic nano-particles can be performed using magnetic protein cages embedded in 0.5% agarose (to mimic tissue viscosity). Targeted cages will be evaluated in cell culture by incubation of the magnetic cages (with and without targeting ligands) with cells (see section IV. Targeting Moieties), followed by extensive washing to remove unbound cages and irradiation with an AC-field to induce hyperthermia.

In other embodiments, targeted magnetic cages exhibit effective tumor necrosis under AC irradiation in cell culture. The magnetic properties of the synthesized magnetic materials have application in hyperthermia and the targeting provide the required cell/tissue specificity. In addition, themagnetic nano-particles have enormous potential as MRI contrast agents.

In one embodiment, the present invention provides a biomimetic encapsulation method with precise control of magnetic nano-particle synthesis within a protein cage. In another embodiment, the protein cages include without limitation the CCMV and sHsp protein cages. In one other embodiment, magnetic nano-particles encapsulated within the protein cages are combined with drug encapsulation-delivery and diagnostic imaging (MRI) to provide a synergistic multifaceted treatment strategy. In early developments of hyperthermia, magnetic particles may be injected into a patient and guided to a target site with an external magnetic field. Alternatively, targeted cellular delivery can be achieved through the incorporation of cell-specific recognition molecules such as for example the peptides described herein (section IV. Targeting Moieties), expressed on the exterior surface of the protein cage nano-particles. One embodiment of the present invention provides compositions for targeted hyperthermia (Jordan, A., R. et al., 1997. International Journal of Hyperthermia 13:587-605) for tumor necrosis.

7. Agents for Photodynamic Therapy (PDT)

In one other aspect, the protein cages of the present invention may be used in photodynamic therapy (PDT). PDT is a therapeutic treatment that utilizes a drug, usually a photosensitizer or photosensitizing agent and a particular type of light. (Dougherty, T. J. et al., J. Nat'l Cancer Inst. (1998) Jun. 17; 90(12):889-905.) Upon exposure to a specific wavelength of light, certain photosensitizers produce a form of oxygen that is cytotoxic to cells in the area of treatment. A given photosensitizer is activated by light of a particular wavelength, which determines how far the light can travel through tissue. Different photosensitizers are therefore suitable for the application of PDT in different areas of the body. PDT is typically performed by administering a photosensitizer to a patient in need followed by exposure of the treated area to light capable of exciting the photosensitizer. In the presence of molecular oxygen, an energy transfer occurs resulting in production of the highly cytotoxic singlet oxygen (¹O₂), which is a very aggressive chemical species capable of reacting with biomolecules in its vicinity. PDT is known to be effective as a cancer treatment in multiple ways, including without limitation killing tumor cells directly, damaging blood vessels in a tumor and activation of the immune system to destroy the tumor cells.

By a “photosensitizing agent” or “photosensitizer” is meant a chemical compound that binds to one or more types of selected target cells and, when exposed to light of an appropriate waveband, absorbs the light, causing substances to be produced that impair or destroy the target cells. Virtually any chemical compound that is absorbed or bound to a selected target and absorbs light causing the desired therapy to be effected may be used in the protein cages of the present invention. As will be appreciated by those of ordinary skill in the art, many different photosensitizers are suitable for use in the present invention. A comprehensive listing of photosensitive chemicals may be found in Kreimer-Bimbaum, Sem. Hematol, (1989) 26:157-73.) Photosensitive agents or compounds include, but are not limited to, chlorins, bacteriochlorins, phthalocyanines, porphyrins, purpurins, merocyanines, psoralens, benzoporphyrin derivatives (BPD), and porfimer sodium (Photofrin®) and pro-drugs such as delta-aminolevulinic acid, which can produce photosensitive agents such as protoporphyrin IX. Other suitable photosensitive compounds include ICG, methylene blue, toluidine blue, texaphyrins, and any other agent that absorbs light in a range of about 500 nm to about 1100 nm, about 550 nm to about 1050 nm, about 600 nm to about 1000 nm, about 650 nm to about 950 nm, about 700 nm to about 900 nm, about 750 nm to about 850 nm, and about 800 nm.

In one embodiment, the photosensitizing agent also can be conjugated to specific ligands known to be reactive with a target tissue, cell, or composition, such as receptor-specific ligands or immunoglobulins or immunospecific portions of immunoglobulins, permitting them to be more concentrated in a desired target cell or microorganism than in non-target tissue or cells. The photosensitizing agent may be further conjugated to a ligand-receptor binding pair. Examples of a suitable binding pair include but are not limited to: biotin-streptavidin, chemokine-chemokine receptor, growth factor-growth factor receptor, and antigen-antibody. (See Chen, J. U.S. Pat. No. 6,602,274, incorporated herein by reference in its entirety).

In one embodiment, the present invention provides a protein cage that includes a PDT agent. The PDT agent may be a photosensitizer. The protein cage may be a cage having cysteine residues suitable for reaction with a photosensitizer in order to attach the photosensitizer to the cage. The photosensitizer may be attached to an internal surface and/or an external surface of the cage. In one embodiment, the protein cage may be a small heat shock protein (sHsp) as described herein modified to incorporate cysteine residues on the internal and/or external surface of the cage and the photosensitizer may be ruthenium(II)bpy₃.

The light required for PDT can be provided by a number of sources known to those of skill in the art, including without limitation a laser and light-emitting diodes (LEDS) (Vrouenraets, M. B. et al., Anticancer Research (2003); 23:505-522; Dickson, E. F. G. et al., Cell. & Molec. Biol. (2003); 48(8):939-954).

In another embodiment, the photosensitizing agent is activated by red light. Such photosensitizing agents may be conjugates. For example, the conjugate may be poly-L-lysine and chlorine(e6) conjugate or a polyethyleneimine (PEI) and chlorine(e6) conjugate as described in Tegos, G. P. et al. (2006) Antimicrob. Agents Chemother. April; 50(4):1402-10, which is incorporated herein by reference in its entirety.

In one aspect, the present invention provides a protein cage having Ru^{∥}bpy₃ as the PDT agent. The protein cage may contain the small heat shock protein (Hsp) from M. jannaschii, as described herein. It will be appreciated by those of skill in the art that Ru^{∥}bpy₃ is a well-characterized photo catalyst for ¹O₂ formation. In one embodiment, Ru^{∥}bpy₃ may be attached to a protein subunit of a M. jannaschii Hsp protein cage as a PDT agent (See Example 8).

8. Vaccine Agents

In one aspect, the protein cages of the present invention may be used as vaccines. In one embodiment, a patient is immunized with protein cages having a vaccine agent. For example, ferritin fusion proteins have potential use as vaccines (See et al. U.S. Pat. No. 7,097,841, incorporated herein by reference in its entirety). In one embodiment, the protein cages of the present invention may have vaccine agents, including but not limited to inactivated vaccines, live vaccines, toxoid vaccines, protein subunit vaccines, polysaccharide vaccines, conjugate vaccines, recombinant vaccines, nucleic acid vaccines, and synthetic vaccines.

In one embodiment, protein cages include an inactivated vaccine agent, which may be a previously virulent micro-organism that has been killed by chemical treatment or heat. Suitable inactivated vaccines include without limitation anthrax, Japanese encephalitis, rabies, polio, diphtheria, tetanus, acellular Pertussis vaccine influenza, cholera, bubonic plague, chicken pox, hepatitis A, Haemophilus influenzae type b, and any combinations thereof.

In another embodiment, the protein cages include an attenuated vaccine agent, which may be a live microorganism cultivated under attenuating conditions which have rendered them non-virulent. Suitable inactivated vaccines include without limitation vaccines for chicken pox, yellow fever, measles, rubella, mumps, typhoid, and combinations thereof.

In one other embodiment, the protein cages include a toxoid vaccine agent, which may be a toxic compound produced a microorganism that has been rendered non-toxic. Suitable toxoid vaccines include without limitation vaccines for tetanus, diphtheria, and pertussis. The tetanus vaccine is derived from the toxin called tentanospasmin produced by Clostridium tetani.

In another embodiment, the protein cages include a subunit vaccine agent, which may be a purified antigenic determinant separate from a pathogen. For example, the subunit of the protein coat of a virus, such as the hepatitis B virus. Generally, viral subunit vaccines are free of viral nucleic acids.

In other embodiments, the protein cages include a polysaccharide vaccine agent. Certain bacteria are encapsulated by polysaccharides. Vaccines have been derived from purified forms of the bacterial outer polysaccharide coat. In particular, vaccines for meningitis have been developed using this approach. For example, the purified polyribosylribitol phosphate (PRP) polysaccharide from the capsule of Haemophilus influenzae type b (Hib) has been purified and used as a vaccine (PRP vaccine). The polysaccharide vaccine, PPV23 (Pneumovax 23), contains 23 antigenically distinct polysaccharides found on the surface capsules of Streptococcus pneumoniae. In one embodiment, polyvalent polysaccharide vaccine agents may be suitable for use with the protein cages described herein. For example, a quadrivalent polysaccharide vaccine, Menomune, is used as a vaccine for meningitis caused by Neisseria meningitides. Meningococcal meningitis is caused by bacteria of five different serogroups: A, B, C, w135, and Y. Menomune targets the capsular antigens on groups A, C, w135, and Y. In addition, a typhoid Vi polysaccharide vaccine has been developed (Typhim Vi®), which includes the cell surface Vi polysaccharide extracted from Salmonella typhi Ty2 strain.

In some embodiments, the protein cages include a conjugate vaccine agent, which is typically an antigenic portion and a polysaccharide portion. These conjugate vaccine agents may also be referred to as polysaccharide conjugate vaccine agents. As described above, bacteria utilize certain polysaccharides as protective coating but these may be difficult for an immune system to recognize and respond to. This is particularly true for infants and children under the age of two, who have immature immune systems that rely on antibodies that are maternally passed down before birth. As such, they are unable to respond to bacteria such as those causing meningitis. The conjugate vaccine may be a toxoid conjugated to a bacteria polysaccharide, which allows the immune system to better react to the polysaccharide. Suitable conjugate vaccines agents include without limitation those vaccines developed to prevent meningitis. For example the PRP polysaccharide of Hib has been used to develop conjugate vaccine agents (Heath PT. Pediatr Infect Dis J 1998; 17:S117-S122). It has been linked to diphtheria toxoid (PRP-D), a diphtheria-like protein (PRP-HbOC), a tetanus-toxoid (PRP-T), or a meningococcal outer membrane protein (PRP-OMP). Conjugage vaccine agents for pneumococcal meningitis caused by Streptococcus pneumoniae have also been developed, such as PCV7 (Prevnar) which contains seven different polysaccharides from seven strains of the bacteria known to cause the disease. In PCV7, each polysaccharide is coupled to CRM197, a nontoxic diphtheria protein analogue. For meningitis caused by Neisseria meningitidis, a number of conjugage vaccine agents have been developed, including a polysaccharide (A/C/Y/W-135) diphtheria conjugate vaccine (Menactra) and a monovalent serogroup C glycoconjugate vaccine (MenC).

In one embodiment, the present invention provides protein cages having polysaccharides attached to them. The polysaccharide attached to the protein cage may be part of a polysaccharide vaccine agent and/or part of a polysaccharide conjugate vaccine agent attached according the methods described herein (Section III. A. 3. Modification of glycosylation patterns).

In additional embodiments, the protein cages include a nucleic acid vaccine. The nucleic acid vaccine may be a DNA vaccine, which may be single genes or combinations of genes. Naked DNA vaccines are generally known in the art. (Brower, Nature Biotechnology, 16:1304-1305 (1998)). Methods for the use of genes as DNA vaccines are well known to one of ordinary skill in the art, and include placing a a gene or a portion of a gene under the control of a promoter for expression in a patient in need of treatment. The gene used for DNA vaccines may encode a full-length protein or portions of a protein, including peptides derived from the full-length protein. In one embodiment a patient is immunized with a DNA vaccine comprising a plurality of nucleotide sequences derived from a gene. Suitable nucleic acid vaccines include without limitation vaccines for malaria, influenza, herpes and HIV.

In other embodiments, the protein cages have vaccine agents for HIV. The vaccine agents may be proteins attached to the protein cages as previously described herein. The proteins may be peptides and/or lipoproteins. Suitable protein HIV vaccine agents include without limitation a lipoprotein-based vaccine agent, such as LIP0-5 containing 5 lipopeptide epitopes from gag, nef, and pol (Sanofi Pasteur), a multi-epitope CTL peptide vaccine containing peptides from clade B Env, gag, nef (Wyeth), the AIDSVAX B/B containing a recombinant form of the protein gp120 having two HIV subtype antigens MN and GNE8 (VaxGen), the gp120 protein having the MN subtype antigen (VaxGen), the Clade C Env subunit (Chiron), the subtype gp120 W61D protein (GlaxoSmithKline), and the NefTat protein (GlaxoSmith Kline).

In additional embodiments, the protein cages of the present invention include one or more vaccine agents as described above. The vaccine agents may be attached to the protein cages by the methods described herein, such as for example by fusion protein methods where appropriate.

B. Imaging Agents

In other embodiments, protein cages comprise a plurality of medical imaging agents. The medical imaging agents may be the same or different. In one embodiment, the medical imaging agents are the same. In another embodiment, the medical imaging agents are different. That is, a protein cage may comprise an MRI agent and an optical agent, or and MRI agent and an ultrasound agent, or an NCT and an optical agent, etc.

Regardless of whether the protein cage comprises the same or different imaging agents, anywhere from 1 to up to up 180 imaging agents may be entrapped within a protein cage. In some embodiments, the imaging agents may be attached to polymers (described below) and under these conditions, from 10 to 1000 imaging agents may be entrapped in a protein cage.

In one aspect, the present invention provides proteins cages wherein the imaging agent is a protein. In one embodiment, radiolabeled proteins are part of a protein cage as described herein and used for detection. The proteins suitable for imaging may be antibodies, including fragments or portions of antibodies. In one embodiment, radiolabeled antibodies are suitable proteins for imaging. Such antibodies include without limitation indium-111 capromab pendetide against prostate-specific membrane antigen (PSMA) (ProstaScint®), indium-111 CYT-103 against the carcinoma antigen TAG-72 (OncoScint®), technetium-99m-arcitumomab against an anti-carcinoembryonic antigen (CEA) (CEA-Scan®), technetium-99m-sulesomab against NCA-90 in granulocytes (LeukoScan®), technetium-99m-IMMU-30 against serum tumor marker alpha-fetoprotein (AFP—SCAN®), technetium-99m-bectumomab against CD-22 (LymphoScan®), technetium-99m-nofetumomab merpentan (Verluma), technetium-99m fanolesomab against the cluster of differentiation 15 (CD15) antigen (NeutroSpec), and indium-111-imciromab pentetate against the heavy chain of myosin (Myoscint®). In such embodiments, the radiolabeled antibody serves as both a targeting moiety (antibody) as described herein and an imaging agent (radioisotope).

In another embodiment, the protein suitable for imaging is a peptide. As described herein, the peptide may be an RGD peptide.

In one aspect, the invention provides protein cages having a positron emission tomography (PET) imaging agent. It has been reported that an 18F-labeled RGD peptide may be used for PET imaging of alpha-v-beta 3 integrin expression. (see Cai, W. et al., (2006) J. Nucl. Med. July; 47(7):1172-80). In one embodiment, a protein cage of the present invention may include a radioisotope-labeled peptide suitable for use as a PET imaging agent. The labeled peptide may be RGD.

One advantage of the present invention is that a combination of medical imaging agents can be loaded into the cage. For example imaging agents for magnetic resonance imaging and x-ray imaging can be combined in one cage thereby allowing the resulting agent to be used with a multiple imaging methods. Another substantial advantage is that protein cages are capable of encapsulating a larger number of molecules than other vehicles, i.e. liposomes, commonly used for the delivery of therapeutic agents. For example, up to 29,600 molecules of H₂WO₄₂¹⁰⁻ have been packaged as a nano size crystalline solid within the cowpea chlorotic mottle virus (CCMV) protein cage. The size and shape of the crystallized nano material is determined by the size and shape of the cavity created by the CCMV protein cage. One other advantage, is that the protein cage can be used to increase the number of introduced materials present in the interior of the cage via crystallization. The crystallization of introduced materials can controlled because the protein cage provides a charged protein interface (on the interior) which can facilitate the aggregation and crystallization of ions.

In another embodiment, a fluorescent dye is attached to the interior surface of a protein cage. The dyes that may be attached include without limitation fluorescein, Texas red, and Lucifer yellow.

In one embodiment, small molecules are attached to the interior of a protein cage that allow binding of at least one multivalent ion. Such protein cage-small molecule complexes that bind multivalent ions may be activated by visible light. In another embodiment, the multivalent ion is a cation. In one other embodiment, the small molecules include without limitation bipyridine and phenanthroline, which bind Ru(II) as Ru(bpy)₃²⁺ analogs respectively. In some embodiments, the protein cage encapsulated Ru(bpy)₃²⁺ can act as an efficient sensitizer for singlet oxygen (¹O₂) production in addition to being a fluorescent material. In some other embodiments, such protein cage-small molecule complexes that bind multivalent ions may be used for fluorescent imaging and/or photodynamic therapy applications.

In one embodiment, a medical imaging agent is introduced into the protein cage. By “medical imaging agent” or “diagnostic agent” or “diagnostic imaging agent” herein is meant an agent that can be introduced into a cell, tissue, organ or patient and provide an image of the cell, tissue, organ or patient. Most methods of imaging make use of a contrast agent of one kind or another. Typically, a contrast agent is injected into the vascular system of the patient, and circulates through the body in, say, around half a minute. An image taken of the patient then shows enhanced features relating to the contrast agent. Diagnostic imaging agents include magnetic resonance imaging (MRI) agents, nuclear magnetic resonance (NMR) agents, x-ray imaging agents, optical imaging agents, ultrasound imaging agents and neutron capture therapy agents.

In another embodiment, the medical imaging agent is a magnetic resonance imaging (MRI) agent. By “MRI agent” herein is meant a molecule that can be used to enhance the MRI image. MRI is a clinical diagnostic and research procedure that uses a high-strength magnet and radio-frequency signals to produce images. The most abundant molecular species in biological tissues is water. It is the quantum mechanical “spin” of the water proton nuclei that ultimately gives rise to the signal in imaging experiments. In MRI the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. MRI is able to generate structural information in three dimensions in relatively short time spans. MRI agents can increase the rate of water proton relaxation and can therefore increase contrast between tissues.

As is known in the art, MRI contrast agents generally comprise a paramagnetic metal ion bound to a chelator. By “paramagnetic metal ion”, “paramagnetic ion” or “metal ion” herein is meant a metal ion which is magnetized parallel or antiparallel to a magnetic field to an extent proportional to the field. Generally, these are metal ions which have unpaired electrons; this is a term understood in the art. Examples of suitable paramagnetic metal ions, include, but are not limited to, gadolinium III (Gd+3 or Gd(III)), iron III (Fe+3 or Fe(III)), manganese II (Mn+2 or Mn(II)), ytterbium III (Yb+3 or Yb(III)), dysprosium (Dy+3 or Dy(III)), and chromium (Cr(III) or Cr+3). In one embodiment, the paramagnetic ion is the lanthanide atom Gd(III), due to its high magnetic moment (u2=63BM2), a symmetric electronic ground state (S8), and its current approval for diagnostic use in humans.

In addition to the metal ion, the MRI contrast agent usually comprise a chelator. Due to the relatively high toxicity of many of the paramagnetic ions, the ions are rendered nontoxic in physiological systems by binding to a suitable chelator. The chelator utilizes a number of coordination atoms at coordination sites to bind the metal ion. There are a large number of known macrocyclic chelators or ligands which are used to chelate lanthanide and paramagnetic ions. See for example, Alexander, Chem. Rev. 95:273-342 (1995) and Jackels, Pharm. Med. Imag, Section III, Chap. 20, p 645 (1990), expressly incorporated herein by reference, which describes a large number of macrocyclic chelators and their synthesis. Similarly, there are a number of patents which describe suitable chelators for use in the invention, including U.S. Pat. Nos. 5,155,215, 5,087,440, 5,219,553, 5,188,816, 4,885,363, 5,358,704, 5,262,532, and Meyer et al., Invest. Radiol. 25: S53 (1990), all of which are also expressly incorporated by reference. There are a variety of factors which influence the choice and stability of the chelate metal ion complex, including enthalpy and entropy effects (e.g. number, charge and basicity of coordinating groups, ligand field and conformational effects, etc.). In general, the chelator has a number of coordination atoms which are capable of binding the metal ion. The number of coordination atoms, and thus the structure of the chelator, depends on the metal ion. Thus, as will be understood by those in the art, any of the known paramagnetic metal ion chelators or lanthanide chelators can be easily modified using the teachings herein to add a functional moiety for covalent attachment to an optical dye or linker.

In one embodiment, the present invention provides protein cages having gadolinium (Gd³⁺) chelates. Such Gd³⁺ chelates are commonly used as a contrast agents in clinical settings (See Aime S, et al. J Magn Reson Imaging 2002; 16(4):394-406 and Meade T J et al. Curr Opin Neurobiol 2003; 13(5):597-602). In general, there are two ways to improve the imaging sensitivity using contrast agents, by either increasing the relaxivity of water protons through direct interaction with the contrast agent or by targeted delivery of the agent to specific locations within the body.

Examples 1 and 9 describe the use of a CCMV cage having gadolinium as a contrast agent. Various strategies may be employed to ensure sufficient gadolinium is attached to the protein cage interior. In one embodiment, the protein cages described herein may include a subunit modified to include unique reactive amino acid residues for the attachment of paramagnetic metal ion chelators. For example, thiol (cysteine) and/or amine (lysine) residues may be selectively introduced by genetic modification onto the exterior and/or interior surfaces of the protein cages. Those of skill in the art will appreciate the number of available chelates, including without limitation DPTA and DOTA. In one embodiment, reactive amino acids are introduced by genetic modification to a protein cage for the attachment of DPTA and/or DOTA. As shown in Table 1 above (Section II.A.1.), the reactive amino acids may be aspartate, glutamate, lysine, tyrosine, and/or cysteine and the protein cage may be a CCMV protein cage. In one embodiment, a tyrosine residue is utilized for attachment (Meunier S et al. (2004) Chem Biol 2004; 11(3):319-326). The multivalent nature of these cages allow the attachment of multiple Gd³⁺-chelates per subunit resulting in a very high Gd³⁺ loading per protein cage. For example, at least 500 Gd³⁺ per CCMV cage is possible.

In one embodiment, the protein cages of the present invention include modified subunits which have a binding affinity for paramagnetic metal ions. For example, it is known in the art that lanthanide ions can bind to calcium-binding proteins or calcium ion conducting membrane proteins. The present invention may provide protein cages with a modified subunit that includes one or more calcium-binding domains or sites. The binding sites may include one or more amino acids having binding affinity for calcium. The modified subunit may be genetically modified to include such amino acid(s). Alternatively, the modified subunit may include a calcium-binding peptide, where the subunit is genetically and/or chemically modified to include the peptide. Paramagnetic metal ions may be contacted with protein cages having subunits modified in this way such that the metal ions bind the calcium-binding sites. In one embodiment, the paramagnetic metal ion is gadolinium.

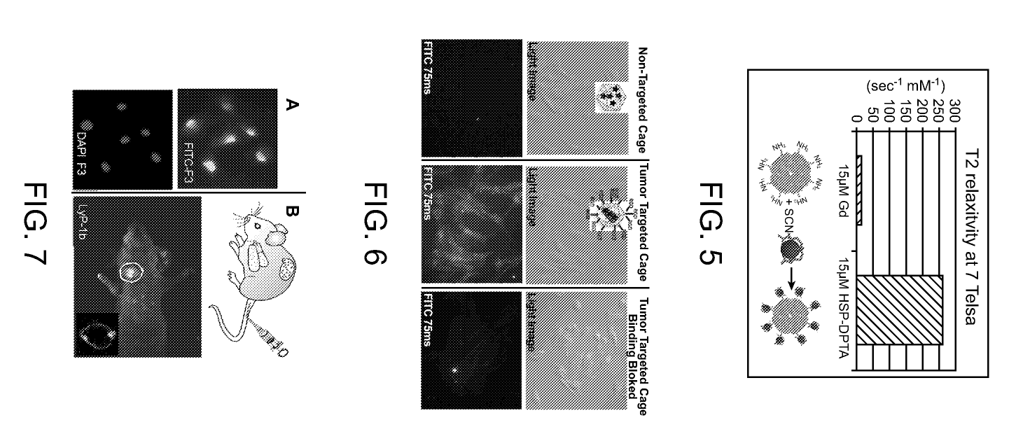

It will be appreciated by those skilled in the art that a number of calcium binding domains are suitable for the present invention. For example, binding domains from calmodulin-related proteins, or Gd³⁺ binding peptides selected from a phage display library may be suitable. The peptides may be introduced as either N- and C-terminal fusions on the protein cage subunits. Gd³⁺ dissociation constants as low as 1 nm have been reported for short peptide fragments. The multivalent nature of the protein cages of the present invention allow effective loading gadolinium. In one embodiment, at least 360 Gd³⁺ ions per protein cage may be loaded in a spatially defined manner. The protein cage may be a CCMV protein cage. The incorporation of binding domains onto the CCMV protein cage has extremely high T1 and T2 relaxivities both on a per protein cage and per Gd³⁺ basis (FIG. 4) In another embodiment, the Gd³⁺ ions may be provided with a protein cage either on the water accessible exterior surface or buried on the interior of the cage with reduced water accessibility.

In one aspect, the protein cages of the present invention include polymers loaded with the agents described herein. In some embodiments, the protein cages include polymers loaded with one or more therapeutic agents, such as the small molecules or drugs described herein. These polymers may be linear or branched in nature and be comprised on conventional water soluble, biodegradable polymers including without limitation hydrogels, dendrimers, PLGA, polylysines and other poly(D, L lactic-co-glycolide) 50:50 (PLGA), poly(D, L lactide) (PDLA) and poly(L lactide) (PLLA). Other suitable polymers are also known in the art (Nuno Silva, J. et al. (2006) Photochem Photobiol Sci January; 5(1):126-33; Duncan, M. J. et al. (2005) Endocrine-Related Cancer (Supplemental 1) S189-199).

A number of drug release strategies are suitable for use with the protein cages described herein. For example the use of macromolecular water-soluble carriers of anti-cancer drugs represents a promising approach to cancer therapy. Release of drugs from the carrier system is a prerequisite for therapeutic activity of most macromolecular anti-cancer conjugates. Incorporation of acid-sensitive spacers between the drug and carrier enables release of an active drug from the carrier in a tumor tissue, either in slightly acidic extracellular fluids or, after endocytosis, in endosomes or lysosomes of cancer cells. Several strategies are suitable including without limitation various acid-sensitive macromolecular drug delivery systems such as simple polymer-drug conjugates and/or site-specific antibody-targeted polymer-drug conjugates. (see Ulbrich, K. (2004) Adv. Drug Deliv. Rev. Apr. 23; 56(7); 1023-50).

Such polymers can be loaded with drugs, such as doxorubicin (see Vasey, P. A. (1999) January:5(1):83-94). Delivery of bioactive molecules such as nucleic acid molecules encoding a protein can be significantly enhanced by immobilization of the bioactive molecule in a polymeric material adjacent to the cells where delivery is desired, where the bioactive molecule is encapsulated in a vehicle such as liposomes which facilitates transfer of the bioactive molecules into the targeted tissue. Targeting of the bioactive molecules can also be achieved by selection of an encapsulating medium of an appropriate size whereby the medium serves to deliver the molecules to a particular target. For example, encapsulation of nucleic acid molecules or biologically active proteins within biodegradable, biocompatible polymeric microparticles which are appropriate sized to infiltrate, but remain trapped within, the capillary beds and alveoli of the lungs can be used for targeted delivery to these regions of the body following administration to a patient by infusion or injection (see U.S. Pat. No. 5,879,713 incorporated by reference).

In one other embodiment, polymers with fatty acid conjugates may be attached to protein cages for drug targeting purposes A general method for incorporating target ligands into the surface of biocompatible polyester poly(lactic-co-glycolic acid) (PLGA) 50/50 materials using fatty acids. Avidin-fatty acid conjugates were prepared and efficiently incorporated into PLGA. Avidin was chosen as an adaptor protein to facilitate the attachment of a variety of biotinylated ligands. It has been shown that fatty acid preferentially associates with the hydrophobic PLGA matrix, rather than the external aqueous environment, facilitating a prolonged presentation of avidin over several weeks. The approach was applied in both microspheres encapsulating a model protein, bovine serum albumin, and PLGA scaffolds fabricated by a salt-leaching method. Because of its ease, generality and flexibility, this strategy promises widespread utility in modifying the surface of PLGA-based materials for applications in drug delivery and tissue engineering (see Fahmy, T. M. et al. (2005) Biomaterials October; 26(28):5727-36).

In one embodiment, the protein cages include polymers loaded with paramagnetic metal ions. Electrostatically driven association of polyanions, including without limitation polydextran sulfate, polyacrylic acid, and polyallylamine amine, may be used to introduce paramagnetic metal ion chelates into protein cages. In one embodiment, a chelate such as DPTA and/or DOTA may be coupled to poly(allylamine) through an amide linkage. Alternatively, a polymer may be introduced to a protein cage having a polyanionic interior surface. In one embodiment, the subE mutant of the CCMV protein cage (Douglas, T. et al., (2002) Adv. Mater 14, 415-418) having a polyanionic interior surface may be loaded with a chelate such as DPTA and/or DOTA by an electrostatically driven method. In general, a series of charged polymers with covalently attached chelates may be synthesized and introduced into the interior of protein cage structures in vitro using techniques known in the art). In one embodiment, the chelate(s) are a gadolinium chelate.

Suitable MRI contrast agents include, but are not limited to, 1,4,7,10-tetraazacyclododecane-N,N′,N″N″′-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (Do3A) and derivatives thereof (see U.S. Pat. Nos. 5,188,816, 5,358,704, 4,885,363, and 5,219,553, hereby expressly incorporated by reference).

In addition, as is known in the art, the use of iron oxides and aggregates of iron oxides as MRI agents is well known.

In one embodiment, the medical imaging agent is a nuclear magnetic resonance imaging agent (NMR). By “NMR agent” herein is meant a molecule that can be used to enhance the NMR image. NMR is a very extensively used method of medical diagnosis, used for in vivo imaging, with which vessels of the body and body tissue (including tumors) can be visualized by measuring the magnetic properties of the protons in the body water. To this end, e.g., contrast media are used that produce contrast enhancement in the resulting images or make these images readable by influencing specific NMR parameters of the body protons (e.g., relaxation times T¹ and T²). Mainly complexes of paramagnetic ions, such as, e.g., gadolinium-containing complexes (e.g., Magnevist™) are used owing to the effect of the paramagnetic ions on the shortening of the relaxation times. A measure of the shortening of the relaxation time is relaxivity, which is indicated in m⁻¹ sec⁻¹.

For use in NMR imaging, paramagnetic ions (see above) are generally complexed with aminopolycarboxylic acids, e.g., with diethylenetriamine-pentaacetic acid [DTPA]). The di-N-methylglucamine salt of the Gd-DTPA complex is known under the name Magnevist™ and is used to diagnose tumors in the human brain and in the kidney. See U.S. Pat. No. 6,468,502 and EP 0 071 564 A1, both of which are incorporated by reference in their entirety.

In one other embodiment, the protein cages may include gold particles for optical imaging. It has been reported that silica nanoshells may be engineered to scatter near infrared (NIR) light to allow for optical molecular cancer imaging and/or engineered to absorb light in order to allow for photothermal therapy (Loo, C. et al. (2005) Nano Letters 5(4):709-711). In one embodiment, the present invention provides protein cages with gold particles attached such that NIR light is scattered upon application. In another embodiment, as described above (Section III.A.6. Inorganic material agents), the protein cage is used for thermal ablation therapy. In yet another embodiment, the protein cages are engineered to include an optical agent and a thermal ablation agent.

In another embodiment, the medical imaging agent is a x-ray agent. By “x-ray agent” herein is meant a molecule that can be used to enhance an x-ray image. Agents suitable for use as x-ray agents include contrast agents such as iodine or other suitable radioactive isotopes. See U.S. Pat. No. 6,219,572, incorporated by reference in its entirety.

In some embodiments, the medical imaging agent is a optical agent. By “optical agent” herein is meant an agent comprising an “optical dye”. Optical dyes are compounds that will emit detectable energy after excitation with light. Optical dyes may be photoluminescent or fluorescent compounds. In other embodiments, the optical dye is fluorescent; that is, upon excitation with a particular wavelength, the optical dye with emit light of a different wavelength; such light is typically unpolarized. In an alternative embodiment, the optical dye is phosphorescent.

Suitable optical dyes include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Suitable optical dyes are described in the 1989-1991 Molecular Probes Handbook by Richard P. Haugland, hereby expressly incorporated by reference.

In other embodiments, the optical dye is functionalized to facilitate covalent attachment. Thus, a wide variety of optical dyes are commercially available which contain functional groups, including, but not limited to, isothiocyanate groups, amino groups, haloacetyl groups, maleimides, succinimidyl esters, and sulfonyl halides, all of which may be used to covalently attach the optical dye to a second molecule, such as other imaging agents or to the protein cages.

In one embodiment, the optical agent is based on gold particles as discussed above. A protein cage having gold particles externally located may be an imaging agent. When gold particle coated nanoshells are observed under darkfield microscopy (sensitive only to scattered light), they can be observed to scatter light strongly throughout the visible and NIR light regions, which can be imaged via reflectance confocal microscopy and optical coherence tomography (OCT) (see Loo, C. (2004) supra; Hirsch, L. R. (2006) supra) In another embodiment, the protein cage having gold particles has the dual function of an imaging agent and upon application of NIR light a thermal ablation agent.

In another embodiment, the medical imaging agent is a ultrasound agent. By “ultrasound agent” herein is meant an agent that can be used to generate an ultrasound image. Generally, for ultrasound, air in small bubble-like cells, i.e. microparticles is used as a contrast agent. See U.S. Pat. Nos. 6,219,572, 6,193,951, 6,165,442, 6,046,777, 6,177,062, all of which are hereby expressly incorporated by reference.

In some other embodiments, the medical imaging agent is a neutron capture therapy agent (NCT). NCT is based on the nuclear reaction produced when a neutron capture agent such as ¹⁰B or ¹⁵⁷Gd isotope (localized in tumor tissues) is irradiated with low energy thermal neutrons. The radiation produced is capable of effecting selective destruction of tumor cells while sparing normal cells. The advantage of NCT is the fact that it is a binary system, capable of independent variation of control of the neutron capture agent and thermal neutrons. The NCT agents may contain either ¹⁰B or ¹⁵⁷Gd. See U.S. Pat. Nos. 5,286,853, 6,248,305, and 6,086,837; all of which are hereby expressly incorporated by reference.

The present invention provides a class of potential MR imaging agents. In one embodiment, protein cages as described herein are engineered by a method as described herein to incorporate high affinity Gd³⁺ binding sites into a protein cage. As illustrated in FIG. 5, Gd³⁺ binding sites may be incorporated into CCMV and/or sHsp protein cages. In additional embodiments, protein cages having high affinity Gd³⁺ binding sites exhibit enhanced T1 and T2 relaxivities. (Basu, G., et al., 2003. Journal of Bioinorganic Chemistry 8:721-725) In another embodiment, enhanced relaxivity is due to the large macromolecular size of the cage which reduces the rotational correlation relative to small molecules. In one other embodiment, the enhanced relaxivity may be due to the precise spatial distribution of a large number (180) of isolated Gd³⁺ ions bound to the cage in a small volume, with access to water.

The present invention provides methods to improve the performance of the Gd-cages for biomedical applications. In one embodiment, the methods include utlitizing endogenous metal binding sites within a protein cage, which have a low Gd³⁺ binding affinity. In other embodiments, the Gd³⁺ binding affinity K_{d} is 30 μM. In one embodiment, the protein cage is a CCMV protein cage.

In another embodiment, the methods include genetically incorporating small peptides (metal-binding motifs from calmodulin) onto the interior or exterior of the protein cages, resulting in significant increase in Gd³⁺ binding affinity to the cage. In some embodiments, the Gd³⁺ binding affinity K_{d} is 0.1 μM. In another embodiment, the methods include chemically modifying protein cages with Gd-DPTA (Magnevist, K_{d}˜10⁻²⁵) to provide a multivalent presentation of the Gd³⁺ contrast agent with enhanced relaxivity properties.

In one embodiment, the methods of incorporating high affinity Gd³⁺ binding sites into a protein cage as described herein result in the binding of at least 180 Gd³⁺ ions per cage. In another embodiment, more than 180 Gd³⁺ ions per cage may be bound.

In one embodiment, the present invention provides protein cages that have multifunctionalities within a single protein cage architecture. In one other embodiment, the present invention provides a protein cages incorporating Gd-DPTA within the cage interior and one or more targeting ligands on the exterior. In some embodiments, the protein cages incorporating Gd-DPTA within the cage interior also include drug molecules within the cage.

III. Modification of Protein Cages

The present invention is directed to the use of novel protein cages and mixtures of cages to form novel compositions, either in solution based systems and/or solid phase systems (e.g. two and three dimensional arrays on solid supports). The nanoparticles, which comprise both a protein “shell” and a “core”, can be mixed together to form novel compositions of either complete nanoparticle or core mixtures. In addition, the shells can be loaded to form the complete nanoparticles with any number of different materials, including organic, inorganic and metallorganic materials, and mixtures thereof. Some embodiments utilize magnetic materials, to allow for high density storage capacities. Furthermore, as the shells are proteinaceous, they can be altered to alter any number of physical or chemical properties by a variety of methods, including but not limited to covalent and non-covalent derivatization as well as recombinant methods.

In one aspect, up to three interfaces in the protein cage architecture may be manipulated to impart biomedical functionality. The three interfaces are the exterior surface, the interior surface, and the interface between subunits. In one other embodiment, chemical and/or genetic alteration of the protein subunits can be used to impart impart novel function to one or more of the three interfaces or surfaces of the protein cages. (See Basu, G., M. A. et al., Journal of Bioinorganic Chemistry 8:721-725; Douglas, T. et al., 2000. Inorg. Chem. 39:1828-1830; Flenniken, M. L. et al., 2005. Chemical Communications:447-449; Flenniken, M. L., et al., 2003. Nano Letters 3:1573-1576; Gillitzer, E., et al., 2002. Chemical Commun.:2390-2391; Klem, M. T., et al., 2003. J. Am. Chem. Soc. 125:1056-1057). In some embodiments, the present invention provides protein cages with novel functionality in high copy number and methods of making the same (See, e.g. Brumfield, S., et al., 2004. Journal of General Virology 85:1049-1053; Zhao, X. et al., 1995. Virology 207:486-494). The present invention provides simultaneous control over size, shape, biocompatibility, and ability to alter functionality using both chemical and genetic techniques.

The protein cage may be unmodified or modified. By “unmodified” or “native” herein is meant a protein cage that has not been genetically altered or modified by other physical, chemical or biochemical means. By “modified” or “altered” herein is meant a protein cage that has been genetically altered or modified by physical, chemical or biochemical means as described herein.

In additional embodiments, the protein cage is modified. In another embodiment, the modification results in protein cages with improved properties for use as delivery vehicles. For example, protein cages can be designed that are more stable than the unmodified cages or to contain binding sites for metal ions. Additionally, protein cages can be designed that have different charged interior surfaces for the selective entrapment and aggregation of medical imaging or therapeutic agents. Other modifications include the introduction of new chemical switches that can be controlled by pH or by redox conditions, the introduction of targeting moieties on the exterior surface, the addition of functional groups for the subsequent attachment of additional moieties, and covalent modifications.

In some embodiments, protein cages are genetically modified to be more stable. Native CCMV virions are stable over a broad pH range (pH 2-8) and temperature (−80 to 72° C.) (Zhao, X., et al., 1995, Virology, 207:486-494). Empty virions (assembled CCMV protein cages) are stable over this range when assembled from mutants of the coat protein. The salt stable coat protein mutation (K42R) (Fox, J. M., et al., 1996, Virology 222:115-122) and the cysteinyl mutation (R26C) (Fox, J., et al., 1997, Virology 227:229-233.32) both result in empty virions that are stable over this broad pH and temperature range.

As will be similarly appreciated by those in the art, the monomers of the protein cages can be naturally occurring or variant forms, including amino acid substitutions, insertions and deletions (e.g. fragments) that can be made for a variety of reasons as further outlined below. For example, amino acid residues on the outer surface of one or more of the monomers can be altered to facilitate functionalization for attachment to additional moieties (targeting moieties such as antibodies, polymers for delivery, the formation of non-covalent chimeras), to allow for crosslinking (e.g. the incorporation of cysteine residues to form disulfides). Similarly, amino acid residues on the internal surfaces of the shell can be altered to facilitate the introduction and/or loading of agents, stability, to create functional groups which may be later modified by the chemical attachment of other materials (small molecules, polymers, proteins, etc.).

A. Chemical Modification

In one aspect, one or more subunits of the protein cages of the present invention are modified chemically. In one embodiment, a subunit is modified in order to attach an agent. The modification may allow attachment of an agent such that it is present on an internal surface of the cage and/or on the external surface of the protein cage.

1. Linkers

In another embodiment, the external and/or internal attachments contemplated are achieved through the use of linkers. It should be appreciated that the imaging agents and therapeutic agents of the present invention may be attached to the protein cage via a linker. Linkers are well known in the art; for example, homo- or hetero-bifunctional linkers as are well known (see 1994 Pierce Chemical Company catalog, technical section on cross-linkers, pages 155-200, incorporated herein by reference). Generally, suitable linker groups include, but are not limited to, alkyl and aryl groups, including substituted alkyl and aryl groups and heteroalkyl (particularly oxo groups) and heteroaryl groups, including alkyl amine groups, as defined above. Suitable linker groups include without limitation p-aminobenzyl, substituted p-aminobenzyl, diphenyl and substituted diphenyl, alkyl furan such as benzylfuran, carboxy, and straight chain alkyl groups of 1 to 10 carbons in length, short alkyl groups, esters, amide, amine, epoxy groups, nucleic acids, peptides and ethylene glycol and derivatives. In some other embodiments, the linkers include without limitation p-aminobenzyl, methyl, ethyl, propyl, butyl, pentyl, hexyl, acetic acid, propionic acid, aminobutyl, p-alkyl phenols, 4-alkylimidazole and polymers. The selection of the linker group is generally done using well known molecular modeling techniques, to optimize the obstruction of the coordination site or sites of the metal ion. In addition, the length of this linker may be very important in order to achieve optimal results.

In one other aspect, the protein cages include cleavable linkers. For example, the present invention may provide a protein cage with a small molecule, the release of which is pH dependent. In one embodiment, the linker may be an acid labile linker, such as for example a hydrazone linkage that is acid labile. In another embodiment, the cleavable linker is a hydrazone linkage and the small molecule is doxorubicin. In another embodiment, a cleavable linker is incorporated into the small molecule covalently attached to the protein cage interior. (see Flenniken, M. L. et al., 2005. Chemical Comm.:447-449; Willner, D., et al., 1993. Bioconjug Chem 4:521-7). Other examples of acid labile linkers include linkers formed by using cis-aconitic acid, cis-carboxylic alkatriene, polymaleic anhydride, and other acid labile linkers, such as those linkers described in U.S. Pat. Nos. 5,563,250 and 5,505,931.

In one embodiment, the linker is a photo-labile linker. Examples of photo-labile linkers include those linkers described in U.S. Pat. Nos. 5,767,288 and 4,469,774, each of which is incorporated by reference in its entirety.

In another embodiment, the linker used to attach an agent to a protein cage is a polymer. As will be appreciated by those of skill in the art, polymers comprising only imaging agents, only therapeutic agents or a combination of both have use in the methods of the invention. Moreover, protein cages comprising imaging agents and therapeutic agents may also be attached to polymers via functional groups introduced on the surface of the cage (see above).

The character of the polymer will vary, but what is important is that the polymer either contain or can be modified to contain functional groups for the attachment of the nanoparticles of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyornithine; and mixtures and derivatives of these. Suitable polycations include without limitation polylysine and spermidine. Both optical isomers of polylysine can be used. The D isomer has the advantage of having long-term resistance to cellular proteases. The L isomer has the advantage of being more rapidly cleared from an animal when administered. As will be appreciated by those in the art, linear and branched polymers may be used.

In another embodiment, the polymer is polylysine, as the —NH2 groups of the lysine side chains at high pH serve as strong nucleophiles for multiple attachment of imaging and therapeutic agents. At high pH the lysine monomers can be coupled to the nanoparticles under conditions that yield on average 5-20% monomer substitution.

The size of the polymer may vary substantially. For example, it is known that some nucleic acid vectors can deliver genes up to 100 kilobases in length, and artificial chromosomes (megabases) have been delivered to yeast. Therefore, there is no general size limit to the polymer. However, suitable sizes for the polymer may be from about 10 to about 50,000 monomer units, from about 2000 to about 5000 units, and from about 3 to about 25 units.

2. Covalent Modifications

In other embodiments, covalent modifications of protein cages are included within the scope of this invention. One type of covalent modification includes reacting targeted amino acid residues of cage residue with an organic derivatizing agent that is capable of reacting with selected side chains or the N- or C-terminal residues of a cage polypeptide. Derivatization with bifunctional agents is useful, for instance, for crosslinking the cage to a water-insoluble support matrix or surface for use in the methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl propionate), bifunctional maleimides such as bis-N-maleimido-1,8-octane and agents such as methyl-3-[(p-azidophenyl)dithio]propioimidate. Crosslinking agents find particular use in 2 dimensional array embodiments.

Alternatively, functional groups may be added to the protein cage for subsequent attachment to additional moieties. Suitable functional groups for attachment include without limitation amino groups, carboxy groups, oxo groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, as well as the 2003 catalog, both of which are incorporated herein by reference). Suitable linkers include, but are not limited to, alkyl groups (including substituted alkyl groups and alkyl groups containing heteroatom moieties), short alkyl groups, esters, amide, amine, epoxy groups and ethylene glycol and derivatives, as well as propyl, acetylene, and C₂ alkene groups.

In other embodiments, protein cages are modified by the introduction of functional groups on the inside of the protein cage. In one embodiment, ion binding sites in the interior of the cage are modified to bind paramagnetic metals, such as gadolinium (Gd(III) or Gd³⁺). In one embodiment, existing Ca²⁺ binding sites are modified to enhance binding of Gd(III) (see Example 1). Through the use and modification of existing metal binding sites from 1 to 180 Gd(III) ions can be incorporated per cage. In addition, cages may include the following ranges of Gd(III) ions: from about 10 to about 180, from about 50 to about 180, from about 75 to about 180, from about 100 to about 180, and from about 150 to about 180 Gd(III) ions.

In one embodiment, the interior of the protein cages of the present invention are modified for covalent attachment of small organic molecules. Spatially controlled chemical linkages may be used for such attachments. In another embodiment, cysteine thiols may be engineered on the interior of a protein cage through a maleimide coupling reaction (Flenniken, M. L., et al., 2005. Chemical Communications:447-449) allowing the attachment of an anti-cancer drug. In other embodiments, the cysteine thiol linker facilitates release of an anti-cancer drug upon cell entry through an endosomal/lysosomal pathway. In another embodiment, the anti-cancer drug is doxorubicin.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “—amino groups of lysine, arginine, and histidine side chains T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

In one embodiment, targeting peptides will be covalently attached to the magnetic nano-particle cages as described herein The in vitro evaluation of the hyperthermia induced by the cage encapsulated magnetic nano-particles may be performed using magnetic protein cages embedded in 0.5% agarose (to mimic tissue viscosity). Targeted cages may be evaluated in cell culture by incubation of the magnetic cages (with and without targeting ligands) with cells (see Table 1), followed by extensive washing to remove unbound cages and irradiation with an AC-field to induce hyperthermia.

3. Modification of Glycosylation Patterns

Another type of covalent modification of cages, if appropriate, comprises altering the native glycosylation pattern of the polypeptide. “Altering the native glycosylation pattern” is intended for purposes herein to mean deleting one or more carbohydrate moieties found in native sequence of the cage monomer, and/or adding one or more glycosylation sites that are not present in the native sequence.

Another means of increasing the number of carbohydrate moieties on the polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981). In one embodiment, the removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophvs., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of cage moieties comprises linking the polypeptide to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337. This finds particular use in increasing the physiological half-life of the composition.

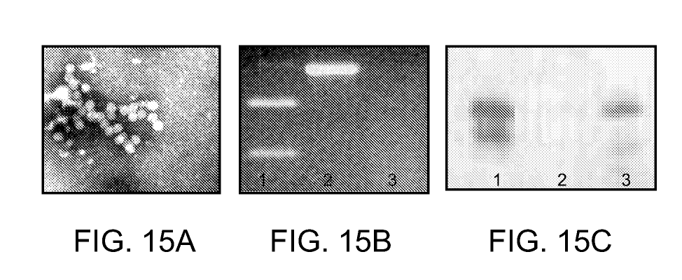

In some embodiments, protein cages are modified to allow for the attachment of functional groups that can be used to attach imaging and therapeutic agents. For example replacement of amino acids on the inner surface of the cage by cysteine residues results in the presentation of reactive —SH groups on the inner surface. In addition to the role of —SH groups in redox activated switching, —SH groups can be reactive with bifunctional agents, such as maleimide to attach diagnostic agents (i.e. MRI imaging agents) and therapeutic agents to the interior of the cage (see FIG. 15).

In another embodiment, targeting peptides may be incorporated by chemical attachment through reaction of surface exposed functional groups (either endogenous or engineered) on the protein cage, as further discussed below.

In one embodiment, the reaction conditions including without limitation molar ratios of peptide:cage, pH, temperature, and time are controlled such that the extent of peptide linkage to an individual cage is controlled allowing the production of materials for testing the effect of multi-valent presentation of targeting peptides on cell/tissue targeting.

In one embodiment, the present invention provides methods including controlling the degree of peptide and small molecule attachment to a particular cage architecture by controlling the reaction conditions.

In other embodiments, targeting moieties are add to the surface of protein cages through the use of functional groups. Functional groups may be added to the protein cage for subsequent attachment to additional moieties. Suitable functional groups for attachment include without limitation amino groups, carboxy groups, oxo groups and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, as well as the 2003 catalog, both of which are incorporated herein by reference). Suitable linkers include, but are not limited to, alkyl groups (including substituted alkyl groups and alkyl groups containing heteroatom moieties), short alkyl groups, esters, amide, amine, epoxy groups and ethylene glycol and derivatives, as well as propyl, acetylene, and C₂ alkene groups.

4. Modifications for Encapsulation and Synthesis

One of the advantages of the present invention is to enable the introduction or synthesis and encapsulation of nanoparticles, which cannot be accomplished through techniques and means disclosed in prior art. Another substantial advantage over prior art is the ability to vary the size of the nanoparticle encapsulated and constrained in the protein cage structure. It should be easily recognized that a portion of the volume within a given cage structure will be filled with ferrihydrite (in the case of ferritin structures) and thus a smaller nanoparticle of, for example CoPt, could be encapsulated compared to said nanoparticle encapsulated in an identical apoferritin structure. Furthermore, in the synthesis of iron containing molecules and structures, for example FePt, one can utilize the ferrihydrite present as one of the starting materials. Another advantage of the present invention is to improve the efficiency for encapsulation of nanoparticles by eliminating processing steps—compared to both apoferritin methods taught in the art.

The present invention also serves to enhance the usefulness of the encapsulated and constrained nanoparticles of the present invention by modification of the surfaces and interfaces of the protein cage structure. It is known in the prior art that various modifications to the outside of ferritins can be accomplished through chemical, physical and/or gene modification technology. These modifications can enable or prohibit attachment of the ferritin or other protein cage structures to other similar structures, can provide a means to bind to targets of interest for medical applications, can provide a means and method of fabricating two and three dimensional arrays of like, similar or different combinations of nanoparticles constrained by ferritin and other protein cage structures.

In the formation of useful arrays of nanoparticles, an essential element is a matrix of material surrounding and joining the nanoparticles, which may be insulating, semiconducting, or conducting. It is an object of this invention to chemically, genetically or physically modify the outside of the protein cages to enable self-assembly of arrays through the utilization of other organic or inorganic materials. This invention discloses the use of a matrix material, which could be self-assembled, that utilizes ferritins, other proteins and other organic macromolecules to fill the interstices between the nanoparticles. Ferritin cages identical to those forming the primary array of nanoparticles but that contain nanoparticles having other desired properties can also be used. The use of identical protein cages containing insulating or semiconducting materials as the intersticial materials could be particularly advantageous.

The present invention also enhances the usefulness of the constrained nanoparticles by modification of the interfaces through chemical or other means as disclosed in prior art to enable opening and closing the structure for introduction or extraction of the materials contained therein.

Thus, the present invention enhances the usefulness of the constrained nanoparticles by employing specific combinations of constrained nanoparticles, surface modification and interface modification to enable specific desired outcomes. For example a FePt core may be constrained within a ferritin cage and through appropriate surface modification arrays can be formed into two-dimensional arrays for use in floating gate magnetic memory applications. Techniques for burning away or otherwise eliminating the protein structure to produce a uniform array of cores are well known in the art and described below.

The encapsulated or constrained nanoparticles and/or nanoparticle cores of the present invention have many utilities including drug delivery, catalysis, semiconductor technology, ultra-high density recording, nanoscale electronics, and permanent magnets.

B. Genetic Modification

In another aspect, the protein cages of the present invention may include subunits that have been genetically modified. By a “genetic modification” or a “mutation” and grammatical equivalents thereof, is meant any alteration of the amino acid sequence of one or more protein subunits by mutation of the nucleic acid sequence.

Addition of glycosylation sites to cage polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Cage polypeptides of the present invention may also be modified in a way to form fusion proteins or chimeric molecules comprising a cage polypeptide fused to another, heterologous polypeptide or amino acid sequence. In one embodiment, such a chimeric molecule comprises a fusion of a cage polypeptide with a tag polypeptide which provides an epitope to which an anti-tag antibody can selectively bind. The epitope tag is generally placed at the amino- or carboxyl-terminus of the polypeptide. The presence of such epitope-tagged forms of a cage polypeptide can be detected using an antibody against the tag polypeptide. Also, provision of the epitope tag enables the cage polypeptide to be readily purified by affinity purification using an anti-tag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In other embodiments, methods are provided that include chemical and genetic modification to introduce cell targeting ligands (RGD-4C, F3, Lyp-1, NGR) and protein cage aggregation ligands (e.g. biotin) to a single cage that also contains an internal fluorophore (e.g. fluorescein). The methods further include the use of cell culture techniques to monitor the association of these target cages to the cell surface as illustrated in FIGS. 6 and 7. Following washing, the cells may be incubated in the presence of protein cage architectures expressing the corresponding aggregation ligand (streptavidin) internally labeled with a different fluorophore (Texas Red). Using a combination of fluorescence/confocal microscopy and FACS analysis, co-localization of the two fluorophores may be examined.

In one embodiment, the N-terminal arm of the coat protein subunit of CCMV is genetically modified to aggregate new classes of materials including the so-called soft metals, including Fe(II) (Cotton, F. A., and G. Wilkinson., 1999, Inorganic Chemistry, John Wiley & Sons), polyanions (i.e. poly(dextran sulfate) and poly(anetholosulfonic acid)), small molecules (i.e., drug and drug analogs), polycationic species (i.e. poly(ethylenimine), poly(lysine), poly(arginine) and poly(vinylimidazoline) (see Example 2).

In one embodiment, the protein cages include genetically modified subunits which have peptides attached as a chimeric molecule or fusion protein. Suitable peptides include those described herein (See Section IV. Targeting moieties)

In another embodiment, magnetic nanoparticles include genetically incorporated peptides specific for a particular inorganic material onto the interior surface of the protein cages. These peptides will be identified and collected from a phage display library (Klem, M. T., et al. 2005. Adv. Funct. Mater. submitted), (Mao, C. B., et al. 2003. Proceedings of the National Academy of Sciences of the United States of America 100:6946-6951), (Mao, C. B., et al. 2004. Science 303:213-217), (Seeman, N. C., et al. 2002. Proceedings of the National Academy of Sciences of the United States of America 99:6451-6455), (Whaley, S. R., et al. 2000. Nature 405:665-668). In another embodiment, a peptide specific for the L1₀ phases of CoPt (KTHEIHSPLLHK) is incorporated on the interior surface of the sHsp cage. In another embodiment, the resulting monodisperse CoPt particles (6±0.8 nm) show ferromagnetic behavior, and high saturation moments prior to thermal annealing.

In another embodiment, magnetic particles utilize a CCMV protein cage in which the size parameter in generating magnetic materials is controlled. This general approach of identifying specific peptides to any mineral phase and incorporating them into a size constrained reaction vessel allows control of nano-particle size, shape and composition. In one embodiment, this approach may be used towards the synthesis of high magnetic moment materials that are biocompatible. In other embodiments, well-defined highly magnetic materials may be used such as for example FePt (HNKHLPSTQPLA) (Mao, C. et al., 2004 Science 303:213-217) where both components of the alloy (Fe and Pt) exhibit very limited cyto-toxicity. These magnetic particles will be powerful materials for assessing in vivo distribution and deposition of targeted protein cages, using MRI, as a step in their development as clinically relevant agents.

As will be appreciated by those in the art, the compositions and delivery agents of the present invention can include a wide variety of different mixtures of protein cages and therapeutic and/or imaging agents. Protein cages with mixed compositions, such as modified and unmodified subunits, are suitable, and matrices of different sized nanoparticles and/or cores with different core compositions are also possible, as outlined herein.

IV. Release Mechanisms

In one aspect, the protein cages of the present invention include a disassembly mechanism. By “disassembly mechanism” is meant a mechanism by which the disassembly of a protein cage is controlled. Control of cage disassembly may be control of the opening and/or closing of the pores present in the protein cage (this may also be referred to herein as a “gating mechanism”) or it may be control of the integrity of the protein cage architecture itself. In one embodiment, the integrity of the protein cage architecture may formulated such that the cage is sensitive to modification by various in vivo endogenous enzymes as discussed below. In one other embodiment, the modification is digestion of the protein cage, particularly the individual subunits forming the cage.

In one embodiment, the disassembly mechanism may include without limitation (i) a pH sensitive mechanism, (ii) a redox sensitive mechanism; (iii) a reversible or irreversible chemical switch mechanism, and (iv) an enzymatic release mechanism. In some embodiments, the pH sensitive mechanism may be a reversible chemical switch mechanism or it may be a reversible redox sensitive chemical switch.

A. Gating of Protein Cages

In one other aspect, the present invention provides protein cages with modified subunits that allow for control of the opening and closing of the cage. For example, amino acid residues may be substituted for existing amino acid residues to alter the pH sensitivity and redox sensitivity. Other modifications include the expression of heterologous amino acid sequences on surface of the cage that can then be used to direct, i.e., target the cage, to a particular location in a cell, tissue or organ.

Static open and static closed cage architectures may also be used for controlling the gating of protein cages. For example, as illustrated in FIG. 2, the sHsp protein cage offers large non-gatable pores, which allow free access between interior and exterior environments while still protecting any entrapped material (See Kim, K., et al., supra 1998). In some embodiments, the present invention provides a protein cage with a static open architecture containing a small molecule. In one other embodiment, the protein cage is a sHsp protein cage. In another embodiment, the protein cage is an sHsp protein cage and the small molecule is doxorubicin. (See Flenniken, M. L., et al., supra 2005).

It has been shown that the sHsp heat shock protein crystal structure has 8, 3 nm pores located at the three-fold axes that allow free and open molecular access between the interior and exterior environments (Kim et al. supra 1998). In one embodiment, CCMV protein cage are compared with open access sHsp cage in order to evaluate controlled access to and release of encapsulated therapeutic and imaging agents.

It has been reporter fundamental understanding of the capsid dynamics in CCMV, we have utilized these aspects to direct packaging of a range of synthetic materials including drugs and inorganic nanoparticles. As discussed herein, the icosahedral Cowpea chlorotic mottle virus (CCMV) is an excellent model for understanding the encapsulation and packaging of synthetic materials.

In one embodiment, the present invention provides a protein cage having a disassembly mechanism that includes a pH dependent reversible structural transition, wherein a plurality of protein cage pores open or close. The protein cage may be a CCMV protein cage where 60 pores open or close based on such a pH dependent mechanism. It has been reported that the CCMV capsid undergoes a pH and metal ion dependent reversible structural transition where 60 separate pores in the capsid open or close, exposing the interior of the protein cage to the bulk medium. In addition, the highly basic N-terminal domain of the capsid, which is disordered in the crystal structure, plays a significant role in packaging the viral cargo. Interestingly, in limited proteolysis and mass spectrometry experiments the N-terminal domain is the first part of the subunit to be cleaved, confirming its dynamic nature (See Liepold, L. O. et al. (2005) Phys. Biol. November 9; 2(4):S166-72).

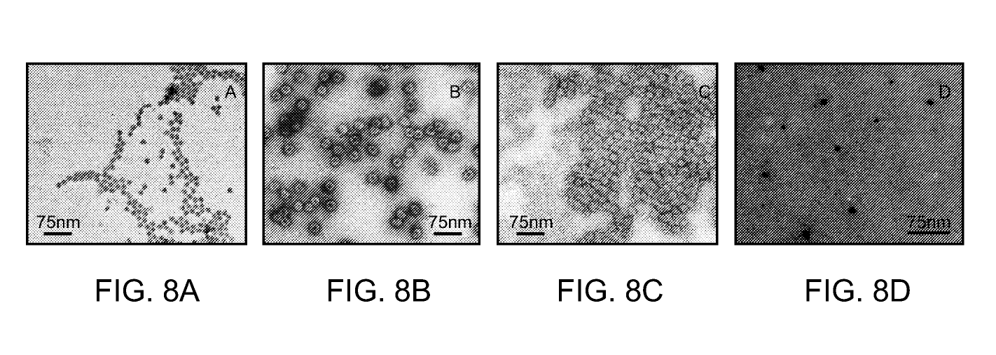

The present invention is directed to the discovery that protein cages can be used as constrained reaction vessels for the selective entrapment and release of materials. A unique aspect of protein cages that makes them attractive as delivery vehicles is there ability to undergo reversible structural changes allowing for the formation of open pores through which material can pass. These reversible changes can be controlled by factors such as pH and ionic strength. For example, pH can be used to control the expansion and contraction of the protein cage (see FIG. 8A-C). When the cage is expanded, i.e., opened, pores are formed allowing for the free exchange of soluble material between the inside and outside of the cage (see FIG. 8A). When the cage is contracted, i.e., closed, the pores are closed and any material in the cage is trapped within (see FIG. 8B). In some embodiments, material trapped within the cage can undergo crystallization, thereby increasing the quantity of material within the cage. The cage can then be isolated as a crystal containing nano-composite. As this process is freely reversible process, the material can be released by placing the cage under conditions that allow for the expansion of the cage and the formation of open pores (see FIG. 8C). This approach is borrowed from the synthesis of nano-phase inorganic materials from solution and applies equally well to inorganic and organic species.

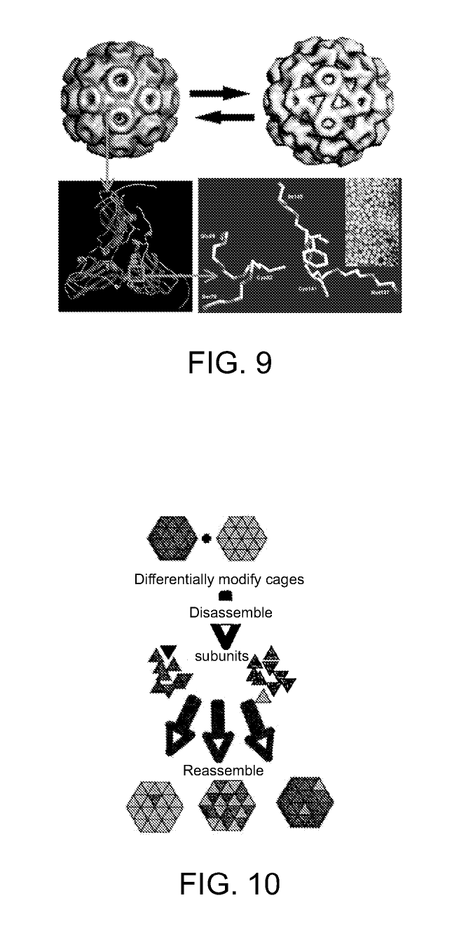

In one embodiment, the present invention provides a protein cage having a reversible gating mechanism. Such a mechanism may provide controlled encapsulation and release of therapeutics. In another embodiment, the present invention provides a protein cage with a reversible, redox-dependent gating mechanism as illustrated in FIG. 9. In another embodiment, the protein cage is a CCMV protein cage. In one other embodiment, at least one CCMV protein subunit is genetically engineered to include thiol-disulfide interaction regions at the protein subunit interfaces in the protein cage. The cage remains in a closed conformation under oxidized conditions (due to the formation of disulfide bonds across the pseudo 3-fold axis of the cage) and this significantly enhances its overall stability. Under mild reducing conditions the cage switches to its open conformation, resulting in the opening of 60, 2 nm pores in the cage architecture. This allows for controlled access between the interior and exterior environments. In some embodiments, a protein cage with a reversible gating mechanism may be set into a closed conformation using a switch, including without limitation reduced pH, redox dependent switches, and glutaraldehyde crosslinking.

An electrostatic model for understanding the reversible gating in CCMV has been developed, which allows the design of mutants that can alter the pH dependence of this gating structural transition. (See Speir, J. A., et al. supra 1995; Zlotnick, A., R. et al., 2000 Virology 277:450-456). The wild type cage remains closed below pH 6.5 due to protonation of acidic residues at the pseudo-3-fold axes of the cage. At higher pH, deprotonation results in an electrostatic repulsion at these sites resulting in a swelling transition. Replacement of the acidic residues with neutral or basic residues may have an effect on pH-dependent gating of the protein cage architecture. Structural transitions may be studied using a suite of techniques including: Mass Spectrometry, site directed spin label EPR spectroscopy, dynamic light scattering, quartz crystal microbalance, and cryo TEM image reconstruction.

In some embodiments, protein cages are modified to provide improved or new chemical switching or gating mechanisms, i.e. chemical switches, that control the reversible swelling of the cages. For example, many viruses are known to undergo reversible structural transitions. The reversible swelling of the CCMV virion is one of the most thoroughly characterized of these structural transitions (Fox, J. M., et al., 1996, Virology 222:115-12235; and Speir, J. A., et al., 1995, Structure. 3:63-78). At pH values<6.5 the virion exists in its compact or closed form. Increasing the pH above 6.5, in the absence of Ca²⁺, results in an 10% expansion (swelling) in the overall dimensions of the virion. Modeling of the CCMV X-ray crystal structure, combined with cryo electron microscopy and image reconstruction of swollen CCMV, indicates that virion swelling is a result of expansion at the quasi three-fold axis of the virion (Speir, J. A., et al., 1995, Structure. 3:63-78). Swelling results in the creation of sixty 20 Å holes which provide access between the interior and exterior of the virion. Thus, one can think of pH as a chemical switch for controlling access to and from the central cavity of the CCMV protein cage (virion). Other means for controlling access to and from the central cavity of protein cages, include redox conditions.

In another embodiment, protein cages are modified to provide improved or new chemical switches for the introduction and delivery of imaging and therapeutic agents. By “chemical switch” herein is meant a factor present in the microenvironment of the protein cage that can be used to control the access to and from the cage's interior. As will be appreciated by those of skill in the art, the switches may be reversible or irreversible (i.e. suicide switches).

By “reversible” herein is a meant a switch that can function in both directions. That is, the switch can be activated to open and close the pores of the cage to allow passage of material in and out of the cage. Examples of chemical switches include pH, ionic strength of the medium, redox conditions, etc.

In some embodiments, protein cages are modified to introduce reversible pH activated switches. In one embodiment, the pH sensitive switch is an acid sensitive switch. Acid sensitive switches may be introduced by adding histidine residues at the Ca²⁺ binding site (see Example 3).

In other embodiments, protein cages are modified to introduce reversible redox activated switches. Redox sensitive switches may be introduced by cysteine residues near the Ca²⁺ binding site (see Example 3).

In one other embodiment, the protein cages are modified to provide irreversible or suicide switches. By “irreversible” or “suicide switches” herein is meant switches that operate in only one direction. In other words, these switches are activated to allow either the entry or exit of materials, but not both. Examples of irreversible switches that may introduced into protein cages include pH switches, redox switches, radiation induction heating switches, near IR switches, radiation induced disassembly, protease sensitive switches and metal dependent switches.

B. Enzymatic Release

In one aspect, the present invention provides a protein cage architecture that may be disassembled via enzymatic action. In one embodiment, a protein cage, which has had one or more amino acid recognition sites introduced into one or more subunits, is provided. The sites may be introduced on the exterior part of the cage. Suitable classes of sites that may be introduced into the protein cages include those sites recognized by a variety of enzymes, including, but are not limited to, hydrolases such as proteases, carbohydrases, lipases and nucleases; isomerases such as racemases, epimerases, tautomerases, or mutases; transferases, kinases and phophatases. In one embodiment, the site is recognized by a hydrolase. In another embodiment, the hydrolase is a protease.

In one embodiment, the present invention utilizes proteases such as serine, cysteine, aspartyl and metalloproteases, including, but not limited to trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; and caspases, such as caspase-3, -5, -8 and other caspases of the apoptotic pathway, and interleukin-converting enzyme (ICE). As will be appreciated in the art, this list is not meant to be limiting.

Many protease cleavage sites are available for use in the present invention including, but not limited to, the 2a site (Ryan et al., J. Gen. Virol. 72:2727 (1991); Ryan et al., EMBO J. 13:928 (1994); Donnelly et al., J. Gen. Virol. 78:13 (1997); Hellen et al., Biochem, 28(26):9881 (1989); and Mattion et al., J. Virol. 70:8124 (1996), all of which are expressly incorporated by reference), prosequences of retroviral proteases including human immunodeficiency virus protease and sequences recognized and cleaved by trypsin (EP 578472, Takasuga et al., J. Biochem. 112(5)652 (1992)) factor Xₐ (Gardella et al., J. Biol. Chem. 265(26):15854 (1990), WO 9006370), collagenase (J03280893, Tajima et al., J. Ferment. Bioeng. 72(5):362 (1991), WO 9006370), clostripain (EP 578472), subtilisin (including mutant H64A subtilisin, Forsberg et al., J. Protein Chem. 10(5):517 (1991), chymosin, yeast KEX2 protease (Bourbonnais et al., J. Bio. Chem. 263(30):15342 (1988), thrombin (Forsberg et al., supra; Abath et al., BioTechniques 10(2):178 (1991)), Staphylococcus aureus V8 protease or similar endoproteinase-Glu-C to cleave after Glu residues (EP 578472, Ishizaki et al., Appl. Microbiol. Biotechnol. 36(4):483 (1992)), cleavage by NIa proteainase of tobacco etch virus (Parks et al., Anal. Biochem. 216(2):413 (1994)), endoproteinase-Lys-C (U.S. Pat. No. 4,414,332) and endoproteinase-Asp-N, Neisseria type 2 IgA protease (Pohlner et al., Bio/Technology 10(7):799-804 (1992)), soluble yeast endoproteinase yscF (EP 467839), chymotrypsin (Altman et al., Protein Eng. 4(5):593 (1991)), enteropeptidase (WO 9006370), lysostaphin, a polyglycine specific endoproteinase (EP 316748), and the like. See e.g. Marston, F. A. O. (1986) Biol. Chem. J. 240, 1-12.

In one embodiment of the present invention, a protein cage is provided having one or more trypsin sites introduced into one or more subunits of the cage. The cage may contain various agents or materials as described herein. In another embodiment, the present invention provides a protein cage with one or more cathepsin sites introduced. The cathepsins belong to the papain superfamily of cysteine proteases. Cysteine or thiol proteases contain a cysteine residue, as well as a histidine and an asparagine, at the active site responsible for proteolysis. This superfamily also has a glutamine at the oxy-anion hole. A number of cathepsin cleavage sites may be utilized in the protein cages provided by present invention.

In one embodiment, a protein cage may include one or more cathepsin B cleavage sites. Cathepsin B is implicated in tumor invasion and progression. Its secretion from cells may be induced by an acidic pH of the medium, although it is functional at physiological pH. It is a protein in the extracellular matrix (ECM) degrading protease cascade and undergoes autodegradation in the absence of a substrate. Cathepsin B has been implicated in breast, cervix, ovary, stomach, lung, brain, colorectal, prostate and thyroid tumors. It is active at the local invasive stage, with stage 1V tumors exhibiting significantly higher concentrations than lower staged tumors. It has been shown to be active at the tumor cell surface, at focal adhesions and invadopodia where the tumor cells contact the basal membrane and ECM. It degrades the ECM, both intracellularly and extracellularly, and includes laminin, fibronectin and collagen IV as its natural substrates. Suitable cathepsin B cleavage sites or substrates include but are not limited to Benzyloxycarbonylarginylarginine 4-methylcoumarin-7-ylamine (Z-Arg-Arg-NH-Mec); trypsinogen; Benzyloxycarbonylphenylarginine 4-methylcoumarin-7-ylamine (Z-Phe-Arg-NH-Mec); N-α-benzyloxycarbonyl-L-arginyl-L-arginine 2-naphthylamide (Z-Arg-Arg-NNap); Benzyloxycarbonylarginylarginine p-nitroanilide (Z-Arg-Arg-p-NA); and Benzyloxycarbonyl-phenylarginine p-nitroanilide (Z-Phe-Arg-p-NA).

In another embodiment, a protein cage may include one or more cathepsin D cleavage sites. Cathepsin D is a 48 kDa aspartyl endoprotease with a classic Asp-Thr-Gly active site. Similar to a variety of other cathepsins, it is made as a 52 kDa precursor, procathepsin D. It is ubiquitously distributed in lysosomes. Cathepsin D has been implicated in breast, renal cell, ovary and melanoma cancers, and appears to be involved in the growth of micrometastases into clinical metastases. In tumor cells, cathepsin D is secreted into the surrounding medium resulting in delivery to the plasma membrane. Similar to cathepsin B, cathepsin D is part of the ECM degrading cascade of proteases. In addition, cathepsin D requires an acidic pH (4.5-5.0) for optimal activity. See Rochefort et al., APMIS 107:86 (1999); Xing et al., Mol. Endo. 12(9): 1310 (1998); Yaziovitskaya et al., Proc. Am. Assoc. Cancer Res. 37:#3553 519 (1996); all of which are expressly incorporated by reference.

In another embodiment, a protein cage may include one or more cathepsin K cleavage sites. Cathepsin K is also an elastolytic cysteine protease, and is considered to be the most potent mammalian elastase, and also has collagenolytic activity. Suitable cathepsin K cleavage sites or substrates include, but are not limited to, Cbz-Leu-Arg-AMC; Cbz-Val-Arg-AMC; Cbz-Phe-Arg-AMC; Cbz-Leu-Leu-Arg-AMC; Tos Gly-Pro-Arg-AMC; Bz-; Phe-Val-Arg-AMC; H-Pro-Phe-Arg-AMC; Cbz-Val-Val-Arg-AMC; Boc-Val-ProArg-AMC; Cbz-Glu-Arg-AMC; Bz-Arg-AMC; Ac-Phe-Arg-AMC; Boc-Val-Leu-Lys-AMC; Suc-Leu-TyrAMC; Boc-Ala-Gly-Pro-Arg-AMC; Cbz-Gly-Pro-Arg-AMC; Z-Leu-Arg-4-methoxy-b-naphthylamide (where Cbz=benzyloxycarbonyl and AMC=aminomethylcoumarin); diaminopropanones, diacylhydrazine and cystatin C. See Bossard, M. J. et al., J. Biol. Chem. 271, 12517-12524 (1996); Aibe, K. et al., Biol. Pharm. Bull. 19, 1026-1031 (1996); Votta, B. J. et al. J. Bone Miner. Res. 12, 13961406 (1997); Yamshita, D. S. et al. J. Am. Chem. Soc. 119, 11351-11352 (1997); DesJarlais, R. L. et al. J. Am. Chem. Soc. 120, 9114-9115 (1998); Marquis, R. W. et al. J. Med. Chem. 41, 3563-3567 (1998); Thompson et al., J. Med. Chem. 41, 3923-3927 (1998); Thompson et al., Bioorg. Med. Chem. 7, 599605 (1999); Kamiya, T. et al. J. Biochem. (Tokyo) 123, 752-759 (1998), Shi et al., J. Clin. Invest. 104:1191 (1999); and Sukhova et al., J. Clin. Invest. 102:576 (1998), all of which are expressly incorporated by reference.

In one embodiment, a protein cage may have one or more cleavage sites for the protease, hepsin. Hepsin has been implicated in ovarian cancer, and appears to be involved in tumor invasion and metastasis by allowing implantation and invasion of neighboring cells. It is a serine protease with a classic catalytic triad (Ser-His-Asn). It degrades the ECM through peptide bond cleavage, and is found extracellularly. See Tantimoto et al., Proc. Am. Assoc. Cancer Res. 38:(#2765):413 (1997).

It has been shown that proteases can direct the disassembly of the protein cage architecture. For example, wild type CCMV is relatively insensitive to cleavage by trypsin. Trypsin sites were introduced into exposed regions of the coat protein and cages were subjected to time-course digestion by trypsin and monitored by TEM, SDS-PAGE and Mass Spectrometry. (See Bothner, B., et al., 1998 J. Biol 273:673-676.) These studies demonstrated that introduced trypsin cleavage sites are active and lead to rapid disassembly of the CCMV protein cage. These results indicate that protease-based release mechanisms may be useful control mechanisms for release of encapsulated therapeutic agents from protein cage structures. In additional embodiments, protease sites may be introduced into one or more subunits in at least one protein cage so as to allow proteases to direct the disassembly of the protein cage architecture. In another embodiment, protease sites for tissue specific proteases may be introduced. In one embodiment, the tissue specific protease is cathepsin B (Dubowchik, G. M. et al., 2002a Bioconjug Chem 13:855-69).

In some embodiments, a protein cage having cathepsin B cleavage sites can release the anti-cancer drug from the N-terminus of the cage. In another embodiment, the protein cage having cathepsin B cleavage sites is rapidly disassembled upon cleavage to facilitate release of the anti-cancer drug. In one embodiment, drug release from a protein cage described herein is dependent on cellular uptake, thus limiting the cytotoxic effect to the cells and tissues targeted via the peptides described herein, for example in Table 1.

V. Targeting Moieties

In one embodiment, a targeting moiety is added to the composition. It should be noted that in the case of polymers, the targeting moiety may be added either to the nanoparticle itself or to the polymer. By “targeting moiety” herein is meant a functional group which serves to target or direct the complex to a particular location, cell type, diseased tissue, or association. In general, the targeting moiety is directed against a target molecule and allows concentration of the compositions in a particular localization within a patient. In some embodiments, the agent is partitioned to the location in a non-1:1 ratio. Thus, for example, antibodies, cell surface receptor ligands and hormones, lipids, sugars and dextrans, alcohols, bile acids, fatty acids, amino acids, peptides and nucleic acids may all be attached to localize or target the nanoparticle compositions to a particular site.

In another embodiment, the targeting moiety allows targeting of the nanoparticle compositions to a particular tissue or the surface of a cell.

In other embodiments, the targeting moiety is a peptide. For example, chemotactic peptides have been used to image tissue injury and inflammation, particularly by bacterial infection; see WO 97/14443, hereby expressly incorporated by reference in its entirety. Peptides may be attached via the chemical linkages to reactive groups on the exterior surface of the protein cage architectures (Flenniken, M. L., et al. 2005. Chemical Communications:447-449), (Flenniken, M. L., et al. 2003. Nano Letters 3:1573-1576), (Gillitzer, E., et al. 2002. Chemical Communications:2390-2391), (Hermanson, G. T. 1996. Academic Press, San Diego), (Wang, Q., et al. 2002. Chemistry & Biology 9:805-811; Wang, Q., et al. 2002. Chemistry & Biology 9:813-819; Wang, Q., et al. 2002. Angewandte Chemie-International Edition 41:459-462)). In some embodiments, peptides are attached to endogenous or engineered reactive functional groups on the exterior surface of each of the protein cage systems.

In other embodiments, the present invention provides methods for the chemical attachment of RGD, NGR, F3, LyP-1 peptides to exterior surfaces of both the CCMV and sHsp protein cage architectures. Multiple chemical approaches may be used for ligand attachment. Activation of carboxylic acid groups and reaction with nucleophiles such as primary amines affords the coupling of ligands through formation of amide linkages. Engineered thiol functional groups (cys) on the protein may be modified by reaction with commercially available maleimide or iodoacetimide bifunctional linkers. In addition, synthetic methodologies developed for attachment through azide groups, and photochemical reactions of nucleophiles with tyrosine residues can be utilized. The range of established synthetic procedures is summarized in Table 1 (See section II.A.1. Small molecules and drugs) listing all reactions previously demonstrated as effective for the attachment of molecules to protein cage architectures.

In another embodiment, the peptide is attached to a protein cage by the mechanism known as “click chemistry” (see Hartmuth, C. et al. (2001) Angewandte Chemie Int'l 40(11):2004-21). Click chemistry is a modular protocol for organic synthesis that utilizes powerful, highly reliable and selective reactions for the rapid synthesis of compounds. In one application involves the use of azides or alkynes as building blocks due to their ability to react with each other in a highly efficient and irreversible spring-loaded reaction. In one embodiment, the attachment to a protein cage of (i) proteins as targeting moieties and/or therapeutic agents and/or (ii) drugs as therapeutic agents, is achieved through the use of an azide linkage.

In one other embodiment, the attachment of proteins is achieved by a form of peptide ligation utilitzing an alkyne-azide cycloaddition reaction (Aucagne, V. et al. (2006) Sep. 28; 8(20): 4505-7).

The present invention provides protein cages having targeting ligands on the exterior surface, as well as chemical and genetic approaches for introducing targeting ligands on to the exterior surfaces of the protein cage architectures. In one embodiment, such a targeting ligand specifically interacts with at least one exposed receptor molecule on cell surfaces. In additional embodiments, a monoclonal antibody specific for cell surface CD4 is chemically linked to the exterior of a protein cage architecture. As described in Example 6, FACS analysis of mouse spleen cells demonstrated the ability of these cages to specifically target a subpopulation of spleen cells expressing CD4 and this interaction was shown to be specific by competition/blocking assays with free monoclonal antibody.

It has been shown that RGD targeting peptides may be incorporated into a protein cage (Arap, W., et al., 1998, Science 279:377-80; Pasqualini, R., E. et al., 1995, J Cell Biol 130:1189-96). For example, the RGD-4C peptide, specific to cell surface exposed αvβ3 and αvβ5 integrins, may be genetically incorporated into the sHsp cage architecture. in tumor cell culture assays such targeted cages were shown to bind to cells (C32 melanoma) known to express these integrins, and the interaction was shown to be specific based on competition assays (See Examples 6 and 6A).

In one aspect of the present invention, the protein cages of the present invention include a plurality of proteins as targeting moieties. In one embodiment, the protein cages includes a plurality of peptides. In another embodiment, the plurality of peptides is 2, 3, 4, 5, 6, 7, 8, 9, 10, 11, 12, 13, 14, 15, 16, 17, 18, 19, 20, 21, 22, 23, or 24, 25, 26, 27, 28, 29, 30. The plurality of peptides may also be more than 30 peptides. In one other embodiment, the peptides may be RGD-4C peptides (CDCRGDCFC), which bind selectively to integrins as previously described herein, which target cancer cells.

In another embodiment, the protein cage having a plurality of peptides as targeting moieities may also include an imaging agent. The imaging agent may be an inorganic material agent as described herein (section III.A.6. Inorganic material agents). In some embodiments, the protein cage is a ferritin protein cage having a plurality of RGD-4C peptides externally located on the cage and ferromagnetic iron oxide particles located on the cage interior. Alternatively, the protein cage may have an optical agent, such as a fluorescent label. (See Example 6A).

In another embodiment, the present invention provides a protein cage containing M. jannaschii small heat shock proteins and having one or more RGD-4C peptides attached by genetic modification to the cage exterior. In one additional embodiment, a small heat shock protein cage includes an antibody conjugated to the exterior surface by chemical modification. (see Flenniken, M. L. et al. (2006) February; 13(2):161-70 and Example 6A).

In some embodiments, protein cages are modified for the attachment of targeting moieties. By the term “targeting moiety” herein is meant a functional group that serves to target or direct the delivery vehicle, i.e., the cage comprising at least one medical imaging agent, to a particular location or association, i.e. a specific binding event. Thus, for example, a targeting moiety may be used to target a molecule to a specific target protein or enzyme, or to a particular cellular location, to a particular cell type, to a diseased tissue. As will be appreciated by those in the art, the localization of proteins within a cell is a simple method for increasing effective concentration. For example, shuttling an imaging agent and/or drug into the nucleus confines them to a smaller space thereby increasing concentration. Finally, the physiological target may simply be localized to a specific compartment, and the agents must be localized appropriately.

Suitable targeting moieties include, but are not limited to, proteins, nucleic acids, carbohydrates, lipids, hormones including proteinaceous and steroid hormones, growth factors, receptor ligands, antigens and antibodies, and the like. Proteins in this context means proteins (including antibodies), oligopeptides and peptides, derivatives and analogs, including proteins containing non-naturally occurring amino acids and amino acid analogs, and peptidomimetic structures. The side chains may be in either the (R) or the (S) configuration. In one embodiment, the amino acids are in the (S) or L-configuration.

Suitable targeting sequences include, but are not limited to, binding sequences capable of causing binding of the moiety to a predetermined molecule or class of molecules while retaining bioactivity of the expression product, (for example by using enzyme inhibitor or substrate sequences to target a class of relevant enzymes); sequences signaling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Suitable proteins include without limitation peptides, antibodies and cell surface ligands.

In one embodiment, the targeting moiety is a peptide. For example, chemotactic peptides have been used to image tissue injury and inflammation, particularly by bacterial infection; see WO 97/14443, hereby expressly incorporated by reference in its entirety.

In one other embodiment, the targeting moiety is laminin peptide 11. Peptide 11 is a well characterized system for understanding cancer cell metastasis (Landowski, T. H., et al., 1995, Biochemistry, 34:11276-11287; Landowski, T. L., et al., 1995, Clin. Exp. Metastasis 13:357-372; Menard, S., et al., 1997, J. Cell Biochem. 67:155-165; J. R. Starkey, et al., 1999, Cytometry 35:37-47; Starkey, J. R., 1994, Human Pathology, 25:1259-1260; and, J. R. Starkey, et al., 1998, Biochim. Biophys. Acta. 1429:187-207). Briefly outlined, the interactions of tumor cells with basement membrane components are considered to be critical determinants of the ability of a tumor to invade and spread to distant sites. The 67 kDa high affinity laminin binding protein (LBP) is a cell surface protein thought to mediate such invasive interactions (Menard, S., et al., 1997, J. Cell Biochem. 67:155-165). The expression of LBP is positively correlated with progression in many solid tumors (Mafune, K., et al., 1990, Cancer Res. 50:3888-3891; Sanjuan, X., et al., 1996, J. Pathol. 179:376-380; and Viacava, P., et al., 1997, J. Pathol. 182:36-44). The major ligand binding for LBP is the laminin-1 protein. A ten amino acid sequence from laminin-1 b chain, CDPGYIGSRC, known as peptide 11, is the primary ligand binding domain for LBP (Graf, J., et al., 1987, Cell, 48:989-996; Iwamoto, Y., et al., 1996, Br. J. Cancer 73:589-595; and Iwamoto, Y., et al., 1987, Science 238:1132-1134). Free peptide 11 effectively blocks invasion of basement membranes by tumor cells, reduces experimental tumor lung colonization, and inhibits tumor angiogenesis in mice (Mafune, K., et al., 1990, Cancer Res. 50:3888-3891; Sanjuan, X. et al., 1996, J. Pathol. 179:376-380; and Viacava, P., et al., 1997, J. Pathol. 182:36-44). Research, using both in vitro binding assays and in situ localization studies, has strongly suggested that the anti-metastatic activity of free peptide 11 is a direct result of binding to the LBP (J. R. Starkey, et al., 1999, Cytometry 35:37-47; and J. R. Starkey, et al., 1998, Biochim. Biophys. Acta. 1429:187-207). Among the known sequences for animal proteins, the peptide 11 sequence is specific to laminin. The 67 kDa LBP is highly conserved in evolution (Bignon, C., et al., 1992, Biochem Biophys Res Commun. 184:1165-1172) and has been shown to be expressed quite early in development where it likely plays a role in the direction of cell migration on laminin containing substrates (Laurie, G. W., et al., 1989, J. Cell Biol. 109:1351-1362). The 67 kDa LBP is also present on platlets and neutrophils, but free peptide 11 has no toxic effect on these cells. The properties of peptide 11 suggest that it could be adapted for tumor targeting. As described in Example 4, we have expressed peptide 11 on the surface of the CCMV protein cage in an effort to target these cages to solid tumor cells.

The present invention provides protein cages incorporating peptide based tumor-targeting ligands, and methods of making thereof. In one embodiment, the protein cages are targeted to a specific cell/tissue type for use in drug delivery/imaging systems. In another embodiment, peptide or antibody-based cell targeting ligands are added on the exterior surface of a protein cage. In some embodiments, the targeting ligand is specific for tumor induced angiogenic vasculature. In other embodiments, the targeting ligand is a tumor vascular homing peptide with tumor cell-penetrating properties. Table 2 provides targeting peptides suitable for the use in the present invention.

The four targeting peptides of Table 2 have been shown to target angiogenic vasculature with high specificity in vivo. Two peptides are not typically internalized (RGD-4C and peptides to aminopeptidase N (NGR) and two peptides are internalized (F3 and LyP-1). RGD-4C is a well-studied targeting peptide that binds to αvβ3 and αvβ5 integrins expressed on angiogenic endothelium present in tumors. Aminopeptidase N is a membrane protein that is expressed on angiogenic vessels within the vasculature. F3 is a 35 amino acid peptide that targets angiogenic endothelium and tumor cells. The receptor for F3 is a cell surface-expressed nucleolin. F3 is internalized to the nuclei in rapidly proliferating cells. Lyp-1 is a nine amino acid cyclic peptide that specifically targets tumor lymphatics and tumor cells. LyP-1 is extraordinarily effective as a tumor homing peptide that interacts with an unknown receptor. This collection of peptide targeting ligands are some of the most effective and well characterized tumor homing peptides known.

TABLE 2
Peptides for use as targeting moieties

| | | | | |
|---|---|---|---|---|
| Peptide | Sequence | Specificity | Receptor | Reference |

| | | | | |
|---|---|---|---|---|
| F3 | KDEPQRRSARL | Angiogenic | Cell surface | 1a |
| | SAKPAPPKPEP | endothelium | nucleolin | |
| | KPKKAPAKK | tumor cells | | |
| LyP-1 | CGNKRTRGC | Tumor | not known | 1b |
| | | lymphatics & | | |
| | | tumor cells | | |
| NGR | CNGRCVS | Angiogenic | Aminopeptidase N | 1c |
| | GCAGRC | endothelium & | | |
| | | & tumor cells | | |
| RGR-4C | CDCRGDCFC | Angiogenic | αvβ3 and αvβ5 | 1d |
| | | endothelium & | integrins | |
| | | & tumor cells | | |

1a. (Christian, S., et al. 2003. J Cell Biol 163:871 -878; Laakkonen, P., et al. 2002. Nat Med 8:751-5)
1b. (Laakkonen, P., et al. 2004. Proc Natl Acad Sci U S A 101:9381-6; Laakkonen, P., et al. 2002. Nat Med 8:751-5)
1c. (Arap, W., et al. 1998. Science 279:377-80; Pasqualini, R., et al. 2000. Cancer Res 60:722-7)
1d. (Arap, W., et al. 1998. Science 279:377-80); Pasqualini, R., et al. 1995. J Cell Biol 130:1189-96)

In one embodiment, the present invention provides methods including the targeting of peptides to the external surface of protein cages, including without limitation CCMV protein cages and sHsp protein cages. In some other embodiments, targeting peptides are incorporated genetically as either N- or C-terminal fusions or by incorporation into surface exposed loops in the subunits of the protein cages. N- and C-terminal fusions as well as fusions into surface exposed loops to protein cage subunits will be used to present peptides on cage surfaces. The DNA oligonucleotides encoding for the RGD, NGR, F3, and LyP-1 may be introduced into surface exposed loops, or N- and C-termini of the two protein cage architectures by site-directed mutagenesis of cloned sHsp and CCMV subunit genes. It has been shown that up to 30 kDa peptides can be expressed on the protein cage architecture.

In one other embodiment, the protein cages may include MCP-1, a chemoattractant peptide used in selective macrophage uptake and targeting of vulnerable plaque (inflammation). MCP-1 is a chemokine that binds to and is internalized by macrophages. It has been shown to be increased in atherosclerosis. It has the advantages of targeting primarily macrophages and being internalized to allow intracellular accumulation. MCP-1 peptide (76 amino acid residues, 8.6 kDa) can be introduced into surface exposed loops, or N- and C-termini of the two protein cage architectures. The MCP-1 may also be attached via the chemical linkages disclosed in Table 1 to reactive groups on the exterior surface of the protein cage architectures. For example antibodies against MCP-1 (MCP-1 antibodies are commercially available from R&D Systems and from Research Diagnostic Inc) can be attached to endogenous or engineered reactive functional groups on the exterior surface of each of the protein cage systems. Activation of carboxylic acid groups and reaction with nucleophiles such as primary amines affords the coupling of ligands through formation of amide linkages. Engineered thiol functional groups (cys) on the protein will be modified by reaction with commercially available maleimide or iodoacetimide bifunctional linkers. In addition, new synthetic methodologies developed for attachment through azide groups, and photochemical reactions of nucleophiles with tyrosine residues may be utilized. The range of established synthetic procedures is summarized in Table 1.

In one embodiment, the targeting moiety is an antibody, as previously described. In one other embodiment, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986); Riechmann et al., Nature 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology, 14:826 (1996); Lonberg and Huszar, Intern. Rev. Immunol. 13:65-93 (1995).

Bispecific antibodies are monoclonal, such as human or humanized, antibodies that have binding specificities for at least two different antigens. In the present case, one of the binding specificities is for a first target molecule and the other one is for a second target molecule.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion may be with an immunoglobulin heavy-chain constant domain, comprising at least part of the hinge, CH2, and CH3 regions. It is possible to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In other embodiments, the antibody is directed against a cell-surface marker on a cancer cell; that is, the target molecule is a cell surface molecule. As is known in the art, there are a wide variety of antibodies known to be differentially expressed on tumor cells, including, but not limited to, HER2.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In one embodiment, antibodies against virus or bacteria can be used as targeting moieties. As will be appreciated by those in the art, antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like) may be used.

In another embodiment, the targeting moiety is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF- and TGF-), EPO receptor (EPO), TPO receptor (TPO), ciliary neurotrophic factor receptor, prolactin receptor, and T-cell receptors. Hormone ligands may be suitable. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathyroid hormone, thyroid-stimulating hormone (TSH), vasopressin, enkephalins, seratonin, estradiol, progesterone, testosterone, cortisone, and glucocorticoids and the hormones listed above. Receptor ligands include ligands that bind to receptors such as cell surface receptors, which include hormones, lipids, proteins, glycoproteins, signal transducers, growth factors, cytokines, and others.

In other embodiments, the targeting moiety is a carbohydrate. By “carbohydrate” herein is meant a compound with the general formula Cx(H2O)y. Monosaccharides, disaccharides, and oligo- or polysaccharides are all included within the definition and comprise polymers of various sugar molecules linked via glycosidic linkages. Suitable carbohydrates are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates. In particular, polysaccharides (including, but not limited to, arabinogalactan, gum arabic, mannan, etc.) have been used to deliver MRI agents into cells; see U.S. Pat. No. 5,554,386, hereby incorporated by reference in its entirety. In addition, N-(2-hydroxypropyl)methacrylamide (HPMA) copolymers have been shown to bind human hepatocarcinoma HepG2 cells. In particular, trivalent galactose and lactose-containing copolymers demonstrated preferential binding of the cells and the level of binding increased as the saccharide moiety content increased. (Kopeckova, P. et al., (2001). Bioconjug. Chem. November-December; 12(6):890-9). In one embodiment, the present invention provides a protein cage having a HPMA targeting moiety.

As outlined herein, targeting moieties can be organic species including biomolecules are defined herein.

In other embodiments, the targeting moiety may be used to either allow the internalization of the nanoparticle composition to the cell cytoplasm or localize it to a particular cellular compartment, such as the nucleus.

In one embodiment, the targeting moiety is a lipid. “Lipid” as used herein includes fats, fatty oils, waxes, phospholipids, glycolipids, terpenes, fatty acids, and glycerides, particularly the triglycerides. Also included within the definition of lipids are the eicosanoids, steroids and sterols, some of which are also hormones, such as prostaglandins, opiates, and cholesterol.

In another embodiment, the targeting moiety is all or a portion of the HIV-1 Tat protein, and analogs and related proteins, which allows very high uptake into target cells. See for example, Fawell et al., PNAS USA 91:664 (1994); Frankel et al., Cell 55:1189 (1988); Savion et al., J. Biol. Chem. 256:1149 (1981); Derossi et al., J. Biol. Chem. 269:10444 (1994); Baldin et al., EMBO J. 9:1511 (1990); Watson et al., Biochem. Pharmcol. 58:1521 (1999), all of which are incorporated by reference.

In one embodiment, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-β nuclear localization signal (ARRRRP); NFkappaB p50 (EEVQRKRQKL; Ghosh et al., Cell 62:1019 (1990); NFkappaB p65 (EEKRKRTYE; Nolan et al., Cell 64:961 (1991); and others (see for example Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann, Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

In some embodiments, targeting moieties for the hepatobiliary system are used; see U.S. Pat. Nos. 5,573,752 and 5,582,814, both of which are hereby incorporated by reference in their entirety.

Targeting moieties may be added to the surface of protein cages either by engineering protein cages to express the targeting moiety or by the addition of functional groups to the surface of the protein cage. In another embodiment, the protein cage is engineered to express the targeting moiety. For example, one or more of the five surface exposed loops of a CCMV protein cage may be used for the expression of the targeting moiety (see Example 4).

VI. Methods of Loading Protein Cages

Once made, the protein cages are loaded with medical imaging agents and/or therapeutic agents. By “loaded” or “loading” or grammatical equivalents herein is meant the introduction of imaging agents, therapeutic agents and other non-native materials into the interior of the protein shell (also referred to herein as “crystallization” or “mineralization” depending on the material loaded). As will be appreciated by those of skill in the art, loading includes the synthesis of materials within the shell.

In some embodiments, the protein shells are empty. By “empty” herein is meant that the cages are prepared lacking materials that would commonly be contained within. For example, if viral protein cages are used, the shells are prepared lacking viral nucleic acids and proteins.

In general, the protein cages are made recombinantly and self assemble upon contact (or by alteration of their chemical environment). As will be appreciated by those in the art, there are a wide variety of available techniques for the production of proteins in a wide variety of organisms.

In addition to protein cages, some embodiments of the invention, for example those utilizing arrays and compositions of mixtures of nanoparticles and nanoparticle cores, the nanoparticles can utilize viral protein cages, such as those of the CCMV virus as well as others, including tobacco mosaic virus (TMV); see U.S. Pat. No. 6,180,389, hereby incorporated by reference in its entirety. TMV serves as a particularly good “spacer” given its size.

In one embodiment, the spacer material comprises dendrimers. As will be appreciated by those in the art, a variety of dendrimeric structures find use in the present invention, in general any dendrimer that can incorporate dopants that allow for the alteration of magnetical and electrical properties as is known in the art can be used.

In another embodiment, the spacer material comprises an insulating material as is known in the art, including organic polymer, SiO₂, Al₂O₃, and any number of well known additives.

The protein cages are loaded with materials. By “loaded” or “loading” or grammatical equivalents herein is meant the introduction of non-native materials into the interior of the protein shell (sometimes referred to herein as “mineralization”, depending on the material loaded). In other embodiments, the protein shells are devoid of their normal cores; e.g. ferritins in the absence of iron (e.g. apoferritins) are loaded; alternatively, additional loading is done in the presence of some or all of the naturally occurring loading material (if any). In general, there are two ways to control the size of the core; by altering the cage size, as outlined herein, or by controlling the material to protein shell ratio (e.g. the loading factor). That is, by controlling the amount of available material as a function of the amount and size of shells to be loaded, the loading factor of each individual particle can be adjusted. For example, as outlined below, mammalian ferritin shells can generally accommodate as many as 4,000 iron atoms, while protein cages containing Dps-like proteins from Listeria innocua can accommodate 500. These presumably maximum numbers may be decreased by decreasing the load factors. In general, the loading is an equilibrium driven passive event or entrapment (although as outlined below, the natural channels or “holes” in the shells can be manipulated to alter these parameters), where physiological buffers, temperature and pH are suitable, with loading times of 12-24 hours.

Typically, for the mineralization of the 24 subunit ferritin, aliquots of Fe2+ (25 mM as (NH₄)Fe(SO4)2.6H2O) are added to a solution of apoferritin (1 mg) in roughly 4 mL of a morpholine sulfonate buffer (MES (0.1M, pH 6.5) and stirred with a magnetic stirrer. The Fe(II) is added in aliquots of 40 μL corresponding to ˜500 Fe2+ atoms/protein cage. The reaction to stir and air oxidize for ˜1 hour between additions and left to stir overnight (±24 hrs) at 4° C. The same procedure is used for mineralizing the other cages (Listeri ferritin-like protein and CCMV) but with slightly different amounts of Fe. Other buffers can be used and the pH of the reaction can be altered between 6 and 9. We can also do the reaction in the absence of any buffer and changes in pH can be titrated using an auto-titrator. In addition, these general conditions work for other metals as well. Some embodiments generally utilize solutions of anywhere from 10000:1 to 1:1 material:shell.

The protein cages are loaded with materials. In this context, “material” includes both inorganic, organic and organometallic materials, ranging from single atoms and/or molecules to large conglomerates of the same. In one embodiment, the protein cages are loaded with inorganic materials, including, but not limited to, metals, metal salts, metal oxides (including neat, doped and alloyed metal oxides), non-metal oxides, metal and non-metal chalcogens, sulfides, selinides, coordination compounds, organometallic species. Suitable metals include, but are not limited to, monovalent and polyvalent metals in any form depending on the end use of the nanoparticle and/or core; e.g. elemental, alloy (where relative concentrations of the elements can vary continuously—(Co/Ni, Co/Fe/Ni etc.)) and intermetallic (which are distinct compounds with definite stoichiometries—(CO₃Pt, FePt, FePt₃ etc.)). For monovalent metal salts, silver chloride may be used to nanoparticles useful in photographic applications. Polyvalent metals include, but are not limited to, transition metals and mixtures, including aluminum, barium, chromium, cobalt, copper, europium, gadolinium, lanthanum, magnesium, manganese, nickel, platinum, neodymium, titanium, yttrium, zirconium, terbium, zinc and iron, as well as other lanthanides. Metals that can possess magnetic properties such as iron are suitable. Some embodiments utilize mixtures of metals, such as Co, Ni, Fe, Pt, etc. as outlined herein.

In one embodiment, as outlined in the examples, the nanoparticles can be made with zero valent metals from oxide precursors. In this embodiment, the shells are loaded with metal oxides such as iron oxide and then reduced using standard techniques.

In general, there are two ways to control the size of the core; by altering the cage size, as outlined herein, or by controlling the material to protein shell ratio (e.g. the loading factor). That is, by controlling the amount of available material as a function of the amount and size of shells to be loaded, the loading factor of each individual particle can be adjusted. For example, as outlined below, mammalian ferritin shells can generally accommodate as many as 4,000 iron atoms, while protein cages from Listeria can accommodate 500. These presumably maximum numbers may be decreased by decreasing the load factors. In general, the loading is an equilibrium driven passive event or entrapment (although as outlined below, the natural channels or “holes” in the shells can be manipulated to alter these parameters), with physiological buffers, temperature and pH being suitable, with loading times of 12-24 hours.

In one aspect, the present invention provides protein cages that can mineralize a metal, such as for example iron to form a size-constrained material. In one embodiment, the metal mineralized is not iron. Such protein cages may be mineralized under physiological conditions (See Yang, X. et al., Iron oxidation and hydrolysis reactions of a novel ferritin from Listeria innocua. Biochem J. 2000 Aug. 1; 349 Pt 3:783-6; Stefanini, S. et al., Incorporation of iron by the unusual dodecameric ferritin from Listeria innocua. Biochem J. 1999 Feb. 15; 338 (Pt 1):71-5. Erratum in: Biochem J 1999 May 1; 339 (Pt 3):775; Bozzi, M., et al. (1997) A Novel Non-heme Iron-binding Ferritin Related to the DNA-binding Proteins of the Dps Family of Listeria innocua. J. Biol. Chem. 272, 3259-3265) or non-physiological conditions (Allen M. et al., (2002). Protein Cage Constrained Synthesis of Ferrimagnetic Iron Oxide Nanoparticles. Adv. Mater. 14, 1562-1565; Allen, M. et al., (2003). Constrained Synthesis of Cobalt Oxide Nano-Materials in the 12-Subunit Protein Cage from Listeria innocua. Inorg. Chem. 42, 6300-6305).

In another embodiment, the present invention provides protein cages formed from Dps proteins that contain a metal mineralized under non-physiological conditions. In one embodiment, the Dps proteins are from L. innocua. Non-physiological conditions include a certain temperature and pH. The temperature for non-physiological conditions may be from about 50° C. to about 70° C. In addition, the temperature may be about 50° C. or greater, about 55° C. or greater, about 60° C. or greater, about 61° C. or greater, about 62° C. or greater, about 63° C. or greater, about 64° C. or greater, and about 65° C. or greater. The pH of non-physiological conditions may be from about 7.5 to about 9. In addition, the pH may be about 7.5, about 8, about 8.5, and about 9.

VII. Methods of Protein Cage Assembly

In one embodiment, CCMV protein cages may be assembled both in vivo and in vitro from 180 identical protein subunits to form empty, non-infectious, protein cage architectures. In one embodiment, a yeast-based heterologous expression system is used for the self-assembly of empty CCMV which allows the separation of viral assembly from other viral functions to produce empty non-infectious particles in large quantities (20, Brumfield, S., D. et al., 2004. Journal of General Virology 85:1049-1053). Such a system expands the range of modifications that can be made to viral based protein cages. The 12 nm heat shock protein cage (sHsp) is assembled from 24 identical subunits.

In some embodiments, in particular with the dodecameric protein cages, the natural channels to the interior formed by the two-, three-, and four-fold symmetry of the dodecameric proteins may be modified to enable either the introduction and/or extraction, or both, of materials through the opening therein.

In one embodiment, the present invention provides methods for in vitro assembly of protein cages. As illustrated in FIG. 10, differentially modified cage subunits can be reassembled to control multifunctional ligand presentation. In other embodiments, the cage subunits are CCMV and/or sHsp cage subunits. In another embodiment, methods for assembly include providing separate populations of protein cages that may be chemically or genetically modified with desired ligands (cell targeting, fluorescent dyes, biotin). For example, one population may have a genetically (or chemically) introduced surface exposed ligand, while a second population presents a different ligand.

Example 7 describes the use of a CCMV cage to assembly asymmetrically functionalized particles.

In one embodiment, the methods for assembly include disassembling the protein cages into subunits, purifying the disassembled subunits, differentially labeling the purified subunits, and mixing the purified subunits in defined molar ratios under conditions that lead to the efficient reassembly of the protein cage architecture. In other embodiments, the re-assembled protein cages incorporate both two or more ligand types. In one embodiment, co-assembled CCMV subunits are differentially labeled with biotin and digoxigenin.

In one other embodiment, the protein cage is loaded via the use of a chemical switch. For example, if a pH sensitive switching mechanism is used, empty cages are dialyzed in a saturated solution comprising an imaging agent, or an imaging agent and a therapeutic agent at room temperature and at pH>6.5 to ensure that that the cage is in an open conformation. Once crystallization has been initiated, the pH of the solution is lowered, i.e. pH<6.5 to switch the cage to a closed formation. The resulting cage with its entrapped imaging agent, etc., is then isolated using gradient centrifugation or column chromatography. If desired, the cages can be isolated prior to bulk crystallization and counter ions, such as Me₄N⁺ added to induce crystal formation. Once isolated, the cages may be stored or used directly.

In alternative methods, the protein cage is loaded using a redox sensitive switch. In this embodiment, the redoxing conditions are altered. For example, reducing conditions should favor cage expansion, i.e., open conformation, via the breaking of disulfide bonds and entrapment of imaging agents. Alternatively, oxidizing conditions should prevent expansion and thereby the release of entrapped agents.

Once made, the compositions of the invention find use in a variety of applications.

VIII. Derivatization of Protein Cages

In some embodiments, the nanoparticles are derivatized for attachment to a variety of moieties, including but not limited to, dendrimer structures, additional proteins, carbohydrates, lipids, targeting moieities, etc. In general, one or more of the subunits is modified on an external surface to contain additional moieties.

In one embodiment, the nanoparticles can be derivatized as outlined herein for attachment to polymers as previously described. One embodiment of the present invention utilizes polylysine as the polymer. The —NH₂ groups of the lysine side chains at high pH serve as strong nucleophiles for multiple attachment of nanoparticles. At high pH the lysine monomers can be coupled to the nanoparticles under conditions that yield on average 5-20% monomer substitution. The size of the polymer may vary substantially as previously described.

Other modifications include the addition of dendrimers to the interstitial space of the cage, further outlined below.

IX. Protein Cage Aggregation

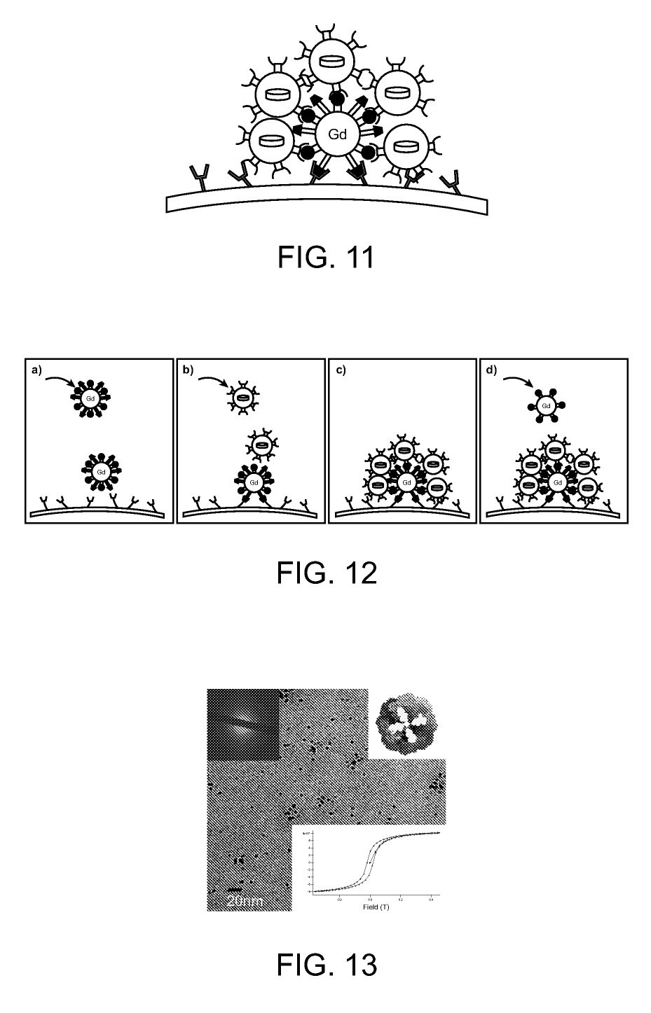

In one embodiment, the present invention provides methods of drug delivery and imaging based on the programmed aggregation of protein cage architectures. In some embodiments, the methods include providing a protein cage as described herein to first target an imaging agent to a particular tissue (without any drug) and visualizing the target (i.e. tumor) in real time. In another embodiment, the methods include providing a plurality of protein cages containing a drug or small molecule and amplifying drug delivery to a target site. As illustrated by FIG. 11 and FIG. 12, protein cage aggregation at a targeted cell surface may be programmed and sequential.

In one embodiment, a CCMV protein cage modified to present biotin on its surface may allow for the controlled aggregation with a second protein cage presenting avidin. In these aggregates each particle is completely surrounded by complementary particles. In addition, it has been demonstrated that two different ligand types may be presented on the same protein cage platform and that a single CCMV protein cage may be modified with up to 180 biotin and 180 fluorophore molecules. (Gillitzer, E., D. et al., 2002. Chemical Communications:2390-2391) In one embodiment, protein cage aggregation ligands include without limitation biotin/avidin. In other embodiments, protein cage cell targeting ligands include without limitation RGD.

In one other embodiment, protein cages are modified to provide an interface for molecular aggregation, i.e., crystallization, based on complementary electrostatic interactions between the protein cage and the entrapped material. Previous work has shown that protein cages are ideal reaction vessels for the constrained crystallization of materials (Douglas, T., and M. J. Young, 1998, Nature 393:152-155). For example, a range of polyoxometalate species (i.e., vanadate, molybdate, tungstate) have been crystallized within CCMV protein cages. Similarly, tungstate has been crystallized with the Norwalk Virus protein cage (see Example 2).

The present invention provides for the controlled fabrication of protein cage nano-particle clusters incorporating functionalized cell targeting ligands and MRI contrast agents for enhanced magnetic properties. The controlled aggregation of magnetic nano-particles may lead to the enhancement of the MR characteristics. In one embodiment, protein cage clustering may be directed such that core shell structures of protein cages are assembled from a defined number of particles.

In one embodiment, the core may be a protein cage reacted with activated streptavidin as succinimidyl ester and the shell may be a symmetry broken protein cage reacted with maleimide activated biotin. Mixing of the core and shell results in a biotin-streptavidin interaction leading to formation of core-shell structures. These cluster structures may be reacted with activated streptavidin and the addition of excess symmetry-broken biotin labeled cages may lead to assembly of additional shells of cages. This approach is analogous to the strategy for molecular core shell synthesis (e.g. dendrimer synthesis) except that the building blocks are large magnetic nano-particles.

This approach utilizes the breakage of functional symmetry of individual protein cages to allow presentation of a single functional group on the exterior surface. Using these protein cages with single functional groups, desired core shell structures can be built. In one embodiment, the exterior of one or more protein cages may be functionalized with an excess of streptavidin, constituting the core of the aggregate structure. The core may be subsequently mixed with an excess of protein cages, each with a single biotin functional group on its exterior surface forming a shell surrounding the core. In an iterative synthetic process, each shell may be used as the core for the development of the next layer. In this way large homogenous aggregates may be fabricated using individual nano-particles as building blocks. The ability to control the aggregate size of the magnetic cluster allows exploration of the size dependent MRI properties of these materials. Characterization of these clusters may be performed using dynamic light scattering, size exclusion chromatography, transmission electron microscopy, sedimentation values by rate-zonal centrifugation on sucrose gradients, AC and DC magnetic characterization and T1 and T2 relaxivities.

The components required to fabricate these aggregates include without limitation biotin and streptavidin. Multiple ligands may be presented on the same protein cage. Both cell targeting ligands and clustering ligands may be presented on the same protein cage. Protein cages may be developed as templates for the synthesis of cell targeted high performance MRI contrast agents. The ability to combine cell targeting, incorporation of magnetic materials and the ability to form homogenously defined clusters provides a basis for the development of a new generation of MRI contrast agents.

X. Applications

In one aspect, the compositions are used in a variety of imaging and therapeutic applications. For example, once synthesized, the metal ion complexes of the invention have use as magnetic resonance imaging contrast or enhancement agents. Specifically, the imaging agents of the invention have several important uses, including the non-invasive imaging of drug delivery, imaging the interaction of the drug with its physiological target, monitoring gene therapy, in vivo gene expression (antisense), transfection, changes in intracellular messengers as a result of drug delivery, etc.

Delivery agents comprising imaging agents comprising metal ions may be used in a similar manner to the known gadolinium MRI agents. See for example, Meyer et al., supra; U.S. Pat. No. 5,155,215; U.S. Pat. No. 5,087,440; Margerstadt et al., Magn. Reson. Med. 3:808 (1986); Runge et al., Radiology 166:835 (1988); and Bousquet et al., Radiology 166:693 (1988). The metal ion complexes are administered to a cell, tissue or patient as is known in the art.

Delivery agents comprising imaging agents that do not use metal ions may be used in a similar manner as described in U.S. Pat. Nos. 6,219,572, 6,219,572, 6,193,951, 6,165,442, 6,046,777, 6,177,062, 5,286,853, 6,248,305, and 6,086,837, all of which are hereby expressing incorporated by reference.

A “patient” for the purposes of the present invention includes both humans and other animals and organisms, such as experimental animals. Thus the methods are applicable to both human therapy and veterinary applications. In addition, the metal ion complexes of the invention may be used to image tissues or cells; for example, see Aguayo et al., Nature 322:190 (1986).

Generally, sterile aqueous solutions of the imaging agent complexes of the invention are administered to a patient in a variety of ways, including orally, intrathecally and intraveneously in concentrations of from about 0.003 to about 1.0 molar, with dosages from about 0.03, about 0.05, about 0.1, about 0.2, and about 0.3 millimoles per kilogram of body weight being suitable. Dosages may depend on the structures to be imaged. Suitable dosage levels for similar complexes are outlined in U.S. Pat. Nos. 4,885,363 and 5,358,704.

Once made, the compositions and delivery agents of the invention find use in a variety of applications. In general, methods, nanoparticles, and arrays, according to the present invention provide a means to generate magnetic materials comprising magnetic clusters of specifically designed size and magnetic composition, and whose mesoscopic magnetic properties may be independently varied over a large range. That is, by choosing the type and size of nanoparticle at each, or at least a plurality of, locations within the magnetic material, or a specific magnetic cluster, the individual cluster moment (dipole), and anisotropy (or tendency of the material to magnetized), as well as other properties, may independently designed and controlled. Further, through the appropriate choice of interstitial materials and the formation of arrays, inter-particle interactions are controlled, allowing for specific design of macroscopic magnetic material properties, such as the coercive field. This overall design capability—that is, the ability to independently vary individual magnetic properties, and/or the ability to design a magnetic material by choosing the size and type of a plurality of nanoparticles that make up the material, as well as the interstitial molecules that govern one or more interparticle interactions—allows for the design of unprecedented magnetic materials. In this general manner, methods, nanoparticles, and arrays of the present invention find use in generally any present or future application requiring or advantageously employing a magnetic material or device, in that the methods, nanoparticles, and arrays of the present invention allow for the precise and independent tailoring of magnetic materials for any application.

The small nanoparticles formed according to embodiments of the present invention further find use in enhancing the rate and specificity of various reactions—including catalytic and stoichiometric reactions. Particles having smaller diameters, such as the 6 nm nanoparticles made according to embodiments of the present invention, enhance reaction rates of certain reactions due to their greater surface area-to-mass ratio.

In some embodiments, the arrays (and solutions) comprising the nanoparticles, particularly the nanoparticle cores, find use as metal catalysts.

In other embodiments, the compositions of the invention are used to deliver therapeutic moieties to patients. In another embodiment, the compositions of the invention further comprise therapeutic agents for administration to patients. The administration of the compositions of the present invention can be done in a variety of ways, including, but not limited to, orally, subcutaneously, intravenously, intranasally, transdermally, intraperitoneally, intramuscularly, intrapulmonary, vaginally, rectally, or intraocularly in concentrations of 0.003 to 1.0 molar, with dosages from 0.03, 0.05, 0.1, 0.2, and 0.3 millimoles per kilogram of body weight being suitable. In some instances, for example, in the treatment of wounds and inflammation, the composition may be directly applied as a solution or spray. Depending upon the manner of introduction, the compositions may be formulated in a variety of ways, including as polymers, etc. The concentration of therapeutically active compound in the formulation may vary from about 0.1-100 wt. %.

Generally, pharmaceutical compositions for use with both imaging and therapeutic agents are in a water soluble form, such as being present as pharmaceutically acceptable salts, which is meant to include both acid and base addition salts.

The pharmaceutical compositions of the present invention comprise nanoparticles loaded with therapeutic moieties in a form suitable for administration to a patient. In one embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Suitable salts include without limitation the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

Combinations of the delivery agents or compositions may be administered. Moreover, the delivery agents or compositions may be administered in combination with other therapeutics.

Generally, sterile aqueous solutions of the nanoparticles of the invention are administered to a patient in a variety of ways, including orally, intrathecally and especially intraveneously in concentrations of 0.003 to 1.0 molar, with dosages from 0.03, 0.05, 0.1, 0.2, and 0.3 millimoles per kilogram of body weight being suitable.

In some embodiments, it may be desirable to increase the blood clearance times (or half-life) of the nanoparticle compositions of the invention. This has been done, for example, by adding carbohydrate polymers, including polyethylene glycol, to other compositions as is known in the art.

As discussed above in section III. A. 6. Inorganic material agents, the protein cages may contain various types of inorganic materials. It has been shown that protein encapsulated Fe-oxides are excellent MR contrast agents. There is potential for using nano-phase Fe₃O₄ in hyperthermia therapy for tumor necrosis as described herein. The Eu₂O₃, ZnSe, and ZnS nano-particles have potential as fluorescent quantum dots for cellular imaging. In addition, due the large neutron capture cross-section of the Eu ion, Eu₂O₃ nano-particles could potentially act as agents for neutron capture therapy (NCT). The present invention provides sufficient capacity to synthesize large quantities of these materials for the use in preparing magnetic particles for in vivo MRI analysis and therapy.

Another embodiment of the present invention provides an expansion of directed synthesis of inorganic nano-particles beyond the electrostatic model. In another embodiment, peptides specific to particular inorganic materials have been incorporated into protein cages (See Section III. A. 6. Inorganic material agents). In one other embodiment, a peptide (KTHEIHSPLLHK) for CoPt may be incorporated into the sHsp. FIG. 13 shows the spatially selective synthesis of this important magnetic material. In one embodiment, the present invention provides methods for selective binding/entrapment/synthesis of therapeutic (and imaging) agents within protein cage architectures. Overall, these results demonstrate significant progress in being able to entrap/synthesize a broad range of biomaterials within a diversity of protein cage architectures. Furthermore, it illustrates the success of the ‘molecular lego set’ approach of the systems, in that the principles can be applied to create a diversity of medically relevant materials.

In another embodiment, the present invention provides methods for directing the synthesis of homogeneously sized magnetic iron oxide (magnetite Fe₃O₄) particles within protein cage systems (Allen, M., et al. 2002. Advanced Materials 14:1562−+), (Bulte, J. W. M., et al. 1994. Jmri—Journal of Magnetic Resonance Imaging 4:497-505), (Douglas, T., et al. 2002. Advanced Materials 14:415−+), (Flenniken, M. L., et al. 2003. Nano Letters 3:1573-1576). Characterization of the magnetic properties of protein encapsulated magnetic nano-particles, their cellular uptake, and behavior as MR contrast agents have been performed in cell culture assays. These results indicate that the protein encapsulated Fe₃O₄ acts as an MR contrast agent with performance comparable to (or better than) commercially available (e.g. Ferridex) superparamagnetic iron oxide systems. The combination of these materials with a demonstrated targeting capacity may provide a dramatic imaging enhancement of these materials relative to currently available materials. In addition, characterization of the magnetic properties of these materials (Bulte, J. W. M., et al. 2004. Methods in Enzymology, vol. 386), (Bulte, J. W. M., et al. 1994. Investigative Radiology 29:S214-S216), (Bulte, J. W. M., et al. 2002. Academic Radiology 9:S332-S335), (Bulte, J. W. M., et al. 2001. Nature Biotechnology 19:1141-1147), (Gilmore, K., et al. Journal of Applied Physics), (Resnick, D., et al. 2004. Journal of Applied Physics 95:7127-7129), (Usselman, R. J., et al. Journal of Applied Physics) make them ideal candidates for applications in targeted hyperthermia for tumor necrosis as described herein.

The following examples serve to more fully describe the manner of using the above-described invention. It is understood that these examples in no way serve to limit the true scope of this invention, but rather are presented for illustrative purposes.

All references cited herein are incorporated by reference in their entirety.

### EXAMPLES

### Example 1

### Modifications to Protein Cages for Enhanced Gd³⁺ Binding

We have taken advantage of our structural knowledge of the Ca²⁺ binding in wild type virions in an attempt to enhance binding of gadolinium (Gd³⁺) for eventual use as a possible MRI contrast agent. The Ca²⁺ binding sites in wild type virions results from the precise orientation of acidic residues contributed from adjacent coat protein subunits at the quasi three-fold axis (Speir, J. A., et al., 1995, Structure 3:63-78; and Zhao, X., 1998, Ph.D. Purdue University). There are 180 Ca²⁺ binding sites per virion. Ca²⁺ binding at these sites is thought to satisfy the charge repulsion created at pH 6.5 by the cluster of acidic residues, and to assist with creating shell curvature during virion assembly. Ca²⁺ is normally required for in vitro assembly of CCMV at >pH 6.5. We have demonstrated that Gd³⁺ can act as a substitute for Ca²⁺ in the pH-dependent assembly assay. We are attempting to enhance assembly-dependent Gd3+ binding by protein engineering of the Ca²⁺ site. The ionic radii of Ca²⁺ and Gd³⁺ are quite similar (0.99 and 0.938 Å respectively) indicating that the Ca²⁺ binding site is already a good starting point for Gd³⁺ binding. In general the Lanthanides prefer O and N donor atoms in their coordination environment and show considerable variability in coordination number. Therefore, we have introduced a combination of glutamic acid and histidine residues at positions 86 (Q86E/H) and/or 149 (Q149E/H) which are in close proximity to the metal and would provide one or two additional coordination sites for the metal. These constructs have been confirmed by DNA sequencing and are in the process of being expressed in the P. pastoris system.

Genetic modifications to the Ca²⁺ binding sites. A collection of coat protein mutations surrounding the CCMV Ca²⁺ binding site (Glu81, Gln85, Glu148, Gln 149, Asp 153) will be produced and assayed for their ability to create high affinity Gd³⁺ binding. All of the Gd³⁺ binding mutations will be made in a R26C/K42R mutant coat protein background that allows for the highly stable assembly of empty particles in the P. pastoris expression system. We propose to add to the R26C/K42R coat protein background a combination of Q85H/E and Q149H/E mutations to facilitate enhanced Gd³⁺ binding. The rationale for this series of mutations is that changing the glutamine positions in the Ca²⁺ binding site to either an acidic glutamic acid or histidine will provide additional coordinating ligands for enhanced binding of Gd³⁺. These mutations will be created as a single mutation and in double mutation combinations using PCR oligonucleotide site-directed mutagenisis protocols that are well established in our laboratories. A total of 8 mutations are to be generated: Q85H, Q85E, Q149H, Q149E, Q85H:Q149H, Q85H:Q149E, Q85E:Q149H, Q85E:Q149E. These modifications involve minimal changes in the coordination geometry of the metal (all modified residues do coordinate the metal in the endogenous site) but rather involve the change from weak electron donor (Gln) to strong electron donor (Glu or His) capable of binding the Gd³⁺. We will confirm each of the mutations by DNA sequencing and by the use of a coupled in vitro translation/transcription assays that determines that each mutation/clone is capable of producing the full-length coat protein. Once confirmed, the clones will be introduced into the P. pastoris expression system by methods well established in our laboratories. The mutated form of empty particles will be purified to near homogeneity on sucrose gradients as previously described. If some or all of the Gd³⁺ mutations fail to assemble into empty particles, the expressed coat protein will be purified by Ni²⁺ affinity chromatography using a poly-histidine tag present on the N-terminus of the coat protein. We have previously demonstrated that the R26C/K42R coat protein containing the poly histidine tag can be purified from P. pastoris to near homogeneity by Ni-affinity chromatography. The purified protein is efficiently assembled in vitro into empty virus particles.

Quantifying Gd³⁺ binding. The ability of the engineered CCMV virion to bind Gd³⁺ will be assessed by in vitro assembly, fluorescence quenching, and isothermal titration calorimetry. Both fluorescence quenching and isothermal titration calorimetry will allow us to determine the binding constant for Gd to the engineered virus as compared to control experiments using the wild type virion and mutants with disrupted metal binding sites. These techniques provide two very sensitive and complementary methods for measuring the binding of Gd³⁺ to the virion—an essential component for developing this technology for medical diagnostic purposes.

In vitro assembly assay. The in vitro assembly assay takes advantage of the fact that particle formation is dependent on Gd³⁺ at values>pH 6.5 (wild-type coat protein is dependent on Ca²⁺). After purification (either as assembled empty cages or as non-assembled protein) the eight Gd³⁺ binding mutants will be disassembled under high ionic strength, elevated pH and temperature, and a reducing environment. (0.5M CaCl₂, 5 mM DTT, pH 8.5 at 50° C.). The disassembled coat protein will be further purified by Sephadex G-100 size exclusion chromatography (the coat protein usually exists in its non-covalent dimer form). All residual Ca²⁺ is removed by extensive dialysis with EGTA. Gd³⁺-dependent assembly of the protein into particles is assayed by dialyzing 0.1 mg/ml (100 μl) of the coat protein-Gd³⁺ binding mutations in the presence of increasing amounts of Gd³⁺ (0-1 mM Gd³⁺) at pH 7.0, 20° C. The amount of assembly is determined by sedimentation on 10-40% sucrose gradients and quantitating the amount of the free coat protein (3S) vs. assembled empty particles (50S). A second quantitative ELISA assay will also be used that only detects assembled virions and not the non-assembled coat protein.

Fluorescence quenching. The endogenous metal binding site in CCMV is roughly 1.4 nm from Trp55 and 1.5 nm from Trp47. The fluorescence behavior of the Trp55 and 47 is expected to be quenched by energy transfer to the metal ion occupying the site close to it as has been demonstrated in related metal binding proteins (Treffry, A., et al., 1998, J. Biol. Inorg Chem. 3:682-688). Excitation of the protein at 280 nm leads to fluorescence emission from Trp at 340 nm. The assembled metal free virion (1 mg in 1.0 ml 0.1M MES, pH 6.5) will be titrated by small additions (2 μL) of Gd³⁺ (20 mM) and the fluorescence will be measured at 340 nm after excitation at 280 nm. The Gd additions will be continued until the metal binding sites are saturated and no further decrease in fluorescence can be detected. In this way we can monitor the steady state fluorescence quenching as a function of [Gd³⁺] concentration. This is expected to give a simple hyperbolic ligand binding curve which can be fit to extract the dissociation (or binding) constant K_{d}. Controls for this measurement will include the wild type virion and CCMV expressing E81T/E85T and E149T modifications which we have shown previously eliminates Ca²⁺ binding. This technique requires only modest amounts of protein which can be easily recovered from the experiment.

Isothermal titration microcalorimetry. Isothermal titration microcalorimetry allows us to measure directly the heat evolved as two or more species interact (Wadsö, I., 1997, Chem. Soc. Rev. 26:79-86). Thus, a solution of the assembled, metal-free virion can be titrated with a solution of Gd³⁺. The empty virion from P. pastoris will be isolated as described previously and then dialyzed extensively against chelating agent (EDTA/EGTA) in low pH buffer (0.1M Ac pH 4.5) to ensure metal removal whilst maintaining virion assembly. Excess chelating agent will then be removed by further dialysis against buffer. Immediately prior to calorimetry experiments, the virion will be re-purified by gel filtration (with 10⁶ M_{w} cut-off) to ensure that only fully assembled virions will be assayed. A solution of the virion (2 ml of 0.5-1 mg/ml) will be titrated with Gd(III) (20 mM) using 5.0 μL injections and allowing 8 minutes between injections for baseline re-equilibration for a total of 20 injections. The heat evolved during the reaction is monitored by heat compensation using a MicroCal titration calorimeter and recorded. The curve can then be fit (using Origin) to accommodate a binding model, and a binding constant (or dissociation constant) can be extracted. By measuring the heat directly this technique allows simultaneous determination of all binding parameters (K, ΔH°, ΔS°, and n the number of sites) in a single experiment (Wiseman, T., et al., 1989, Analytical Biochemistry 179:131-137). In the case of the CCMV virion we expect 180 equivalent Gd³⁺ binding sites.

Evaluation of Gd-bound CCMV virion as a potential candidate for MRI contrast agent. Empty virions (those with and without the Pep-11 fusion protein) will be purified from P. pastoris as previously described. Endogenous Ca²⁺ will be removed from the virion by extensive chelation with EDTA/EGTA and the metal-free virion loaded with Gd(III). Gd loaded virions will be isolated by either gel filtration or gradient centrifugation. The virions will initially be characterized by their effect on the values for T1 relaxation of water protons, which will be determined by ¹H NMR. This will require approximately 0.5 ml of 2 mg/ml Gd loaded virion. T1 will be measured by inversion recovery experiment (180°ₓ- [symbol] -90°ₓ-FID) on a GE 400 MHz NMR. However, this T1 is measured at a single field strength and it is of particular interest for the development of these materials as MRI contrast agents to measure T1 (and T2) as a function of field strength.

Animal Studies and Scintigraphic imaging. A limited number of animal studies are proposed for the express purpose of determining the biodistribution and blood half life of the virions. We have chosen to perform these initial experiments with ^{99m}Tc because the Tc radioactive assay is a very sensitive technique that is preferred for determining the biodistribution of wild-type and pep-11 modified virions in a mouse model system. Synthesis and attachment of a specific ligand for Tc (nicotinyl hydrazine (2)) to exposed lysine residues on the wild-type virion has already been achieved. Initial ^{99m}Tc radioisotope labeling studies have shown that we can incorporate significant amount of ^{99m}Tc into the nicotinyl hydrazine modified virion of CCMV.

After modification and radiolabeling with ^{99m}Tc, CCMV protein cages (both wild-type and those showing good cell binding activity and competition with free peptide 11 in vitro) will be tested and imaged in mice. The animals will each be given a single injection of radiolabeled protein cages via the tail vein, then returned to their cages. Three different radiolabeled forms will be tested on groups of mice: ^{99m}Tc-protein cages consisting of wild-type virions, ^{99m}Tc-protein cages which have been modified with Pep-11, and ^{99m}Tc-labeled free peptide II. The radiolabeling of these forms will be carried out as described above using the well established nicotinyl hydrazine ligand (Abrams, M. J., et al., 1990, J. Nucl. Med. 31:2022-2028). At various times after dosing, groups of animals will be euthanized with an overdose of halothane in a chamber. Each animal will be placed on the surface of the gamma camera for collection of a scintigraphic image of the distribution of radioactivity in its body. The camera will be fitted with a low-energy all-purpose collimator and the energy discriminator of the camera will be set to acquire the 140 keV photon of Tc-99m with a 20% window. The images will be captured in digital form by a NucLear Mac computer which is interfaced to the gamma camera. Immediately afterward, the major organs and tissues will be removed, weighed and counted in a scintillation counter to assess radiotracer content. Organs and tissues to be collected will include: liver, spleen, lungs, kidneys, stomach, small and large intestine, bone, muscle, and blood. The amount of radioactivity in each tissue will be expressed as a percent of the injected dose, determined from an appropriately diluted standard of the initial radiotracer obtained before injection.

### Example 2

### Electrostatic Modifications to Protein Cages

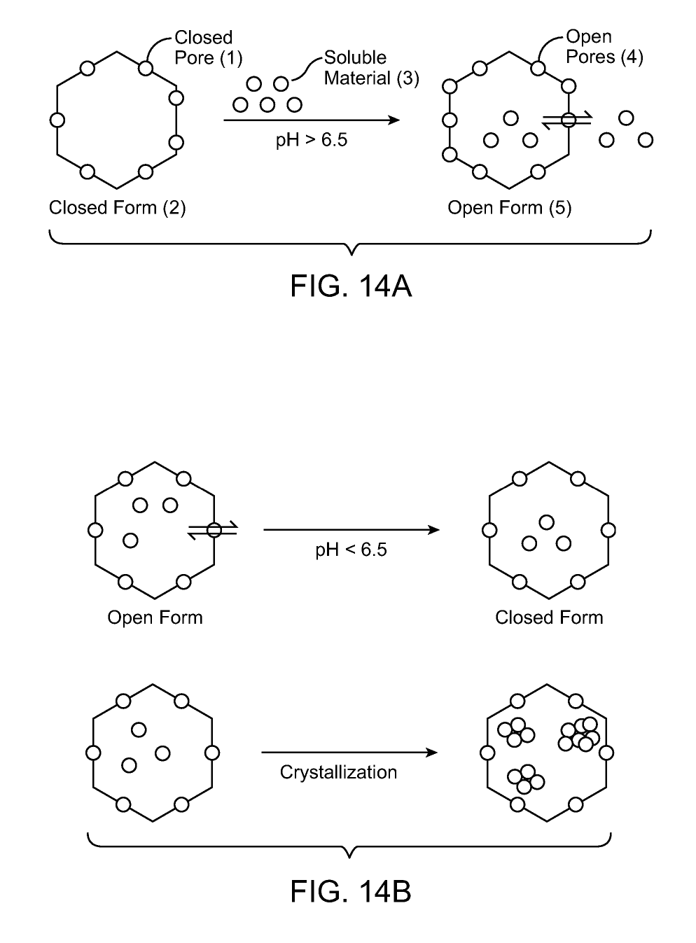

Entrapment and Growth of Anionic Metal Species. We have crystallized a range of polyoxometalate species in CCMV and the Norwalk Virus. This was accomplished by providing an interface for molecular aggregation, based on complementary electrostatic interactions between the protein and the anion metal species, which creates a locally high concentration at the protein interface. Briefly outlined, the empty virions were incubated with the precursor ions (W₄²⁻, VO₃⁻, MoO₄²⁻) at approximately neutral pH. Under these conditions the virus exists in its open (swollen) form and allows all ions access to the cavity. The pH of the virus solution was then lowered to approximately pH 5.0. This induced two important complementary effects i) The inorganic species underwent a pH dependent oligomerization to form large polyoxometalate species such as H₂WO₄₂¹⁰⁻ (Douglas, T., and M. J. Young., 1998, Nature 393:152-155) which were readily crystallized as ammonium salts ii) the viral capsid particle underwent a structural transition in which the pores in the protein shell closed, trapping crystallized mineral or mineral nuclei within the virus. Crystal growth of the polyoxometallate salt continued until the virion container was filled. Thus, the material synthesized is both size and shape constrained by the size and shape of the interior of the viral protein cage. The resulting product(s) could be easily purified (by sedimentation velocity centrifugation on sucrose gradients, density centrifugation on cesium gradients or size exclusion chromatography), as it maintained all the same physical characteristics of the virion itself, and was visualized by transmission electron microscopy (FIGS. 14A and 14B). Experimental conditions were adjusted so that mineralization occurred selectively only within the viral capsid and no bulk mineralization was observed in solutions containing assembled viral capsids or virion-free controls. We postulate that the role of the virion is to provide a highly charged interface capable of binding the polyoxometalate polyanions and thus providing stabilization of incipient crystal nuclei. The protein interface thus acts as a nucleation catalyst (Hulliger, J., 1994, Angew. Chem. Int. Ed. 33:143-162) based on the electrostatic potential generated by Arg and Lys residues which constitute the native RNA binding sites.

Entrapment and Growth of Soft Metal Species. Recently, we have begun to investigate how changes in the electrostatic nature of the N-terminus affect the type of materials that can be entrapped within the virus particles. As an example, we have investigated the catalytic binding properties of a mutant form of CCMV which has a specific metal binding site (6 histidines) engineered onto the N-terminus of the coat protein. Histidine has very high affinity for so-called soft metals including Fe(II) (Cotton, F. A., and G. Wilkinson., 1999, Inorganic Chemistry, John Wiley & Sons). We have found that this mutant is able to selectively bind Fe(II), facilitate its autoxidation to Fe(III) and subsequent hydrolysis to form a ferric oxide mineral within the virion (FIG. 14D). Once again, we have observed that the nano material within the cage is both size and shape constrained by the internal dimensions of the particle. Wild type CCMV shows none of this catalytic activity and when incubated in the presence of Fe(II) all that was observed was autoxidation and hydrolysis in an uncontrolled fashion and no virion encapsulated mineral was detected. Thus, we have selectively engineered the virion to interact in a chemically specific manner which is very different from the wild type virus and which goes beyond purely electrostatic interactions.

Engineering of the N-terminal region of the coat protein subunit. We have focused our efforts on the electrostatic modifications to the N-terminal region of the coat protein subunit. From previous studies we know that the first 25 residues of the N-terminal arm are not involved in the structural integrity of the virion and are highly disordered in the crystal structure. The role of the N-terminus in the normal viral replication cycle is to package the viral RNA (Fox, J. 1997. Ph.D. Purdue University; and Fox, J. M., et al., 1994, Sem. in Vir. 5:51-60. 34) and to release the RNA during virion disassembly (Albert, F., et al., 1997, J. Virol. 71:4296-4299; and Zhao, X. 1998. Ph.D. Purdue University). We have demonstrated previously that drastic modifications to this region still allows for the in vitro assembly and isolation of empty virions (Zhao, X., et al., 1995, Virology 207:486-494; and Zhao, X., 1998, Ph.D. Purdue University). For example, the first 25 residues can be deleted with no ill effects on in vitro assembly of empty particles. Likewise, an additional 44 non-viral residues can be added to the N-terminus without compromising in vitro virion assembly. Finally, the substitution of 6 histidine residues to the N-terminus produces assembled virions. In all cases, the empty virions assembled from the altered N-terminus are structurally similar to wild type virions. Clearly, the N-terminus of the CCMV coat protein can undergo significant manipulations without preventing empty virion formation.

We are currently in the process of further manipulations to the coat protein N-terminus. We have recently completed the construction of a mutant in which all 9 basic residues (Arg, Lys) in the first 25 residues of the N-terminal arm have been replaced by glutamic acid residues. This will effectively change the entire electrostatic character of the virion interior by approximately 3240 units of charge (9×180×2) at neutral pH. Mutagenesis of this construct has been completed and the DNA has been sequenced. The mutant protein has been expressed in the E. coli expression system and soon in the P. pastoris system as well. Work is currently underway to assess the assembly of the purified coat proteins into empty virions.

Polyanionic encapsulation in wild type protein cages. We have shown that the empty CCMV virion will uptake and encapsulate synthetic polyanions such as poly(dextran sulfate) and poly(anetholsulfonic acid) in addition to non-genomic polynucleic acid (both single and double stranded RNA and DNA) (Douglas, T., and M. J. Young, 1998, Nature 393:152-155). This interaction appears to be electrostatically driven, probably through cooperative binding which minimizes the unfavorable entropy effects.

Based on our initial experiments (see FIG. 14C) showing selective encapsulation of both poly(dextran sulfate) and poly(anetholsulfonic acid), we will investigate the range of relevant polyanion species which can be encapsulated into wild-type virions and then study their release as a function of time and environmental conditions which control the gating response. For example, suramin (Church, D., Y., et al., 1999, Cancer Chemother. Pharmacol. 43:198-204; Firsching, A., et al., 1995, Cancer Res. 55:4957-4961; Gagliardi, A. R. et al., 1998, Cancer Chemother. Pharmacol. 41:117-124; Hutson, P. R., et al., 1998, Clin. Cancer Res. 4:1429-1436; Khaled, Z., et al., 1995, Clin Cancer Res. 1:113-122; and Vassiliou, G., 1997, Eur. J. Biochem. 250:320-325), heparin (Engelberg, H., 1999, Cancer 85:257-272), the copolymer of divinyl ether-maleic anhydride, poly(acrylic acid), and the copolymer of ethylene and maleic anhydride are all therapeutically important polyanions (although not necessarily associated with any single therapeutic treatment).

We will use the commercially available polyanions: suramin, heparin (and the heparin analogs chondroitin sulfate, dermatan sulfate and mesoglycan) and the synthetic poly(acrylic acid) and poly(maleic anhydride). Briefly outlined, the empty assembled virions (0.5 mg/ml) purified from P. pastoris will be incubated with an excess of the anionic polymers at pH 7.5 (50 mM Tris) where the virions exist in their open, swollen form. Free exchange can occur between the outside and the interior of the virion. After material loading, the pH will be lowered to below the gating threshold of the virion (pH<6.5), trapping the polymer inside. This is easily achieved by rapid exchange of buffers (into 100 mM NaOAc, pH 4.5) using ultrafiltration (centricon 100). The polymer containing virion will be subsequently purified by either gradient centrifugation on 10-40% sucrose gradients or by gel filtration (medium pressure liquid chromatography). Differences in sedimentation between empty and filled virions will allow us to quantitate the virion loading. This will be compared with spectroscopic analysis (UV-V is, 315 nm) on the previously established entrapment of poly(anetholsulfonic acid) where aromatic functionality (and associated large molar absorbtivity) allows very accurate determination of virion loading.

We will monitor the release of the polymers as a function of time by analyzing aliquots of the loaded virion by analytical centrifugation or gel filtration. By integration of peaks we can quantitate the distribution of polymer associated with the virion (high MW) and polymer which is free (low MW). Alternatively, we can employ equilibrium dialysis to spectroscopically monitor the diffusion of the individual polymers from within the virion. These time dependent release assays will also be investigated as a function of pH to determine the role played by virion gating/swelling. Controls for the uptake and release will include polymers of similar chemical composition and size but differing in charge (i.e. poly(ethylenimine) a cationic polymer, and poly(vinyl alcohol) a neutral polymer).

Small molecule crystallization. We propose to apply similar approaches and techniques as those used for metal anions for the crystallization of small molecules (with biomedical applications) at the protein interface within the virion. For example, we will utilize the inherently low aqueous solubility of many organic drug compounds as a means to drive the spatially selective crystallization, through control of the level of supersaturation. Encapsulation of agents or materials is an entropically unfavorable event (decreased freedom) which must be offset by a favorable enthalpy (binding) of interaction. This interaction will rely in part on complementary electrostatic interactions between the drug and the protein interface as has already been shown with a number of organic and inorganic analogs. In the case of crystallization, once an initial aggregate has formed the crystal growth process is self-perpetuating because of the high affinity the molecules have for the crystal lattice and the ever increasing surface area of the growing crystal. Thus, the protein interface acts only as a nucleation catalyst by providing an interface favorable for aggregation.

As the most simple example for this model study we will first use wild-type virus and the common drug “aspirin” (acetylsalicylic acid) which carries a carboxylic acid functionality making it negatively charged at any pH close to neutral. In addition, the aromatic ring, and associated strong UV absorbance (λₘₐₓ=229 nm ε=4.8×10⁴M⁻¹cm⁻¹), allows us to monitor this molecule very easily. The solubility of acetylsalicylic acid is moderate at room temperature (saturation ˜18.5 mM) and shows a dramatic temperature dependence. Lowering the temperature even a few degrees will induce a condition of supersaturation, from which it is thermodynamically possible for crystals to form. Exploratory experiments in the absence of virion will be performed to determine the conditions for “bulk” crystallization on a reasonable timescale (i.e. a few hours). Then, the empty virions (0.5 mg/ml) will be dialyzed into a saturated solution of acetylsalicylic acid at room temperature and pH>6.5 where the virion is in its swollen conformation. The temperature of this solution will be lowered and before bulk crystallization occurs the virion will be isolated. The inner protein surface of the virion, rich in arginine and lysine residues has already been shown to induce selective crystallization of anionic molecules and we expect the virion to catalyze the crystallization of acetylsalicylic acid from slightly supersaturated solutions in a similar fashion. In addition, we will use counter ions such as Me4N+ to reduce the solubility even further and to induce crystal formation. Once the nano-crystal of the organic material has been formed the pH of the solution will be lowered to below the gating threshold (<6.5) and the crystal encapsulated virion will be isolated by gradient centrifugation or column chromatography.

Other candidate drugs and drug analogs will be tested for their ability to be crystallized within the virus protein cage. These include the antineoplastic drug diethylstilbestrol, bis-naphthalene disulfonate tetraanion (Khaled, Z., et al., 1995, Clin Cancer Res. 1:113-122), the analgesic flurbiprofen, as well as 5-fluoro-2′deoxyuridine mono(or di)phosphate. These compounds have been chosen as models because they all have a unique and sensitive analytical detection (aromatic—strong UV absorbance or F-¹⁹F NMR). Similar experimental approaches as those described for aspirin will be attempted. However, we will take advantage of the unique solubility properties of each drug to enhance the likelihood of success.

Electrostatic modifications to the virion interior. Using our well established methods for site directed mutagenesis of the coat protein, we will engineer the protein so as to change the electrostatic nature of the interior protein interface while leaving structural portions of the coat protein unchanged. Thus we will change the overall charge on the interior from net positive (rich in Arg and Lys) to net negative (rich in Glu or Asp). This will allow us to broaden the range of materials which the virion can selectively entrap. The first coat protein mutant will be the subE mutant where substitutions of the nine arginine and lysine residues for glutamic acid have been made on the non-structural N-terminus. We will continue our isolation and assembly of this virion from both E. coli and P. pastoris expression systems.

The second set of modifications will involve the complete substitution of the first 25 N-terminal amino acids with a series of varying lengths of glutamic acid-aspartic acid repeats. We have previously determined that the first 25 N-terminal amino acids can be deleted or that an additional 44 amino acids can be added, without affecting empty particle assembly. We propose to express increasing units of glutamic acid-aspartic acid repeats (2, 4, 6 etc. repeating units) to the N-terminus, deleted of its first 25 amino acids. Addition of the glutamic acid-aspartic acid repeats will be accomplished by PCR based site directed mutagenesis, a technique with which we are quite familiar. We will add (n+2) glutamic acid-aspartic acid repeats until addition of more repeats prevents protein cage assembly in P. pastoris and/or in vitro assembly using E. coli expressed modified coat protein. At this point we do not know how many glutamic acid-aspartic acid repeat units we will be able to add to the N-terminus, but we estimate the number to be between 5-20 repeats. In addition to adding the repeat units to the N-terminus deleted of its natural 25 amino acids, a similar series of glutamic acid-aspartic acid repeat units will be added directly to the coat protein with an intact wild type N-terminus.

The third set of mutations will involve point mutations of residues exposed on the inner surface of the protein cage, but not part of N-terminal 25 amino acids. The list of these point mutations include V34E/D, K42E/D, W47E/D. All of these point mutations will be made singlely or in combinations with each other using PCR based site-directed mutagenesis. In addition, all of these point mutations will be made in a background in which the first 25 N-terminal amino acids have been deleted. The N-terminal deletion removes 9×180=1620 positively charged residues without disrupting the protein cage structure. As an example, the K42E mutation alters the charge at the interface by 2×180=360. Thus the overall effect of this mutation (in the N-terminal deletion background) is to alter the charge on the interior surface by up to 1980 charges. We have shown that alterations at the region around residue 42 do not disrupt the structure and from the crystal structure it appears that K42 does not form salt bridges to mediate the charge. Thus the structure might be expected to also accommodate the close positioning of negative charge in the replacement of Lys by Glu (or Asp). As described below, all three series of mutations will be assayed for their ability to selectively entrap cationic therapeutic agents. Once these mutations have been achieved and expressed in either the P. pastoris or E. coli expression system, the modified coat protein (either as assembled protein cages or as free coat protein) will be isolated by techniques well established in our laboratories (centrifugation, PEG precipitation, column chromatography). They will then be assembled into empty particles by dialysis into an assembly buffer system.

Encapsulation of cationic species (polymeric and molecular). Assembled empty particles of the modified coat proteins expressing a negatively charged interior surface will be investigated for their ability to bind and entrap cationic and polycationic species with therapeutic relevance. Essentially the same methodologies as described above for the entrapment/crystallization and release of anionic species will be used to entrap/crystallize cationic species within the virion. The relevant polymeric species to be studied include poly(ethylenimine), poly(lysine), poly(arginine) and poly(vinylimidazoline). The relevant monomeric cationic species include simple molecules to begin with such as benzanthine dihydrochloride, benzalkonium chloride and then continue to include more complex molecules such as methotrexate HCl, tamoxifen HCl and doxirubicin HCl. The aromatic nature of the simple model compounds such as benzanthine dihydrochloride (and associated high molar absorbtivities in the UV) will be used as an efficient tool for monitoring these species both in solution and as nano-crystals packaged within the virion.

### Example 3

### Bioengineering of New Chemical Switches for the Regulated Entrapment/Release of Materials

We have demonstrated that pH dependent expansion at the quasi three-fold axes is the result of deprotination of the acidic residues comprising the Ca²⁺ binding sites. The loss of protons at the elevated pH results in a close cluster of negative charges that must be accommodated either by the binding of Ca2+ or by the physical expansion (i.e. swelling) induced by electrostatic repulsion. We have taken advantage of CCMV's reversible swelling properties as a control mechanism to introduce and to release materials from the central cavity of the protein cage (see e.g. Examples 1 and 2). This reversible switching property of CCMV provides an exciting opportunity for development of elegant control mechanisms for entrapment and release of therapeutic agents.

pH Activated Chemical Switches. Gating in the wild-type virion results from electrostatic repulsion of carboxylate groups in the absence of the mediating Ca²⁺. We plan to alter the pH sense of this gating mechanism by altering the responsible carboxylate groups to histidines. Thus, the histidines are geometrically aligned for metal binding at that site and should be expected to bind well to soft metals such as Ni(II), Cu(II), and Co(II). In addition, protonation of the imidazole ring will compete with metal binding and once the metal has been lost, the close proximity of these cationic species is expected to cause a similar repulsion and opening of pores at the quasi three-fold axes. This provides a rational design for acidic switching of the gating mechanism of the virion. The mutations for this acid sensitive switch are E81H, E148H and D153H. As described above, each mutation will be generated by PCR oligonucleotide site-directed mutagenesis and introduced into both plant and P. pastoris expression systems. The empty virus particles will be and assessed for the ability to swell in response to lowered pH by changes in sedimentation velocities in 10-40% sucrose gradients (88S vs. 78S).

Redox Activated Chemical Switches. We plan to take advantage of our detailed structural knowledge of the quasi-three fold axis (the location of the Ca²⁺ binding sites) to engineer disulfide cross links between subunits (at the interface between A-B, B-C, C-A subunits in the asymmetric unit). In an oxidizing chemical environment disulfide bond formation at the quasi three-fold axis should prevent virion expansion (swelling) and thus limit entrapment or release of large molecules from the virion's interior. A change to a reducing chemical environment (like that present in the cytosol of an eukaryotic cell) breaks disulfide bonds, resulting in the expansion of the virion and the release of its entrapped molecules. The sites that have been selected have a distance between their Ca atoms of 6.4-6.5 Å which is optimal for proper disulfide bridge formation. In general, computer modeling indicates that the lines marking the Cα to Cβ bond from each residue of the selected pairs are nearly parallel and thus close to being directly in line with one another. This leads to a 900 dihedral angle around the S—S bond that is energetically favored. The mutation pairs at the quasi-three fold axis that meet these criteria are R82C/K143C, R82C/A141C, R82C/F142C, E81C/K143C, E81C/A141C, E81C/F142C. Each set of mutations will be generated by PCR oligonucleotide site-directed mutagenesis. All mutant pairs will be confirmed by DNA sequencing and by our coupled in vitro transcription/translation assay. Each set of mutations will be introduced into both a full-length CCMV RNA 3 cDNA for expression in plant cells (when introduced into cells along with in vitro RNA transcribed with CCMV cDNAs for RNAs 1 and 2) and the P. pastoris expression system. The reduced environment of the cytosol where the empty viral particles accumulate is unlikely to facilitate disulfide bond formation. Empty virus particles expressed in P. pastoris will be purified under either oxidizing (the normal purification procedure) or under reducing conditions (in the presence of 5 mM DTT). The extent of disulfide bond formation will be assessed by mobility on SDS-PAGE gels (+/−reducing agent), quantitative reaction with Ellman's reagent (5,5′-dithiobis(2-nitrobenzoic acid)) and/or monobromobimane (quantitative assays for the presence of SH groups), as well as the ability to undergo swelling at different redox potentials (as determined by changes in sedimentation velocity on sucrose gradients).

### Example 4

### Engineering Protein Cages that Express the Laminin Peptide 11 Targeting Moiety

Preliminary results already indicate that laminin peptide 11 can be expressed on the surface of the CCMV protein cage (see Results below). The proposed experiments are aimed at extending these initial results to 1) determine which of five surface exposed loops is most suitable for high-level stable expression of peptide 11, and 2) determine which of the five positions for expressing peptide 11 is most effective for in vivo targeting of cells lines expressing laminin binding protein (LBP). Briefly outlined, peptide 11 expression in P. pastioris will be analyzed in the three remaining surface loops (coat protein amino acid positions 61, 102, 161). To evaluate stable peptide 11 expression, a time course of the steady-state accumulation of empty protein cages in P. pastoris will be determined by quantitative ELISA using both CCMV antibodies specific for the assembled virion (already in use) and peptide 11 specific polyclonal antibodies (currently in production). Quantitative Western blot analysis will be used to evaluate the integrity of the coat protein-peptide 11 chimeras at each insertion site. We are also in the process of initial crystallization experiments of virions expressing peptide 11 for structural determination using X-ray crystallography.

Coat protein-peptide 11 chimeras will be analyzed for cell-targeting activity using both a cell-invasion assay and a direct competition assay for binding to cells up regulated in LBP on their cell surface. Briefly outlined, protein cages expressing peptide 11 will be tested for their ability to inhibit invasion of tumor cells through EHS basement membrane matrix. To measure invasion, a ‘Transwell’ two chamber assay system where the 8μ pore barrier is impregnated with EHS matrigel basement membrane matrix (J. R. Starkey, et al., 1999, Cytometry 35:37-47). 5×104 tumor cells are seeded into the upper well and the chambers are incubated for 3 days or one week depending on the test cell line. The upper transwells are then removed and the number of cells which have invaded into the lower well quantitated. Positive control assays with free peptide 11 and negative controls (CCMV protein cages lacking peptide 11) will be compared with CCMV protein cages expressing peptide 11.

A second independent assay will also be used to assess the cell-targeting ability of CCMV protein cages expressing peptide 11. The second assay is a competitive binding assay for cells up regulated in laminin binding protein expression. Briefly outlined, an engineered DG44CHO variant cell line with up regulated LBP expression on its surface will be used (J. R. Starkey, et al., 1999, Cytometry 35:37-47). A peptide 11 based photoprobe can be used to directly image the specific binding of peptide 11 to the surface of DG44CHO cells by confocal microscopy. This analog is biotinylated allowing for detection and quantitation using FITC-avidin. In addition, FACScan can also be used to quantitatively follow binding of peptide 11 to DG44CHO cells. Alternatively, Scatchard analysis could be utilized with ¹²⁵I-labeled peptide 11. We propose to take advantage of these well developed assays to determine if CCMV protein cages expressing peptide 11 will compete with the free peptide 11 based photoprobe for binding to DG44CHO cells. Increasing concentrations of the CCMV protein cage expressing peptide 11 will be added to a fixed amount of free peptide 11 based photoprobe and qualitatively assayed for inhibition of the photoprobe using confocal microscopy. More quantitative assays will be carried out by FACScan analysis. A corresponding analysis will also be carried out where the amount of the CCMV protein cage expressing peptide 11 will be held constant and the amount of the free peptide 11 based photoprobe will be varied. The positive control in these experiments will be the free peptide 11 lacking the photoprobe. The negative control will be CCMV protein cages lacking peptide 11.

Delivery and release of entrapped therapeutic agents at the site of attachment will be examined. For example, CCMV protein cages expressing peptide 11 demonstrating the highest affinity for LBP on DG44CHO cells will be loaded with entrapped/crystallized cytotoxic therapeutic agents. Initial studies will be performed with entrapment of suramin which is know to have cytotoxic effects on tumor cells in culture (Church, D., et al., 1999, Cancer Chemother. Pharmacol. 43:198-204). The polyanion will be entrapped as described above (see Examples 2 and 3) in protein cages where the switching mechanism is under either pH or redox control (see Example 3). After attachment to DG44CHO cells, the pH or redox environment will be changed to favor opening of the protein cage to release the entrapped material. In the case of pH mediated switching, the pH of the medium will be increased to >6.5. In the case of redox controlled gating, the medium will be reduced by the addition of DTT. Cell death will be determined by addition of viability stains (tryptophan blue) and counting both viable and non-viable cells. In addition, quantitative MTT dye reduction assays will be performed. A varying range of the polymer loaded protein cages will be attached to DG44CHO cells to determine if there is a correlation between the number of particles attached and the cell death. The appropriate negative controls of free peptide 11 alone, CCMV protein cages loaded with the polymer but lacking peptide 11, and CCMV protein cages expressing peptide 11 but not loaded with entrapped/crystallized materials will be included in all assays. This general approach will also be used to release entrapped/crystallized cationic therapeutic agents (for example methotrexate HCl, doxirubicin HCl, and tamoxifen HCl) from protein cages with modified internal electrostatic surfaces.

Results. We have recently been successful at expressing a potential cell targeting and therapeutic agent on the surface of CCMV particles. Peptide 11 region of laminin has been successfully expressed on the surface of CCMV particles. The first step was the creation of plasmid-based vectors with general utility for cloning of DNA sequences encoding for heterologous proteins as fusion proteins into the surface exposed loops of CCMV. This was accomplished by performing PCR oligonucleotide-directed mutagenesis to introduce a unique BamH1 restriction site into the regions of the CCMV coat protein cDNA corresponding to each of the five surface exposed loops (βB-βC, βD-βE, βF-βG, βC-αCD1, βH-βI). These unique, in frame, BamH1 sites were introduced at coordinates corresponding to amino acid positions 61, 102, 114, 129, and 161 in plasmid backgrounds for expression in in vivo. Expression of these BamH1H constructs, either in plant cell culture, or in the P. pastoris expression system, results in coat protein accumulation at the level similar to wild type coat protein controls. In the second step, an oligonucleotide encoding for peptide 11 (CDPGYIGSRC) with engineered BamH1H ends was cloned into the each of the BamH1H sites corresponding to the five CCMV surface loops (βB-βC, βD-βE, βF-βG, βC-αCD1, βH-βI). Each of the peptide 11 constructs was confirmed by a coupled in vitro transcription/translation assay for full-length coat protein production and by DNA sequencing. All five constructs are currently being expressed in the P. pastoris system. To date, two of the constructs (Pep11-114 and Pep11-129; inserts into the βF-βG and βC-αCD1 loops respectively) have been initially evaluated. Expression in P. pastoris results in production of empty particles expressing peptide 11 at near wild type control levels (FIG. 15). Western blot analysis using CCMV specific antibodies demonstrate that the coat protein mobility is slightly altered due to the additional 1 kDa of peptide 11 sequence and it is intact (in contrast to other coat protein based presentation systems where the coat protein is often proteolytically cleaved at the site of foreign protein expression). We are presently re-sequencing the coat protein DNA from expressing lines to confirm that peptide 11 sequence is still present. We are currently producing a peptide 11 specific antibody to directly confirm and localize the expression of peptide 11 on the surface of the empty virus particles. We expect to begin analysis of the other three peptide 11 constructs, inserted into the remaining three surface loops expressed in P. pastoris, in the near future.

### Example 5

### Heterologous Expression Systems for Production of Viral Protein Cages

We have recently established a yeast-based heterologous protein expression system for the large scale production of modified CCMV protein cages. This is a major technical advance for our system, since it allows us to produce large quantities of protein cages independent of other viral functions (i.e. virus replication and movement). We have previously reported on the development of an E. coli-based CCMV coat protein expression system (Zhao, X., et al., 1995, Virology 207:486-494). Using this system, denatured coat protein can be purified to 90% homogeneity, renatured, and assembled into empty particles which are indistinguishable from native particles (Fox, J. M., et al., 1998, Virology 244:212-218; and Zhao, X., et al., 1995, Virology 207:486-494). Unfortunately, the yields are low and the purification/in vitro assembly procedure is time consuming. In an effort to circumvent the limitations of the E. coli system, we have recently developed a second expression system based on Pichia pastoris. A full-length CCMV coat protein gene has been cloned into the pPICZ shuttle vector (InVitrogen Inc.) and integrated into the P. pastoris genome. Expression of the coat protein is under the control of a strong methanol inducible promoter (the AOX1 promoter). Methanol induction results in the high level expression of the coat protein that self-assembles into empty virus particles within P. pastoris. TEM analysis indicates that the empty virus particles are identical to native virus particles. The empty particles are efficiently purified to >99% homogeneity by lysis of P. pastoris, selective PEG precipitation of the empty particles, followed by purification on 10-40% sucrose gradients. The isolated particles were confirmed to be empty by their sedimentation velocity (50S vs. 83S for full particles), UV spectroscopy characteristics (A260/A280=0.98), and TEM staining characteristics (UA stain intrusion). Typical yields range from 1-2 mg/g FW cells. We are currently optimizing conditions for large-scale fermentation production which should dramatically increase our production of protein cages. We have already demonstrated that the empty protein cages isolated from P. pastoris can be used as constrained reaction vessels. The formation of the ferric oxide mineral within the virion (described above) was performed in N-terminal histidine modified empty protein cages isolated from P. pastoris. In addition, we have preliminary data that modified protein cages expressing peptide 11 on the particle surface can be efficiently assembled in the P. pastoris system (described above).

### Example 6

### Melanoma & Lymphocyte Cell-Specific Targeting Incorporated into a Heat Shock Protein Cage Architecture

Protein cages, including viral capsids, ferritins and heat shock proteins can serve as spherical nano-containers for biomedical applications. They are genetically and chemically malleable platforms with potential to serve as therapeutic and imaging agent delivery systems. In this work, both genetic and chemical strategies were used to impart mammalian cell specific targeting to the 12 nm diameter small heat shock protein (Hsp) architecture from Methanococcus jannaschii. The tumor vasculature targeting peptide RGD-4C (CDCRGDCFC) was genetically incorporated onto the exterior surface of the Hsp cage. This ‘tumor targeting’ protein cage bound to αvβ3 expressing C32 melanoma cells. In a second approach, cellular tropism was chemically imparted by conjugating anti-CD4 antibodies (Ab) to the exterior surface of Hsp cage architectures. These Ab-Hsp cage conjugates bound to CD4+ cells present in a population of murine splenocytes. In order to demonstrate that protein cages can simultaneously incorporate multiple functionalities including cell specific targeting, imaging and therapeutic agent delivery, fluorescein and the anti-tumor agent doxorubicin were covalently bound within Hsp cages.

We and others have demonstrated that protein cages are robust platforms for chemical derivatization, genetic manipulation, metal chelation and encapsulation (Bulte J W M et al. (1993). Inv. Rad., S214-S216; Bulte J W M et al. (1994). J. Magn. Res. Imag. 4, 497-505; Douglas T et al. (1998) Nature (London) 393, 152-155; Flenniken M L et al. (2005). Chem Commun (Camb), 447-9; Flenniken M L et al. (2003) NanoLetters 3, 1573-1576; Chatterji A et al. (2002) Intervirology 45, 362-70; Allen M et al. (2002) Adv. Mater. 14, 1562-1565; Allen M et al. (2003) Chem. 42, 6300-6305; Allen T M et al. (2004) Science 303, 1818-22; Douglas T. Biomimetic synthesis of nanoscale particles in organized protein cages. In: Mann S, editor. Biomimetic Approaches in Materials Science. New York: VCH Publishers; 1996. p. 91-115; Douglas T et al., Self-assembling Protein Cage Systems and Applications in Nanotechnology. In: Fahnestock S R, Steinbuchel A, editors. Polyamides and Complex Proteinaceous Materials I. Weinheim: Wiley-VCH; 2002. p. 517; Douglas T et al. (2002) Advanced Materials 14, 415-418; Douglas T et al. (1999). Advanced Materials 11, 679-681; Hooker J M et al. (2004) J Am Chem Soc 126, 3718-9; Gillitzer E et al. (2002) Chem Commun (Camb), 2390-1; Klem M T et al. (2005). Adv. Funct. Mater. 15:1489-94); Mao C et al. (2004) Science 303, 213-7; Koivunen E et al. (1995). Biotechnology (N Y) 13, 265-70; Wang Q et al. (2002). Chemistry & Biology 9, 805-811; Wang Q et al. (2002). Chemistry & Biology 9, 813-819. We are exploring the potential medical applications of protein cage architectures.

Other nano-scale therapeutic delivery systems also being explored include: lipid micelles, silica nanoparticles, polysaccharide colloids, pegylated liposomes, polyamidoamine dendrimer clusters and hydrogel dextran nanoparticles (Allen T M et al. (2004). Science 303, 1818-22; Roy I et al. (2003). J Am Chem Soc 125, 7860-5; Arap W et al. (1998). Curr Opin Oncol 10, 560-5; Arap W et al. (1998) Science 279, 377-80; Braslawsky G R et al. (1990). Cancer Res 50, 6608-14; Braslawsky G R, et al. (1991). Cancer Immunol Immunother 33, 367-74; Brigger I et al. (2002). Adv Drug Deliv Rev 54, 631-51; Ellerby H M et al. (1999) Nat Med 5, 1032-8; Emerich D F et al. (2003) Expert Opin Biol Ther 3, 655-63; Fundaro A et al. (2000). Pharmacol Res 42, 337-43; Hallahan D et al. (2003) Cancer Cell 3, 63-74; Jana S S et al. (2002). FEBS Lett 515, 184-8; Janes K A et al. (2001) Adv Drug Deliv Rev 47, 83-97; Janes K A et al. (2001) J Control Release 73, 255-67; Laakkonen P et al. (2004) Proc Natl Acad Sci USA 101, 9381-6; Laakkonen P et al. (2002) Nat Med 8, 751-5; Lanza G M et al. (2002) Circulation 106, 2842-7; Muller K et al. (2001) Cancer Gene Ther 8, 107-17; Na K, et al. (2003). J Control Release 87, 3-13; Na K et al. (2002). Pharm Res 19, 681-8; Nahde T et al. (2001) J Gene Med 3, 353-61; Wickline S A et al. (2003). Circulation 107, 1092-5; Wickline S A et al. (2002). J Cell Biochem Suppl 39, 90-7; Yoo H S et al. (2000). J Control Release 68, 419-31; Choi Y et al. (2005). Chem Biol 12, 35-43). Additionally, antibody mediated therapeutic delivery has proven successful in both lab and clinical settings and antibodies themselves may serve as therapeutic agents (Dubowchik et al. (2002) Bioorg Med Chem Lett 12, 1529-32; Hurwitz H et al. (2004). N Engl J Med 350, 2335-42; Jendreyko N. et al. (2003) J Biol Chem 278, 47812-9; King H D et al. (2002) J Med Chem 45, 4336-43; King H D et al. (1999) Bioconjug Chem 10, 279-88; Trail P A et al. (1999) Clin Cancer Res 5, 3632-8; Trail P A et al. (1992) Cancer Res 52, 5693-700; Cortez-Retamozo V et al. (2004) Cancer Res 64, 2853-7; Doronina S O et al. (2003) Nat Biotechnol 21, 778-84; Francisco J A et al. (2003) Blood 102, 1458-65; Hellstrom I et al. (2001). Methods Mol Biol 166, 3-16; Williner D et al. (1993) Bioconjug Chem 4, 521-7; Rader C et al. (2003) Proc Natl Acad Sci USA 100, 5396-400; Trail P A et al. (1993) Science 261, 212-5; Ludwig D L et al. (2003) Oncogene 22, 9097-106; Brooks P C et al. (1994) Cell 79, 1157-64; Brooks P C et al. (1995) J Clin Invest 96, 1815-22; Rader C et al. (2002) Faseb J 16, 2000-2). In this work we demonstrate that protein cage platforms are an additional system to which medically relevant functionalities may be incorporated.

The field of cell specific targeting has been significantly advanced by the in vivo use of phage display techniques to identify targeting peptides (Arap W et al. (1998) Curr Opin Oncol 10, 560-5; Arap W et al. (1998). Science 279, 377-80; Ellerby (1999) Nat Med supra; Laakkonen P et al. (2004) Proc Natl Acad Sci USA 101, 9381-6; Laakkonen P et al. (2002) Nat Med 8, 751-5; 64. Brown KC (2004). Chem Biol 11, 1033-5; Ruoslahti E (1996). Annu Rev Cell Dev Biol 12, 697-715; Ruoslahti E (2000). Semin Cancer Biol 10, 435-42; Ruoslahti E (2002). Nat Rev Cancer 2, 83-90). Small peptides have been identified that target to the vasculature of a variety of tissues, organs and tumors (Pasqualini R et al. (1996). Mol Psychiatry 1,423; Pasqualini R et al. (1996). Nature 380, 364-6; Arap W et al. (2002). Proc Natl Acad Sci USA 99, 1527-31; Rajotte D et al. (1999) J Biol Chem 274, 11593-8). Targeting peptides linked to specific cargo molecules such as therapeutic agents, pro-apoptotic peptides, and quantum dots were able to localize the cargo to the desired in vivo target (Arap W et al. (1998). Science 279, 377-80; Ellerby (1999). Nat Med supra; Arap W et al. (2002). Proc Natl Acad Sci USA 99, 1527-31; Ruoslahti E (2002). Cancer Cell 2, 97-8. Akerman M E et al. (2002). Proc Natl Acad Sci USA 99, 12617-21; de Groot F M et al. (2002). Mol Cancer Ther 1, 901-11; Wermuth J et al. (1997) Journal of the American Chemical Society 119, 1328-1335). One characterized example is the targeting peptide RGD-4C (CDCRGDCFC) which binds αvβ3 and αvβ5 integrins that are prevalently expressed within tumor vasculature (Koivunen E. (1995). Biotechnology (NY) 13, 265-70; Arap W et al. (1998). Science 279, 377-80; Brooks P C, et al. (1994). Cell 79, 1157-64; Pasqualini R et al. (1995). J Cell Biol 130, 1189-96; Friedlander M et al. (1995). Science 270, 1500-2). Work by Arap et al. demonstrated that RGD-4C targeted doxorubicin enhanced tumor regression at therapeutic concentrations less than that required to demonstrate therapeutic efficacy with non-targeted doxorubicin ((1998). Science 279, 377-80). Subsequently, many researchers have utilized the RGD-4C peptide motif for tumor targeting of liposomes, radiolabels, therapeutics, and adenoviral gene therapy vectors (Brooks P C et al. (1994) Cell 79, 1157-64; de Groot F M et al. (2002). Mol Cancer Ther 1, 901-11; Wermuth J et al. (1997). Journal of the American Chemical Society 119, 1328-1335; Winter P M et al. (2003) Cancer Res 63, 5838-43; Kim J W et al. (2004). Int J Mol Med 14, 529-35; Chen X, et al. (2005) J Med Chem 48, 1098-106; Wickham T J et al. (1995). Gene Ther 2, 750-6; Dmitriev I et al. (1998). J Virol 72, 9706-13; Chen L et al. (2004). Chem Biol 11, 1081-91; Grifman M et al. (2001) Mol Ther 3, 964-75; Burkhart D J et al. (2004). Mol Cancer Ther 3, 1593-604; Su Z F et al. (2002). Bioconjug Chem 13, 561-70; Zitzmann S et al. (2002). Cancer Res 62, 5139-43; Fahr A, et al. (2002) J Liposome Res 12, 37-44; DeNardo S J et al. (2000) Cancer Biother Radiopharm 15, 71-9; Burke P A et al. (2002). Cancer Res 62, 4263-72; Smith J W (2003). Curr Opin Investig Drugs 4, 741-5). The effects of RGD-4C targeted therapeutics are augmented due to the antiangiogenic property of RGD-4C itself (Arap W et al. (1998). Science 279, 377-80; Brooks P C et al. (1994). Cell 79, 1157-64; Wermuth J et al. (1997). J. American Chemical Society 119, 1328-1335; Kim J W et al. (2004). Int J Mol Med 14, 529-35; Burke P A et al. (2002). Cancer Res 62, 4263-72; Smith J W (2003). Curr Opin Investig Drugs 4, 741-5). Due to the prior success of RGD-4C, it was chosen as a “proof of concept” targeting peptide for genetic incorporation into a small heat shock protein (Hsp) cage architecture.

In this study we have investigated the ability to introduce cell targeting capacity to protein cage architectures. Both peptides and antibodies were incorporated on the exterior surface of Hsp cages and tested for their ability to bind cell surface ligands. In addition, the ability to simultaneously encapsulate cargo molecules on the Hsp cage interior was investigated.

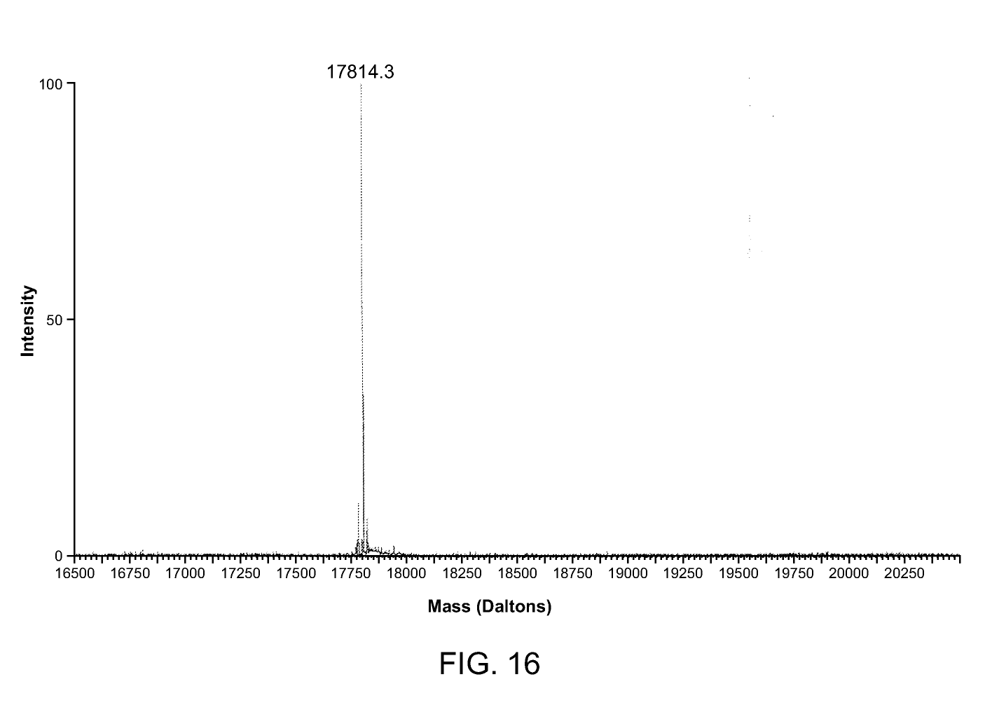

Results and Discussion. We have demonstrated that genetic addition of the RGD-4C peptide or chemical conjugation of an anti-CD4 monoclonal antibody (mAb) onto the exterior surface of the small heat shock protein (Hsp) cage architecture confers specific cell targeting capacity. In addition, we were able to load cargo molecules, either a fluorescent dye (fluorescein) or an antitumor therapeutic agent (doxorubicin), within the interior cavity of the Hsp cage. These results demonstrate the multifunctional capacity of protein cage architectures and their potential utility in medicine. For these studies we chose to use a small heat shock protein cage, which we previously established as a robust platform for genetic and chemical manipulation (Flenniken (2005). Chem Commun (Camb) supra; Flenniken (2003) NanoLetters supra). The small heat shock protein (Hsp) cage from the hyperthermophilic archaeon, Methanococcus jannaschii assembles from 24 identical subunits into a 12 nm diameter empty sphere (Kim K K et al. (1998) Nature 394, 595-9). In order to impart ‘tumor targeting’ capabilities to the Hsp cage, the RGD-4C peptide was genetically incorporated into a previously described Hsp variant (HspG41C) (Flenniken (2005) Chem Commun (Camb) supra Flenniken (2003) NanoLetters supra). The HspG41C mutant presents unique reactive cysteine residues on the interior surface of the assembled cage for attachment of cargo molecules. Protein modeling, based on crystallographic data, indicate that C-terminal amino acid residues 140-146 are found on the exterior surface of the Hsp cage (Kim K K (1998) Nature supra; Kim C K et al. (2002). Arch Pharm Res 25, 229-39). An Hsp C-terminal RGD-4C fusion protein was genetically engineered to present exposed RGD-4C loops on the exterior of the protein cage. Glycine residues (SGGCDCRGDCFCG) were added both before and after the RGD-4C insert to extend the peptide away from the C-terminus and allow for some structural flexibility. The insert was confirmed by DNA sequencing and the new ‘tumor targeting’ Hsp cages were expressed and purified from an E. coli expression system. Mass spectrometry verified the average subunit mass of HspG41CRGD-4C to be 17814.3 compared to the predicted mass of 17814.6. (FIG. 16) The experimental mass of 17814.3 correlates well with the predicted mass of 17,814.6 (see Supplemental Data in published article Flenniken, M. L. et al. (2006) Chemistry & Biology 13, 161-170 incorporated herein by reference in its entirety). This deconvoluted data was produced from the raw data by the software MaxEnt1, (Waters). The HspG41CRGD-4C mutant assembled as well as the wildtype protein cage (FIG. 16). HspG41CRGD-4C protein cage purification did not require reducing agents to prevent inter-cage aggregation, suggesting that the four cysteines present in each RGD-4C loop are disulfide bonded.

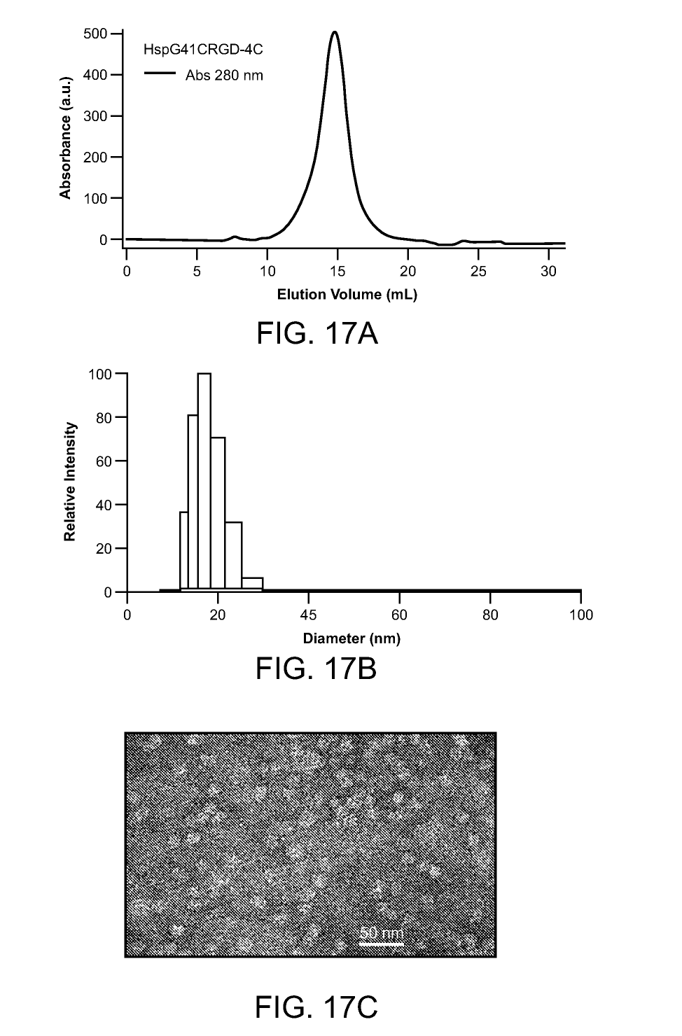

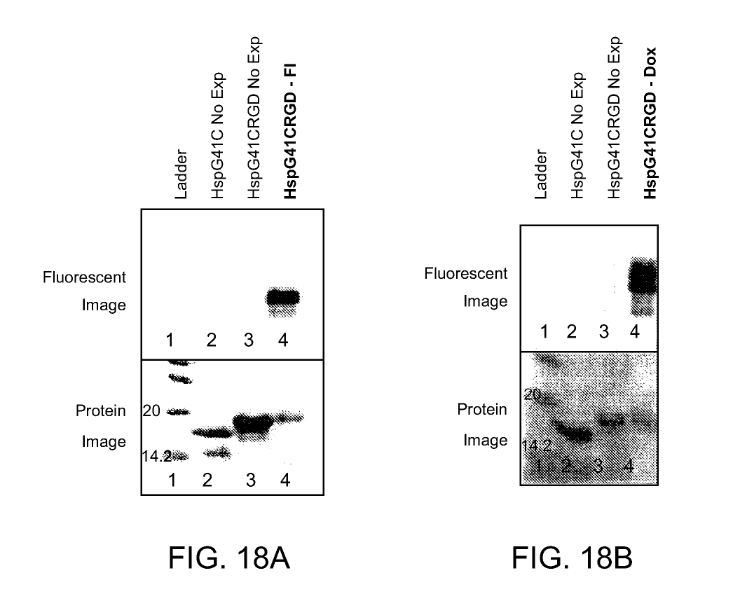

Characterization of recombinant ‘tumor targeting’ HspG41CRGD-4C protein cages by size exclusion chromatography, dynamic light scattering, and transmission electron microscopy (TEM) demonstrated that the overall spherical structure of the Hsp cage was not compromised due to the incorporation of the RGD-4C peptide (FIG. 17C). Size exclusion chromatography elution profiles of HspG41CRGD-4C cages indicate that the cages are slightly larger than the HspG41C parent cage lacking the targeting peptide (FIG. 17A)(Flenniken (2003) NanoLetters supra). This observation was supported by dynamic light scattering (DLS) which indicated a larger average diameter for the HspG41 CRGD-4C cages (15.4+0.3 nm) as compared to HspG41C (12.7+0.5 nm) (FIG. 17B). Transmission electron microscopy images of the HspG41CRGD-4C cages and HspG41C cages are indistinguishable (FIG. 17C)(Flenniken (2003) NanoLetters supra). We chemically attached fluorescein molecules to cysteine residues on Hsp cages in order to study cell-specific targeting of HspG41CRGD-4C cages. This demonstrates that both cell targeting and imaging functionalities can be simultaneously engineered into the Hsp protein cage architecture. The HspG41CRGD-4C has a total of 120 cysteines per cage (5 cysteines per subunit). Sub-stoichiometric labeling with fluorescein-5-maleimide ensured that every cage displayed a significant fraction of unmodified cysteines within the RGD-4C sequence. The original RGD-4C peptides discovered by phage display were in a cyclic conformation due to intrapeptide disulfide bond formation (Arap (1998) Science supra; Ellerby (1999). Nat Med supra). Likewise, we predicted that RGD-4C peptides presented on HspG41CRGD-4C architectures would also cyclize due to intrapeptide disulfide bond formation. Hsp-fluorescein conjugated cages were purified from free fluorescein via size exclusion chromatography and the covalent nature of the fluorescein-Hsp subunit linkage was demonstrated by SDS-polyacrylamide gel electrophoresis (SDS-PAGE) (FIG. 18). The fluorescently imaged gels illustrate covalent linkage of fluorescein (A—Lane 4) or doxorubicin (B—Lane 4) to HspG41CRGD-4C monomers whereas unlabeled Hsp subunits exhibit no fluorescence (Lanes 2 & 3). After fluorescent imaging, the proteins in gels A & B were stained with Coomassie Brilliant blue. HspG41CRGD-4C, 17816.6 Daltons (Lane 3) runs higher than HspG41C, 16498.2 Daltons (Lane 2) due to the addition of RGD-4C. The molecular weight standard (Lane 1) indicates the relative positions of 20 and 14.2 kDa proteins.

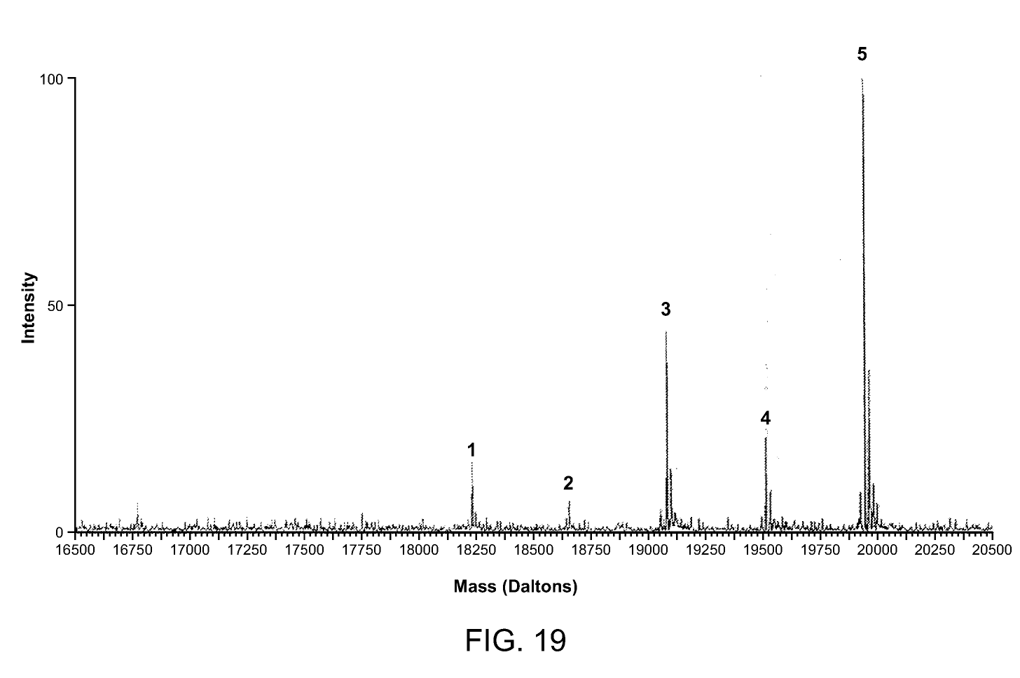

Mass spectrometry analysis of fluorescein labeled HspG41CRGD-4C subunits indicated that there are between one and five fluoresceins per subunit (See FIG. 19). The deconvoluted Mass spectrum of HspG41 CRGD-4C-Fluorescein shows from 1 to 5 fluoresceins covalently attached to one subunit indicated by the number. This deconvoluted data was produced from the raw data by the software MaxEnt1, (Waters).

Absorbance spectroscopy determined that on average there were 26 out of the 120 possible cysteines per cage labeled with fluoresceins, therefore only a fraction of the cysteines within the RGD-4C were bound to fluorescein. Some of the RGD-4C peptides were presumably in ‘loop’ conformation whereas others were potentially linearized depending on the number of conjugated fluorescin-5-maleimides per subunit (Flenniken (2003) NanoLetters supra). Data from mass spectrometry indicated the presence of both the disulfide and the free thiol form of the RGD-4C peptide. Previous structural studies of synthetic RGD-4C peptides in solution revealed that there are two predominant cyclic conformations both of which bind αvβ3 although one (RGD-A) exhibited higher binding affinity (Assa-Munt N, et al. (2001). Biochemistry 40, 2373-8). However, in a second report, synthetically produced acyclic RGD-4C was shown to have higher binding affinity than the cyclic form (Burkhart D J et al. (2004). Mol Cancer Ther 3, 1593-604). Since the fluorescein labeled HspG41CRGD-4C constructs most likely had a mixed presentation of ‘loop’ and linearized RGD-4C targeting peptides we hypothesized that they would bind αvβ3 expressing cells.

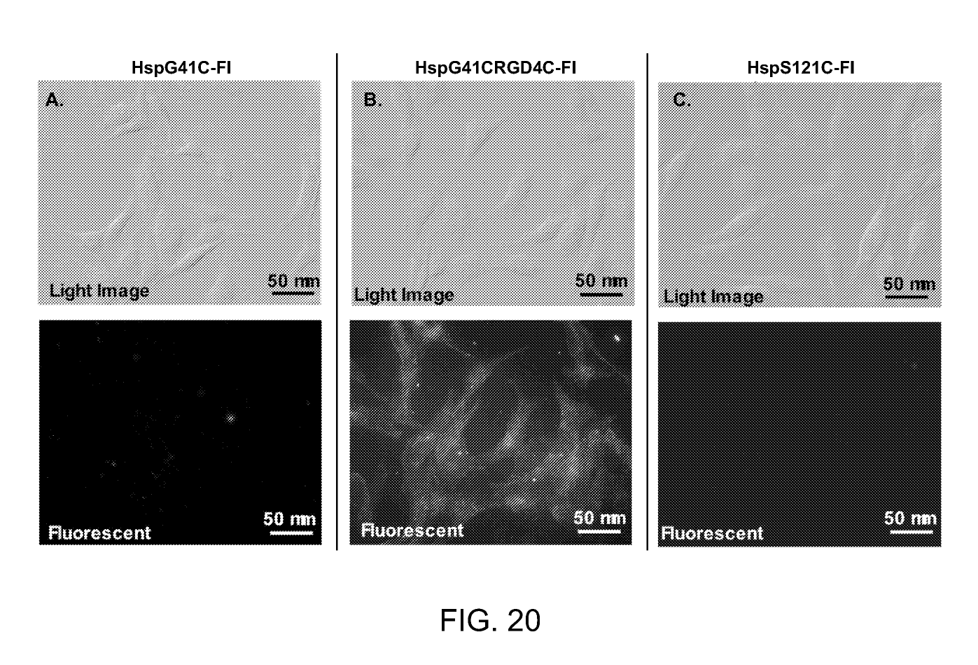

Epifluorescence microscopy was used to visualize fluorescently labeled HspG41CRGD-4C cages bound to αvβ3 integrin expressing C32 melanoma cells in vitro (Gao A G et al. (1996) J Cell Biol 135, 533-44; Hellwage J, (1997). Biochem J 326 (Pt 2), 321-7). For all microscopy studies, C32 melanoma cells were grown on glass coverslips. Cell surface expression of αvβ3 integrin on C32 cells was verified by immunofluorescence utilizing a fluorescein-conjugated anti-αvβ3 monoclonal antibody (mAb) (LM609). HspG41CRGD-4C-fluorescein cages were observed to efficiently bind to C32 cells as compared to control samples of Hsp cages without the RGD-4C peptide (FIG. 20). For direct comparison of epifluorescence data the concentration of fluorescein was normalized (2.5 #M), and the illumination intensity and the camera exposure were held constant. HspG41CRGD-4C-fluorescein protein cages were observed to efficiently bind to C32 cells as compared to controls of Hsp protein cages lacking the RGD-4C peptide (FIG. 20). Cells were incubated with (A) non-targeted HspG41C-Fl cages with interiorly bound fluorescein, (B) ‘tumor targeted’ HspG41CRGD4C-Fl cages and (C) non-targeted HspS121C-Fl cages with exteriorly bound fluorescein. C32 melanoma cells grown on coverslips were incubated with Hsp cage-fluorescein conjugates and imaged by both light (top) and fluorescent microscopy (bottom). The fluorescein concentration for cage-cell incubations was 2.5 μM and all fluorescent images were taken at a standardized camera exposure time of 50 ms. Scale bar=50 μm.

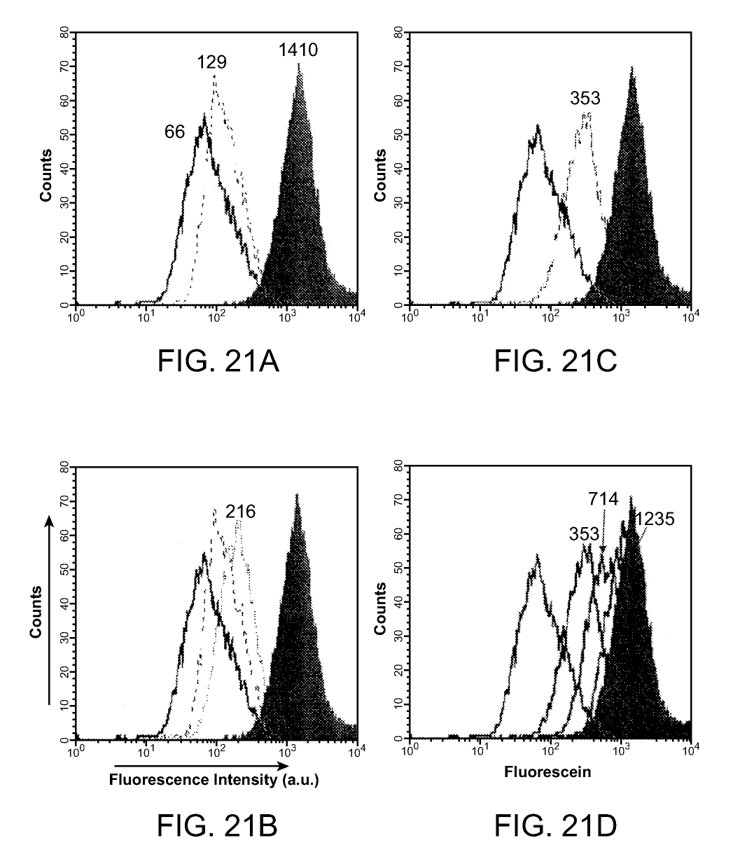

In order to ensure that HspG41CRGD-4C-fluorescein interaction with C32 cells was not mediated solely by exterior RGD-4C bound fluorescein, an additional mutant with surface exposed cysteine residues (HspS121C) was also tested. The HspS121C-fluorescein cages bind fluorescein-5-maleimide via externally facing cysteines but lack the RGD-4C targeting sequences. These control cages did not bind C32 cells at a level detectable by epifluorescence microscopy, indicating the cell binding observed for HspG41CRGD-4C was due to the presence of the RGD-4C peptide (FIG. 21).

Fluorescence activated cell sorting (FACS) was used to quantitate the ability of fluorescein labeled HspG41CRGD-4C cages to bind to C32 melanoma cells. Adherent C32 melanoma cells were non-enzymatically disassociated from cell culture dishes and suspended in DPBS+Ca2+/Mg2+. Fluorescently labeled Hsp cage preparations were incubated with cells on ice at a normalized fluorescein concentration of 2 #M and cells were washed prior to FACS. C32 cell associated fluorescence was dramatically increased after incubation with HspG41CRGD-4C-fluorescein cages.

The geometric mean (geo. mean) fluorescence intensity value of 1410 clearly indicated that ‘tumor targeted’ Hsp cages exhibited cell binding in vitro (FIG. 21A). The FACS data of C32 cells incubated with Hsp-fluorescein cages are plotted as histograms labeled with their corresponding geometric mean fluorescence intensity values (geo. mean). The background level of C32 cell associated fluorescence (blue solid line; geo. mean 66) and the increased level of C32 cell associated fluorescence due to binding of ‘tumor targeted’ HspG41CRGD4C-Fl cages (green filled plot; geo. mean 1410) are combined with additional FACS data to facilitate direct comparison of the data. (A) non-targeted cages HspG41C-fluorescein labeled interiorly (red dashed line; geo. mean 129); (B) nontargeted HspS121C-Fl cages with exteriorly bound fluorescein (orange dotted line; geo. mean 216); (C) Anti-αvβ3 integrin mAb blocked C32 melanoma cells subsequently incubated with HspG41CRGD4C-Fl cages (dashed purple line; geo. mean 353) (D) mAb concentration dependent blocking of αvβ3 integrin on C32 cells demonstrated by subsequent incubation of blocked cells with HspG41CRGD4C-Fl; 200 μg/mL mAb (purple solid line; geo. mean 353), cells blocked with 100 μg/mL (green solid line; geo. mean 714), 10 μg/mL mAb (solid black line; geo. mean 1235).

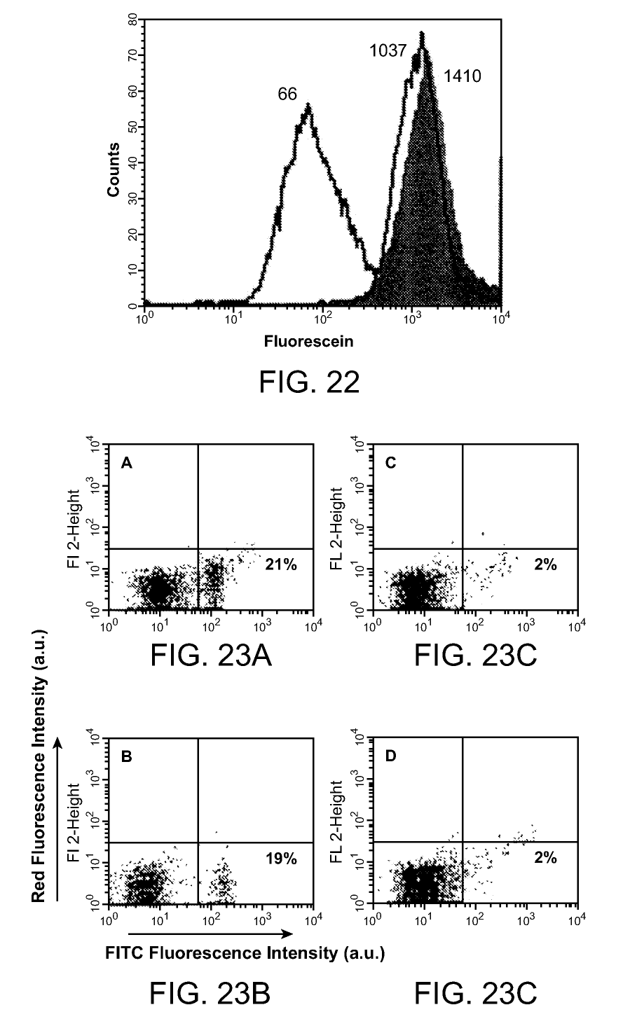

C32 melanoma cells do exhibit a background level of auto-fluorescence with geometric mean fluorescence intensity value of 66 (FIG. 21A). Control experiments in which C32 cells were incubated with a fluorescein conjugated anti-αvβ3 mAb also showed the expected increase in fluorescence (geo. mean 1037) associated with cell specific binding of the antibody (FIG. 22). FACS analysis of HspG41CRGD-4C-fluorescein and anti-avb3 antibody-fluorescein interaction with C32 melanoma cells. The FACS data of C32 cells incubated with Hsp-fluorescein cages are plotted as histograms labeled with their corresponding geometric mean fluorescence intensity values (geo. mean). The background level of C32 cell associated fluorescence (blue solid line; geo. mean 66) and the increased level of C32 cell associated fluorescence due to binding of ‘tumor targeted’ HspG41CRGD4C-Fl cages (green filled plot; geo. mean 1410) and anti-αvβ3 antibody-fluorescein positive control (black line; geo. mean 1037).

FACS analysis of non-targeted cages (HspG41C fluorescein inside; HspS121C-fluorescein outside) indicated low levels of non-specific binding of protein cages and fluorescein to C32 cells (geo. mean intensities of 129 and 216 respectively, FIG. 23A-B). In all cases these results were independent of incubation times, which ranged from 20 minutes to 2 hours (data not shown). The binding specificity of RGD-4C targeted Hsp cages was demonstrated with antibody blocking experiments. Blocking αvβ3 integrin present on C32 cells with an unlabeled anti-αvβ3 integrin mAb prior to incubation with fluorescently labeled HspG41CRGD-4C cages resulted in a reduction of RGD-4C targeted cage binding to levels similar to those observed with non-targeted cages (FIG. 23C). The degree of antibody blocking was concentration dependant. At 10 #g/mL minimal inhibition was observed, at 100 #g/mL some inhibition was evident, and finally at 200 #g/mL binding was inhibited to levels corresponding to that of non-targeted cages (FIG. 23D). These data illustrate the effective genetic introduction of ‘tumor targeting’ capacity to the Hsp cage architecture.

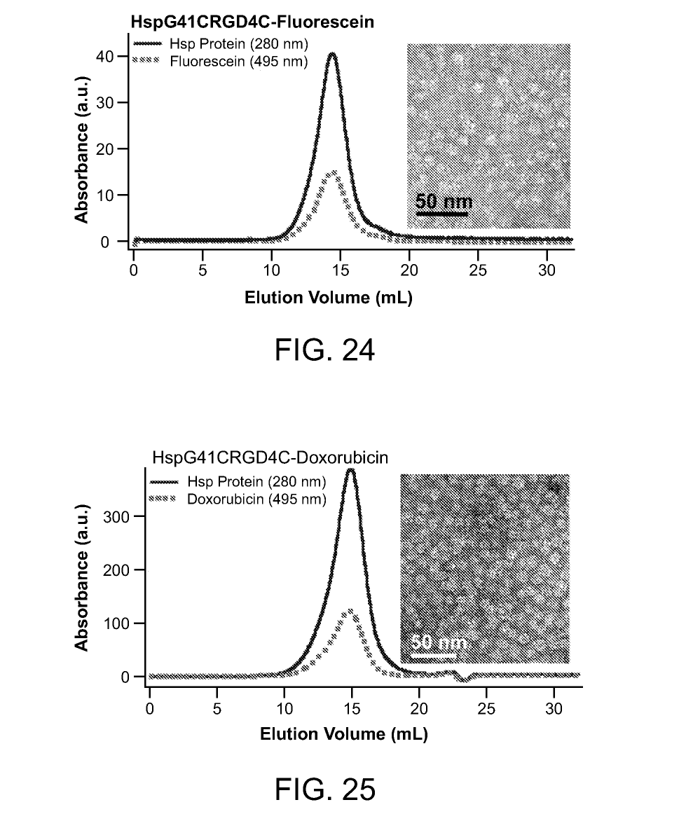

FIG. 24 shows the size exclusion chromatography elution profile of HspG41CRGD-4C cages. Co-elution of the absorbance at 495 nm (fluorescein) and 280 nm (protein) illustrate that fluorescein bound to intact Hsp cages. TEM micrograph (inset) also demonstrates the presence of intact Hsp cages.

Toward the eventual goal of developing targeted drug delivery systems, HspG41CRGD-4C cages were conjugated to the (6-maleimidocaproyl) hydrazone of doxorubicin (Flenniken (2005) Chem Commun (Camb) supra; Willner D et al. (1993) Bioconjug Chem 4, 521-7). Size exclusion chromatography illustrated the co-elution of absorbance at 280 nm (protein) and 495 nm (doxorubicin) indicative of their association (FIG. 25). FIG. 25 shows the size exclusion chromatography elution profile of HspG41CRGD-4C cages. Co-elution of the absorbance at 495 nm (doxorubicin) and 280 nm (protein) illustrate that doxorubicin bound to intact Hsp cages. TEM micrograph (inset) also demonstrates the presence of intact Hsp protein cages.

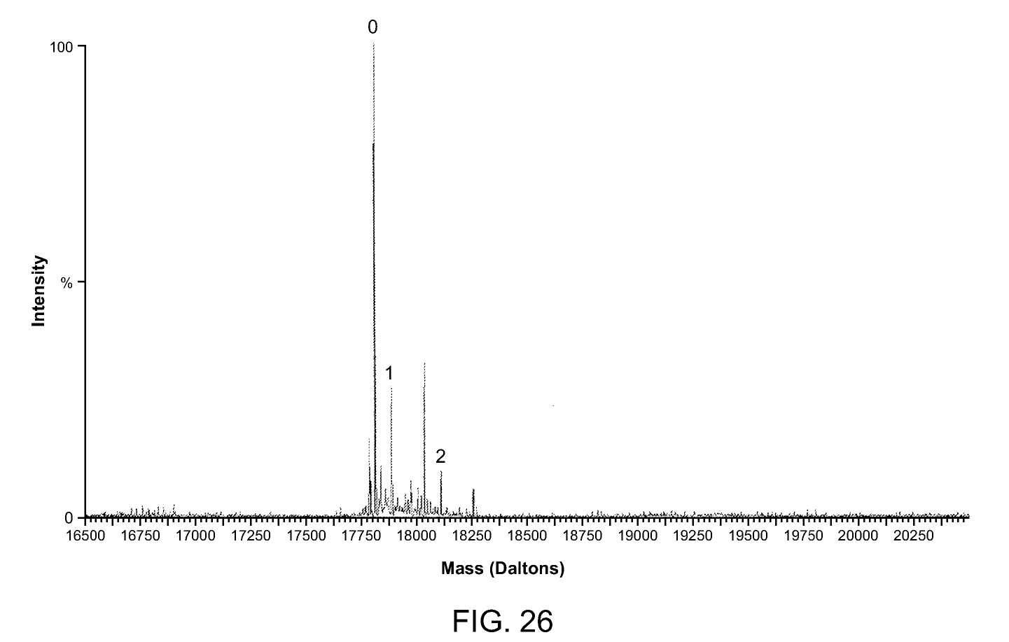

The covalent linkage of doxorubicin to HspG41CRGD-4C subunits was demonstrated by SDS-PAGE and mass spectrometry (FIG. 26). FIG. 26 shows the deconvoluted mass spectrum for HspG41CRGD-4C-Doxorubicin showing from 0 to 2 hydrazone linkages covalently attached to one subunit indicated by the number. The hydrazone linkage attached to the subunit, with the loss of doxorubicin, is expected since the liquid chromatography conditions are acidic and the doxorubicin is connected through an acid labile bond. The extra peaks are BME (β-mercaptoethanol) adducts of the sample. This deconvoluted data was produced from the raw data by the software MaxEnt1, (Waters).

Studies investigating the efficacy of targeted protein cages for therapeutic delivery are underway. The versatility of protein architectures for cell specific targeting was demonstrated by the chemical introduction of lymphocyte targeting to Hsp architectures. An anti-CD4 mAb (ATCC GK1.5) was conjugated to fluorescein labeled HspG41C cages via the heterobifunctional cross-linker SMCC (sulfosuccinimidyl 4-(N-maleimidomethyl) cyclohexane-1-carboxylate). Size exclusion chromatography was utilized to purify anti-CD4-HspG41C-fluorescein cage conjugates (Ab-Hsp-Fl) from HspG41C-fluorescein cages. The elution profile indicated that the Ab-Hsp-Fl cage conjugates were larger than HspG41C-Fl cages and DLS confirmed the size increase from 12.2 nm to 22+0.1 nm diameter (Supplemental Data in published article previously mentioned).

As shown in FIG. 23 and described above, FACS was used to investigate binding of anti-CD4-HspG41C-fluorescein (Ab-Hsp-Fl) cages to CD4+ cells within a murine splenocyte population. FACS Analysis of murine splenocytes incubated with: (A) anti-CD4 mAb HspG41C-fluorescein cage conjugates (Ab-Hsp-Fl) bound 21% of cells within this population (B) anti-CD4 mAb-fluorescein demonstrated that 19% of this splenocyte population is CD4+, (C) CD4+ cells were blocked with unlabeled anti-CD4 mAb, then subsequently exposed to Ab-Hsp-Fl cage conjugates demonstrated a low level of non-specific binding (2%), corresponding to (D) non-targeted HspG41C-fluorescein cages (2%).

Ab-Hsp-Fl cages specifically bound to 21% of the total cells within this population (FIG. 23A). This level of binding is consistent with the percentage of CD4+ cells within this murine splenocyte population as determined by binding of a fluorescein conjugated anti-CD4 mAb to 19% of this murine splenocyte population (FIG. 23B). Further confirmation of binding specificity was obtained from antibody blocking experiments. Splenocytes were incubated with unlabeled anti-CD4 mAb, washed to remove unbound blocking antibody, and subsequently incubated with Ab-Hsp-Fl cage conjugates. FACS analysis of this blocking experiment demonstrated that only 2% of the population exhibited cell associated fluorescence (FIG. 23C). This percentage of cell fluorescence corresponds to that observed with non-targeted HspG41C-fluorescein cages (2%) (FIG. 23D). The observed binding of Ab-Hsp-Fl cages to 21% of the murine splenoctye population encompasses both the specific binding to CD4+ lymphocytes (19%) on top of a small non-specific level of background association (2%). This indicates that immuno-targeted protein cages effectively target to specific cells within a mixed population. In this work, the 12 nm diameter Hsp cage was both genetically and chemically modified to incorporate cell specific targeting properties. Genetic incorporation of αvβ3 binding RGD-4C peptide onto the exterior surface of Hsp cages conferred tumor cell specific targeting. It is expected that many other cell targeting peptides, especially those discovered by in vivo phage display library techniques, could also be incorporated into this and other protein cage architectures. Chemical linkage of an anti-CD4-antibody to Hsp cages and subsequent targeting of a subset of CD4+ cells within a population of murine splenocytes, demonstrated the success and possibilities of immuno-targeted Hsp cages. Fluorescein, an imaging agent, and doxorubicin, an anti-tumor agent, were covalently linked to protein cages demonstrating that these architectures can simultaneously incorporate multiple functionalities including cell specific targeting, imaging and therapeutic agent delivery.

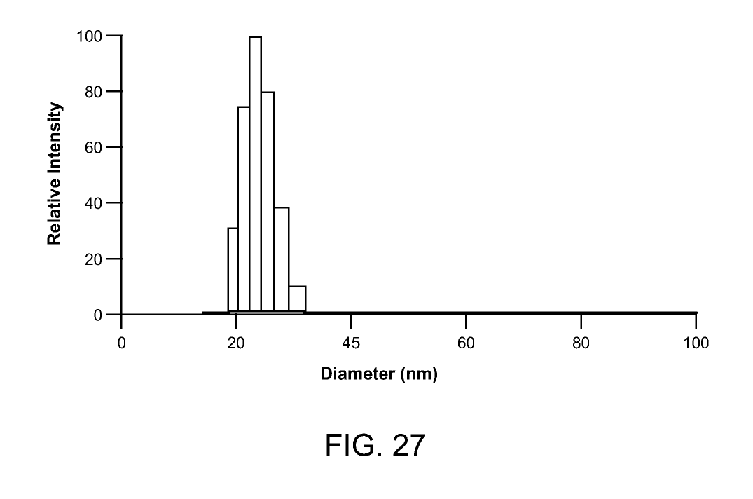

FIG. 27 shows the dynamic light scattering analysis of anti-CD4 mAb HspG41C. Cage conjugates dynamic light scattering (DLS) indicates that anti-CD4 mAb conjugated HspG41C fluorescein cages have an average diameter of 22.4+0.1 nm.

This research advances the utility of protein cage nano-containers as platforms for combined targeted therapeutic and imaging agent delivery systems with broad medical applications. It demonstrates that the Hsp cage architecture can be genetically and chemically modified to impart mammalian cell targeting capacity. This work also demonstrates the ability to simultaneously incorporate cell targeting, imaging and therapeutic agents within a single protein cage. Protein cage architectures are precisely defined monodisperse molecular platforms with inherent genetic and chemical versatility. A library of protein cage architectures is available ranging in size from 9 to >100 nm diameter extending the utility of this approach to diverse applications.

Experimental Procedures. Genetic Engineering of ‘Tumor-Targeting’ HspG41C-RGD4C. Methanococcus jannaschii genomic DNA was obtained from the American Type Culture Collection (ATCC 43607). As described previously, the gene encoding the small heat shock protein (Mj HSP16.5) was polymerase chain reaction (PCR) amplified and cloned into Ndel/BamH1 restriction sites of the PET-30a(+) vector (Novagen, Madison, Wis.) for expression of the full-length protein with no additional amino acids. PCR mediated sitedirected mutagenesis was employed to replace the glycine at position 41 with a unique cysteine residue, therefore generating the HspG41C mutant (Flenniken ML WD, Brumfield S, Young M J, Douglas T (2003). The Small Heat Shock Protein Cage from Methanococcus jannaschii Is a Versatile Nanoscale Platform for Genetic and Chemical Modification. NanoLetters 3, 1573-1576). Deletion of the HSPstop codon directly upstream of the BamH1 site was also accomplished by PCR mediated site directed mutagenesis. This deletion allowed for the insertion of additional sequence into the BamH1 site to create the RGD-4C (CDCRGDCFC) carboxyl-terminal fusion protein engineered to present exposed RGD-4C loops on the exterior of the protein cage. The HspG41CRGD-4C fusion protein was engineered to have extra glysine residues (italicized below) both before and after the RGD-4C insert to extend the insert away from the C-terminus and allow some structural flexibility. Complimentary RGD-4C encoding primers with gatc overhangs for cloning into the BamHI site (+sense primer: 5′ ga tct gga gga tgc gac tgc cgc gga gac tgc ttc tgc gga taa gga 3′; encoding—S G G C D C R G D C F C G stop) were mixed at a 1:1 molar ratio, annealed and treated with kinase (Promega, Madison, Wis.). These inserts were subsequently ligated into an alkaline phosphatased, BamH1 digested vector overnight at 17° C. and transformed into XL-2 ultracompetant E. coli (Stratagene, La Jolla, Calif.). Transformants were screened for the presence of the RGD-4C insert and confirmed by sequencing the PCR amplified product on an ABI 310 automated capillary sequencer using Big Dye Chain termination sequence technology (Applied Biosystems, Foster City, Calif.).

HspG41CRGD-4C Cage Purification and Characterization. All small heat shock protein cages (HspG41C, HspS121C, HspG41CRGD-4C) were purified from an E. coli heterologous expression system as previously described (Flenniken (2003) NanoLetters supra). One liter cultures of E. coli (BL21(DE3) B strain) containing pET-30a(+) MjHsp16.5 plasmid were grown overnight in LB+kanamycin medium (370 C, 220 rpm). Cells were harvested by centrifugation 3700×g for 15 minutes (Heraeus #3334 rotor, Sorvall Centrifuge) and resuspended in 30 mL of 100 mM HEPES 50 mM NaCl, pH 8.0. Lysozyme, DNase, and RNAse were added to final concentrations of 50, 60, and 100 #g/mL, respectively. The sample was incubated for 30 minutes at room temperature, French pressed (American Laboratory Press Co., Silver Springs, Md.) and sonicated on ice (Branson Sonifier 250, Power 4, Duty cycle 50%, 3×5 minutes with 3 minute intervals). Bacterial cell debris was removed via centrifugation for 20 minutes at 12,000×g. The supernatant was heated for 15 minutes at 65° C. thereby denaturing many E. coli proteins. The supernatant was centrifuged for 20 minutes at 12,000×g and purified by gel filtration chromatography (Superose-6, Amersham-Pharmacia, Piscataway, N.J.; Bio-Rad Duoflow, Hercules, Calif.). Recombinant HspG41CRGD-4C protein cages were routinely characterized by size exclusion chromatography (Superose 6, Amersham Pharmacia), dynamic light scattering (Brookhaven 90Plus, Brookhaven, N.Y.), transmission electron microscopy (TEM) (Leo 912 AB), SDS poly-acrylamide gel electrophoresis (SDS-PAGE) and mass spectrometry (Acquity/Q-Tof micro, Waters, Milford, Mass.). Protein concentration was determined by absorbance at 280 nm divided by the published extinction coefficient (9322-M−1 cm−1)(Kim K K, Kim R, Kim S H (1998). Crystal structure of a small heat-shock protein. Nature 394, 595-9).

Labeling Hsp with Activated Fluorescein Dye. Cysteine containing Hsp cages (100 mM HEPES 50 mM NaCl pH 6.5) were reacted with fluorescein-5-maleimide (Molecular Probes, Eugene, Oreg.) in concentrations ranging from 1-6 molar equivalents per Hsp subunit for 30 minutes at room temperature, followed by overnight incubation at 4° C. Fluorescein labeled Hsp cages were purified from free dye by size exclusion chromatography (DPBS pH 7.4). The number of fluorescein molecules per cage was calculated from absorbance spectra (Flenniken (2005) Chem Commun (Camb) supra; Flenniken et al. (2003). NanoLetters supra). For example, HspG41CRGD-4C (2 mg/mL; 112 #M subunit) reacted with 2.2 molar equivalents of fluorescein-5-maleimide (246 #M) per Hsp subunit resulted in HspG41CRGD-4C cages with an average of 26.2 fluoresceins per cage (or 1.09 fluoresceins per subunit). The number of fluoresceins per cage was quantified by absorbance spectroscopy (Flenniken (2003) NanoLetters supra).

C32 amelanotic melanoma Cell Culture. Human amelanotic melanoma cell line, C32, was obtained from the American Type Culture Collection (ATCC CRL-1585) (ATCC Manassas, Va.). C32 cells were propagated in Minimum Essential Medium Eagle (MEME) (ATCC 30-2003) supplemented with 10% fetal bovine serum (Atlanta Biologicals, Norcross, Ga.), Penicillin (100 units/mL) and Streptomycin (100 #g/mL) (Sigma, St. Louis, Mo.) at 370 C, in a 5% CO2 incubator.

Mass Spectrometry. Hsp samples were analyzed by liquid chromatography/electrospray mass spectrometry (LC/MS) (Acquity/Q-Tof micro, Waters; Milford, Mass.). HspG41CRGD-4C and derivatized HspG41CRGD-4C (5-15 μL, 0.3-0.5 mg/mL) were injected onto a C8 column (208TP5115, Vydac) and eluted with a H2O-acetonitrile gradient. The aqueous solvent contained 0.1% formic acid and the acetonitrile contained 0.05% trifluoro acetic acid.

Epifluorescence Microscopy. Epifluorescence microscopy was performed on an Axioscope 2-Plus microscope (Zeiss) utilizing version 4.1 software and an Axiocam High resolution camera (Hrc). For all microscopy studies, C32 melanoma cells were grown on glass coverslips to ˜60% confluency (MEME+10% FBS), in the presence of penicillin (100 units/mL) and streptomycin (100 #g/mL) (Sigma, St. Louis, Mo.). C32 expression of the RGD-4C target receptor, αvβ3 integrin, was verified by immunofluorescence utilizing a fluorescein-conjugated anti-αvβ3 monoclonal antibody (mAb) (LM609) (Chemicon MAB1976F, Temecula, Calif.). C32 cells grown on coverslips were incubated with Hsp cages in serum free medium for 30 minutes at 370 C, in a 5% CO2 incubator. The fluorescein concentration of the Hspfluorescein preparations was normalized to 2.5 #M to facilitate comparison. After incubation, the cells were washed 5 times with Dulbecco's Phosphate Buffered Saline (DPBS) (Sigma, St. Louis, Mo.), fixed with 4% paraformaldehyde for 10 minutes, washed with DPBS and then mounted on slides in Vectashield mounting medium (Burlingame, Calif.). Illumination intensity and camera exposure times were held constant.

Fluorescence Activated Cell Sorting (FACS) Analysis of C32 cells Incubated with Fluorescein Conjugated Hsp Cages. Flow cytometry was performed on a FACSCalibur, (Becton Dickinson, Mountain View, Calif.) and analyzed using Cell Quest software (Becton Dickinson, Mountain View, Calif.). Adherent C32 melanoma cells were non-enzymatically disassociated from cell culture dishes with DPBS without Ca2+ or Mg2++1% EDTA (˜25 mM) (for about 2 minutes at room temperature), washed once with serum containing medium, and finally suspended in DPBS+Ca2+/Mg2+ at 2.1×106 cells/mL. Fluorescently labeled cage preparations (normalized to 2 #M fluorescein) were incubated with cells on ice from 20 minutes to 2 hours. After incubation the cells were washed 5 times with DPBS (both with and without Ca2+/Mg2+), and suspended in DPBS+1% FBS for FACS analysis. Both the anti-αvβ3 monoclonal antibody (mAb) (LM609) (Chemicon MAB1976Z, Temecula, Calif.) and the corresponding fluorescein-conjugated anti-αvβ3 mAb (Chemicon MAB1976F) were used for FACS analysis.

HspG41CRGD-4C Doxorubicin Conjugation. HspG41CRGD4-C cages (2 mg/mL; 112 #M subunit) in 100 mM HEPES 50 mM NaCl pH 6.5 were reacted with a 2.2 fold excess of the (6-maleimidocaproyl) hydrazone of doxorubicin (246 #M) for 30 minutes followed by purification via size exclusion chromatography. The average number of doxorubicin molecules per cage was 10 (an average of 0.42 doxorubicin molecules per subunit) as calculated from absorbance spectra (Flenniken M L et al. (2005) Chem Commun (Camb) supra; Willner (1993) Bioconjug Chem supra). HspG41C-Fluorescein anti-CD4 Antibody Conjugation. Fluorescein labeled HspG41C protein cages were conjugated to anti-CD4 monoclonal antibodies (generated from ATCC GK1.5) via a heterobifunctional cross-linker SMCC (sulfosuccinimidyl 4-(N-maleimidomethyl)cyclohexane-1-carboxylate) (Pierce, Rockford, Ill.). First, the antibodies (6.5 mg/mL in PBS pH 7.4) were partially reduced with 10 mM TCEP (tris(2-carboxyethyl)phosphine) in the presence of 10 mM EDTA (ethylenediaminetetraacetic acid) with the final pH adjusted to 6.5 and incubated for 2 hours at room temperature (Hermanson GT (1996). Bioconjugate Techniques. (San Diego: Academic Press)). Simultaneously, the exposed lysines (amines) of HspG41C-fluorescein cages (0.25 mg/ml/15 #M subunit in 500 #L DPBS pH 7.4; 11 fluoresceins per cage) were reacted with the sulfo-NHS-ester component of the SMCC linker (added in excess 0.5 mg). The Hsp cage plus linker reaction was incubated at room temperature for one hour followed by the removal of free SMCC linker by size exclusion chromatography (Desalting Column, Pierce, Rockford, Ill.). The reduced anti-CD4 antibodies were combined with the HspG41C-fluorescein-SMCC cages and incubated for 3 hours before final purification of anti-CD4-HspG41C-fluorescein cage (Ab-Hsp) conjugates by size exclusion chromatography (Superose 6, Amersham-Pharmacia, Piscataway, N.J.).

Murine Splenocyte Preparation. A Balb/c mouse spleen was homogenized in Hanks Balanced Salt Solution (Mediatech, Herndon, Va.) by pushing it through a 60 gauge stainless steel mesh, the homogenate was filtered through 100 #m nylon mesh, and centrifuged (200×g for 10 minutes). The supernatant was discarded and the cell pellet suspended in 5 mL ACK lysis buffer (150 mM NH4Cl, 1 mM KHCO3, 0.1 mM Na2EDTA) for 5 minutes at room temperature to lyse unwanted red blood cells. Lysis was stopped via the addition of PBS+2% donor calf serum (25 mL). The remaining white blood cells were pelleted by centrifugation (200×g for 10 minutes) and suspended in PBS+2% donor calf serum (2×107 cells/ml) containing anti-mouse Fc receptor antibody (from HB-197, ATCC) in order to prevent non-specific binding of antibodies to the Fc receptor on lymphocytes. Cells were incubated on ice until binding assays were performed.

Fluorescence Activated Cell Sorting (FACS) Analysis of C32 Cells Incubated with Anti-CD4 mAb Conjugated Hsp Cages. FACS was performed on a FACSCalibur, (Becton Dickinson, Mountain View, Calif.) and analyzed using Cell Quest software (Becton Dickinson, Mountain View, Calif.). Aliquots of murine splenocytes were combined with equal volumes of each of the following: A) anti-CD4-HspG41C-fluorescein (Ab-Hsp-Fl) cages, B) fluorescein conjugated anti-CD4 mAb (positive control), and C) HspG41C-fluorescein cages (non-targeted control), and incubated for 30 minutes on ice. Following incubation the cells were washed in PBS containing 2% donor calf serum in preparation for FACS. FACS analysis was performed on a gated murine splenocyte cell population. For anti-CD4 mAb blocking experiments. splenocytes were incubated for 30 minutes with unlabeled anti-CD4 mAb and washed to remove unbound blocking antibody prior to incubation with Ab-Hsp-Fl cage conjugates. Supplemental Data. including Figures S1-S6, are available at http://www.chembiol.com/cgi/content/full/13/2/161/DC1.

### Example 6A

### Cancer Cell Targeting Ferrimagnetic Iron Oxide Nanoparticle Incorporated into a Ferritin Cage Architecture

Protein cage architectures such as virus capsids and ferritins are versatile nanoscale platforms amenable to both genetic and chemical modification. Incorporation of multiple functionalities within these nanometer sized protein architectures demonstrate their potential to serve as functional nanomaterials with applications in medical imaging and therapy. In the present study, we synthesized an iron oxide (magnetite) nanoparticle within the interior cavity of a genetically engineered human H-chain ferritin (HFn). A cell-specific targeting peptide, RGD-4C which binds αvβ3 integrins upregulated on tumor vasculature, was genetically incorporated on the exterior surface of HFn. Both magnetite containing and fluorescently labeled RGD4C-Fn cages bound C32 melanoma cells in vitro. Together these results demonstrate the capability of a genetically modified protein cage architecture to serve as a multifunctional nanoscale container for simultaneous iron oxide loading and cell-specific targeting.

Protein cage architectures such as virus capsids and ferritins are versatile nanoscale platforms amenable to both genetic and chemical modification. Incorporation of multiple functionalities within these nanometer sized protein architectures demonstrate their potential to serve as functional nanomaterials with applications in medical imaging and therapy. In the present study, we synthesized an iron oxide (magnetite) nanoparticle within the interior cavity of a genetically engineered human H-chain ferritin (HFn). A cell-specific targeting peptide, RGD-4C which binds αvβ3 integrins upregulated on tumor vasculature, was genetically incorporated on the exterior surface of HFn. Both magnetite containing and fluorescently labeled RGD4C-Fn cages bound C32 melanoma cells in vitro. Together these results demonstrate the capability of a genetically modified protein cage architecture to serve as a multifunctional nanoscale container for simultaneous iron oxide loading and cell-specific targeting.

Mutagenesis of RGD-4C peptide conjugated ferritin (RGD4C-Fn). The N-terminal of the HFn are exposed on the exterior surface of the assembled ferritin cage. Therefore, the RGD-4C peptide was incorporated at the N-terminus of the HFn subunit to present 24 copies of the targeting peptide. To accomplish this, an AatII restriction site was introduced into the native HFn for subsequent incorporation of the RGD-4C peptide sequence. Primers were designed as follows to change a tyrosine (ACC) to a serine (TCC) at amino acid position 3 of HFn: (+) 5′ GAA GAA GAT ATA CAT ATG ACG TCC GCG TCC ACC TCG CAG GTG 3′; (−) 5′ CAC CTG CGA GGT GGA CGC GGA CGT CAT ATG TAT ATC TCC TTC 3′. To incorporate the RGD-4C peptide sequence onto the N-terminus of the HFn, the complimentary primer 5′ GC GAC TGC CGC GGA GAC TGC TTC TGC GGA GGC GGA ACG T 3′ and 5′ TCC GCC TCC GCA GAA GCA GTC TCC GCG GCA GTC GCA CGT 3′ were designed and inserted into the introduced AatII site of the plasmid. Three glycine residues and one threonine residue were added after the RGD-4C peptide to allow for some structural flexibility. These mutagenesis experiments resulted in the change of the original HFn amino acid sequence MTTAS to MTCDCRGDCFCGGGTSAS. The RGD4C-Fn plasmid was isolated and sequenced as previously described for HFn.

Purification and Characterization of Ferritin. The HFn and RGD4C-Fn were expressed in E. coli where they self-assembled into the 24 subunit cages. One liter cultures of E. coli (BL21 (DE3): Novagen) containing pET-30a(+) HFn or RGD4C-Fn plasmid were grown overnight in LB medium with 30 mg/L kanamycin. RGD4C-Fn protein production was induced by IPTG (1 mM) and cells were incubated for an additional 4 h. After the incubation, cells were collected by centrifugation and then the pellets were resuspended in 45 ml of lysis buffer (100 mM HEPES, 50 mM NaCl, pH 8.0). Lysozyme, DNAse and RNAse were added to final concentrations of 50, 60 and 100 μg/ml, respectively. After 30 min incubation at room temperature, the solution was subjected to French press followed by sonication on ice. The solution was centrifuged to remove E. coli debris. The supernatant was heated at 60° C. for 10 min, precipitating many of the E. coli proteins, which were removed by centrifugation. The supernatant was subjected to size exclusion chromatography (SEC:Amersham-Pharmacia, Piscataway, N.J., USA) with a Superose 6 column to purify HFn or RGD4C-Fn. The protein cages were characterized using SEC, dynamic light scattering (DLS: Brookhaven, 90Plus particle size analyzer) and transmission electron microscopy (TEM: LEO 912AB). Protein concentration was determined by absorbance at 280 nm. Typically yields were 100 mg for HFn and 10 mg for RGD4C-Fn (isolated protein) per 1 liter batch.

Iron oxide mineralization and characterization. A degassed solution (8.0 ml of 100 mM NaCl) was added to a jacketed reaction vessel under an N₂ atmosphere followed by addition of HFn (2.0 mg, 3.9 nmol) or RGD4C-Fn (2.1 mg, 3.9 nmol) in 100 mM NaCl (either of HFn or RGD4C-Fn is 1.0 mg/ml) to the vessel. The temperature of the vessel was kept at 65° C. using circulating water through the jacketed flask. The pH was titrated to 8.5 using 50 mM NaOH (718 Auto Titrator, 8 Brinkmann). Fe(II) was added (12.5 mM (NH₄)₂ Fe(SO₄).6H₂O) to attain a theoretical loading factor of 1000 Fe (313 μl), 3000 Fe (939 μl) or 5000 Fe (1566 μl) per protein cage. Stoichiometric equivalents (1H₂O₂:3 Fe(II)) of freshly prepared degassed H₂O₂ (4.17 mM) was also added as an oxidant (reaction 1). The Fe(II) and H₂O₂ solutions were added simultaneously at a constant rate of 31.3 μl/min (100 Felprotein·min) using a syringe pump (Kd Scientific). H+ generated during the reaction was titrated dynamically using 50 mM NaOH to maintain a constant pH 8.5. The reaction was considered complete 5 min after addition of all the iron and oxidant solutions. After the completion of the reaction, 200 μl of 300 mM sodium citrate was added to chelate any free iron. Horse spleen apo ferritin (Sigma Chemicals Ltd., USA) was also subjected to the mineralization reaction with a theoretical loading factor of 1000 Fe per cage using the same procedure. The mineralized sample was analyzed using SEC with Superose 6. Absorbance at 280 nm and 410 nm were simultaneously monitored for protein and mineral, respectively. The sample was imaged by TEM, and electron diffraction and electron energy loss spectroscopy (EELS) data were collected on all samples.

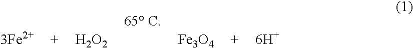

Magnetic characterizations of the mineralized samples were performed on a physical properties measurement system (PPMS: Quantum Design). Dynamic and static magnetic measurements were carried out using an alternating current magnetic susceptibility (ACMS) and a vibrating sample magnetometer (VSM) option, respectively. ACMS measurements were performed under a 100e field at frequencies of 100, 500, 1000, 5000, and 10000 Hz, with no dc background, while the temperature was varied from 100 to 4 K. The superparamagnetic blocking temperature was determined from susceptibility curves. VSM measurements were performed under a magnetic field up to 80 kOe at 5 K.

C32 amelanotic melanoma cell culture. Human amelanotic melanoma cell line, C32, was purchased from the American Type Culture Collection (ATCC CRL-1585). C32 cells were cultured in Minimum Essential Medium Eagle (MEME, ATTC 30-2003) supplemented with 10% fetal bovine serum (Atlanta Biologicals, Norcross, Ga.), 100 units/ml of penicillin and 100 μg/ml of streptomycin at 37° C. in 5% CO₂ atmosphere.

Cell targeting assay of mineralized RGD4C-Fn. The mineralized RGD4C-Fn with a loading factor of 3000 Fe/cage was used to evaluate the ability of the protein cage to target cancer cells. The mineralized HFn cages (3000 Fe/cage) was used as a control. C32 cells were cultured in a six-well polystyrene plate. After 2 days of incubation, MEME was removed from the well followed by addition of 1 ml of mineralized protein (200 μg/ml) in Dulbecco's Phosphate Buffered Saline (DPBS) and then incubated at 37° C. for 30 min. After the incubation, the solution was aspirated and washed 2 times with DPBS. Cells were fixed in 3% glutalaldehyde in 0.1 M potassium sodium phosphate buffer (PSPB) at pH 7.2 for 10 min at room temperature. Following removal of gultalaldehyde, 1 ml DPBS was added and cells were scraped using a rubber policeman and then centrifuged. Cell pellets were mixed with 50 μl of 4% agar solution and allowed to solidify. Agar pellets were then placed in 3% gultalaldehyde in PSPB overnight at 4° C. Glutalaldehyde was removed and pellets were rinsed 2× with PSPB for 10 min each time. The pellets were then fixed for 4 h at room temperature followed by dehydrations steps of 50%, 50%, 70%, 95%, 100%, 100% and 100% ethanol for 10 min each. Propylene oxide was used as a transitional solvent before infiltration with Spurr's embedding resin. (Spurr, A. R. J. Ultrastruct. Res. 1969, 26, (1-2), 31) Cell pellets were thick and thin sectioned prior to imaging by TEM. The surface length of the cells were measured and the number of iron oxide clusters were counted on each image, to estimate the number of cluster per μm of cell surface.

Labeling of ferritin with activated fluorescein dye. Fluorescein-5-maleimide, which is capable of reactivity with cysteine residues presenton proteins, was used for fluorescence labeling of ferritin. Either HFn or RGD4C-Fn in buffer (100 mM HEPES, 50 mM NaCl, pH6.5) was reacted with fluorescein-5-maleimide (Molecular Probes, Eugene, Oreg.) in a concentration of 3 molar equivalents pre ferritin subunit at room temperature for 30 min followed by overnight incubation at 4° C. (Flenniken (2003) Nano lett. supra; Flenniken (2005) Chem. Commun. supra) Fluorescein labeled protein cages were purified from free dye using SEC as described above. The number of fluorescein linked per protein cage was determined using absorbance spectroscopy as previously described ((Flenniken (2003) Nano lett. supra)

Fluorescence activated cell sorting (FACS) analysis of C32 cell s incubated with fluorescein conjugated RGD4C-Fn. Flow cytometry was performed on a FACSCalibur, (Becton Dickinson, Mountain View, Calif.) and analyzed using Cell Quest software (Becton Dickinson, Mountain View, Calif.). In order to perform the analysis, C32 cells were non-enzymatically removed from cell culture dishes by DPBS (without Ca₂₊ and Mg₂₊) with 1% ethylenediaminetetraacetic acid (EDTA), washed once with serum containing MEME and then suspended in DPBS (with Ca₂₊ and Mg₂₊). The cells were incubated with fluorescently labeled protein cages (normalized to 2 μM fluorescein) in DPBS (with Ca₂₊ and Mg₂₊) at a concentration of 2.5×10₆ cell/ml on ice for 20 min. After the incubation, the cells were washed 2 times with DPBS (with Ca₂₊ and Mg₂₊) and then resuspended in DPBS (with Ca₂₊ and Mg₂₊). To demonstrate selective binding of RGD4C-Fn to cancerous cells, a control experiment using non-cancerous T cells, which do not express □ᵥ□₃ integrins, were subjected to FACS analysis in the same manner as the C32 cell experiments described earlier. Binding specificity of RGD4C-Fn was also investigated in competition experiments, in which C32 cells were pre-incubated with increasing concentrations (55 μg/mL to 1.1 mg/mL) of either RGD4C-Fn or HFn (unlabeled) prior to incubation with fluorescently labeled RGD4C-Fn.

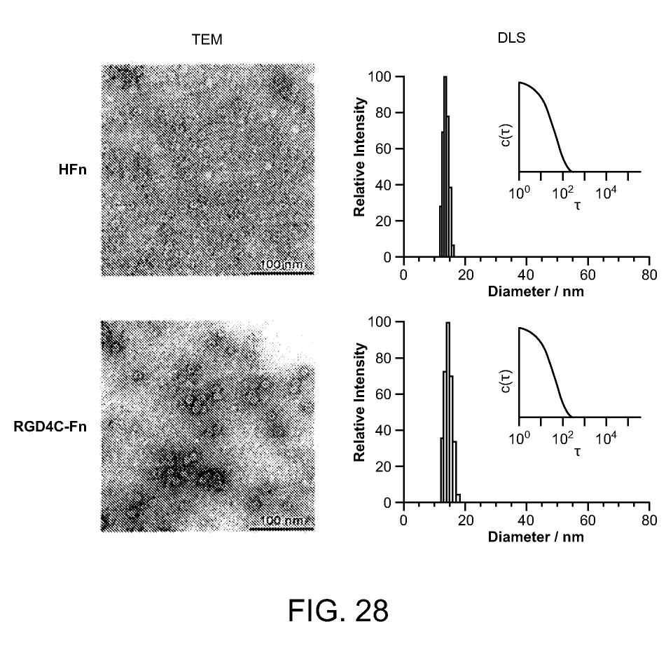

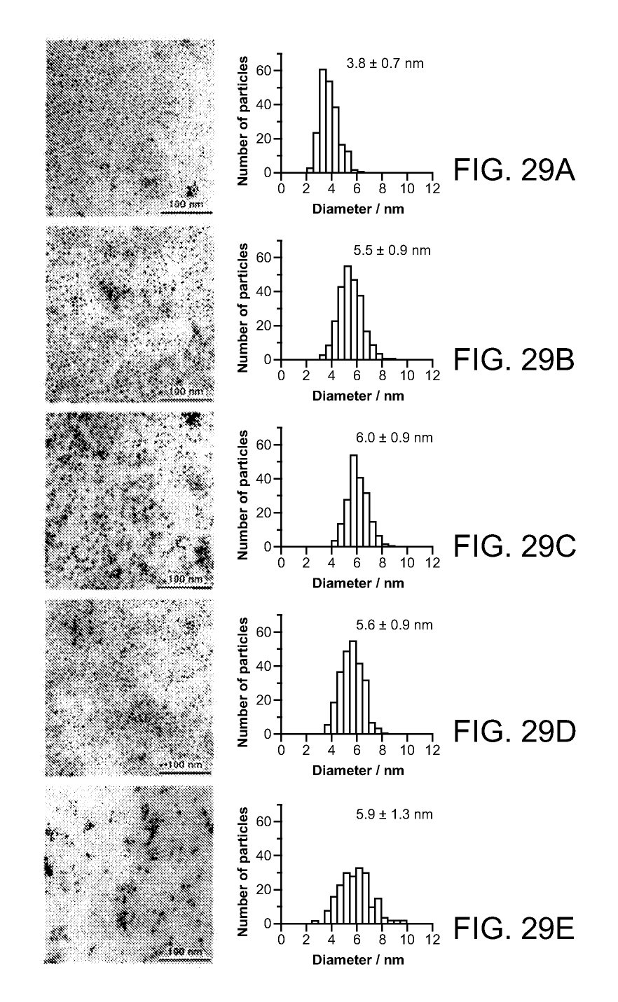

Results and Discussion. We have combined two important aspects of biological approaches to materials synthesis to form functional magnetic materials with the ability to target specific cells. Cell and tissue specific targeting peptides (RGD-4C), identified by in vivo phage display, have been genetically incorporated into the subunits of human H-ferritin (HFn) giving rise to a self-assembled cage architecture capable of both cell targeting and biomimetic mineralization. In this study, we present data on the ferrimagnetic iron oxide nanoparticle synthesis and characterization using both the HFn cage architectures and the cancer cell targeting variant of the HFn cage architecture, RGD4C-Fn. In addition, the efficacy of cell targeting is demonstrated on fluorescently labeled HFn and RGD4C-Fn using FACS analysis. Both the HFn and RGD4C-Fn were heterologously expressed in E. coli. The purified proteins were analyzed by transmission electron microscopy (TEM), which revealed that both HFn and RGD4C-Fn adopted the expected spherical cage-like structures with a diameter of approximately 12 nm, and that the two cages were indistinguishable (FIG. 28). The self-assembled cages were further assessed using dynamic light scattering (DLS) and found to be monodisperse in solution (FIG. 28) with no significant difference in size between HFn (mean=13.4 nm) and RGD4C-Fn (mean=14.0 nm). Likewise, analysis of both HFn and RGD4C-Fn protein cage architectures by size exclusion chromatography (SEC) exhibited identical elution times (data not shown). These results indicate that fusion of the RGD4C peptide to the N-terminus of the HFn subunit does not interfere with the self-assembly of the subunits to form the characteristic 24 subunit protein cage architecture of ferritin. The purified protein cages were subjected to synthetic iron oxide mineralization under conditions of elevated pH and temperature in order to direct the formation of the ferrimagnetic phase Fe₃O₄ (or □-Fe₂O₃). Briefly outlined, solutions of Fe(II) and oxidant (H₂O₂) were added via syringe pump to the apoferritin cages under an atmosphere of N₂ at pH 8.5 and elevated temperature (65° C.) over a defined time period (10, 30 or 50 minutes). The reaction was performed using a range of stoichiometric ‘loading factors’ ranging from 1000 Fe per cage to 5000 Fe per cage. In the presence of the HFn, or RGD4C-Fn, a homogeneous brown colored solution was obtained after the reaction for all loading factors. Control reactions, run in the absence of the protein cages, resulted in bulk precipitation of the iron oxide from solution. Unstained products of protein-mediated mineralization were observed by TEM and showed electron-dense nano-particles (FIG. 29). The average diameter of the particles increased from 3.8 to 6.0 nm as the loading factor was increased from 1000 to 5000 Fe per cage. There was no significant difference in particle size between the products mediated by the HFn and RGD4C-Fn. Table 1 shows the d-spacing for maghemite, magnetite and measured d-spacing for mineralized RGD4C-Fn with loading factor of 3000 Fe/cage.

TABLE 1

| d-spacing of maghemite | d-spacing of magnetite | Measured d-spacing |
|---|---|---|
| 2.518 | 2.532 | 2.54 |
| 1.476 | 1.485 | 1.49 |

Table 2 shows a comparison of measured iron core diameter in RGD4C-Fn, previously reported iron core diameter in horse spleen Fn and calculated theoretical diameter.

TABLE 2

| | | | Calculated |
|---|---|---|---|
| Theoretical iron | Mean iron core | Mean iron core | theoretical iron |
| loading; atoms/ | diameters in | diameters in horse | core |
| protein cage | RGD4C-Fn;/nm | spleen Fn;/nm | diameters;/nm |
| 260 | — | 5.7** | 2.3 |
| 1000 | 3.8* | 5.9*, 7.5** | 3.6 |
| 3000 | 5.5* | 8.4** | 5.2 |
| 5000 | 6.0* | — | 6.2 |

*The data was obtained in this experiment.
**The data was from reference (24)

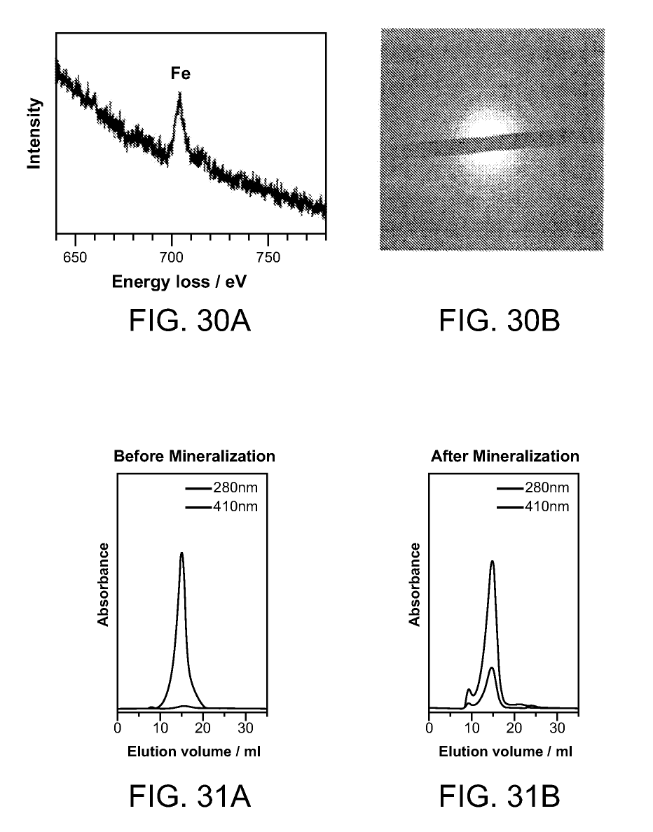

This indicates that RGD-4C peptide, on the exterior surface of the cage, had little effect on the mineralization process, even though there are four cysteine residues present in the peptide. Electron energy loss spectroscopy (EELS) of the particles showed the iron L23 spectrum (FIG. 30) and selected area electron diffraction from a collection of particles in both the mineralized RGD4C-Fn and HFn exhibited powder diffraction patterns, which could be ascribed to either maghemite or magnetite (FIG. 30 and Table 1).

It should be noted that the size (volumes) of the Fe3O4 cores formed inside of the HFn and RGD4C-Fn are smaller than those formed inside horse spleen ferritin (both prepared in this experiment and in a previous report—Wong, K. K. et al. (1998) S. Chem. Mater. 10, 279) and closer to the theoretical core diameters calculated for a uniform spherical particle of the cubic iron oxide (either Fe3O4 or □-Fe2O3) phase at these loading stoichiometries (FIG. 29 and Table 2). This suggests that the HFn and the RGD4C-Fn mutant are superior platforms for the homogeneous nucleation of iron oxide inside of the protein cage as compared to horse spleen apo-ferritin. This interesting difference is probably due to the difference of subunit composition between HFn and horse spleen ferritin. Native mammalian ferritins consist of mixtures of two different subunit types; H (heavy) and L (light) chain. Horse spleen ferritin is a mixture of about 15% H chain and 85% L chain subunits, while HFn is a homopolymer of only H chain (Harrison, P. M. et al. (1996) Biochim. Biophs. Acta, Bioenerg. 1275, (3), 161). The H chain contains a catalytic ferroxidase site (absent in L chain), which is responsible for Fe (II) oxidation to Fe (III) (Lawson, D. M. et al. (1991) Nature 349, (6309), 541). In ferritin, oxidation of Fe (II) is followed by spontaneous nucleation due to extremely low Fe (III) solubility. The enhanced control over particle size exhibited by our results with HFn suggests that the ferroxidase site plays an important role in iron oxide nucleation even under these fairly harsh synthetic conditions. It further suggests also that under these (non physiological) conditions the ferroxidase site in HFn is able to utilize H2O2 a function previously only ascribed to related bacterioferritins. For both the HFn and RGD4C-Fn the mineralization occurred in a spatially selective manner and the reaction did not disrupt the protein cage in any measurable way. SEC analysis of the RGD4C-Fn before and after mineralization with 3000 Fe/cage (FIG. 31) showed co-elution of the protein cage (280 nm) and the mineral core (410 nm), whereas before the mineralization, protein shows no absorption at 410 nm. In addition, retention time of mineralized protein cage was identical to the cage before mineralization. This elution behavior indicates the protein-mineral composite nature of the material and suggests that the overall structure of the protein cage has not been significantly perturbed by the mineralization process. The iron oxide mineral is clearly encapsulated and sequestered within the protein cage as a result of the mineralization reaction.

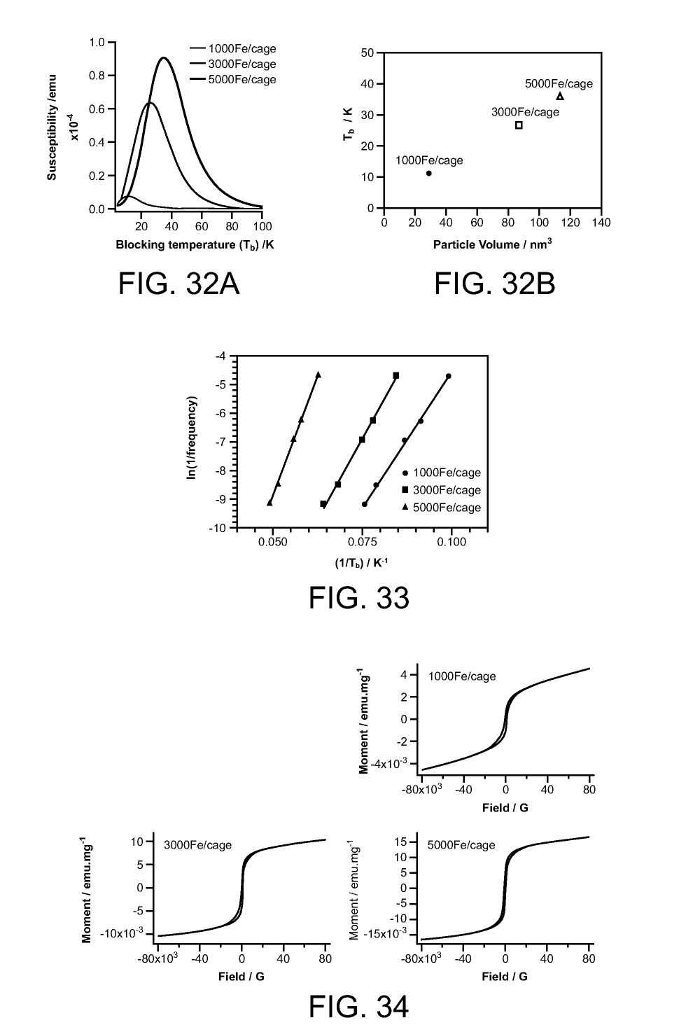

Magnetic characterization using a physical properties measurement system (PPMS) was used to probe the size dependent magnetic properties of the mineralized cages. Using alternating current magnetic susceptibility (ACMS), the blocking temperature (Tb) of the samples was found to increase with increasing particle size from 11K for the 1000 Fe loading to 27K for the 3000 Fe loading to 36 K for Fe loading factor of 5000 ions, as shown in FIG. 32. Data was analyzed using the Neel-Arrhenius equation (2).

$$ln ( 1 / f ) = ln ( 1 / {Γ}_{0} ) + \frac{{E}_{a}}{{k}_{B}} \frac{1}{{T}_{b}} \qquad ( 2 )$$

Where f is measurement frequency, Γ₀ is the attempt frequency, Eₐ is the anisotropy energy and T_{b} is the blocking temperature. By measuring the susceptibility of a sample at multiple frequencies, a fit to the Neel-Arrhenius equation was obtained. As shown in FIG. 33, Neel-Arrhenius fits showed linear behavior for all the three samples. Since Eq. (1) is the expected behavior of an isolated particle, the liner behavior indicates that the particles are encapsulated within the protein cages and clearly not interacting with each other. Vibrating sample magnetometry (VSM) measurements revealed that all samples exhibited superparamagnetic behavior at room temperature since no hysteresis was observed at 300K. At 5 K, ferrimagnetic components were evident in the magnetic behaviors of all samples (FIG. 34). Coercive fields (Hc) of 720 G, 550 G and 620 G was observed for 1000 Fe/cage, 3000 Fe/cage and 5000 Fe/cage, respectively. These results indicate that magnetic properties of the mineralized ferritin can be adjusted according by controlling synthesis conditions, in particular the Fe to protein stoichiometry.

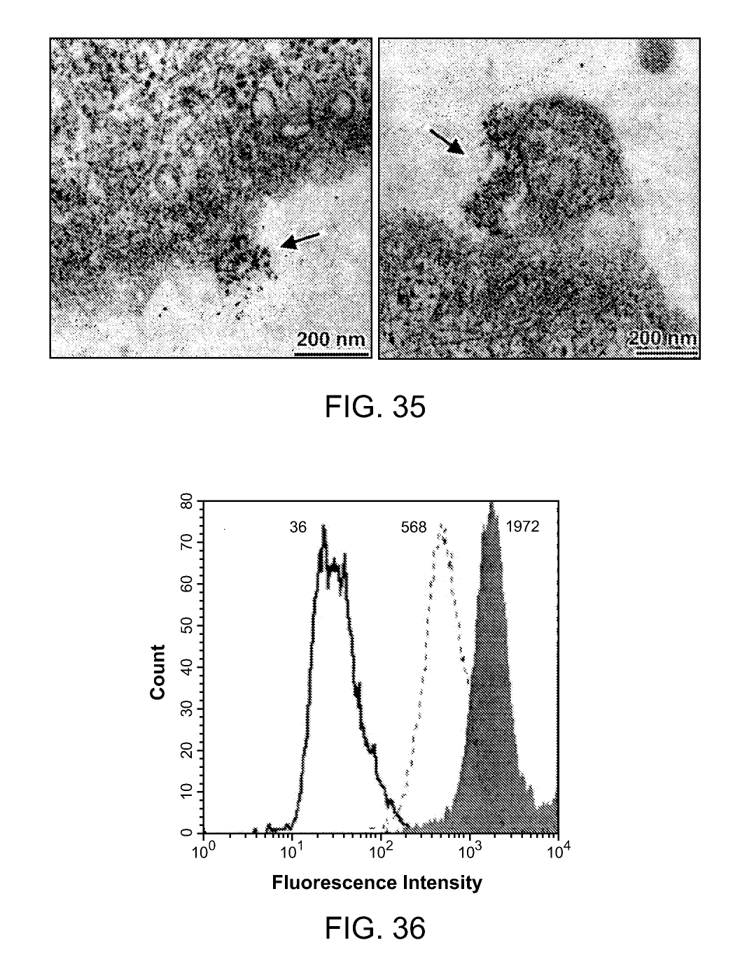

To test the targeting efficacy of the RGD4C-Fn construct we compared its binding to C32 amelanotic melanoma cells. C32 cells overexpress the alphavbeta3 integrins on their cell surface and these are known to be the recognition sites for the RGD-4C (CDCRGDCFC) peptide.43 Cells were incubated with either the RGD4C-Fn or HFn and subsequently washed, thin sectioned and imaged by TEM. As shown in FIG. 35, clusters of small electron dense particles were easily found on the surface of the cells, corresponding to the mineral core within the ferritin cages. These clusters were absent in cells not incubated with mineralized RGD4C-Fn or HFn. While some clusters were observed in cells incubated with HFn, their number density was significantly lower than for cells incubated with RGD4C-Fn cages (roughly 50% lower). This indicates that the RGD-4C peptide moiety affords enhanced targeting capacity to the mineralized Fn cage.

In order to quantitate cancer cell binding ability of the RGD4C-Fn fluorescence activated cell sorting (FACS) analysis was performed using fluorescein labeled cages (RGD4C-Fn and HFn). The number of fluorescein molecules conjugated to the protein cages was determined by absorbance spectroscopy to be 0.6 per subunit (14.4 per cage) for HFn and 0.8 per subunit (19.2 per cage) for RGD4C-Fn (data are not shown), whereas the number of exterior surface exposed cysteine residues was 2 and 6, respectively. These data suggest that the four additional cysteines of the RGD4C peptide do not participate in the conjugation reaction. The result of FACS analysis indicates that RGD4C-Fn exhibit enhanced C32 melanoma cell targeting ability (FIG. 36). C32 cells incubated with fluorescein-labeled RGD4C-Fn had a geometric (geo.) mean fluorescence intensity value of 1972, whereas the cells exhibit autofluorescence with geo. mean fluorescence intensity value of 36. Although the C32 cells incubated with non-targeted ferritin cage (HFn) did exhibit some non specific interaction (geo. mean fluorescence intensity value of 568) this is significantly smaller than for cells incubated with RGD4C-Fn. These data are consistent with those obtained from TEM observation of C32 cells incubated with the ferritin cages.

To demonstrate the specificity for the alphavbeta3 integrins control (non-cancerous) T cells were analyzed for their ability to bind RGD4C-Fn. Cells were incubated with either RGD4C-Fn or HFn and both exhibited a similar background level of non-specific binding, several orders of magnitude below the binding observed in C32 experiments. Geo. mean fluorescence intensity values of these two cases (15 and 25, respectively) were close to that of the T cells without incubation of any fluorescently labeled protein cage (Supporting information figure S1). This result clearly indicates that RGD4C-Fn cages lack specific binding ability to non-cancerous T cell. In addition, competition experiments were performed to evaluate the specificity of binding.

C32 cells were pre-incubated with increasing amounts (55 μm/mL to 1.1 mg/mL) of either RGD4C-Fn or HFn (unlabeled) prior to incubation with fluorescently labeled RGD4C-Fn. FACS analysis revealed that unlabeled RGD4C-Fn could effectively compete for binding with the labeled RGD4C-Fn, whereas HFn was far less effective in competitive binding (Supporting information figure S2). Together these results indicate that RGD4C-Fn exhibits specific targeting capacity towards cancerous cells expressing high levels of alphavbeta3 integrins. These results hold the promise for successful targeting to angiogenic tumor vasclutature. The result of C32 cell specific binding of RGD-4C conjugated ferritin is similar to a previous report in which the ability of small heat shock protein genetically modified to incorporate the RGD-4C peptide to bind cancer cells was investigated (Flenniken, M. L. et al. (2006) Chem. Biol. 13, (2), 161). This illustrates the versatility of our approach, which has wide applicability for a variety of protein cages. A library of protein cage architectures having a range of sizes is available and thus extends the utility of this approach for size dependent cage-property selection. Furthermore, it is expected that genetic incorporation of cell binding peptide onto the exterior surface of protein cages has the potential for applications in cell specific therapeutic and imaging delivery system because not only the RGD-4C peptide but also many other cell targeting peptides could be incorporated into protein cage architectures using essentially the same approach.

Conclusion. We have demonstrated that the human H-chain ferritin cage can serve as a multifunctional platform for the biomimetic synthesis of magnetic nanoparticles and can be engineered for use as a cell specific targeting moiety. The present work reveals two important points. First, both HFn and RGD4C-Fn can be used as a constrained reaction environment for the synthesis of superparamagnetic magnetite and/or maghemite without perturbing its cage-like architecture. This means that the exterior surfaces of protein cage architectures can be modified without altering the function of the cage as a size constrained reaction vessel. Second, the mineralized RGD4C-Fn exhibits increased specific targeting interaction with a cancer cell as compared to the control HFn. The magnetic and cell targeting capabilities engineered into this protein cage makes it ideal as a new diagnostic imaging agent. This demonstrates the ability to add multi-functionality such as cell targeting, imaging, and perhaps therapeutic agents simultaneously to a single protein cage. The utility of these materials for in vivo targeted diagnostic imaging are currently being evaluated. This work extends the diverse applications of protein cages in the field of biomedicine because the current approach is applicable in modifying other protein cages or introducing other cell targeting peptides to a protein cage. Supplemental Data is available at http://www.chembiol.comlcgi/content/full/13/21161/DC1/.

### Example 7

### Functional Asymmetry in a Protein Cage Architecture Through Controlled Assembly

Protein cages derived from viral architectures have emerged as robust templates for nano-materials fabrication and are typically assembled from a single or limited number of subunits into highly symmetrical structures. We have developed an approach to introduce functional asymmetry into an icosahedral protein cage while maintaining the underlying structural symmetry. Differentially modified subunits were assembled into icosahedral structures with introduced asymmetry determined by the stoichiometry of the subunits present during the assembly process.

Spherical virus architectures and other protein cages such as ferritins and heat shock proteins offer possibilities for nano-scale fabrication (Kramer, R. M. et al. (2004) J. American Chem. Soc. 2004, 126, (41), 13282-13286; Klem, M. T. et al. (2003) J. American Chem. Soc., 125, (36), 10806-10807; Douglas, T. et al. (2002); Advanced Materials, 14, (6), 415; Douglas, T. et al. (1998) Nature 393, (6681), 152-155; Douglas, T. et al. (1999) Advanced Materials 11, (8), 679; Ensign, D. et al. (2004). Inorg Chem 43, (11), 3441-6; Raja, K. S. et al. (2003) Chembiochem 4, (12), 1348-51; Flenniken, M. L. et al. (2003); Nano Letters 3, (11), 1573-1576; Flenniken, M. L. et al. (2005) Chem Commun (Camb) (4), 447-9; Wang, Q. et al. (2002) Angewandte Chemie-International Edition 41, (3), 459-462; Chatterji, A. et al. (2005) Nano Left 5, (4), 597-602; Niemeyer, C. M. et al. (2001) Angewandte Chemie-International Edition 40, (22), 4128-4158). These protein structures all share the common characteristics of self-assembly from a limited set of subunits into precisely defined, high symmetry architectures. In addition, protein cages provide scaffolding for spatially specific functionalization (Douglas (2002) Advanced Materials supra; Ensign (2004) Inorg. Chem. supra; Flenniken (2005) Chem Commun (Camb) supra; Gillitzer, E. et al. (2002) Chemical Communications (20), 2390-2391; Wang, Q. et al. (2002) Chem Biol 9, (7), 813-9; Wang, Q. et al. (2002) Chem Biol 9, (7), 805-11; Blum, A. S. et al. (2004) Nano Letters 4, (5), 867-870).

However, subunit modification typically results in a highly symmetrical, multivalent presentation of ligands distributed over the entire cage architecture Flenniken (2003) Nano Letters supra; Wang (2002) Angewandte Chemie-International Edition supra; Wang, Q. et al. (2002) Chem Biol 9, (7), 813-9; Wang, Q. et al. (2002) Chem Biol 9, (7), 805-11). While the high symmetry and associated multivalent presentation of spherical protein cages provides advantages for both biological function and synthetic utility, their potential for inducing formation of architecturally complex arrays of particles could be further enhanced if particles could be asymmetrically functionalized. We have previously described a solid phase approach to introducing functional asymmetry into a highly symmetrically protein cage architecture (Klem (2003) J. American Chem. Soc. supra) and others have used a similar approach (Dabrowski, M. J. et al. (1998) Chem Biol 5, (12), 689-97). Here we describe a second approach, using in vitro self-assembly, by which the high symmetry of an icosahedral virus particle can be broken to exhibit controlled functional asymmetry.

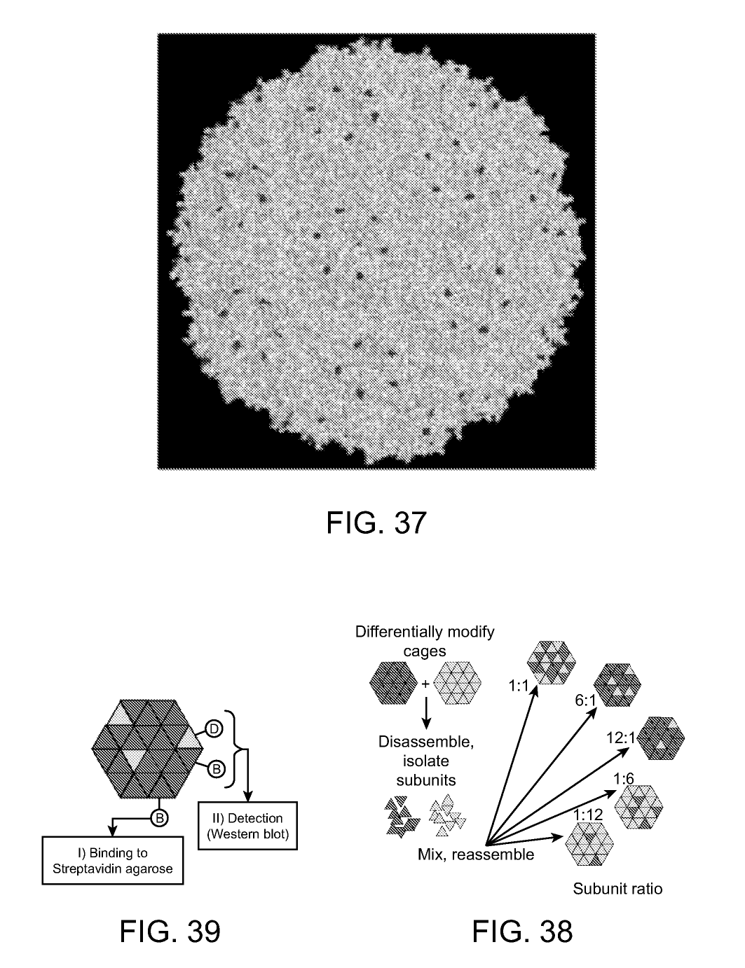

Cowpea chlorotic mottle virus (CCMV) is an ideal model for introducing functional asymmetry into a nano-architecture. CCMV is composed of 180 identical protein subunits that are arranged with icosahedral symmetry. (Speir, J. A. et al. (1995) Structure 3, (1), 63-78; Reddy, V. S. et al. (2001) J. Virology 75, (24), 11943-11947) (FIG. 37). The interior, exterior, and the interface between CCMV subunits can be chemically and or genetically modified to rationally impart function by design to the cage structure (Klem (2003) J. American Chem. Soc. supra; Douglas (2002) Advanced Materials supra; Gillitzer (2002) Chemical Communications supra; Fox, J. M. et al. (1996) Virology 222, (1), 115-122). An additional advantage of CCMV is that it can be disassembled in vitro into subunits and subsequently reassembled forming particles with the same icosahedral symmetry as wild type particles (Bancroft, J. B. et al. (1967) Virology 32, (2), 354; Bancroft, J. B. et al. (1967) Virology 31, (2), 354; Fox, J. M. et al. (1998). Virology 244, (1), 212-218; Zlotnick, A. et al. (2000) Virology 277, (2), 450-456; Zhao, X. X. et al. (1995) Virology 1995, 207, (2), 486-494; Johnson, J. M. et al. (2004) J. Molecular Biology 335, (2), 455-464). The in vitro assembly and disassembly process can be controlled by varying parameters such as pH, ionic strength, and metal ion concentration (see supplemental material). FIG. 37 shows a spacefilling representation of the exterior surface of CCMV (left) with reactive surface exposed lysines (K54, K84, K87, K65, K106, K131) 13 indicated in red illustrating their highly symmetric presentation on the icosahedral protein cage.

Spatially defined, chemical functionalization of subunits coupled with controlled in vitro particle disassembly and reassembly provides the basis for producing functionally asymmetric particles (FIG. 37 and FIG. 38). Conceptually, by controlling the ratio of chemically modified subunits in the reassembly reaction, one can exert control over the display of the ligands in the final assembled cage architecture. Due to the inherent symmetry of the particles, reassembly of a particle using subunits homogeneously modified with a single ligand will produce homogeneous particles displaying that ligand on the surface with icosahedral symmetry. Particles assembled from populations of subunits functionalized with two different ligands will exhibit asymmetry within individual icosahedral particles depending on the stoichiometry of the subunits. Alternatively, it is possible that the reassembly process would be biased toward formation of particles displaying a single ligand type. This would lead to two populations where each ligand is independently displayed with icosahedral symmetry, thus lacking the desired functional asymmetry.

FIG. 38 shows a schematic for the assembly of asymmetrically functionalized particles. Two populations of particles are labeled, disassembled, and subunits purified. The differentially labeled subunits are subsequently mixed together at different ratios during reassembly, resulting in functionally asymmetric particles. An advantage of producing particles that incorporate two different functional groups via subunit reassembly is that, assuming the subunits incorporate at random, the probability or expected fraction of particles (P) having a specified number of monomers functionalized with one of the groups (x) can be described by a binomial distribution (Eq. 1).
P(x;n,p)=c(n,x)p^{x}(1−p)^{n-x} Eq. 1

where n is the number of monomers per CCMV (180), p is the fraction of monomers functionalized with the specified group in the input mixture and c(m,x) is the number of combinations of x monomers selected from a set of n objects (total monomers).

For p<0.05, Eq. 1 can be approximated by the Poisson distribution (Eq. 2) (Johnson, R. A., (1994) Miller & Freund's probability and statistics for engineers. In Pearson Prentice Hall: Upper Saddle River, N.J.)
P(x,λ)=λ^{x}exp(−λ)/x! Eq. 2

where λ=np. 5

Eq. 2 can be used to predict the level of asymmetry imposed on a population of CCMV particles assembled from mixtures containing various proportions of monomers functionalized with two different ligands. For imposition of functional asymmetry we are interested in assembly mixtures composed of a small proportion of one of the ligands (type 1), with the other ligand (type 2) making up the balance. Formally, symmetry is broken for virus particles incorporating less than 60 ligands of type 1 since T=3 virus particles are composed of 60 asymmetrical units. For assembly mixtures with a ratio of less than 1:25 (type 1:type 2 input ratio) the probability of assembling a virus that incorporates 60 or more type 1 ligands is negligible (<10-15). From an engineering perspective, some useful behavior resulting from imposition of functional asymmetry (such as preferential orientation on a surface) should be observed when the icosahedral (20-fold symmetry) is broken. The fraction of virus particles incorporating 20 or more type I ligands is expected to be less than 10−4 for assembly mixtures with an input ratio of 1:25.

Thus, essentially all particles assembled from this mixture have the 20-fold symmetry broken. As expected, as the contribution of type I ligand is reduced in the assembly mixture the fraction of virus particles that incorporate this ligand is diminished (Table A, row 1). The fraction of virus particles incorporating only one type 1 monomer becomes significant for input ratios less than 1:100 (Table A, row 2). Within the subpopulation of assembled virus particles that incorporate both ligands the fraction of virus particles for which symmetry is completely broken (one type 1 ligand) becomes very substantial as the input ratio is reduced (Table A, row 3). For an input ratio of 1:400, almost 80% of particles that contain both types of ligands are expected to incorporate only one type 1 ligand. Thus, preferential orientation of virus particles assembled from this mixture on a surface designed to exclusively bind type I ligands would be superb. In addition, the yield of particles that incorporate both ligands at this input ratio (estimated from row 1 of Table A) is approximately 36%, suggesting that assembly of this oriented film would be practical. Table A shows the expected fractions of reassembled CCMV incorporating numbers of type 1 functionalized monomers for various input ratios of type 1 to type 2a.

TABLE A

| Number type | Input ratio (type 1:type 2)b | | |
|---|---|---|---|
| 1 ligand | 1:25 | 1:100 | 1:400 |
| 0 | 0.0007 | 0.1653 | 0.6376 |
| 1 | 0.0054 | 0.2975 | 0.2869 |
| 1 (excl 0d) | 0.0054 | 0.3565 | 0.7918 |

a predicted by the Poisson distribution (Eq.2)
bratio of type 1 to type 2 functionalized monomers in reassembly mixture (input)
c number type 1 functionalized subunits per 180 monomers in reassembled virus (output)
d fractions are those expected among the virus subpopulation that excludes virus with no type 1 functionalized monomers

To demonstrate the feasibility of imposing functional asymmetry on CCMV we created two populations of subunits, differentially labeled with two different ligands (FIG. 38). This was accomplished by first independently functionalizing exposed lysine residues on intact CCMV13 with either biotin (type 1) or digoxigenin (type 2) ligands to generate two differentially labeled populations of CCMV. These two populations were identical by transmission electron microscopy (TEM), dynamic light scattering (DLS), and size exclusion chromatography (SEC) analysis. The respective labels were detectable, as expected, by Western blot analysis. Subsequently, these intact particles were disassembled in vitro, and the resulting subunits were independently purified by SEC. Finally, purified subunits were mixed in defined stoichiometric ratios under in vitro assembly conditions. The resulting icosahedral particles were indistinguishable by TEM from 7 particles prior to disassembly. We examined whether both ligands were successfully incorporated into the same particle during the in vitro assembly process. This was accomplished by varying ratios of purified biotin-labeled and digoxigenin-labeled subunits used in the in vitro assembly reaction, outlined in FIG. 38. After assembly, any remaining free subunits were removed and analysis, by particle sedimentation on sucrose gradients, confirmed that only assembled protein cages were present with no detectable free subunits remaining. Reassembled particles were subsequently mixed with streptavidin agarose to selectively bind all particles containing at least one biotin ligand, eliminating any particles that only contained the digoxigenin ligand. After binding particles to the streptavidin agarose, the beads were extensively washed to eliminate any non-specific particle binding.

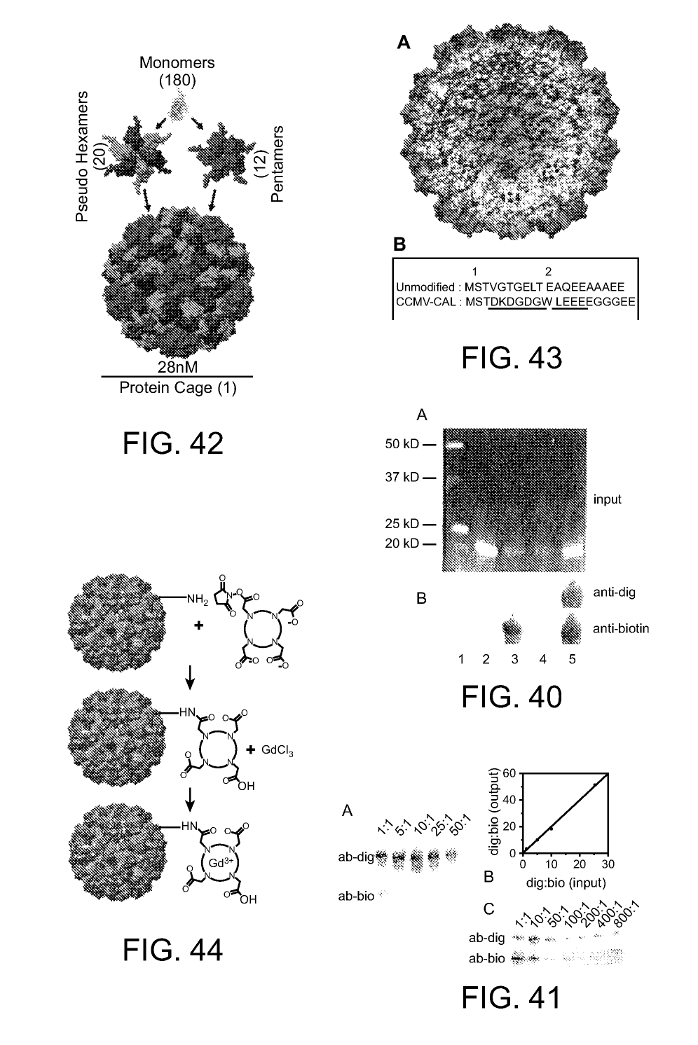

Bound CCMV was subjected to SDS PAGE analysis and the displayed ligands probed by Western blot analysis with either a biotin-specific antibody or a digoxigenin-specific antibody (FIG. 39). Blots were first probed with the antidigoxigenin antibody and subsequently re-probed with the anti-biotin antibody. Biotin labeled subunits were detected when particles were reassembled solely from biotin labeled subunits (FIG. 40, lane 3). As expected, there was no (or only background levels) digoxigenin labeled protein detected in the controls of non-labeled wild type CCMV (FIG. 40, lane 2) or in particles reassembled solely from digoxigenin labeled subunits (FIG. 40, lane 4), since neither of these particles bind to the streptavidin agarose. However, when equal molar amounts of differentially labeled subunits were used in the reassembly reaction, digoxigenin labeled particles were detected (FIG. 40, lane 5). Thus, both the digoxigenin and biotin ligands were present on the same particle. This demonstrates the co-assembly of digoxigenin and biotin labeled subunits into a common icosahedral particle, independent of the displayed ligand.

We have established that the proportion of digoxigenin relative to biotin functionalized subunits in the reassembled particles can be regulated by the subunit stoichiometry (outlined in FIG. 38). The ratio of digoxigenin to biotin labeled subunits in the assembly reaction was varied from 1:1 to 800:1. TEM analysis after assembly confirmed that all reassembly reactions resulted in the formation of 28 nm icosahedral particles. Subsequent streptavidin agarose binding and Western blot analyses of the reassembly experiments indicated a direct relationship between proportion of digoxigenin to biotin in the reassembly mixture (input) and relative band intensity of digoxigenin to biotin in the assembled particles (FIG. 41A). This relationship is linear (FIG. 41B) until the input reaches 25:1 (digoxigenin:biotin). The linear relation between the ratio of monomers functionalized with the two groups contained in the reaction mixture and the relative abundance of the two groups in the reassembled particles (FIG. 41B) is expected. While the theoretical slope of the curve is one, the data curve has a slope of approximately two. We suspect that this discrepancy may originate from a difference in the efficiency of detection by the two different antibodies. At ratios greater than 50:1 the ability to accurately determine the small number of biotin ligands per particle (estimated the be ≦3 biotins per particle) limits quantification of the digoxigenin:biotin ratios. Even at higher ratios of digoxigenin to biotin (up to 800:1) we still observed the digoxigenin ligand even though we were no longer able to detect the presence of the biotin label by Western blotting (FIG. 41C). In these high ratio assemblies we are confident that biotin is present due to the observed particle binding to the streptavidin agarose. This clearly demonstrates that particle composition can be controlled by input ratios of differentially labeled subunits.

We have demonstrated a conceptual approach for imposing a predictable level of functional asymmetry onto an ensemble of icosahedral CCMV particles. These results demonstrate that particles obtained from reassembly from differentially modified subunits are structurally indistinguishable from native CCMV, that they incorporate both ligands, and that the relative abundance of the two ligands can be controlled by the subunit stoichiometry in the assembly reaction. The disassembly/reassembly method described here has the advantage of simplicity and scalability and offers the potential to produce a high yield of particles possessing functional asymmetry. Inherent in this approach is the ability to reassemble functionally asymmetrical particles composed of two or more sets of differentially labeled subunits. In combination with previous work using a solid phase approach for introducing asymmetry into CCMV2, we have expanded the range of approaches by which many high symmetry nanoarchitectures can be asymmetrically functionalized.

### Example 8

### Ru^{∥}bpy₃ as a PDT Agent in an Hsp Protein Cage

A Ru^{∥}bpy₃ analog with a reactive iodoactamide was synthesized in order to covalently attach the reactive dye to the protein. An iodoactamide on the phenanthroline Ru(bpy)₂phen-I specifically reacts with thiols on the Hsp cage and allows direct attachment of the photocatalyst to the protein. An Hsp cage has no endogenous cysteines in its native structure, which makes it convenient for genetically engineering the protein cage. We engineered two mutants of Hsp G41C which has a cysteine on the interior surface and Hsp S121C which has a cysteine on the exterior surface of the protein cage. These two genetic mutants were then used as templates for attachment of the Ru(bpy)₂-phen-I to either the interior or exterior surfaces of the cage. Briefly, the functionalization reaction was done on 1-2 mg/mL Hsp in deoxygenated 50 mM Hepes pH 8.0 at 40° C. for two hours with 5×ruthenium complex per subunit. The reaction was concentrated run over size exclusion chromatography (SEC) to remove unreacted Ru(bpy)₂-phen-I. Size exclusion chromatography analysis indicated that the Hsp G41C and Hsp G41C functionalized with Ru(bpy)₂-phen-I (Hsp G41C-Ru) had the same retention volume suggesting that there was no change in the overall architecture of the protein cage. The increase of the absorption in the Hsp G41CRu at 450 nm compared shows that the protein and the dye are coeluting. SEC of the Hsp S121C and the RuIphen functionalized Hsp S121C (Hsp S121CRu) have the same retention volume showing no perceivable change in the size of the protein cage from attaching the dye molecule to the exterior. SEC of the Hsp S121CRu shows the coelution of intact protein (280 nm) and dye (450 nm). Transmission electron microscopy (TEM) analysis of Hsp G41CRu and Hsp S121CRu stained with 2% uranyl acetate displays ˜12 nm voids where the electron dense stain was not present because of the attachment of the protein cage to the EM grid. SDS PAGE analysis shows the migration of the unlabeled and labeled Hsp G41C and Hsp S121C was conducted with the same gel being analyzed by fluorescence and Coomassie staining. While the labeled and unlabeled protein subunits migrate similar amounts, the fluorescent analysis of the gel clearly shows the fluorescence from the RuIphen associated with the label Hsp G41C and Hsp S121C while no fluorescence is associated with the unlabeled protein. Also displayed by the gel analysis is the degradation of the RuIphen after illumination. The fluorescent analysis of the gel displays minimal fluorescence compared to the labeled protein before illumination. The labeling reaction was also analyzed by liquid chromatography/mass spectrometry (LCMS). The LCMS analysis of the functionalized protein determined that both Hsp G41C and Hsp S121C were labeled with only one RuIphen. While the reaction was not complete, Hsp G41C and Hsp S121C could be labeled to 82 and 85% loading respectively, determined by UV-VIS analysis.

The LCMS of slightly photolyzed, ambient light at 4° C., Hsp G41CRu displayed that the protein undergoes a series oxygen additions to the protein without the degradation of the RuIphen. While the specific sites of oxidation are not known, there are a number of easily oxidized amine acids in each subunit of Hsp, methionine and cysteine. The complete photolysis of Hsp G41CRu and Hsp S121CRu show a complex mix of subunit degradation which is to be expected. The incomplete oxidation of the protein subunit and the photodegradation of the RuIphen leave a protein that is at different levels of degradation giving a number of different masses which is displayed by the envelope of masses detected by the mass spectrometer.

In order to analyze the singlet O₂ production by protein RuIphen composite, the conversion of TEMP to TEMPO by singlet O₂ was monitored by EPR. Briefly, the light induced production of singlet O₂ assay was conducted in a serum vial open to the air with vigorous stirring. The reaction was conducted in 50 mM TEMP with DPBS at pH 7.4. The each of the reactions were normalized to 20 μM reactive dye (RuIphen or Rose Bengal). At each time point, 100 μL of the solution was removed and added to 10 μL of 1M sodium azide to quench the singlet O₂ production. Hsp G41CRu was labeled with two different loadings of RuIphen 51% and 82%. The TEMPO production curves determine that there is minimal difference in the amount of TEMPO produced between the two loadings of Hsp G41C. Which means that the amount of singlet O₂ that reacts with the cage is only a fraction of the total produced. If the light is turned off for ten minutes during the experiment then turned back on, the TEMPO production is similar but not as much is produced showing that the light is needed to continually produce singlet O₂ and that some autocatalytic process is not generating TEMPO. While the TEMPO production from Hsp S121CRu is slightly lower than Hsp G41CRu, the production curves are remarkably similar displaying that the position of the RuIphen on the protein cage (interior or exterior) does not dramatically effect the production of singlet O₂. The control reactions with free Ru^{∥}bpy₃ show similar TEMPO production; however, the reaction kinetics are different. The free Ru^{∥}bpy₃ quickly produces a maximum of TEMPO then the signal from the TEMPO goes away. When singlet O₂ production reaction from free Ru^{∥}bpy₃ is conducted in the presence of Hsp G41C, the amount of protein added is equivalent to the 85% loading, the reaction kinetics are become similar to that of Hsp G41 CRu and Hsp S121CRu showing that the protein is playing some role in the reaction. In order to probe the loss of EPR signal in the free Ru^{∥}bpy₃ control, free Ru^{∥}bpy₃ was illuminated in the presence of purchased TEMPO both in the presence and absence of molecular O₂. Both of the TEMPO reaction conditions show that the free Ru^{∥}bpy₃ degrades the EPR signal, which indicates a O₂-free mechanism. The free Ru^{∥}bpy₃ TEMPO controls explain the loss of signal in the free Ru^{∥}bpy₃ singlet O₂ production reaction by showing that the free Ru^{∥}bpy₃ degrades TEMPO while the addition of the protein provides some level of protection for the TEMPO through an unknown mechanism.

Rose Bengal was used as a comparison to gauge the production singlet O₂. Free Rose Bengal showed a 4-fold increase in the amount of TEMPO generated; however, the EPR signal was quickly quenched comparable to the free Ru^{∥}bpy₃. The singlet O₂ production reaction from Rose Bengal in the presence of Hsp G41C, the amount of protein added is equivalent to the 85% loading, the TEMPO production is suppressed but the reaction kinetics are almost identical. Analysis of the reaction between purchased TEMPO and Rose Bengal upon illumination in the presence and absence of O₂ show that Rose Bengal does not degrade TEMPO in the presence of O₂, however, quickly degrades it in the absence of O₂. Leading to the hypothesis that Rose Bengal preferentially reacts with O₂ in a non-TEMPO degrading reaction while in the absence of O₂ Rose Bengal degrades TEMPO in a O₂-free reaction. In an attempt to test our hypothesis, we tested the TEMPO production under varying O₂ concentrations, atmospheric, bubbling air through the solution, and under an O₂ atmosphere. The amount of TEMPO produced and the length of time that the TEMPO generated lasted increased with increasing O₂ concentration which supporting our hypothesis that Rose Bengal energy quenching by O₂ is faster than the TEMPO degrading.

Materials. All materials were obtained from Sigma-Aldrich and used as received with no further purification. All water used was purified through a Nanopure system to 18.2 MΩ resistivity.

Synthesis of 5-Iodoacetoamino-1,10-phenathroline (Iphen). Iphen was synthesized by modification of previously reported procedure¹. A solution of 1,3-dicyclohexylcarbodiimide (5.29 g, 26 mmol) and iodoacetic acid (4.76 g, 26 mmol) in 50 mL dry ethyl acetate was stirred for 3 hours at room temperature. The resulting solution was filtered to remove the urea. The solution was dried by rotary evaporation and redissolved in 25 mL acetonitrile. The solution was added to 25 ml of acetonitrile containing 5-amino-1,10-phenanthroline (1.0 g, 0.005 mol) and stirred overnight at room temperature. The product was collected be centrifugation and washed with cold 5% sodium bicarbonate and water. The product was dried under vacuum and confirmed by mass spectroscopy. (Yield: 1.32 g).

Synthesis of Ru(bpy₂)Cl₂. Ru(bpy₂)Cl₂ was synthesized according to literature procedures². RuC1₃.3H₂0 (7.8 g, 29.8 mmol), bipyridine (9.36 g, 60.0 mmol), and LiCl (8.4 g, 2.0 mmol) were refluxed dimethylformamide (50 mL) for 8 h. The reaction was cooled to room temperature, 250 mL of acetone was added and the solution was stored at 4° C. overnight. The resultant product was filtered and washed with water and ether and dried by suction.

Synthesis of Ru^{∥}(bpy₂)Iphen. Ru^{∥}(bpy₂)Iphen was synthesized by modification of previously reported procedure³. Ru(bpy)₂Cl₂ (0.7 g, 1.45 mmol) and Iphen (0.5 g, 1.38 mmol) were refluxed in 50 mL MeOH for 3 h with stirring. The solution was filtered. The product was precipitated by the addition of a concentrated aqueous solution of NH₄ PF₆ to a warm solution. The orange solid was collected by filtration and washed with cold water and ether and dried in a desiccator. (Yield: 1.18 g).

Protein Functionalization. The protein solution to be labeled (small heat shock protein mutants Hsp G41C or Hsp S121C) were dialyzed into deoxygenated buffer (50 mM HEPES 100 mM NaCl pH 8.0) overnight. The protein was transferred to a jacketed reaction vessel at 40° C. under nitrogen. The sample was diluted to ˜1 mg/mL with deoxygenated buffer and a 5-fold excess of Ru^{∥}(bpy₂)Iphen dissolved in minimal DMF was slowly added. The solution was protected from light and allowed to react for 3 hours. After the reaction was completed the protein was concentrated and passed over size exclusion chromatography (Dulbecco's phosphate buffered solution (DPBS) pH 7.4) to separate unreacted dye and to the exchange buffer.

Singlet Oxygen Production Assay. In a 3 mL clear glass serum vial (Wheaton), 20 □M ruthenium (either free or attached to protein cage) and 50 mM 2,2,6,6-tetramethyl-4-piperidone (TEMP) final concentration was added to DPBS pH 7.4. The reaction was illuminated by a Xe arc lamp (175 W, Lambda-LS, Sutter Instruments) with an water filter to remove the IR radiation and an UV-absorbing glass (<360 nm) to remove the UV radiation. The reactions are maintained at 37° C. and vigorously stirred. At each time point, a 100 □L sample is removed and added to 10 □L of 1M sodium azide to quench the reaction. For the oxygen-free reaction, the serum vials are sealed and degassed under nitrogen The light was quantitated using an Extech Instrument EasyView light meter to be 511, 700 I×.

Electron Paramagnetic Reasonance (EPR). EPR data was collected on a Bruker X-band EMX spectrometer. The instrumental conditions were as follows: microwave frequency, 9.84 GHz; modulation frequency, 100 kHz; modulation amplitude, 1 G; time constant, 81.92 msec; sweep time, 81.92 msec; sweep width, 80 G; and center field, 3510 G. 100 uL of sample was loaded into a flat-cell microslide (0.3×6.0 mm I.D., VitroCom Inc.) for the analysis. The results were compared to standard curve using purchased 4-oxo-TEMPO (TEMPO) using signal amplitude difference in the highest field peak.

Transmission Electron Microscopy (TEM). TEM data were obtained on a Leo 912 AB, with Ω filter, operating at 100 keV. The samples were concentrated using microcon ultrafilters (Microcon YM-100) with 100 kDa nominal molecular weight cutoff and transferred to carbon coated copper grids. Samples were imaged negatively stained with 2% uranyl acetate.

Dynamic Light Scattering (DLS). DLS measurements were carried out on a Brookhaven Instrument Corporation 90-PALS at 90 degrees using a 661 nm diode laser, and the correlation functions were fit using a non-negatively constrained least-squares analysis.⁴

UV-Vis Spectroscopy. UV-V is spectroscopy measurements were carried out on a Agilent 8453 UV-V is spectrometer.

Size Exclusion Chromatography (SEC). SEC was performed on a Biologic Duo-Flow fast protein liquid chromatography system equipped with a Quad-Tec UV-V is detector and using a Superose 6 size exclusion chromatography column.

Expression, Purification. One liter cultures of E. coli (BL21(DE3) B strain) containing pET-30a(+) Hsp16.5 G41C or S121C plasmid were grown overnight in M9 salts+10 g NaCl+10 g Bactotrypotone+kanamycin medium (37° C., 220 rpm). Cells were harvested by centrifugation 3700×g for 20 minutes (Heraeus #3334 rotor, Sorvall Centrifuge) and re-suspended in 80 mLs of 50 mM MES, pH 6.5. Lysozyme, RNAse A, and DNAse I were added to final concentrations of 0.041 mg/mL, 0.055 mg/mL and 0.08 mg/L respectively and incubated for 30 minutes on ice. The sample was French pressed (American Instrument Co., Inc) and sonicated (Branson Sonifier 250, Power 4, Duty cycle 50%, 3×5 minutes with 5 minute rest intervals). Bacterial cell debris was removed via centrifugation for 45 minutes at 12,000×g. The supernatant was heated for 10 minutes at 60° C. and centrifuged for 20 minutes at 12,000×g thereby removing many heat labile E. coli proteins. The remaining cell extract was purified by gel filtration chromatography (Superose-6, Amersham-Pharmacia; BioRad Duoflow). The subunit molecular weight was verified by SDS poly-acrylamide gel electrophoresis (SDS-PAGE) and mass spectrometry (Waters MicroMass Q-TOF). The assembled protein was imaged by transmission electron microscopy (TEM) (LEO 912 AB) (stained with 2% uranyl acetate on formvar carbon coated grids), and analyzed by dynamic light scattering (DLS) (90 plus Brookhaven Instruments). Protein concentration was determined by absorbance at 280 nm divided by the published extinction coefficient (9322 M⁻¹ cm⁻¹).

### Example 9

### CCMV Protein Cages Having Gd³⁺ as MRI Contrast Agents

The purpose of this study was to take advantage of the CCMV architecture while enhancing the Gd³⁺ binding constant of the lanthanide metal ion in the development of two nanoparticle contrast agents. The first approach was to genetically incorporate a nine residue peptide sequence, from the Ca²⁺ binding protein calmodulin, as a genetic fusion to the N-terminus of the CCMV subunit (CCMV-CAL). Characterization of the metal binding to the genetically engineered cage was undertaken using FRET analysis. The second approach was to covalently attach the clinically relevant contrast agent GdDOTA to reactive lysine residues on CCMV via an NHS ester coupling reaction (CCMV-DOTA). Both R1 and R2 relaxivity data were measured for the genetic and chemically modified viral capsid contrast agents.

Engineering CCMV to express the metal binding sequence of calmodulin. The SubE/R26C/K42R gene, a mutant of the coat protein of CCMV, cloned into the Pichia pastoris vector; pPICZA (Invitrogen) was used as the template (Brumfield S, et al. (2004) J Gen Virol 85:1049-1053) QuikChange site-directed mutagenesis (Stratagene) using the primer; (5′cgaggaattcatgtctacagacaaa gatggtgatggatggttagaattcgaagagggtgggggcgaagagaacgaggagaacac3′), and it's reverse complement was used to insert the Calmodulin DNA sequence into the N terminus of the coding region of the capsid protein. The mutagenized vector from CCMV was confirmed by DNA sequencing (Applied Biosystems). This modified CCMV protein was called CCMV-CAL.

Expression and purification of CCMV-CAL. The mutagenized CCMV capsid protein gene was expressed and purified in a Pichia pastoris heterologous protein expression system as previously described (Brumfield (2004) J Gen Virol supra). High levels of coat protein expression were induced and yielded assembled viral protein capsids devoid of nucleic acid. These protein cages were purified to near homogeneity by lysis of cells, followed by ion exchange chromatography. Size exclusion chromatography (SEC) was used to further purify the protein cage and to eliminate any aggregates or subunit disassembly products potentially present in the samples (Superose 6, Amersham Biosciences; 50 mM HEPES, pH 6.5). Both ion exchange and SEC were performed on an Amersham Akta purifier FPLC. The twelve residue binding sequence and three glycine residues, shown in (FIG. 43), replaced the original residues 4-18 of the SubE/R26C/K42R CCMV mutant. This replacement was confirmed at the protein level by Liquid Chromatography/Mass Spectrometry (LC/MS) of the purified protein (Agilent Technologies 1100 LC system coupled to an Esquire 3000 ion trap mass spectrometer, Bruker Daltonics). The theoretical mass was calculated by considering loss of the N-terminal methionine and acetylation of the second residue (serine). Protein concentration was determined by the absorbance at 280 nm (ε=29280 M⁻¹ cm⁻¹ for CCMV-CAL, ε's were calculated by inserting amino acid sequences into the ProtParam tool at: (http://ca.expasy.org/tools/protparam.html).

Synthesis of CCMV-DOTA protein cages The following buffers were used in the synthesis of CCMV-DOTA; Labeling Buffer (100 mM HEPES, 100 mM NaCl, pH 7.2), and Storage Buffer (100 mM HEPES, 100 mM NaCl, pH 6.5). A lysine reactive form (NHS-ester) of the metal chelator DOTA was used in the synthesis (Macrocyclics, B-280). (FIG. 44) outlines the general reaction scheme. The K42R mutant of the CCMV (0.5-3 mg/mL, 25-150 μM subunit) virus particle was purified from infected plants (as previously described) and dialyzed into 500 mL of Labeling Buffer for 3 hours (Bancroft J B, et al. (1968) Virology 34(2):224). The concentration of plant virus was calculated by multiplying the A₂₆₀ by 6.4 to yield a concentration of CCMV subunit in μM units. A concentration of 1 to 2 mg/mL (˜50 to 100 μM subunit) was typically used in the reaction. A 20× (mole/mole) of NHS-ester DOTA was next added. The pH was maintained at 7.0 by additions (1-5 μL) of 0.5M NaOH. The reaction mixture was monitor by LC/MS (both Standard and Nano Aquity LC systems and both Q-Tof Micro and Q-Tof Premier mass spectrometers were used). SEC and reverse phase separation techniques were used. The deconvolution program MaxEnt1 (Waters) was used to determine the percent of subunits with DOTA covalently linked to them. Equation [1.1] was used to approximate the average labeling of subunits with DOTA, where D is the number of DOTA(s) attached to the subunit and I_{D} is the intensity of the ion corresponding to a subunit with D DOTA(s) attached.

$$\frac{AverageDOTA}{Subunit} = \frac{{\sum }_{0}^{D} {DI}_{D}}{{\sum }_{0}^{D} {I}_{D}} \qquad [ 1.1 ]$$

The reaction was allowed to proceed for 2 hours at 25° C. At that point, LC/MS analysis revealed that the majority of NHS-DOTA reactant was hydrolyzed. The reaction was repeated until there was, on average, one DOTA covalently attached per subunit. Unreacted DOTA was removed by dialyzing the reaction mixture into Labeling Buffer. Next the CCMV-DOTA was dialyzed into Labeling Buffer with 10× GdCl₃ (moles Gd³⁺:moles subunit) and a pH of 7.0. The progression of the metal loading onto the CCMV-DOTA was monitored by LC/MS. Free Gd³⁺ was separated from the CCMV-DOTA-Gd by dialyzing the labeled cage into Storage Buffer that contained 5 mM EDTA. Then multiple dialysis steps were performed into Storage Buffer. Alternatively the CCMV-DOTA-Gd was separated from free Gd³⁺ by running the reaction product over SEC using Storage Buffer as the eluent.

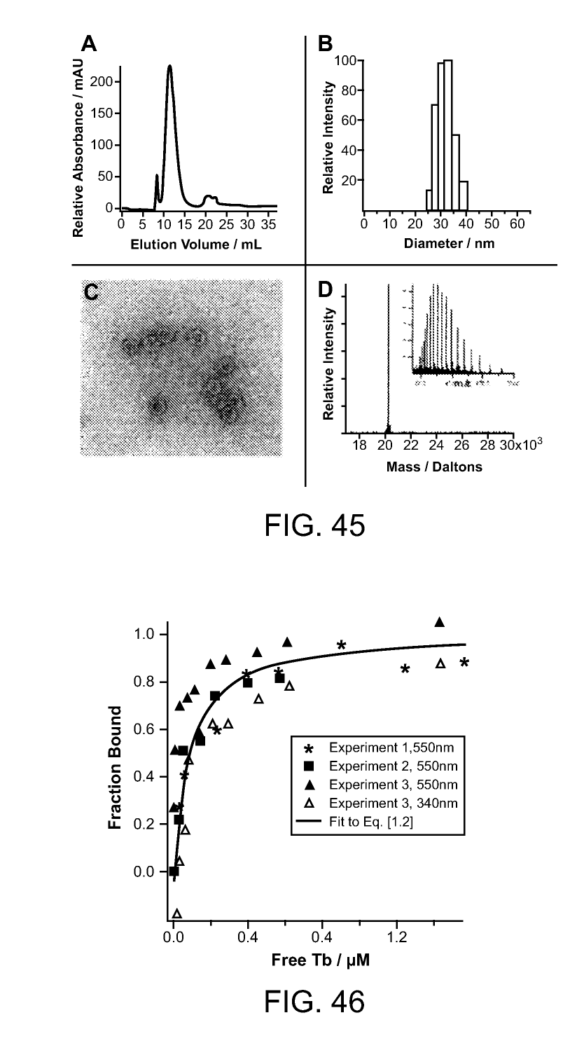

Characterization of the modified CCMV capsids. Characterization of both purified cages was performed by SEC, dynamic light scattering (DLS), transmission electron microscopy (TEM) and LC/MS. Typical data from these characterization techniques is shown in (FIG. 45) DLS analysis was performed on a ZetaPlus (Brookhaven Instruments). Transmission electron microscopy (TEM) of negatively stained samples (1% uranyl acetate) was performed on a (Leo 912AB).

Characterization GdDOTA reaction site in CCMV-DOTA-Gd. After removal of unbound Gd³⁺ from a solution of CCMV-DOTA-Gd, inductively coupled plasma mass spectrometry (ICP-MS) analysis was used to determine the total concentration of Gd³⁺ bound to the protein cage (7500, Agilent Technologies). Protein concentrations of the CCMV-DOTA-Gd samples were determined by the BCA assay (Pierce). Protease digestion (Trypsin Gold, Promega) of CCMV-DOTA-Gd was carried out (1 mg/mL protein subunit, 1:200 subunit:trypsin, 37° C., 12 hours). LC/MS/MS was performed on the digested sample (nanoAquity coupled to a Q-Tof Premier). Data was analyzed with PLGS2 and MassLynx (Waters).

Generation of Binding Isotherms for CCMV-CAL. The lanthanides ions Gd³⁺ and terbium (Tb³⁺) are known to bind with similar affinities however Gd³⁺ does not undergo fluorescence resonance energy transfer (FRET) and therefore cannot be used to probe metal binding. Therefore Tb³⁺ was used as a Gd³⁺ mimic to study metal binding. All fluorescence experiments were performed on a (SPEX Fluorolog) spectrophotometer at 25° C. The fluorescence spectrum of CCMV-CAL-Tb was measured (λ max=340 nm) following excitation at 295 nm, with a Tb³⁺ emission max was near 550 nm. The 340 nm peak was recorded by scanning from 305 nm to 575 nm in 1 nm steps. The 550 peak was monitored by scanning from 525 nm to 575 nm in 0.2 nm steps. Excitation and emission slit widths were set to 4 and 8 nm, respectively. Fluorescence was measured on 500 μL solutions of protein cage to which metal ion was added in 5-20 μL increments from 10-500 μM solution standards. Subunit protein concentrations of 0.05 μM and 0.1 μM for the CCMV-CAL mutant were used.

Calculation of K_{d} In analyzing the data for Tb³⁺ binding to the CCMV-CAL capsid, we have assumed that when metal ions are titrated to the CCMV-CAL capsid, they first bind completely to the inserted sites. Once the inserted sites are completely occupied then the endogenous sites starts to bind the additional metal ions that are added. Eq. [1.2] was used to analyze the data where 0 is the fraction of protein cage subunits with bound Tb³⁺, [Tb³⁺_{Free}] is the free Tb³⁺ concentration, and K_{d} is the dissociation constant for subunit binding Tb³⁺ ions.

$$θ = \frac{[ T {b}_{Free}^{3 +} ]}{[ T {b}_{Free}^{3 +} ] + {K}_{d}} \qquad [ 1.2 ]$$

Baseline correction was performed on the 550 nm Tb³⁺ spectra. To find the maximum intensity of the 550 nm peak a Gaussian function was individually fit to each spectra. The maximum intensity of the 550 nm peak (1550) vs. the total Tb³⁺ in solution ([Tb³⁺ₜₒₜₐₗ]) was plotted and fit to Eq. [1.3] where K_{d}_{_{—}}ᵢₙᵢₜᵢₐₗ and I_{550Limit} were the fitting parameters.

$${I}_{550} = {I}_{550 Limit} ( \frac{[ T {b}_{Total}^{3 +} ]}{[ T {b}_{Total}^{3 +} ] + {K}_{d\_Initial}} ) \qquad [ 1.3 ]$$

Fractions bound terms (θ) were calculated by dividing I₅₅₀ values by 1_{550Limit} determined from Eq. [1.3]. Values for [Tb³⁺_{Free}] were then calculated by Eq. [1.4].

$$⌊ T {b}_{Free}^{3 +} ⌋ = ⌊ T {b}_{Total}^{3 +} ⌋ - ( θ [ {Subunit}_{Total} ] ) \qquad [ 1.4 ]$$

A plot of θ vs. [Tb³⁺_{free}] was then fit with the Eq. [1.2] and K_{d} was determined along with an error associated with the fit.

Stiochiometric Titration of CCMV-CAL Again Tb³⁺ ions were used as Gd³⁺ ion mimics. Terbium titrations were performed with the condition of [Subunit]>>K_{d} to determine the number of Tb³⁺ bound per CCMV-CAL subunit. Two titrations were performed in which the protein concentration used was 2.6 μM and 10 μM. The cage was titrated to −20 μM total Tb³⁺ in both experiments. The data, from both the beginning and end portions of the titration, were fit to linear functions.

Relaxometry and Gd³⁺ Quantitation. For relaxometry experiments, fully assembled CCMV-CAL (60 μM subunit) containing 200 μM GdCl₃ was prepared in pH 6.5 buffer (50 mM Hepes, 150 mM NaCl). As a control 200 μM GdCl₃ was prepared in the same buffer. CCMV-DOTA-Gd was prepared in pH 6.5 buffer (100 mM Hepes, 100 mM NaCl) with a subunit concentration of 101 μM (determined by the BCA assay) and 34 μM Gd³⁺ (determined by ICP-MS). Using a custom-designed variable field relaxometer, T1 relaxivity was measured using a saturation recovery pulse sequence with 32 incremental T values. The range of Larmor frequencies was 2-62 MHz (0.05-1.5 T) and the measurements were carried out at a temperature of 23° C. T1 values were determined by fitting data into Eq. [1.5] with A and B as fitting parameters.

$$f ( Seconds ) = A ( 1 - {ⅇ}^{( \frac{- Seconds}{\frac{1}{T 1}} )} ) + B \qquad [ 1.5 ]$$

T2 was measured using a CPMG pulse sequence with 500 echoes and an interecho time of 2 ms. T2 values were determined by fitting data into Eq. [1.6] with A, B and N as fitting parameters.

$$f ( Seconds ) = \sqrt{A ( {ⅇ}^{( \frac{- 2 Seconds}{\frac{1}{T 2}} ) + N} ) + B} \qquad [ 1.6 ]$$

Since the R1 and R2 relaxivities are expressed in units of (mM⁻¹ of bound Gd^{3+*}seconds⁻), it was necessary to determine the mM concentration of bound Gd³⁺. The calculation of the fraction of CCMV-CAL with bound Gd³⁺ turned into an approximation since this capsid contains two types of binding sites. First the parameters for the higher affinity binding site or “CAL” were input into Eq. [1.7]. The concentration of binding sites [BS], [Gd³⁺_{Total}] and the K_{d} are all values input into this equation.

$$θ = \frac{\begin{matrix}[ BS ] + [ T {b}_{Total}^{3 +} ] + {K}_{d} - \\ \sqrt{{( [ BS ] + [ T {b}_{Total}^{3 +} ] + {K}_{d} )}^{2} - ( 4 [ BS ] [ {Tb}_{Total}^{3 +} ] )}\end{matrix}}{2 [ BS ]} \qquad [ 1.7 ]$$

This resulted in a fraction bound term for the CAL binding site (θ_{CAL}). From this result Eq. [1.8] was used to calculate the [Gd³⁺_{Free}] left after CAL was bound.
Gd_{Free}³⁺=Gd_{Total}³⁺−([CAL]·θ_{CAL}) [1.8]

The fraction of Gd³⁺ bound to the endogenous site (θEndogenous) was calculated by setting [Gd³⁺_{Total}], which was input into Eq. [1.7], equal to [Gd³⁺_{Free}], which was determined by Eq. [1.8]. K_{d} and [BS] values for the endogenous site were input into Eq. [1.7] to determine the fraction bound for the endogenous site (θ_{Endogenous}). Finally the faction bound terms for both the endogenous site (θ_{Endogenous}) and the engineered site (θ_{CAL}) were multiplied by the mM concentration of their respective binding sites and then added together resulting in an mM concentration of total bound Gd³⁺.

Results. CCMV modified architecture. The CCMV protein cage architecture (FIG. 42) has been modified for enhanced Gd³⁺ binding, using two complementary approaches, while maintaining the advantages of the large molecular platform. In the first approach, a Gd³⁺ binding peptide from calmodulin, was genetically introduced onto the N-terminus of the CCMV viral capsid subunit (CCMV-CAL-Gd). This modified viral capsid has an increased affinity for Gd³⁺ in comparison with wild type CCMV. In a second approach, GdDOTA was conjugated to CCMV resulting in high affinity Gd³⁺ binding and imparting highly efficient relaxivity properties to the CCMV capsid (CCMV-DOTA-Gd).

Genetic modification of CCMV—Attachment of calmodulin peptide. A peptide sequence from the Ca²⁺ binding portion of the protein calmodulin (DKDGDGWLEFEEGGG) was genetically fused to the N-terminus of CCMV (FIG. 43). Interestingly, this construct with a nine residue peptide incorporated as an N-terminal fusion did not disrupt the ability of CCMV to self assemble as shown in the SEC, DLS and TEM analyses (FIG. 45). The mutation of the coat protein subunit gene was confirmed by DNA sequencing. Also, LC/MS of the purified protein cage subunit produced an experimental average mass of 20234 Da compared to a calculated average mass of 20232 Da (supplemental data) for the mutant protein subunit confirming the identity of the recombinant protein.

K_{d} determination of CCMV-CAL The metal binding affinity of the CCMV-CAL mutant was probed by FRET using excitation of endogenous tryptophan residues. The lanthanides ions Gd³⁺ and terbium (Tb³⁺) are known to bind with similar affinities and both show preference for Ca²⁺ binding sites in proteins although with significantly higher affinities than Ca²⁺ binding (Allen M, et al. (2005) Magnet Reson Med 54(4):807-812; Vazquez-Ibar J L et al. (2002) Proc Natl Acad Sci USA 99(6):3487-3492). Titration of the CCMV-CAL mutant with increasing Tb³⁺ revealed a decrease in the tryptophan fluorescence (340 nm) and concomitant increase in the Tb³⁺ fluorescence (at 550 nm) indicating energy transfer between these sites (FIG. 46). The complete data set was fit to Eq. [1.2] and an average K_{d} of 82±14 nM for Tb³⁺ binding to CCMV-CAL was determined. This indicates an enhancement in the metal binding affinity of 378 fold over binding to endogenous sites in the wild type (Basu G et al. (2003) J Biol Inorg Chem 2003; 8(7):721-725).

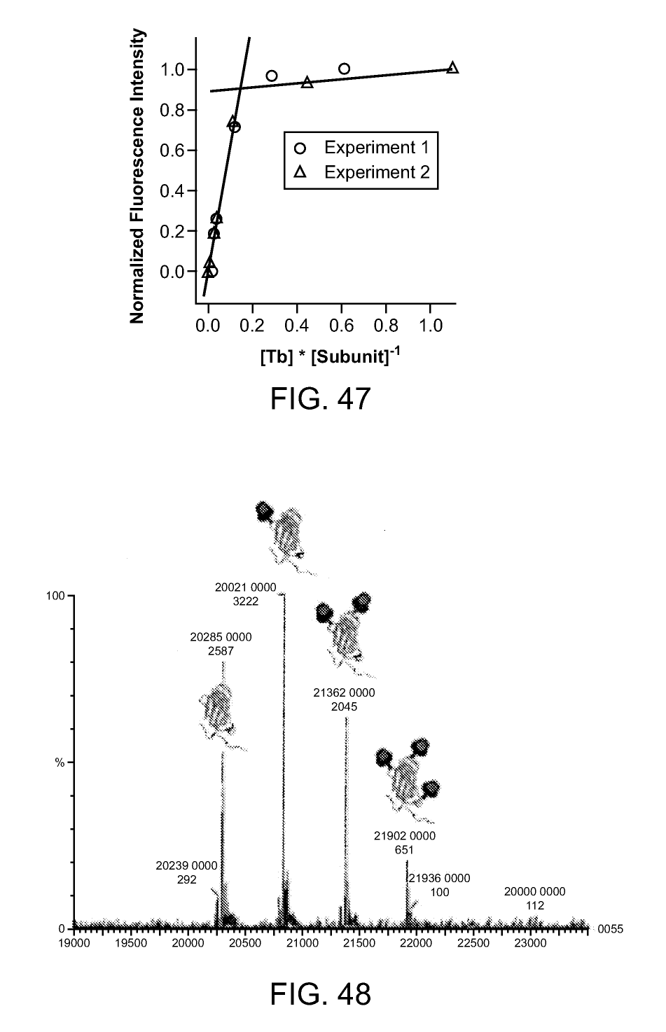

Stoichiometric titrations of CCMV-CAL A stiochiometric titration of Tb³⁺ was performed to determine the number of ions bound per CCMV-CAL subunit. When normalized fluoroscence intensity was plotted against the ratio of Subunit:Tb³⁺ ([Subunit]/[Tb³⁺_{Total}]) as shown in (FIG. 47), the data shows two distinct regions. The fluorescence response during the first part of the titration ([Binding Site]>>>[metal]) increases linearly with Tb³⁺ addition. The second linear portion of the titration ([metal>>>[Binding Site]) shows a relatively constant fluorescent response with a smaller slope. The x-intersection of the fits to each of these two regions indicates the point at which the binding sites are maximally occupied. Our data indicate a value of 7.3±1.1 ([Subunit]/[Tb³⁺_{Total}]) for this intersection. At the end of the Tb³⁺ titration, there is one Tb³⁺ ion for every 7.3±1.1 subunits or each CCMV cage has approximately 25 metal ions bound at the introduced peptide sites.

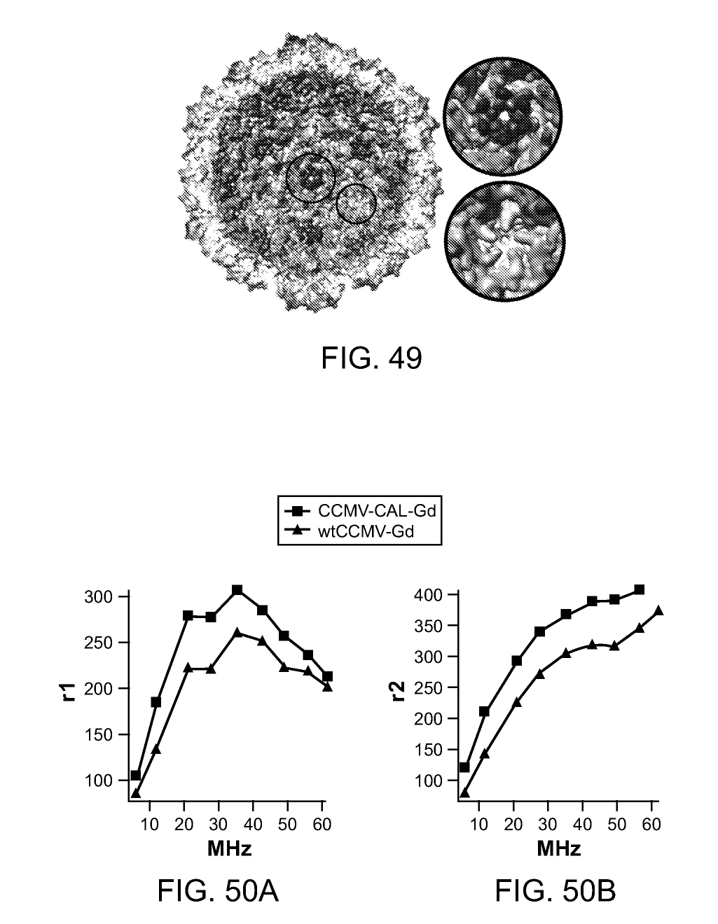

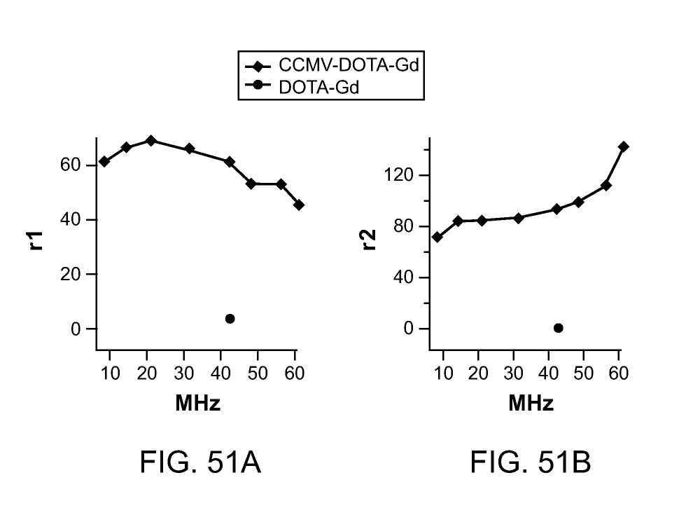

Chemical modification of CCMV—Attachment of GdDOTA. GdDOTA was covalently attached to the surface of CCMV via reaction with surface exposed lysine residues (FIG. 44). FIG. 48 shows a deconvoluted mass spectrum after a routine CCMV-DOTA-Gd synthesis and removal of unbound Gd³⁺ showing a distribution of 0 to 3 GdDOTA per subunit. The addition of a single GdDOTA added an experimental mass of 544 daltons to the subunit molecular weight, which corresponds well with the theoretical value of 543.7 daltons per GdDOTA. It was qualitatively observed by LC/MS that up to two DOTAs on average per subunit produced a stable, fully assembled protein cage yielding up to 360 GdDOTAs per cage. Using mass spectrometry we were able to map the lysines which are the sites of labeling. LC/MS/MS analysis indicates that GdDOTA is primarily attached through Lys 8 and Lys 45 (shown in supplemental data). The distribution of GdDOTA labeling on CCMV-DOTA-Gd suggests that there is not complete occupancy of either Lys 45 or Lys 8 within the cage and that additional unidentified residues are also labeled. However, residues 8 and 45 are likely the most prevalently labeled lysines in CCMV. FIG. 49 shows the inside view of the CCMV capsid with GdDOTA modeled onto residue 45. The position of lysine 45 in the structure suggests that the attached GdDOTA resides on the interior of the cage. Residue 8 is not shown since the N-terminus is disordered in the X-ray crystal structure of CCMV and its position is therefore uncertain.

Relaxometry of CCMV-CAL-Gd and CCMV-DOTA-Gd nanoparticles. Highly efficient T1 and T2 relaxivity properties were observed in both nanoscale assemblies (CCMV-CAL-Gd and CCMV-DOTA-Gd). Table 1 summarizes R1 and R2 values for Gd³⁺/virus systems including; wild type CCMV-Gd, CCMV-CAL-Gd, CCMV-DOTA-Gd and MS2-DTPA-Gd (Anderson E A et al. (2006) Nano Letters 6(6):1160-1164; Allen (2005) Magnet Reson Med supra). The ionic R1 and R2 trends are CCMV-CAL-Gd>CCMV-Gd>CCMV-DOTA-Gd>MS2-DTPA-Gd. The R1 and R2 trends relative to the particle are CCMV-CAL-Gd>wild type CCMV-Gd>MS2-DTPA-Gd>CCMV-DOTA-Gd. In the genetic approach, the CCMV-CAL mutant exhibited approximately the same T1 and T2 relaxivity as wild type CCMV-Gd previously reported Allen (2005) Magnet Reson Med supra). FIG. 50 shows the similarities between the T1 and T2 relaxivity values of wild type CCMV-Gd and CCMV-CAL-Gd for field strengths ranging from 0.2 to 1.5 T. This construct not only has an increased affinity for Gd³⁺, but also maintains the very high relaxivity required for clinically relevant contrast agents.

The increased T1 and T2 relaxivity afforded by the chemical conjugation of GdDOTA to CCMV's capsid is shown in (FIG. 10). This conjugation resulted in increased T1 and T2 relaxivities by a factor of ten relative to free GdDOTA. By chemically attaching GdDOTA to the CCMV capsid we have engineered a protein cage with clinically relevant binding and high relaxivity.

The R1 dependence on field strength, for both wild type CCMV-Gd and CCMV-CAL-Gd, varies in manner typical of nanoscale systems with a relaxivity maximum near 1 T (Laus S et al. (2005) Chemistry—a European Journal 11(10):3064-3076). CCMV-DOTA-Gd has R1 field strength dependence more similar to small molecule systems with a maximum near 0.5 T. All CCMV/Gd systems exhibit a positive correlation between field strength and T2 relaxivity values as expected.

Discussion. The major findings of this study are: 1) the development of a genetically engineered protein cage platform, specifically a viral capsid, that binds Gd³⁺ (CCMV-CAL-Gd) and 2) a CCMV capsid to which multiple GdDOTA are chemically attached (CCMV-DOTA-Gd). Both these nanoparticle contrast agents are water soluble and have high ionic and particle relaxivites. These viral capsids have the ability to have multiple Gd³⁺ ions attached to them resulting in high contrast per tissue specific localization events via active targeting or other passive means.

It is surprising that we observed sub-stiochometric binding of Gd³⁺ to the introduced CAL peptide in the CCMV-CAL-Gd construct. The data suggest that only 25 out of 180 sites bind Gd³⁺ at saturating conditions. A likely possibility is that not all the introduced sites are accessible to bind metal ions due to different chemical environments of the CAL peptides. It is also worth mentioning the following: (FIG. 2A) shows that the N-terminus is grouped in two environments, twenty pseudo 6-fold and twelve 5-fold environments. The combinations of these groupings yields an average of 5.6 subunits per N terminus grouping. This value is reasonably close to the experimentally determined value of 7.3±1.1 CAL peptides per bound metal ion. It is possible that single metal ions are bound by multiple CAL peptides grouped at these 5 and pseudo 6-fold environments. Further studies would have to be carried out to explain this curious result. The dissociation constant of 82±14 nM for CCMV-CAL-Gd³⁺ in comparison to dissociation constants in the range of 10-20 molar for approved contrast agents indicates that the CAL peptide binds Gd³⁺ too weakly for clinical application. Therefore, this approach of genetically attaching metal binding peptides to the CCMV capsid is not likely to be as fruitful as chemical modification approaches.

It was expected that all three CCMV/Gd constructs (CCMV-Gd, CCMV-CAL-Gd, CCMV-DOTA-Gd) would have similar relaxivity values since size was thought to be the dominate factor in determining relaxivity rates. However, CCMV-DOTA-Gd has ionic relaxivity values that are approximately 25% of the values for CCMV-Gd and CCMV-CAL-Gd, indicating that factors other than size can influence the relaxivity of these cages. The endogenous Gd³⁺ binding pocket in wild type CCMV is at the interface of three subunits and contains side chains from each subunit. The result of this inter-subunit binding pocket is that the overall motion of the Gd³⁺ ion is identical to the overall motion of the entire capsid and there is no additional motion of the Gd³⁺ ion. This is in contrast to the DOTA bound Gd³⁺ of CCMV-DOTA-Gd in which other factors add additional motion to the Gd³⁺ ion. LC/MS/MS data of a CCMV-DOTA-Gd trypsin digest indicates that the GdDOTA is primarily attached on the N-terminus end of the capsid subunit through Lys 8 and Lys 45. It is known that the N terminus of CCMV is mobile and can occupy both the interior and exterior of the CCMV cage architecture (Liepold L O et al. (2005) Phys Biol 2(4):S166-S172; Speir J A, et al. J Virol 80(7):3582-3591). This increased local mobility of the region of the protein cage that is labeled with GdDOTA may cause a reduction in relaxivity. Additional reduction in relaxivity may come from flexibility in the chemical linker of DOTA and lysine's side chain. EPR studies of CCMV labeled with another small molecule concluded that there was local mobility within the spin label itself which decreased the rotational correlation time when compared to the predicted value for the CCMV capsid (Vriend G, et al. (1985) J Magn Reson 64(3):501-505; Vriend G et al. (1981) Febs Lett 134(2):167-171; Vriend G et al. (1982) Febs Lett 146(2):319-321; Vriend G et al. (1984) J Magn Reson 58(3):421-427; Vriend G et al. J Mol Biol 191(3):453-460; Vriend G et al. (1982) Febs Left 145(1):49-52; Hemming a M A et al. (1986) J Magn Reson 66(1):1-8).

This local mobility of the label may also exist in CCMV-DOTA-Gd that would lead to an additional decrease in relaxivity rates. Additional evidence that there is more local mobility in the CCMV-DOTA-Gd is that the R1 field dependence has a maximum at lower field strengths than the other two systems (CCMV-Gd and CCMV-CAL-Gd). Nanoscale systems tend to have R1 maximums at higher field strengths (Laus (2005) Chemistry—a European Journal supra). Local mobility from flexible regions of the protein as well as the GdDOTA itself could explain the lower relaxivity values of CCMV-DOTA-Gd compared to CCMV-Gd and CCMV-CAL-Gd.

The consideration of gadolinium's ligands offers another explanation for the relaxivity differences between CCMV-DOTA-Gd and the two other CCMV/Gd³⁺ systems (CCMV-Gd and CCMV-CAL-Gd). It was found that a peptide similar to the genetically fused sequence used in this work, resulted in unexpectedly high relaxivity values. These authors suggested that the high relaxivity values were a result of having the Gd³⁺ coordinated primarily with oxygen atoms (Caravan P, et al. (2003) Chemical Communications (20):2574-2575; Lauffer R B et al. (1987) Chemical Reviews 87(5):901-927; Raymond K N et al. Bioconjugate Chem 16(1):3-8). Similarly in CCMV-Gd and CCMV-CAL-Gd oxygen atoms are the ligands whereas in the DOTA system oxygen and nitrogen are both ligands of Gd³⁺. These nitrogen ligands could also account for some reduction of the relaxivity rates of CCMV-DOTA-Gd compared to CCMV-CAL-Gd.

Another possible example of how local mobility can affect relaxivity rates is revealed by comparing two different, Gd³⁺ chelated, protein cage systems (CCMV-DOTA-Gd and MS2-DTPA-Gd). Anderson and co-workers have attached up to 500Gd³⁺ ions to the MS2 capsid yielding T1 ionic relaxivity rates that are approximately three times lower than the T1 ionic relaxivity rates for CCMV-DOTA-Gd (Table 1). This deviation is larger than expected since the relaxivity difference between DOTA and DTPA is small, the two cages are the same size and in both systems the chelators are attached to endogenous lysines. However, the chemical linkers used in the two systems differ. The NHS-ester used in the DOTA system results in a short linker with three rotatable bonds. This is in contrast to the DTPA system where the linker is longer and has four rotatable bonds. (The linker was measured from the lysine's amine nitrogen to the nitrogen that coordinates the Gd³⁺ ion on either chelator.) The longer and more rotatable linker in the DTPA system may result in more mobility and this could account for the higher ionic relaxivity rates for CCMV-DOTA-Gd compared to MS2-DTPA-Gd.

Finally protein cages offer advantages over other macromolecular contrast agents. Protein cage scaffolds are generally more rigid than liposome or dendrimer systems. Protein cages are homogeneous whereas both dendrimers and liposomes are heterogeneous mixtures. Also the ability to accurately determine which residues are modified along with the availability of the near atomic resolution crystal structure provides information about the arrangement and environment of each modification within the protein cage structure. This information aids in the design of the agent since the spatial arrangement, structural rigidity and chemical environment of these modifications can be taken into account. In conclusion, the work presented here shows the potential of protein cages as MRI contrast agents and will direct the design of the next generation of these imaging agents.

What is claimed is:

1. A delivery agent comprising a self-assembling protein cage comprising
a) a plurality of subunits, wherein at least one of said subunits is a modified subunit;
b) a first agent; and
c) a targeting moiety.

2. The delivery agent according to claim 1, wherein said first agent comprises a therapeutic agent.

3. The delivery agent according to claim 2, wherein said therapeutic agent comprises a photosensitizing agent.

4. The delivery agent according to claim 1, wherein said therapeutic agent comprises a thermal ablation agent.

5. The delivery agent according to claim 4, wherein said thermal ablation agent comprises a magnetic material.

6. The delivery agent according to claim 1, wherein said modified subunit is chemically modified.

7. The delivery agent according to claim 1, wherein said modified subunit is genetically modified.

8. The delivery agent according to claim 6, wherein said chemical modified subunit comprises a linker.

9. The delivery agent according to claim 6 or 7, wherein said modified subunit comprises a protein.

10. The delivery agent according to claim 9, wherein said protein further comprises a radioisotope.

11. The delivery agent according to claim 9, wherein said protein is a targeting moiety.

12. The delivery agent according to claim 9, wherein said protein is a therapeutic agent.

13. The delivery agent according to claim 1, wherein said first agent is an imaging agent.

14. The delivery agent according to claim 13, wherein said imaging agent is an MRI agent.

15. The delivery agent according to claim 13, wherein said modified subunit comprises a chelator.

16. The delivery agent according to claim 15, wherein said chelator is a paramagnetic metal ion chelator.

17. The delivery agent according to claim 13, wherein said modified subunit comprises a paramagnetic metal ion binding site.

18. The delivery agent according to claim 13, wherein said imaging agent is an optical agent.

19. The delivery agent according to claim 1, wherein said protein cage further comprises a disassembly mechanism.

20. The delivery agent according to claim 19, wherein said disassembly mechanism comprises a reversible switch.

21. The delivery agent according to claim 19, wherein said mechanism comprises an enzymatic cleavage site.

22. The delivery agent according to claim 21, wherein said enzymatic cleavage site is a hydrolase cleavage site selected from the group consisting of a protease cleavage site, a carbohydrase cleavage site, and a lipase cleavage site.

23. The delivery agent according to claim 21, wherein said hydrolase cleavage site is a protease cleavage site.

24. The delivery agent according to claim 1, wherein said protein cage comprises a viral subunit.

25. The delivery agent according to claim 24, wherein said viral subunit comprises a cowpea chlorotic mottle virus (CCMV) protein.

26. The delivery agent according to claim 1, wherein said protein cage comprises a non-viral subunit.

27. The delivery agent according to claim 26, wherein said non-viral subunit comprises a heat shock protein.

28. The delivery agent according to claim 27, wherein said heat shock protein is a Methanococcus jannaschii protein.

29. The delivery agent according to claim 26, wherein said non-viral subunit comprises a Dps-like protein.

30. The delivery agent according to claim 26, wherein said non-viral subunit comprises a mammalian ferritin protein.
